(19) United States
(12) Patent Application Publication
(10) Pub. No.: US 2023/0374116 A1
(43) Pub. Date: Nov. 23, 2023

(54) SINGLE DOMAIN ANTIBODIES THAT TARGET SARS-COV-2

(71) Applicants: THE ROSALIND FRANKLIN INSTITUTE, Didcot, Oxfordshire (GB); OXFORD UNIVERSITY INNOVATION LIMITED, Oxford, Oxfordshire (GB); THE CHANCELLOR MASTERS AND SCHOLARS OF THE UNIVERSITY OF OXFORD, Oxford, Oxfordshire (GB)

(72) Inventors: Jiandong Huo; Raymond Owens, Didcot, Oxfordshire (GB); James Naismith, Didcot, Oxfordshire (GB)

(21) Appl. No.: 18/044,897

(22) PCT Filed: Sep. 14, 2021
(86) PCT No.: PCT/GB2021/052383

§ 371 Date: Mar. 10, 2023

(30) Foreign Application Priority Data
Sep. 14, 2020 (GB) 2014451.5
Sep. 14, 2020 (GB) 2014453.1
Jun. 10, 2021 (GB) 2108319.1

(51) Int. Cl. C07K 16/10; G01N 33/569; A61P 31/14

(52) U.S. Cl. CPC C07K 16/1003; G01N 33/56983; A61P 31/14; C07K 2317/569; C07K 2317/31; C07K 2317/22; C07K 2317/35; C07K 2317/76; C07K 2317/92; G01N 2333/165; A61K 2039/505

(57) ABSTRACT

The present invention relates to improved single domain antibodies that target SARS-CoV-2, multivalent polypeptides and fusion proteins comprising the single domain antibodies. The present invention also provides coronavirus binding molecules that bind two different epitopes on the receptor binding domain of a spike protein of a coronavirus. The coronavirus binding molecules are based on joining two antigen binding molecules together via a linker. The present invention provides the use of said single domain antibodies, multivalent polypeptides, fusion proteins and coronavirus binding molecules in treating and/or preventing coronavirus, as well as the use of said single domain antibodies, multivalent polypeptides, fusion proteins and coronavirus binding molecules in the detection and diagnosis of coronavirus using various methods, assays and kits.

### FIELD OF THE INVENTION

The invention provides improved single domain antibodies that target SARS-CoV-2, multivalent polypeptides and fusion proteins comprising the single domain antibodies, the use of said single domain antibodies, multivalent polypeptides and fusion proteins in treating and/or preventing coronavirus, as well as the use of said single domain antibodies, multivalent polypeptides and fusion proteins in the detection and diagnosis of coronavirus using various methods, assays and kits. The present invention also provides coronavirus binding molecules that bind two different epitopes on the receptor binding domain of a spike protein of a coronavirus, the use of said coronavirus binding molecules in treating and/or preventing coronavirus, as well as the use of said coronavirus binding molecules in the detection and diagnosis of coronavirus using various methods, assays and kits. The coronavirus binding molecules are based on joining two antigen binding molecules together via a linker.

### BACKGROUND OF THE INVENTION

The imperative to identify ways of combating the recently emerged SARS-Cov-2 virus has led to the search for antibodies that can neutralize the virus and therefore be used as a treatment for acute infections by passive immunotherapy. Much of the attention has focused on identifying neutralising monoclonal antibodies from patients who have recovered from COVID-19 (e.g. (Rogers, Zhao et al. 2020). However nanobodies or VHHs (Variable Heavy-chain domains of Heavy-chain antibodies) derived from the heavy chain-only subset of camelid immunoglobulins offer an alternative with advantages over conventional antibodies. In contrast to conventional antibodies that comprise two disulphide-linked polypeptides, heavy and light chain, typically requiring mammalian cells for production, single-domain antibodies can be manufactured with lower costs using microbial systems. Their small molecular size and stability also means that nanobodies could be formulated for topical delivery directly to the airways of infected patients. The potential of single-domain antibodies as inhibitors of SARS-CoV-2 infection has recently been demonstrated in cell-based assays (Huo, Le Bas et al. 2020, Wrapp, De Vlieger et al. 2020).

COVID-19, the disease caused by SARS-CoV-2, is a major global health problem and therefore a critical need for effective treatments exists. Further, suitable tools for the rapid and efficient detection of SARS-CoV-2 are required to enable accurate diagnosis and monitoring of the virus. The present invention describes the isolation of single-domain antibodies that bind different epitopes on the receptor-binding domain of SARS-CoV-2 with high affinity and are highly potent in wild-type virus neutralization assays. Furthermore, various polypeptides have been generated that show an enhancement in binding affinity over the separate components.

Linking two nanobodies that bind to two different epitopes on the same antigen, into a single polypeptide may offer the potential of benefitting from the so-called “chelate effect”. In chemistry, this describes the enhanced affinity of chelating ligands for a metal ion compared to single site binding and is different from the effect of avidity gained from multivalency. In the simplest example, a bidentate molecule gains the chelate effect when both its binding groups bind to the same target. Crucial to gaining the boost of the chelate effect is that the enthalpy of each interaction is preserved when the components are joined. Thus joining two binders with a spacer (also known as a linker) which is too short or too long or with the wrong angular arrangement will mean the two sites cannot bind at the same time without losing significant enthalpy. This will decrease and potentially eliminate any gain from the chelate effect. A solution to this is to introduce a linker that is more flexible and thus allows each to bind gaining its full enthalpy. However, the introduction of flexibility leads to a problem since binding results in rigidification and thus an entropic penalty. Therefore a fine balance needs to be achieved between flexibility and rigidity of spacer elements (von Krbek, Achazi et al. 2017). Although there are many ways of designing a linker that trades off enthalpy with entropy, there is often very limited scope for a linker that gains enthalpy without paying a high entropic cost. Thus gaining the chelate effect (which is thermodynamic) as opposed to simply avidity, requires careful design and insight.

The present invention also provides coronavirus bidentate molecules that bind to two different epitopes on the receptor-binding domain of a coronavirus spike protein with high affinity. These demonstrate a surprisingly enhanced binding affinity over the separate components.

### SUMMARY OF THE INVENTION

The present invention provides single domain antibodies that specifically bind to the receptor biding domain of the S-protein of SARS-CoV-2.

In a first aspect, a single domain antibody comprising a complementary determining region, complementary determining region 3 (CDR3), is provided.

In a second aspect, a single domain antibody comprising a CDR2 and a CDR3 is provided.

In a third aspect, a single domain antibody comprising a CDR1, a CDR2 and a CDR3 is provided.

In a further aspect, an anti-SARS-CoV-2 single domain antibody comprising an amino acid sequence having at least 70% identity to a sequence selected from the group consisting of: SEQ ID NO: 19, 213, 18, 16, 17, 20, 209, 211, 215, 217, 219, 220 and 239 is provided.

In one aspect, the present invention provides coronavirus binding molecules that bind two different epitopes on the receptor binding domain of the spike protein of a coronavirus, preferably a SARS-CoV-2 spike protein.

In one aspect, a coronavirus binding molecule is provided comprising:
(a) a first antigen binding molecule that binds to all or part of a first epitope comprised within a coronavirus protein;
(b) a second antigen binding molecule that binds to all or part of a second epitope comprised within a coronavirus protein; and
(c) a linker,
wherein the linker comprises:
(a) a ubiquitin or a ubiquitin-like protein;
(b) a further optional spacer of between 4 and 50 amino acids joined to the n-terminal of the ubiquitin or ubiquitin-like protein; and
(c) further optional spacer of between 4 and 50 amino acids joined to the c-terminal of the ubiquitin or ubiquitin-like protein;
and wherein the linker joins the first antigen binding molecule to the second antigen binding molecule,
and optionally wherein the first and second epitopes are substantially non-overlapping. In one embodiment, the coronavirus protein is the Spike Protein, optionally the Receptor Binding Domain (RBD) of the Spike Protein. The coronavirus can be a coronavirus selected from the group consisting of SARS-CoV-1, SARS-CoV-2 and MERS, preferably SARS-CoV-2.

In one aspect, a coronavirus binding molecule is provided comprising:
(a) a first antigen binding molecule that binds to all or part of a first epitope comprised within SEQ ID NO: 232;
(b) a second antigen binding molecule that binds to all or part of a second epitope comprised within SEQ ID NO: 233; and
(c) a linker,
wherein the linker comprises:
(i) a ubiquitin or a ubiquitin-like protein;
(ii) further optional spacer of between 4 and 50 amino acids joined to the n-terminal of the ubiquitin or ubiquitin-like protein; and
(iii) further optional spacer of between 4 and 50 amino acids joined to the c-terminal of the ubiquitin or ubiquitin-like protein;
and wherein the linker joins the first antigen binding molecule to the second antigen binding molecule,
and optionally wherein the first and second epitopes are substantially non-overlapping.

In one aspect, a coronavirus binding molecule is provided comprising:
(a) a first antigen binding molecule that competes with C5, H3, H11-D4, H11-H4, H11-A10, H11-B5, H11-H6 or VHH_H6 for binding to the SARS-CoV-2 receptor-binding domain;
(b) a second antigen binding molecule that competes with A8, CR3022, VHH72 or EY6A, F2, C1 or B12 for binding to the SARS-CoV-2 receptor-binding domain; and
(c) a linker,
wherein the linker comprises:
(a) a ubiquitin or a ubiquitin-like protein;
(b) further optional spacer of between 4 and 50 amino acids joined to the n-terminal of the ubiquitin or ubiquitin-like protein; and
(c) further optional spacer of between 4 and 50 amino acids joined to the c-terminal of the ubiquitin or ubiquitin-like protein;
and wherein the linker joins the first antigen binding molecule to the second antigen binding molecule,
and optionally wherein the first and second epitopes are substantially non-overlapping.

In a further aspect, a polynucleotide sequence is provided encoding a single domain antibody, multivalent polypeptide or coronavirus binding molecule of the invention.

In a further aspect, a multivalent polypeptide comprising one or optionally two or more of the single domain antibodies of the invention is provided.

In a further aspect, an affinity matured mutant of a single domain antibody, multivalent polypeptide or coronavirus binding molecule of the invention is provided.

In a further aspect, a method for producing a single domain antibody, multivalent polypeptide or coronavirus binding molecule of the invention is provided.

In a further aspect, a pharmaceutical composition comprising a single domain antibody, multivalent polypeptide or coronavirus binding molecule of the invention is provided.

In a further aspect, a single domain antibody, multivalent polypeptide or coronavirus binding molecule of the invention or a pharmaceutical composition of the invention for use in medicine is provided.

In a further aspect, a method for the treatment of a coronavirus in a subject is provided, comprising administering to a subject a therapeutically active amount of a single domain antibody, multivalent polypeptide or coronavirus binding molecule of the invention.

In a further aspect, the use of a single domain antibody, multivalent polypeptide or coronavirus binding molecule of the invention in the manufacture of a medicament for use in the treatment and/or prevention of a coronavirus is provided.

In a further aspect, methods for the detection of a coronavirus protein are provided.

In a further aspect, methods for diagnosing a coronavirus infection in a subject are provided.

### DESCRIPTION OF FIGURES

FIG. 1. Crystal structure of:
(a) RBD bound to C5 (solved by molecular replacement using PDB id 6YZ5 as the search model);
(b) RBD bound to F2. Contrasting (a) with (b) shows the binding location of F2 on RBD (b) to be distinct from that of C5 (a).

FIG. 2. Electron microscopy structure showing three C5 bound each to one RBD of the S1 trimer complex.

FIG. 3. Crystal structures of:
(a) RBD bound to C5. Shown superimposed are the ACE2 (from RBD: ACE2 complex PD id 6M0J) and H11-H4 (from the H11-H4:RBD complex PDB id 6YLA (Huo, Zhao et al. 2020).
(b) RBD bound to CR3022 and H11-H4 (from PDB id 6Z2M) with structure of VHH72 superimposed (from PDB id 6WAQ (Wrapp, De Vlieger et al. 2020)) showing that the region of RBD that binds to H11-H4 (and C5) is different from CR3022 (and VHH72).

FIG. 4. Crystal structure of the RBD bound both to C1 and H3. The C terminus of C1 is shown with a dark grey sphere and the N terminus of H3 with a light grey sphere. The distance between the C terminus of C1 (dark grey sphere) and the N terminus of H3 (light grey sphere) is 63 Å.

FIG. 5. Composite crystal structure of:
(a) RBD bound to C1 and C5, constructed by superimposing the RBD portions of C1-RBD-H3 (FIG. 4), C1-RBD and C5-RBD (FIG. 3a). The C terminus of C1 is shown with a dark grey sphere and the N terminus of C5 with a light grey sphere. The distance between the C terminus of C1 (dark grey sphere) and the N terminus of C5 (light grey sphere) is 57 Å.
(b) RBD bound to C1 and H11-H4. The distance between the C terminus of C1 (dark grey sphere) and the N terminus of H4 (light grey sphere) is 60 Å.

FIG. 6. Composite crystal structure of RBD bound to VHH72 and C5. The C terminus of VHH77 is shown with a dark grey sphere and the N terminus of C5 with a light grey sphere. The distance between the C terminus of VHH72 (dark grey sphere) and the N terminus of C5 (light grey sphere) is 52 Å.

FIG. 7. Binding kinetics between RBD and various C5 polypeptides. Surface plasmon resonance (SPR) was performed using a Biacore T200 (GE Healthcare) and results are provided as a plot of Response (RU) against Time (seconds).
(a) Monomeric C5
(b) C5-Fc
(c) C5-AAA-C5
(d) C5-9GS-C5
(e) C5-6GS-SUMO-6G5-C5

FIG. 8. Micro-titre neutralisation curves for:
(a) C1-Fc and C5-Fc;
(b) Monomeric C5 and C5-Fc

FIG. 9. Neutralisation of SARS-CoV-2 strains in vitro Neutralisation curves of the anti-RBD nanobody trimers for (a) Victoria (B) (b) Alpha or Kent (B.1.1.7) and (c) Beta or South Africa (B.1.351) strains of SARS-CoV-2 measured in a microneutralisation assay. Data are show as the mean+/−95% Cl.

FIG. 10. C5-Fc Neutralisation of SARS-CoV-2 in Syrian hamster model.
(a) Animals were challenged with SARS-CoV-2 (B Victoria 5×10⁴ pfu) at day 0 and then treated with either C5-Fc (IP 4 mg/kg) or PBS, delivered by the intraperitoneal route 24 hours post-challenge. (b) Body weight was recorded daily and the mean percentage weight change from baseline was plotted (+/−1 SE). (c) lung histopathology image analysis showing percentage of the lung area affected and percentage of lung area with viral RNA ISH staining with representative images of lung stained with H & E and ISH. Box and bars show median and 95% C.I. Mann-Whitney's U test for median comparisons.

FIG. 11. C5 Trimer Neutralisation of SARS-CoV-2 in Syrian hamster model.
(a) Animals were challenged on day 0 and then treated either 2 h (4 mg/kg) or 24 h (0.4 and 4 mg/kg) later with C5-trimer by nasal installation or after 24 h IP (4 mg/kg). The control arm was treated with vehicle alone (PBS) Daily average body weight change (+/−SEM) were calculated from day 0 (b) viral load in the lung (c) Infiltration of the airways (d) representative images of lung stained with H & E and anti-SARS-Cov-2 NP.

FIG. 12. (a) & (b) Sensorgrams showing the binding kinetics of NbSA_A10 (a) or NbSA_D10 (b) to the Beta (South African strain). (c) & (d) Sensorgrams showing that incubation of the RBD with the nanobody NbSA_A10 (c) or NbSA_D10 (d) did not prevent binding of the RBD to either ACE2 or CR3022.

FIG. 13. (a) Sensorgram showing the binding kinetics of A8 to the Beta (South African strain) RBD (b) Sensorgrams showing that incubation of the RBD with the nanobody A8 prevents binding of the RBD to both ACE2 and CR3022.

FIG. 14. Neutralisation of Victoria and South African SARS-CoV-2 strains by A8.

FIG. 15. Immobilising the capture agent by passive absorption. (a) Using H4 passively absorbed onto plate as capture agent and C1-Fc as the probe is shown in green. Reversing capture and probe antibodies is shown in red. (b) C1-Fc as capture and H4-HRP as the probe but using different ELISA plates. In red is the passive absorption of C1-Fc (essentially the same result as par a. Shown in blue is using biotinx-C1-Fc bound to streptavidin plates. For both experiments actual absorbance values over 1 were obtained from a dilution then multiplied.

FIG. 16. Pairings of biotinylated nanobodies as capture (biotinx-XX-Fc) and probe (YY-Fc-HRP).

FIG. 17. Using the biotinx-F2-Fc and C5-HRP combination we were able to measure antigen when presented in viral form. (a) Pseudotyped virus was detected to 21 TCID50/mL. (b) Heat/empigen inactivated virus at 103 ffu/mL.

FIG. 18. Using the site selective biotin-SS-F2-Fc attachment and C5-HRP improved detection sensitivity. (a) Spike protein was detected to 147 pg/mL. (b) RBD was detected to 33 pg/mL (c) Pseudotyped virus was detected to 16 TCID50/mL. (d) Empigen inactivated virus was detected to 16 ffu/mL.

FIG. 19. Sensorgram of ACE-2 or CR3022 binding to RBD in competition with VHH_H6.

FIG. 20. Crystal structures of C5, H3 and H6 RBD complexes.

FIG. 21. Crystal structure of A8 bound to RBD. Shown superimposed is ACE2.

### DETAILED DESCRIPTION OF THE INVENTION

“Antibody” as used herein refers to an immunogenic protein that recognizes a specific antigen. Each antibody has an antigen binding site that specifically binds an antigen. Antibodies can be natural or partly or wholly synthetic. The term antibody also encompasses any polypeptide or protein having an antigen binding site which is, or is homologous to, an antigen binding site of an antibody. Antibodies may be polyclonal or monoclonal. Traditional antibodies comprise two identical heavy chains and two identical light chains, each chain comprising a variable region and a constant region. Each of the variable regions of the heavy and light chains comprise three complementarity determining regions (CDRs), CDR1, CDR2 and CDR3. Antibody as used herein also encompasses antibody fragments comprising an antigen binding domain, such as Fab, F(ab′)2, Fv, scFv, dAb, Fd; and diabodies.

“Bidentate” as used herein refers to a polypeptide comprising two different single domain antibodies, i.e. two single domain antibodies having two different antigen binding sites. Preferably, the bidentate molecule is designed such that two different single domain antibodies bind two different epitopes on the same antigen.

“Monomer” as used herein refers to one single unit, for example a single domain antibody (either a known single domain antibody or a single domain antibody of the invention), fusion protein or antibody.

“Polymer” as used herein refers to molecule comprised of multiple monomers covalently and/or non-covalently bound together. For example, a single domain antibody of the invention can be covalently linked to one or more additional single domain antibodies, such as the single domain antibodies of the invention, within a single polypeptide chain. In this case, a single gene can encode multiple linked single domain antibodies. Alternatively, a polymer can be formed by linking two or more separate synthesised single domain antibodies with a covalent linkage and/or non-covalent linkages. In one example, a synthesised single domain antibody fused to a Fc molecule can covalently link to a separate synthesised single domain antibody fused to a Fc molecules through the formation of disulphide bridges between the Fc molecules. Single domain antibodies of the invention can also be non-covalently linked to other single domain antibodies (for example those of the invention) exclusively via non-covalent linkage, for example through the use of a dimerization or trimersation domain. An example of this would be fusing the single chain antibody to a collagen derived trimerisation domain. For example, a “Dimer” as used herein refers to two monomers covalently or non-covalently bound together. A “homodimer” is formed of two identical monomers. A “heterodimer” is formed of two different monomers. A “trimer” as used herein refers to three monomers covalently or non-covalently bound together.

“Dimerization domain” or “trimerization domain” as used herein refers to a sequence protein or motif that permits dimerization, or trimerization respectively, between single domain antibodies. The sequence protein or motif can be fused to the single domain antibody. For example, fusing a single domain antibody sequence to a collagen-derived trimerisation domain yields a gene product which codes for polypeptide chain with a single antibody sequence, however, when the protein is synthesised, the actual product is a trimer (Compte et al).

“Monospecific” as used herein refers to a polypeptide having antigen binding sites that all bind to the same epitope.

“Multispecific” as used herein refers to the number of different antigen biding site specificities present. For example, a “bispecific” polypeptide has antigen binding sites that bind to two different epitopes (either on the same or on different antigens), a “trispecific” polypeptide has antigen binding sites that bind to three different epitopes (either on the same or on different antigens), a “tetraspecific” polypeptide has antigen binding sites that bind to four different epitopes (either on the same or on different antigens).

“Monovalent” as used herein refers to a single domain antibody having one antigen binding site.

“Multivalent” as used herein refers to a polypeptide that has multiple antigen binding sites. The term multivalent is interchangeable for the term “polyvalent”. For example, “bivalent” as used herein refers to a polypeptide that has two antigen binding sites, “trivalent” as used herein refers to a polypeptide that has three antigen binding sites and “tetravalent” as used herein refers to a polypeptide that has four antigen binding sites. A multivalent antibody can be monospecific, bispecific, trispecific, tetraspecific or multispecific, as defined herein. For example a “monospecific multivalent” polypeptide has multiple antigen binding sites that all bind to the same epitope. A “bispecific multivalent” polypeptide has multiple antigen binding sites, a number of the antigen binding sites bind to a first epitope and a number of the antigen binding sites bind to a second epitope (that is different to the first epitope).

“Conservative substitution” as used herein refers to amino acid substitutions that do not materially affect the function of a protein (for example the ability to bind to a specific target, in particular the coronavirus spike protein of SARS-CoV-2 in the context of the invention, or the ability to elicit an immune response in a subject). The skilled person readily understands the properties of amino acids and can readily make a conservative substitution without materially altering the properties of the resulting polypeptide. Examples of conservative substitutions are provided in the table below.

| Class | Exchangeable amino acids |
|---|---|
| Aliphatic | Glycine, Alanine, Valine, Leucine, |
| | Isoleucine |
| Hydroxyl or Sulfur/Selenium- | Serine, Cysteine, Threonine, |
| containing | Methionine |
| Aromatic | Phenylalanine, Tyrosine, Tryptophan |
| Basic | Histidine, Lysine, Arginine |
| Acidic and their Amide | Aspartate, Glutamate, Asparagine, |
| | Glutamine |

“Deletion” as used herein refers to the removal of an amino acid in a polypeptide sequence (i.e. the replacement of one amino acid with no amino acid such that the amino acid sequence is one amino acid shorter in length). Deletion can also refer to polynucleotide sequences and the removal of one nucleic acid from a polynucleotide sequence (the replacement of one nucleic acid with no nucleic acid such that the polynucleotide sequence is one nucleic acid shorter in length).

“Identity” as used herein is the degree to which two sequences are related, as determined by comparing two or more polypeptide of polynucleotide sequences. Identity can be determined using the degree of relatedness of two sequences to provide a measurement of to what extent the two sequences match. Numerous programs are well known by the skilled person for comparing polypeptide or polynucleotide sequences, for example (but not limited to the various BLAST and CLUSTAL programs. Percentage identity can be used to quantify sequence identity. To calculate percentage identity, two sequences (polypeptide or nucleotide) are optimally aligned (i.e. positioned such that the two sequences have the highest number of identical residues at each corresponding position and therefore have the highest percentage identity) and the amino acid or nucleic acid residue at each position is compared with the corresponding amino acid or nucleic acid at that position. In some instances, optimal sequence alignment can be achieved by inserting space(s) in a sequence to best fit it to a second sequence. The number of identical amino acid residues or nucleotides provides the percentage identity, i.e. if 9 residues of a 10 residue long sequence are identical between the two sequences being compared then the % identity is 90%. Percentage identity is generally calculated along the full length of the two sequences being compared.

“Insertion” refers the addition of an amino acid in a polypeptide sequence (i.e. insertion of one amino acid means one new amino acid is added into in an existing amino acid sequence such that the amino acid sequence is one amino acid longer in length). Insertion can also refer to polynucleotide sequences and the addition of one nucleic acid to a polynucleotide sequence (i.e. insertion of one nucleic acid means one new nucleic acid is added into in an existing polynucleotide sequence such that the nucleic acid sequence is one amino acid longer in length).

“Modification” as used herein refers to an alteration of an amino acid residue in a polypeptide sequence. The modification can be a substitution, deletion or insertion, as defined herein. Modification can also refer to polynucleotide sequences.

“Single domain antibody” as used herein refers to a variable region of a heavy chain of an antibody, wherein the variable region is derived from a heavy chain only (i.e. devoid of a light chain) subset of camelid immunoglobulins. The term single domain antibody can be used interchangeably with (variable domain of camelid heavy-chain-only antibody, VHH) and Nanobody®. In the context of the invention a single domain antibody is used to refer to a single heavy chain variable region that can bind the spike protein of a coronavirus, preferably SAR-CoV-2. The antibody can be affinity matured, humanized or modified, as described herein. This single domain antibody can be conjugated to other components.

“Substitution” as used herein refers the replacement of amino acid with a different amino acid. Substitution can also refer to polynucleotide sequences, i.e. the replacement of one nucleic acid with a different nucleic acid. A substitution can be a conservative substitution, as defined above.

“Tetravalent” as used herein refers to a polypeptide that has four antigen binding sites.

The single domain antibodies of the invention are based on 12 VHH sequences having positive binding to the receptor binding domain (“RBD”) of the S protein of SARS-CoV-2, namely B12, F2, C1, C5 H3, NbSA_A10, NbSA_D10, A8, 3_05, 8_G1, 12_F11 and VH_H6 (amino acid sequences provided as SEQ ID NOs: 16-20, 209-220 and 239), polynucleotide sequences provided as SEQ ID NOs: 21-25, 208-218 and 238 respectively).

Specific amino acid sequences are provided herein to define the amino acid sequences of specified CDRs. For convenience, these are listed in the table below. Single domain antibodies of the invention comprising these specified CDR sequences can comprise one or more modifications, as detailed herein, and will retain binding affinity for a coronavirus peptide, preferably the receptor binding domain of the S protein of SARS-CoV-2.

| | | SEQ | | SEQ | | SEQ |
|---|---|---|---|---|---|---|
| | | ID | | ID | | ID |
| ID | CDR1 | NO | CDR2 | NO | CDR3 | NO |
| B12 | GGTFSTY | 1 | IRRSGST | 2 | AARRAGREYEY | 3 |
| F2 | GRTFHSYV | 4 | ISWSSTPT | 5 | AADRGESYYYT | 6 |
| | | | | | RPTEYEF | |
| C1 | GFTNDFYS | 7 | LSVSDNTP | 8 | AAGRFAGRDTW | 9 |
| | | | | | PSSYDY | |
| C5 | GVTLGRHA | 10 | IRTFDGIT | 11 | ALGVTAACSDN | 12 |
| | | | | | PYF | |
| H3 | GRTFSTYS | 13 | MRWTGSST | 14 | AITTIVRAYY | 15 |
| | | | | | TEYTEADFGS | |
| NbSA_ | GRTFSTTR | 190 | IFLNTGTT | 191 | AAGRFSAAPL | 192 |
| A10 | | | | | TQSTAFES | |
| NbSA_ | GRTSSDYS | 193 | LAWTVGAT | 194 | AGRYGAGLGF | 195 |
| D10 | | | | | TERIYDY | |
| A8 | GGTFSTAA | 196 | IGWRGVRT | 197 | AASVGNYGLPW | 198 |
| | | | | | AHFEYDF | |
| 3_C5 | GRTLSMR | 199 | INWSSGSI | 200 | AVQVDIGGYL | 201 |
| | | | | | DGYDY | |
| 8_ | GRTITEYT | 202 | ISKSTDST | 203 | AAGSFYGRDSD | 204 |
| G11 | | | | | AGGYDY | |
| 12_ | GGAFSTSA | 205 | IGWRGVRT | 206 | AASDGNYGLPW | 207 |
| F11 | | | | | AHFEYDF | |
| VHH_ | ESSLAPYR | 235 | ISRDAHPT | 236 | ATDLGGYCSDS | 237 |
| H6 | | | ST | | NYPRAW | |

CDR Alignments of Nanobodies

Amino acid and nucleotide sequences of B12, C1, C5, F2, H3, NbSA_A10, NbSA_D10, A8, 3_C5, 8_G11, 12_F11 and VHH_H6 (CDR highlighted in bold)

> B12
(SEQ ID NO: 21)
CAGGTGCAGCTGGTGGAGTCTGGAGGAGGCTTCGTGCAGCCTGGGGGCTCTCTGAGACTCTCCTGCGCCGTTTCT
GGAGGCACCTTCAGTACCTATGGCATGGGCTGGTTCCGCCAGGCTCCAGGGAAGGAGCGTGAGATTGTAGCAGCG
ATTAGACGGAGTGGTAGCACATACTATGCAGACTCCGTGAGGGGCCGATTCACCATCTCCAGAGACAACGCCAAG
AACTCGGTGCTCCTGCAAATGAACAGCCTGAAACCTGAGGACACGGCCGTTTATTACTGTGCAGCCAGGAGAGCG
GGTAGGGAGTATGAGTACTGGGGCCAGGGGACCCAGGTCACCGTCTCCTCA
> B12
(SEQ ID NO: 16)
QVQLVESGGGFVQPGGSLRLSCAVSGGTFSTYGMGWFRQAPGKEREIVAAIRRSGSTYYADSVRGRFTISRDNAK
NSVLLQMNSLKPEDTAVYYCAARRAGREYEYWGQGTQVTVSS
> F2
(SEQ ID NO: 22)
CAGGTGCAGCTGGTGGAGTCTGGGGGAGGATTGGTGCAGGCTGGGGGCTCTCTAAGACTCGCTTGTATAGCCTCT
GGACGCACCTTCCATAGCTATGTCATGGCCTGGTTCCGCCAGGCTCCAGGGAAGGAGCGTGAGTTTGTAGCAGCT
ATTAGTTGGAGTAGTACACCGACATACTATGGAGAATCCGTGAAGGGCCGATTCACCATCTCCAGAGACAACGCC
AAGAACACGGTGTATCTGCAAATGAACCGCCTGAAACCTGAGGACACGGCCGTTTATTTCTGTGCAGCAGACCGG
GGTGAAAGTTACTACTACACTCGACCCACCGAGTATGAATTCTGGGGCCAGGGGACCCAGGTCACCGTCTCCTCA
> F2
(SEQ ID NO: 17)
QVQLVESGGGLVQAGGSLRLACIASGRTFHSYVMAWFRQAPGKEREFVAAISWSSTPTYYGESVKGRFTISRDNA
KNTVYLQMNRLKPEDTAVYFCAADRGESYYYTRPTEYEFWGQGTQVTVSS
> C1
(SEQ ID NO: 23)
CAGGTGCAGCTGGTGGAGTCTGGGGGAGGCTTGGTGCAGCCTGGGGGCTCTCTGAGACTCTCCTGTGCAGCCTCT
GGATTCACTAATGATTTTTATAGCATCGCGTGGTTCCGCCAGGCCCCAGGAAAGGAGCGTGAGGGGGTCTCATGG
CTTAGTGTCAGTGATAATACCCCAACCTACGTAGACTCCGTGAAGGACCGGTTCACCATCTCCAGACACAACGCC
AACAACACCGTGTACCTGCAAATGAACATGCTGAAACCTGAGGACACGGCCATTTACTATTGTGCAGCAGGACGC
TTCGCGGGAAGGGATACTTGGCCCTCGTCCTATGATTACTGGGGCCAGGGGACCCAGGTCACCGTCTCCTCA
> C1
(SEQ ID NO: 18)
QVQLVESGGGLVQPGGSLRLSCAASGFTNDFYSIAWFRQAPGKEREGVSWLSVSDNTPTYVDSVKDRFTISRHNA
NNTVYLQMNMLKPEDTAIYYCAAGRFAGRDTWPSSYDYWGQGTQVTVSS
> C1 alternative sequence
(N76Q), difference to C1 shown with underline
(SEQ ID NO: 220)
QVQLVESGGGLVQPGGSLRLSCAASGFTNDFYSIAWFRQAPGKEREGVSWLSVSDNTPTYVDSVKDRFTISRHNA
QNTVYLQMNMLKPEDTAIYYCAAGRFAGRDTWPSSYDYWGQGTQVTVSS
> C5
(SEQ ID NO: 24)
CAGGTGCAGCTGGTGGAGTCTGGGGGAGGCTCGGTGCAGGCTGGGGGGTCTCTGACACTCTCCTGTGTCGCCTCT
GGAGTCACTTTGGGACGTCATGCCATAGGCTGGTTCCGCCAGGCCCCCGGGAAGGAGCGTGAGAGAGTCTCGTGT
ATTAGAACATTTGATGGCATCACAAGTTATGTAGAGTCCACGAAGGGCCGATTCACCATCTCCAGTAACAATGCC
ATGAACACGGTGTATCTGCAAATGAATAGCCTCAAACCTGAAGACACGGCCGTTTATTTCTGTGCACTGGGAGTG
ACTGCAGCCTGTTCAGATAATCCCTACTTCTGGGGCCAGGGGACCCAGGTCACCGTCTCCTCA
> C5
(SEQ ID NO: 19)
QVQLVESGGGSVQAGGSLTLSCVASGVTLGRHAIGWFRQAPGKERERVSCIRTFDGITSYVESTKGRFTISSNNA
MNTVYLQMNSLKPEDTAVYFCALGVTAACSDNPYFWGQGTQVTVSS
> H3
(SEQ ID NO: 25)
CAGGTGCAGCTGGTGGAGTCTGGGGGAGGATTGGTGAAGACTGGGGGCTCTCTGAGACTCTCCTGTGCAGCCTCT
GGCCGCACCTTCAGTACCTACAGCATGGGCTGGTTCCGCCAGGCTCCAGGGAAGGAGCGTGAGTTTGTAGCAGGT
ATGCGCTGGACGGGTAGTAGTACATTCTACTCAGACTCCGTGAAGGGCCGATTCACCGTCTCCAGAAACAACGCC
AAGGACACGGTGTATCTGCACATGAACAGCCTGAAACCTGAGGACACGGCCGTTTATTACTGTGCAATCACGACT
ATCGTAAGAGCTTACTATACTGAGTATACCGAAGCTGACTTTGGTTCCTGGGGCCAGGGGACCCAGGTCACCGTC
TCCTCA
> H3
(SEQ ID NO: 20)
QVQLVESGGGLVKTGGSLRLSCAASGRTFSTYSMGWFRQAPGKEREFVAGMRWTGSSTFYSDSVKGRFTVSRNNA
KDTVYLHMNSLKPEDTAVYYCAITTIVRAYYTEYTEADFGSWGQGTQVTVSS
>NbSA_A10
(SEQ ID NO: 208)
CAGGTGCAGCTGGTCGAGTCTGGGGGAGGCTTGGTGCAGGCTGGGGACTCTCTGAGACTCTCCTGTGCAGCCTCT
GGACGTACCTTCAGTACCACTCGCATGGCCTGGTTCCGCCAGCCTCCAGGGAAGGAGCGCGAGTTTGTAGCGGCG
ATTTTTCTGAATACTGGCACGACATACTATGCAGATCCCGTGAAGGACCGATTCGCCATCTCCAGAGACAATGCC
AAAAACACGGTGTATCTGCAAATGAACAGCCTGAAACCTGAGGACACGGCCGTTTATTACTGCGCAGCGGGTCGG
TTCAGCGCTGCTCCGCTTACACAGTCAACTGCCTTTGAGTCCTGGGGCCAGGGGACCCAGGTCACCGTCTCC
>NbSA A10
(SEQ ID NO: 209)
QVQLVESGGGLVQAGDSLRLSCAASGRTFSTTRMAWFRQPPGKEREFVAAIFLNTGTTYYAD
PVKDRFAISRDNAKNTVYLQMNSLKPEDTAVYYCAAGRFSAAPLTQSTAFESWGQGTQVTVS
A
>NbSA_D10
(SEQ ID NO: 210)
CAGGTGCAGCTGGTCGAGTCTGGGGGAGGATTGGTACAGGTTGGGGGCTCTCTGAGACTCTCCTGTGCAGTCTCT
GGACGCACCAGCAGTGACTATTCCGTGGGCTGGTTCCGCCGGGCTCAAGGGAAGGAGCGTGAGTTTGTGGCTGCT
CTGGCGTGGACTGTTGGCGCCACACACTATGGAGACTCCGTGAAGGGCCGATTCACCGTCTCCAGAGACAACGCC
AAGAACATGGTGTATCTGCAAATAGACAGCCTGAAACCTGAAGACACGGGCGTTTATTACTGTGCAGGTCGATAT
GGAGCGGGATTGGGGTTCACCGAAAGAATCTATGACTACTGGGGCCAGGGGACCCAGGTCACCGTTTCCTCA
>NbSA_D10
(SEQ ID NO: 211)
QVQLVESGGGLVQVGGSLRLSCAVSGRTSSDYSVGWFRRAQGKEREFVAALAWTVGATHYGD
SVKGRFTVSRDNAKNMVYLQIDSLKPEDTGVYYCAGRYGAGLGFTERIYDYWGQGTQVTVSS
>A8
(SEQ ID NO: 212)
CAGGTGCAGCTGGTCGAGTCTGGGGGAGGATTGGTGCAGGCTGGAGGCTCTCTGAGACTCTCCTGTGCAGCCTCT
GGAGGCACCTTCAGTACCGCTGCCATGGGCTGGTTCCGCCAGGCTCCAGGCGAGGAGCGTGAGTGTGTAGCATCT
ATAGGCTGGAGAGGTGTTAGAACATGGTATGCAGACTCCGTGAAGGGCCGATTTACCATCTCCAGAGATAATCCC
CAGAATACGGTGTATTTGCAGATGAACAACCTGAAATCTGGCGACACGGCCGTTTATTACTGCGCAGCCTCAGTC
GGCAACTACGGGTTGCCGTGGGCCCACTTCGAATATGACTTCTGGGGCCAGGGGATCCAGGTCACCGTGTCGTCA
>A8
(SEQ ID NO: 213)
QVQLVESGGGLVQAGGSLRLSCAASGGTFSTAAMGWFRQAPGEERECVASIGWRGVRTWYAD
SVKGRFTISRDNPQNTVYLQMNNLKSGDTAVYYCAASVGNYGLPWAHFEYDFWGQGIQVTVS
S
>3_C5
(SEQ ID NO: 214)
CAGCCGGCCATGGCCCAGGTGCAGCTGGTCGAGTCTGGGGGAGGATTGGTGCAGGCTGGGGGCTCCCTGAGACTC
TCCTGTGCAGCCTCTGGACGCACCTTGAGTATGCGTCGCATGAGCTGGTTCCGCCAGGCTCCAGGGAAGGAGCGT
GAGTTTGTAGCCACTATTAACTGGAGTAGTGGTAGTATATACGTTACAGACTCCGTGAAGGACCGATTCACCATC
TCCAGAGACAACGCCGAGAACACGGTGCATCTGCAAATGAACATGCTGAAACCTGAGGACACGGCCGTTTATTCG
TGTGCAGTCCAAGTCGATATTGGGGGGTACCTCGACGGCTATGACTACTGGGGCCAGGGGACCCAGGTCACCGTC
TCCTCA
>3_C5
(SEQ ID NO: 215)
QVQLVESGGGLVQAGGSLRLSCAASGRTLSMRRMSWFRQAPGKEREFVATINWSSGSIYVTD
SVKDRFTISRDNAENTVHLQMNMLKPEDTAVYSCAVQVDIGGYLDGYDYWGQGTQVTVSS
>8_G11
(SEQ ID NO: 216)
CAGGTGCAGCTGGTCGAGTCTGGGGGAGGATTGGTGCAGGCTGGGGGCTCTCTGAGACTCTCCTGTGCAGCCTCT
GGACGCACCATCACTGAATATACCATAGGGTGGTTCCGCCAGGCTCCAGGGAAGGAGCGTGAGTTTGTAACACTT
ATTAGCAAGAGTACTGATAGTACATGGGATGCAGACTCCGTGAAGGGCCGATTCACCATCGCCAGAGATTACGCC
AAGAACACGGTGTATCTGCAAATGAACAGCCTGAAACCTGAGGACACGGCCGTTTATTACTGTGCAGCTGGCTCC
TTTTACGGGCGCGATTCGGATGCTGGGGGCTATGACTACTGGGGCCAGGGGACCCAGGTCACCGTCTCCTCA
>8_G11
(SEQ ID NO: 217)
QVQLVESGGGLVQAGGSLRLSCAASGRTITEYTIGWFRQAPGKEREFVTLISKSTDSTWDAD
SVKGRFTIARDYAKNTVYLQMNSLKPEDTAVYYCAAGSFYGRDSDAGGYDYWGQGTQVTVSS
>12_F11
(SEQ ID NO: 218)
CAGGTGCAGCTGGTCGAGTCTGGAGGAGGATTGGTGCAGGCTGGAGGCTCTCTGAGACTCTCCTGTGCAGCCTCT
GGAGGCGCCTTCAGTACCTCTGCCATGGGCTGGTTCCGCCAGGCTCCAGGGAAGGAACGTGAGTTTGTCGCAGTT
ATAGGCTGGAGAGGTGTTAGAACATACTATGCAGACTCCGTGAAGGGCCGTTTCACCATCGCCAGAGATAATCCC
CAGAACAGGGTGTATCTGGAGATGAGCAGCCTGAAATCTGACGACACGGGCGTTTATTTCTGTGCAGCCTCAGAC
GGCAACTACGGATTGCCGTGGGCCCATTTCGAGTATGACTTCTGGGGCCAGGGGATCCAGGTCACCGTCTCCTCA
>12_F11
(SEQ ID NO: 219)
QVQLVESGGGLVQAGGSLRLSCAASGGAFSTSAMGWFRQAPGKEREFVAVIGWRGVRTYYAD
SVKGRFTIARDNPQNRVYLEMSSLKSDDTGVYFCAASDGNYGLPWAHFEYDFWGQGIQVTSS
VHH_H6
(SEQ ID NO: 238)
CAGGTGCAGCTGGTCGAGTCTGGGGGAGGCTTGGTGCAGCCTGGGGGGTCTCTGACACTCTC
CTGTGTAGCGTCGGAATCATCTTTGGCGCCTTATCGCGTAGCCTGGTTCCGTCAGGCCCCAG
GGAAGGAGCGCGAGGGGGTCTCATGTATTAGTCGTGATGCACATCCTACTAGTACATACTAT
ACAGCCTCCGTGAAGGGCCGATTCACCATGTCCAGAGACAATGCCAAGAACACGGTGTATCT
GCAAATGAACAGCCTGAAACCATCGGACACGGCCGTTTATTACTGTGCGACAGATTTGGGAG
GATACTGTTCGGATTCTAATTATCCCCGCGCCTGGTGGGGCCAGGGGACCCAGGTCACCGTC
TCCTCA
VHH_H6
(SEQ ID NO: 239)
QVQLVESGGGLVQPGGSLTLSCVASESSLAPYRVAWFRQAPGKEREGVSCISRDAHPTSTYY
TASVKGRFTMSRDNAKNTVYLQMNSLKPSDTAVYYCATDLGGYCSDSNYPRAWWGQGTQVTV
SS

In one aspect, a single domain antibody comprising a complementary determining region, complementary determining region 3 (CDR3), is provided. In one embodiment, a single domain antibody comprises a complementary determining region selected from CDR1, complementary determining region 2 (CDR2) or complementary determining region 3 (CDR3) is provided. In one embodiment, a single domain antibody comprises at least one complementary determining region selected from CDR1, CDR2 or CDR3 is provided. In one embodiment, a single domain antibody comprises at least two complementary determining regions selected from CDR1, CDR2 or CDR3. In one embodiment, single domain antibody comprises three complementary determining regions: CDR1, CDR2, and CDR3 is provided.

In one embodiment, a single domain antibody comprising a complementary determining region 3 (CDR3) selected from the group consisting of SEQ ID NOs: 12, 198, 9, 3, 6, 15, 192, 195, 201, 204, 207 and 237 is provided, wherein the amino acid sequences of CDR3 comprise between 0 and 7 amino acid modifications. In one embodiment the CDR3 regions comprise between 0 and 6, 0 and 5, 0 and 4, 0 and 3, 0 and 2 and 0 and 1 amino acid modifications. The modifications can be substitutions, deletions or insertions. In one embodiment, the modifications are substitutions.

In one embodiment, a single domain antibody comprising a complementary determining region 3 (CDR3) selected from the group consisting of SEQ ID NOs: 12, 9, 3, 6 and 15 is provided, wherein the CDR3 regions of amino acid sequences of SEQ ID NOs: 6, 9 and 15 comprise between 0 and 7 amino acid modifications, optionally between 0 and 2 modifications; and wherein the CDR3 regions of amino acid sequences of SEQ ID Nos: 3 and 12 comprise between 0 and 5 amino acid modifications, optionally between 0 and 2 amino acid modifications.

In one embodiment, the complementary determining region 3 (CDR3) is SEQ ID NO: 3. In one embodiment, the complementary determining region 3 (CDR3) is SEQ ID NO: 6. In one embodiment, the complementary determining region 3 (CDR3) is SEQ ID NO: 15. In a preferred embodiment, the complementary determining region 3 (CDR3) is SEQ ID NO: 12 or 198. In a most preferred embodiment, the complementary determining region 3 (CDR3) is SEQ ID NO: 12. In one embodiment the CDR3 regions comprise between 0 and 6, 0 and 5, 0 and 4, 0 and 3, 0 and 2 or 0 and 1 amino acid modifications. The modifications can be substitutions, deletions or insertions. In one embodiment, the modifications are substitutions.

In one embodiment the single domain antibody of the invention may further comprise a CDR2 region. The CDR2 region may be defined according to a SEQ ID NO disclosed herein. In a further embodiment, the single domain antibody of the invention may further comprise a CDR1 region and CDR2 region. The CDR1 region and the CDR2 region may be defined according to a SEQ ID NO disclosed herein. In each embodiment, the single domain antibody may further comprise four framework regions (FR1, FR2, FR3 and FR4).

In one aspect, an anti-SARS-CoV-2 single domain antibody is provided, wherein the single antibody domain comprises
(a) a CDR2 comprising SEQ ID NO:11 and a CDR3 comprising SEQ ID NO:12;
(b) a CDR2 comprising SEQ ID NO:197 and a CDR3 comprising SEQ ID NO:198;
(c) a CDR2 comprising SEQ ID NO:8 and a CDR3 comprising SEQ ID NO:9;
(d) a CDR2 comprising SEQ ID NO:2 and a CDR3 comprising SEQ ID NO:3;
(e) a CDR2 comprising SEQ ID NO:5 and a CDR3 comprising SEQ ID NO:6;
(f) a CDR2 comprising SEQ ID NO:14 and a CDR3 comprising SEQ ID NO:15;
(g) a CDR2 comprising SEQ ID NO:191 and a CDR3 comprising SEQ ID NO:192;
(h) a CDR2 comprising SEQ ID NO:194 and a CDR3 comprising SEQ ID NO:195;
(i) a CDR2 comprising SEQ ID NO:200 and a CDR3 comprising SEQ ID NO:201;
(j) a CDR2 comprising SEQ ID NO:203 and a CDR3 comprising SEQ ID NO:204;
(k) a CDR2 comprising SEQ ID NO:206 and a CDR3 comprising SEQ ID NO:207; or
(l) a CDR2 comprising SEQ ID NO:236 and a CDR3 comprising SEQ ID NO:237;
wherein the amino acid sequence of CDR3 comprises between 0 and 7 amino acid modifications and wherein the amino acid sequence of CDR2 comprises between 0 and 4 amino acid modifications. In one embodiment the CDR3 regions comprise between 0 and 6, 0 and 5, 0 and 4, 0 and 3, 0 and 2 or 0 and 1 amino acid modifications. In one embodiment the CDR2 regions comprise between 0 and 3, 0 and 2, 0 and 4, 0 and 1 amino acid modifications.

In a preferred embodiment, an anti-SARS-CoV-2 single domain antibody is provided, wherein the single antibody domain comprises a CDR2 comprising SEQ ID NO:197 and a CDR3 comprising SEQ ID NO:198; and wherein the amino acid sequence of CDR3 comprises between 0 and 7 amino acid modifications and wherein the amino acid sequence of CDR2 comprises between 0 and 4 amino acid modifications. In preferred embodiment, an anti-SARS-CoV-2 single domain antibody is provided, wherein the single antibody domain comprises a CDR2 comprising SEQ ID NO:11 and a CDR3 comprising SEQ ID NO:12; and wherein the amino acid sequence of CDR3 comprises between 0 and 7 amino acid modifications and wherein the amino acid sequence of CDR2 comprises between 0 and 4 amino acid modifications. In one embodiment the CDR3 regions comprise between 0 and 6, 0 and 5, 0 and 4, 0 and 3, 0 and 2 or 0 and 1 amino acid modifications.

In one embodiment the single domain antibody of the invention may further comprise a CDR1 region. The CDR1 region may be defined according to a SEQ ID NO disclosed herein. In each embodiment, the single domain antibody may further comprise four framework regions (FR1, FR2, FR3 and FR4).

In one aspect, an anti-SARS-CoV-2 single domain antibody is provided, wherein the single antibody domain comprises
(a) a CDR1 comprising SEQ ID NO:10, a CDR2 comprising SEQ ID NO:11 and a CDR3 comprising SEQ ID NO:12;
(b) a CDR1 comprising SEQ ID NO:196, a CDR2 comprising SEQ ID NO:197 and a CDR3 comprising SEQ ID NO:198;
(c) a CDR1 comprising SEQ ID NO:7, a CDR2 comprising SEQ ID NO:8 and a CDR3 comprising SEQ ID NO:9;
(d) CDR1 comprising SEQ ID NO:1, a CDR2 comprising SEQ ID NO:2 and a CDR3 comprising SEQ ID NO:3;
(e) a CDR1 comprising SEQ ID NO:4, a CDR2 comprising SEQ ID NO:5 and a CDR3 comprising SEQ ID NO:6;
(f) a CDR1 comprising SEQ ID NO:13, a CDR2 comprising SEQ ID NO:14 and a CDR3 comprising SEQ ID NO:15;
(g) a CDR1 comprising SEQ ID NO:190, a CDR2 comprising SEQ ID NO:191 and a CDR3 comprising SEQ ID NO:192;
(h) a CDR1 comprising SEQ ID NO:193, a CDR2 comprising SEQ ID NO:194 and a

CDR3 comprising SEQ ID NO:195;
(i) a CDR1 comprising SEQ ID NO:199, a CDR2 comprising SEQ ID NO:200 and a CDR3 comprising SEQ ID NO:201;
(j) a CDR1 comprising SEQ ID NO:202, a CDR2 comprising SEQ ID NO:203 and a CDR3 comprising SEQ ID NO:204; or
(k) a CDR1 comprising SEQ ID NO:205, a CDR2 comprising SEQ ID NO:206 and a CDR3 comprising SEQ ID NO:207; or
(l) a CDR1 comprising SEQ ID NO:235, a CDR2 comprising SEQ ID NO:236 and a CDR3 comprising SEQ ID NO:237;
wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications. In one embodiment the CDR3 regions comprise between 0 and 6, 0 and 5, 0 and 4, 0 and 3, 0 and 2 or 0 and 1 amino acid modifications. In one embodiment the CDR2 regions comprise between 0 and 3, 0 and 2, 0 and 4, 0 and 1 amino acid modifications. In one embodiment the CDR1 regions comprise between 0 and 3, 0 and 2, 0 and 4, 0 and 1 amino acid modifications.

In a preferred embodiment, an anti-SARS-CoV-2 single domain antibody is provided, wherein the single antibody domain comprises a CDR1 comprising SEQ ID NO:196, a CDR2 comprising SEQ ID NO:197 and a CDR3 comprising SEQ ID NO:198; and wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications. In one embodiment the CDR3 regions comprise between 0 and 6, 0 and 5, 0 and 4, 0 and 3, 0 and 2 or 0 and 1 amino acid modifications.

In a most preferred embodiment, an anti-SARS-CoV-2 single domain antibody is provided, wherein the single antibody domain comprises a CDR1 comprising SEQ ID NO:10, a CDR2 comprising SEQ ID NO:11 and a CDR3 comprising SEQ ID NO:12; and wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications. In one embodiment the CDR3 regions comprise between 0 and 6, 0 and 5, 0 and 4, 0 and 3, 0 and 2 or 0 and 1 amino acid modifications.

In one embodiment, the single chain antibody or antigen binding molecule comprises four framework regions. The framework regions separate the CDR sequences. In one embodiment, the four framework regions are framework region 1 (FR1), framework region 2 (FR2), framework region 3 (FR3) and framework region 4 (FR4) and are interspersed between the CDR1, CDR2 and CDR3 (i.e FR1-CDR1-FR2-CDR2-FR3-CDR3-FR4). In one embodiment, the single domain antibody or antigen binding molecule of the invention comprises or essentially consists of four framework regions (FR1, FR2, FR3 and FR4) and three CDRs (CDR1, CDR2 and CDR3). In one embodiment, the single domain antibody or antigen binding molecule of the invention consists of four framework regions (FR1, FR2, FR3 and FR4) and three CDRs (CDR1, CDR2 and CDR3).

In one aspect, an anti-SARS-CoV-2 single domain antibody comprising an amino acid sequence having at least 70% identity to a sequence selected from the group consisting of: SEQ ID NO: 19, 213, 18, 16, 17, 20, 209, 211, 215, 217, 219, 220 and 239 is provided. In one aspect, an anti-SARS-CoV-2 single domain antibody comprising an amino acid sequence having at least 70% identity to a sequence selected from the group consisting of: SEQ ID NO: 19, 18, 16, 17 and 20 is provided. Each of these sequences comprises three CDR regions (CDR1, CDR2 and CDR3) and four framework regions (FR1, FR2, FR3 and FR4). In one embodiment, the amino acid sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity to a sequence selected from the group consisting of: SEQ ID NO: 19, 213, 18, 16, 17, 20, 209, 211, 215, 217, 219, 220 and 239. In one embodiment, an anti-SARS-CoV-2 single domain antibody comprising a sequence selected from the group consisting of SEQ ID NO: 19, 213, 18, 16, 17, 20, 209, 211, 215, 217, 219, 220 and 239 is provided. In one embodiment, an anti-SARS-CoV-2 single domain antibody comprising a sequence selected from the group consisting of SEQ ID NO: 19, 18, 16, 17 and 20 is provided. In one embodiment, an anti-SARS-CoV-2 single domain antibody consisting or essentially consisting a sequence selected from the group consisting of SEQ ID NO: 19, 213, 18, 16, 17, 20, 209, 211, 215, 217, 219, 220 and 239 is provided. In one embodiment, an anti-SARS-CoV-2 single domain antibody consisting or essentially consisting a sequence selected from the group consisting of SEQ ID NO: 19, 18, 16, 17 and 20 is provided.

At least herein and throughout means, in some embodiments, the recited percentage up to 100%. For example, at least 75% can mean, in some embodiments, 75% to 100%.

In one embodiment, an anti-SARS-CoV-2 single domain antibody comprising an amino acid sequence having at least 70% identity to SEQ ID NOs: 16 is provided. In one embodiment, the amino acid sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 16. In one embodiment, the amino acid sequence is SEQ ID NO: 16.

In one embodiment, an anti-SARS-CoV-2 single domain antibody comprising an amino acid sequence having at least 70% identity to SEQ ID NOs: 17 is provided. In one embodiment, the amino acid sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 17. In one embodiment, the amino acid sequence is SEQ ID NO: 17.

In one embodiment, an anti-SARS-CoV-2 single domain antibody comprising an amino acid sequence having at least 70% identity to SEQ ID NOs: 18 is provided. In one embodiment, the amino acid sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 18. In one embodiment, the amino acid sequence is SEQ ID NO: 18.

In a most preferred embodiment, an anti-SARS-CoV-2 single domain antibody comprising an amino acid sequence having at least 70% identity to SEQ ID NOs: 19 is provided. In one embodiment, the amino acid sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 19. In one embodiment, the amino acid sequence is SEQ ID NO: 19.

In one embodiment, an anti-SARS-CoV-2 single domain antibody comprising an amino acid sequence having at least 70% identity to SEQ ID NOs: 20 is provided. In one embodiment, the amino acid sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 20. In one embodiment, the amino acid sequence is SEQ ID NO: 20.

In one embodiment, an anti-SARS-CoV-2 single domain antibody comprising an amino acid sequence having at least 70% identity to SEQ ID NOs: 209 is provided. In one embodiment, the amino acid sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 209. In one embodiment, the amino acid sequence is SEQ ID NO: 209.

In one embodiment, an anti-SARS-CoV-2 single domain antibody comprising an amino acid sequence having at least 70% identity to SEQ ID NOs: 211 is provided. In one embodiment, the amino acid sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 211. In one embodiment, the amino acid sequence is SEQ ID NO: 211.

In one embodiment, an anti-SARS-CoV-2 single domain antibody comprising an amino acid sequence having at least 70% identity to SEQ ID NOs: 213 is provided. In one embodiment, the amino acid sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 213. In one embodiment, the amino acid sequence is SEQ ID NO: 213.

In one embodiment, an anti-SARS-CoV-2 single domain antibody comprising an amino acid sequence having at least 70% identity to SEQ ID NOs: 215 is provided. In one embodiment, the amino acid sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 215. In one embodiment, the amino acid sequence is SEQ ID NO: 215.

In one embodiment, an anti-SARS-CoV-2 single domain antibody comprising an amino acid sequence having at least 70% identity to SEQ ID NOs: 217 is provided. In one embodiment, the amino acid sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 217. In one embodiment, the amino acid sequence is SEQ ID NO: 217.

In one embodiment, an anti-SARS-CoV-2 single domain antibody comprising an amino acid sequence having at least 70% identity to SEQ ID NOs: 219 is provided. In one embodiment, the amino acid sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 219. In one embodiment, the amino acid sequence is SEQ ID NO: 219.

In one embodiment, an anti-SARS-CoV-2 single domain antibody comprising an amino acid sequence having at least 70% identity to SEQ ID NOs: 220 is provided. In one embodiment, the amino acid sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 220. In one embodiment, the amino acid sequence is SEQ ID NO: 220.

In one embodiment, an anti-SARS-CoV-2 single domain antibody comprising an amino acid sequence having at least 70% identity to SEQ ID NOs: 239 is provided. In one embodiment, the amino acid sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 239. In one embodiment, the amino acid sequence is SEQ ID NO: 239.

In one aspect, a polynucleotide sequence is provided encoding a single domain antibody of the invention. In one embodiment, the polynucleotide is DNA or RNA. Such nucleic acid sequences may be in the form of a genetic construct.

In one embodiment, an anti-SARS-CoV-2 single domain antibody comprises a polynucleotide sequence having at least 70% identity to a sequence selected from the group consisting of: SEQ ID NO: 24, 212, 23, 21, 22, 25, 208, 210, 214, 216, 218 and 238 is provided. In one embodiment, an anti-SARS-CoV-2 single domain antibody comprises a polynucleotide sequence having at least 70% identity to a sequence selected from the group consisting of: SEQ ID NO: 24, 23, 21, 22 and 25 is provided. In one embodiment, the polynucleotide sequence has at least 75%, 80%, 85%, 90%, 91%, 92%, 93%, 94%, 95%, 96%, 97%, 98% or at least 99% identity to a sequence selected from the group consisting of: SEQ ID NO: 24, 23, 21, 22, 25, 208, 210, 212, 214, 216, 218 and 238. In one embodiment, an anti-SARS-CoV-2 single domain antibody comprises a sequence selected from the group consisting of SEQ ID NO: 24, 23, 21, 22 and 25 is provided. In one embodiment, an anti-SARS-CoV-2 single domain antibody comprises a sequence selected from the group consisting of SEQ ID NO: 24, 23, 21, 22, 25, 208, 210, 212, 214, 216, 218 and 238 is provided. In one embodiment, an anti-SARS-CoV-2 single domain antibody consisting or essentially consisting of SEQ ID NO: 24, 23, 21, 22 and 25 is provided. In one embodiment, an anti-SARS-CoV-2 single domain antibody consisting or essentially consisting of SEQ ID NO: 24, 23, 21, 22, 25, 208, 210, 212, 214, 216, 218 and 238 is provided.

In one embodiment, an anti-SARS-CoV-2 single domain antibody comprising a polynucleotide sequence having at least 70% identity to SEQ ID NOs: 21 is provided. In one embodiment, the polynucleotide sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 21. In one embodiment, the polynucleotide sequence is SEQ ID NO: 21.

In one embodiment, an anti-SARS-CoV-2 single domain antibody comprising a polynucleotide sequence having at least 70% identity to SEQ ID NOs: 22 is provided. In one embodiment, the polynucleotide sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 22. In one embodiment, the polynucleotide sequence is SEQ ID NO: 22.

In one embodiment, an anti-SARS-CoV-2 single domain antibody comprising a polynucleotide sequence having at least 70% identity to SEQ ID NOs: 23 is provided. In one embodiment, the polynucleotide sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 23. In one embodiment, the polynucleotide sequence is SEQ ID NO: 23.

In a preferred embodiment, an anti-SARS-CoV-2 single domain antibody comprising a polynucleotide sequence having at least 70% identity to SEQ ID NOs: 24 is provide. In one embodiment, the polynucleotide sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 24. In one embodiment, the polynucleotide sequence is SEQ ID NO: 24.

In one embodiment, an anti-SARS-CoV-2 single domain antibody comprising a polynucleotide sequence having at least 70% identity to SEQ ID NOs: 25 is provided. In one embodiment, the polynucleotide sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 25. In one embodiment, the polynucleotide sequence is SEQ ID NO: 25.

In one embodiment, an anti-SARS-CoV-2 single domain antibody comprising a polynucleotide sequence having at least 70% identity to SEQ ID NOs: 208 is provided. In one embodiment, the polynucleotide sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 208. In one embodiment, the polynucleotide sequence is SEQ ID NO: 208.

In one embodiment, an anti-SARS-CoV-2 single domain antibody comprising a polynucleotide sequence having at least 70% identity to SEQ ID NOs: 210 is provided. In one embodiment, the polynucleotide sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 210. In one embodiment, the polynucleotide sequence is SEQ ID NO: 210.

In a preferred embodiment, an anti-SARS-CoV-2 single domain antibody comprising a polynucleotide sequence having at least 70% identity to SEQ ID NOs: 212 is provided. In one embodiment, the polynucleotide sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 212. In one embodiment, the polynucleotide sequence is SEQ ID NO: 212.

In one embodiment, an anti-SARS-CoV-2 single domain antibody comprising a polynucleotide sequence having at least 70% identity to SEQ ID NOs: 214 is provided. In one embodiment, the polynucleotide sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 214. In one embodiment, the polynucleotide sequence is SEQ ID NO: 214.

In one embodiment, an anti-SARS-CoV-2 single domain antibody comprising a polynucleotide sequence having at least 70% identity to SEQ ID NOs: 216 is provided. In one embodiment, the polynucleotide sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 216. In one embodiment, the polynucleotide sequence is SEQ ID NO: 216.

In one embodiment, an anti-SARS-CoV-2 single domain antibody comprising a polynucleotide sequence having at least 70% identity to SEQ ID NOs: 218 is provided. In one embodiment, the polynucleotide sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 218. In one embodiment, the polynucleotide sequence is SEQ ID NO: 218.

In one embodiment, an anti-SARS-CoV-2 single domain antibody comprising a polynucleotide sequence having at least 70% identity to SEQ ID NOs: 238 is provided. In one embodiment, the polynucleotide sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity SEQ ID NO: 238. In one embodiment, the polynucleotide sequence is SEQ ID NO: 238.

In one aspect, the present invention provides coronavirus binding molecules that bind two different epitopes on the receptor binding domain of the spike protein of a coronavirus, preferably a SARS-CoV-2 spike protein. The coronavirus binding molecules are based on joining two antigen binding molecules together via a linker. The linker comprises a protein from the ubiquitin-like protein superfamily, either ubiquitin (Ub) itself or a ubiquitin-like protein (ULP). The linker optionally further comprises a spacer comprising 4 to 50 amino acid residues at the n-terminal of the ubiquitin (Ub) or ubiquitin-like protein and optionally a spacer comprising 4 to 50 amino acid residues at the c-terminal of the ubiquitin (Ub) or a ubiquitin-like protein. The antigen binding molecules target two different epitopes on the receptor binding domain of the spike protein of a coronavirus, preferably a SARS-CoV-2 spike protein. The first antigen binding molecule binds to all or a part of a first epitope comprised within SEQ ID NO: 232. The second antigen binding molecule binds to all or a part of a first epitope comprised within SEQ ID NO: 233. By combining two antigen binding molecules that bind to different epitopes on the receptor-binding domain a significant gain in overall binding affinity has been surprisingly demonstrated.

The coronavirus binding molecules are made according to the following general design:

N′-ABM(1)-SPACER 1-LINKER-SPACER 2-ABM(2)-C′
ABM(1)—Antigen binding molecule targeting epitope 1
ABM(2)—Antigen binding molecule targeting epitope 2
LINKER—ubiquitin or a ubiquitin-like protein
SPACER 1 (optional)—5-50 amino acids
SPACER 2 (optional)—5-50 amino acids

The coronavirus binding molecule can be ordered such that the first antigen binding molecule is positioned at, or nearest, the n-terminal end of the coronavirus binding molecule and the second antigen binding molecule is positioned at, or nearest, the c-terminal end of the coronavirus binding molecule. Alternatively, the coronavirus binding molecule can be ordered such that the second antigen binding molecule is positioned at, or nearest, the n-terminal end of the coronavirus binding molecule and the first antigen binding molecule is positioned at, or nearest, the c-terminal end of the coronavirus binding molecule, for example:

N′-ABM(1)-SPACER 1-LINKER-SPACER 2-ABM(2)-C′

N′-ABM(2)-SPACER 1-LINKER-SPACER 2-ABM(1)-C′

The coronavirus binding molecules of the invention comprise first and second antigen binding molecules. An antigen binding molecule as used herein can be an antibody or fragment thereof, a single domain antibody or fragment thereof. In one embodiment, the first antigen binding molecule is an antibody, or fragment or variant thereof. In one embodiment, the first antigen binding molecule is a single-chain variable fragment (scFv). In one embodiment, the second antigen binding molecule is an antibody, or fragment or variant thereof. In one embodiment, the second antigen binding molecule is a single-chain variable fragment (scFv). In one embodiment, the first antigen binding molecule is a single domain antibody. In one embodiment, the second antigen binding molecule is a single domain antibody. Preferably, the first antigen binding molecule and the second antigen binding molecules are single domain antibodies.

The first and second antigen binding molecules bind to all or part of a first and second epitope, respectively, wherein the first and second epitopes are substantially non-overlapping. The first and second epitopes are both located on the receptor binding domain (RBD) of the spike of a coronavirus, preferably SARS-CoV-2. Given the degree of homology between spike proteins of coronaviruses, the coronavirus binding molecules of the invention comprising two antigen binding molecules joined together via a linker are spatially configured such that they can advantageously target two non-overlapping epitopes on the spike protein of a range of coronaviruses, including SARS-CoV-1, SARS-CoV-2 and MERS. In this respect, the coronavirus binding molecules of the invention may target more than one type of coronavirus, thereby providing a pan-coronavirus binding molecule. In one embodiment, the coronavirus binding molecules of the invention may bind to both a first and second epitope on SARS-CoV-1 and also bind to the corresponding first and second epitope on SARS-CoV-2.

In one embodiment, the first antigen binding molecule binds to all or part of a first epitope located on the receptor binding domain (RBD) of the spike protein of a coronavirus. The coronavirus can be a coronavirus selected from the group consisting of SARS-CoV-1, SARS-CoV-2 and MERS, preferably SARS-CoV-2, most preferably human SARS-CoV-2. In a one embodiment, the first antigen binding molecule binds to all or part of a first epitope located on the receptor binding domain (RBD) of Spike of SARS-CoV-1. In a preferred embodiment, the first antigen binding molecule binds to all or part of a first epitope located on the receptor binding domain (RBD) of Spike of SARS-CoV-2. Epitope 1 comprises the surface of the RBD that binds to the ACE2 receptor. Antigen binding molecules binding to this epitope therefore directly block the receptor binding domain of the coronavirus binding to human ACE2 protein. This epitope is targeted by single domain antibodies C5, H11-H4, H11-D4, H3, H11-A10, H11-B5, H11-H6 and VHH_H6 as described herein.

The Spike glycoprotein sequence for SARS-CoV-2 is provided below with the region for epitope 1 highlighted and shown in bold:

Spike glycoprotein (UniProt,
https://www.uniprot.org/uniprot/P59594)
(SEQ ID NO: 231)
MFVFLVLLPLVSSQCVNLTTRTQLPPAYTNSFTRGVYYPD
KVFRSSVLHSTQDLFLPFFSNVTWFHAIHVSGTNGTKRFD
NPVLPFNDGVYFASTEKSNIIRGWIFGTTLDSKTQSLLIV
NNATNVVIKVCEFQFCNDPFLGVYYHKNNKSWMESEFRVY
SSANNCTFEYVSQPFLMDLEGKQGNFKNLREFVFKNIDGY
FKIYSKHTPINLVRDLPQGFSALEPLVDLPIGINITRFQT
LLALHRSYLTPGDSSSGWTAGAAAYYVGYLQPRTFLLKYN
ENGTITDAVDCALDPLSETKCTLKSFTVEKGIYQTSNFRV
QPTESIVRFPNITNLCPFGEVFNATRFASVYAWNRKRISN
CVADYSVLYNSASFSTFKCYGVSPTKLNDLCFTNVYADSF
VIRGDEVRQIAPGQTGKIADYNYKLPDDFTGCVIAWNSNN
LDSKVGGNYNYLYRLFRKSNLKPFERDISTEIYQAGSTPC
NGVEGFNCYFPLQSYGFQPTNGVGYQPYRVVVLSFELLHA
PATVCGPKKSTNLVKNKCVNFNFNGLTGTGVLTESNKKFL
PFQQFGRDIADTTDAVRDPQTLEILDITPCSFGGVSVITP
GTNTSNQVAVLYQDVNCTEVPVAIHADQLTPTWRVYSTGS
NVFQTRAGCLIGAEHVNNSYECDIPIGAGICASYQTQTNS
PRRARSVASQSIIAYTMSLGAENSVAYSNNSIAIPTNFTI
SVTTEILPVSMTKTSVDCTMYICGDSTEFIEDLLFNKVTL
ADAGFIKQYGDCLGDIAARDLICAQKFNGLTVLPPLLTDE
MIAQYTSALLAGTITSGWTFGAGAALQIPFAMQMAYRFNG
IGVTQNVLYENQKLIANQFNSAIGKIQDSLSSTASALGKL
QDVVNQNAQALNTLVKQLSSNFGAISSVLNDILSRLDKVE
AEVQIDRLITGRLQSLQTYVTQQLIRAAEIRASANLAATK
MSECVLGQSKRVDFCGKGYHLMSFPQSAPHGVVFLHVTYV
PAQEKNFTTAPAICHDGKAHFPREGVFVSNGTHWFVTQRN
FYEPQIITTDNTFVSGNCDVVIGIVNNTVYDPLQPELDSF
KEELDKYFKNHTSPDVDLGDISGINASVVNIQKEIDRLNE
VAKNLNESLIDLQELGKYEQYIKWPWYIWLGFIAGLIAIV
MVTIMLCCMTSCCSCLKGCCSCGSCCKFDEDDSEPVLKGV
KLHYT

Epitope 1 is defined as spanning residues 346 to 505 of the Spike glycoprotein of SARS-CoV-2, provided as SEQ ID NO:232 below:

RFASVYAWNRKRISNCVADYSVLYNSASFSTFKCYGVSPT
KLNDLCFTNVYADSFVIRGDEVRQIAPGQTGKIADYNYKL
PDDFTGCVIAWNSNNLDSKVGGNYNYLYRLFRKSNLKPFE
RDISTETYQAGSTPCNGVEGFNCYFPLQSYGFQPTNGVGY

In particular, epitope 1 as targeted by the coronavirus binding molecules of the invention is non-linear and is comprised of the following amino acids of the Spike glycoprotein of SARS-CoV-2:
Arg 346, Arg 403, Lys 444, Gly 446, Gly 447, Tyr 449, Asn 450, Leu 452, Tyr 453, Leu 455, Phe 456, Thr 470, Ile 472, Gly 482, Val 483, Glu 484, Gly 485, Phe 486, Cys 488, Tyr 489, Phe 490, Ley 492, Gln 493, Ser 494, Tyr 495, Gly 496, Gln 498, Thr 500, Asn 501, Tyr 505

In one embodiment, the second antigen binding molecule binds to all or part of a second epitope located on the receptor binding domain (RBD) of the spike protein of a coronavirus. The coronavirus can be a coronavirus selected from the group consisting of SARS-CoV-1, SARS-CoV-2 and MERS, preferably SARS-CoV-2, most preferably human SARS-CoV-2. In a one embodiment, the second antigen binding molecule binds to all or part of a first epitope located on the receptor binding domain (RBD) of Spike of SARS-CoV-1. In a preferred embodiment, the second antigen binding molecule binds to all or part of a second epitope located on the receptor binding domain (RBD) of Spike of SARS-CoV-2. Epitope 2 is located remotely to the ACE2 binding site (i.e. the region of epitope 1) and is targeted by A8, F2, VHH72, CR3022, EY6A, C1 and B12.

The Spike glycoprotein sequence is provided below with the region for epitope 2 highlighted and shown in bold:

Spike glycoprotein (UniProt,
https://www.uniprot.org/uniprot/P59594)
(SEQ ID NO: 231)
MFVFLVLLPLVSSQCVNLTTRTQLPPAYTNSFTRGVYYPD
KVFRSSVLHSTQDLFLPFFSNVTWFHAIHVSGTNGTKRFD
NPVLPFNDGVYFASTEKSNIIRGWIFGTTLDSKTQSLLIV
NNATNVVIKVCEFQFCNDPFLGVYYHKNNKSWMESEFRVY
SSANNCTFEYVSQPFLMDLEGKQGNFKNLREFVFKNIDGY
FKIYSKHTPINLVRDLPQGFSALEPLVDLPIGINITRFQT
LLALHRSYLTPGDSSSGWTAGAAAYYVGYLQPRTFLLKYN
ENGTITDAVDCALDPLSETKCTLKSFTVEKGIYQTSNFRV
QPTESIVRFPNITNLCPFGEVFNATRFASVYAWNRKRISN
CVADYSVLYNSASFSTFKCYGVSPTKLNDLCFTNVYADSF
VIRGDEVRQIAPGQTGKIADYNYKLPDDFTGCVIAWNSNN
LDSKVGGNYNYLYRLFRKSNLKPFERDISTEIYQAGSTPC
NGVEGFNCYFPLQSYGFQPTNGVGYQPYRVVVLSFELLHA
PATVCGPKKSTNLVKNKCVNFNFNGLTGTGVLTESNKKFL
PFQQFGRDIADTTDAVRDPQTLEILDITPCSFGGVSVITP
GTNTSNQVAVLYQDVNCTEVPVAIHADQLTPTWRVYSTGS
NVFQTRAGCLIGAEHVNNSYECDIPIGAGICASYQTQTNS
PRRARSVASQSIIAYTMSLGAENSVAYSNNSIAIPTNFTI
SVTTEILPVSMTKTSVDCTMYICGDSTEFIEDLLFNKVTL
ADAGFIKQYGDCLGDIAARDLICAQKFNGLTVLPPLLTDE
MIAQYTSALLAGTITSGWTFGAGAALQIPFAMQMAYRFN
GIGVTQNVLYENQKLIANQFNSAIGKIQDSLSSTASALGK
LQDVVNQNAQALNTLVKQLSSNFGAISSVLNDILSRLDKV
EAEVQIDRLITGRLQSLQTYVTQQLIRAAEIRASANLAAT
KMSECVLGQSKRVDFCGKGYHLMSFPQSAPHGVVFLHVTY
VPAQEKNFTTAPAICHDGKAHFPREGVFVSNGTHWFVTQR
NFYEPQIITTDNTFVSGNCDVVIGIVNNTVYDPLQPELD
SFKEELDKYFKNHTSPDVDLGDISGINASVVNIQKEIDR
LNEVAKNLNESLIDLQELGKYEQYIKWPWYIWLGFIAGLI
AIVMVTIMLCCMTSCCSCLKGCCSCGSCCKFDEDDSEPVL
KGVKLHYT

Epitope 2 is defined as spanning residues 368 to 519 of the Spike glycoprotein of SARS-CoV-2, provided as SEQ ID NO:233 below:

LYNSASFSTFKCYGVSPTKLNDLCFTNVYADSFV
IRGDEVRQIAPGQTGKIADYNYKLPDDFTGCVIA
WNSNNLDSKVGGNYNYLYRLFRKSNLKPFERDIS
TETYQAGSTPCNGVEGFNCYFPLQSYGFQPTNGV
GYQPYRVVVLSFELLH

Epitope 2 as targeted by the coronavirus binding molecules of the invention is non-linear and is comprised of the following amino acids of the Spike glycoprotein of SARS-CoV-2:
Leu 368, Tyr 369, Asn 370, Ser 371, Ala 372, Phe 374, Ser 375, Thr 376, Phe 377, Lys 378, Cys 379, Tyr 380, Gly 381, Val 382, Ser 383, Pro 384, Thr 385, Lys 386, Asn 388, Asp 389, Leu 390, Phe 392, Arg 408, Ala 411, Pro 412, Gly 413, Gln 414, Asp 427, Asp 428, Phe 429, Thr 430, Phe 515, Glu 516, Leu 517, Leu 518, His 519

In one aspect, a coronavirus binding molecule is provided comprising:
(a) a first antigen binding molecule that binds to all or part of a first epitope comprised within a coronavirus protein;
(b) a second antigen binding molecule that binds to all or part of a second epitope comprised within a coronavirus protein; and
(c) a linker,
wherein the linker comprises:
(a) a ubiquitin or a ubiquitin-like protein;
(b) a further optional spacer of between 4 and 50 amino acids joined to the n-terminal of the ubiquitin or ubiquitin-like protein; and
(c) further optional spacer of between 4 and 50 amino acids joined to the c-terminal of the ubiquitin or ubiquitin-like protein;
and wherein the linker joins the first antigen binding molecule to the second antigen binding molecule,
and optionally wherein the first and second epitopes are substantially non-overlapping. In one embodiment, the coronavirus protein is the Spike Protein, optionally the Receptor Binding Domain (RBD) of the Spike Protein. The coronavirus can be a coronavirus selected from the group consisting of SARS-CoV-1, SARS-CoV-2 and MERS, preferably SARS-CoV-2.

In one aspect, a coronavirus binding molecule is provided comprising:
(a) a first antigen binding molecule that binds to all or part of a first epitope comprised within SEQ ID NO: 232;
(b) a second antigen binding molecule that binds to all or part of a second epitope comprised within SEQ ID NO: 233; and
(c) a linker,
wherein the linker comprises:
(i) a ubiquitin or a ubiquitin-like protein;
(ii) further optional spacer of between 4 and 50 amino acids joined to the n-terminal of the ubiquitin or ubiquitin-like protein; and
(iii) further optional spacer of between 4 and 50 amino acids joined to the c-terminal of the ubiquitin or ubiquitin-like protein;
and wherein the linker joins the first antigen binding molecule to the second antigen binding molecule,
and optionally wherein the first and second epitopes are substantially non-overlapping.

In one embodiment, the coronavirus protein is the Spike Protein, optionally the Receptor Binding Domain (RBD) of the Spike Protein. The coronavirus can be a coronavirus selected from the group consisting of SARS-CoV-1, SARS-CoV-2 and MERS, preferably SARS-CoV-2.

The antigen binding molecules bind to either all or part of their target epitopes. In one embodiment, the first antigen binding molecule is selected from the group consisting of C5, H4, H3, D4, A10, B5 and H6. In one embodiment, the second antigen binding molecule is selected from the group consisting of C1, B12, F2, Vhh72, CR3022, EY6A. In one embodiment, the second antigen binding molecule is located remotely that of epitope 1

In one embodiment, the coronavirus binding molecule further comprises a third and optionally a fourth or fifth antigen binding molecule that binds to all or part of a third, fourth or fifth epitope comprised within the coronavirus protein, optionally a SARS-CoV-2 protein, optionally wherein the third, fourth or fifth antigen binding molecules are linked to the first and second antigen binding molecules via one of more linkers and the linkage is either directly to the first or the second antigen binding molecules or to the ubiquitin or a ubiquitin-like protein or one of the further optional spacers.

In one embodiment, the first antigen binding molecule binds to at least 10 amino acids selected from the group consisting of residues 346, 403, 444, 446, 447, 449, 450, 452, 453, 455, 456, 470, 472, 482, 483, 484, 485, 486, 488, 489, 490, 492, 493, 494, 495, 496, 498, 500, 501 and 505 of the Spike glycoprotein of SARS-CoV-2 (SEQ ID NO: 231). In one embodiment, the first antigen binding molecule binds to at least 20 amino acids selected from the group consisting of residues 346, 403, 444, 446, 447, 449, 450, 452, 453, 455, 456, 470, 472, 482, 483, 484, 485, 486, 488, 489, 490, 492, 493, 494, 495, 496, 498, 500, 501 and 505 of the Spike glycoprotein of SARS-CoV-2 (SEQ ID NO: 231). In one embodiment, the first antigen binding molecule binds to at least 25 amino acids selected from the group consisting of residues 346, 403, 444, 446, 447, 449, 450, 452, 453, 455, 456, 470, 472, 482, 483, 484, 485, 486, 488, 489, 490, 492, 493, 494, 495, 496, 498, 500, 501 and 505 of the Spike glycoprotein of SARS-CoV-2 (SEQ ID NO: 231). In one embodiment, the first antigen binding molecule binds to at least 26, 27, 28 or 29 amino acids selected from the group consisting of residues 346, 403, 444, 446, 447, 449, 450, 452, 453, 455, 456, 470, 472, 482, 483, 484, 485, 486, 488, 489, 490, 492, 493, 494, 495, 496, 498, 500, 501 and 505 of the Spike glycoprotein of SARS-CoV-2 (SEQ ID NO: 231). In one embodiment, the first antigen binding molecule binds to residues 346, 403, 444, 446, 447, 449, 450, 452, 453, 455, 456, 470, 472, 482, 483, 484, 485, 486, 488, 489, 490, 492, 493, 494, 495, 496, 498, 500, 501 and 505 of the Spike glycoprotein of SARS-CoV-2 (SEQ ID NO: 231).

In one embodiment, the second antigen binding molecule binds to at least 10 amino acids selected from the group consisting of residues 368, 369, 370, 371, 372, 374, 375, 376, 377, 378, 379, 380, 381, 382, 383, 384, 385, 386, 388, 389, 390, 392, 408, 411, 412, 413, 414, 427, 428, 429, 430, 515, 516, 517, 518 and 519 of the Spike glycoprotein of SARS-CoV-2 (SEQ ID NO: 231). In one embodiment, the second antigen binding molecule binds to at least 20 amino acids selected from the group consisting of residues 368, 369, 370, 371, 372, 374, 375, 376, 377, 378, 379, 380, 381, 382, 383, 384, 385, 386, 388, 389, 390, 392, 408, 411, 412, 413, 414, 427, 428, 429, 430, 515, 516, 517, 518 and 519 of the Spike glycoprotein of SARS-CoV-2 (SEQ ID NO: 231). In one embodiment, the second antigen binding molecule binds to at least amino acids selected from the group consisting of residues 368, 369, 370, 371, 372, 374, 375, 376, 377, 378, 379, 380, 381, 382, 383, 384, 385, 386, 388, 389, 390, 392, 408, 411, 412, 413, 414, 427, 428, 429, 430, 515, 516, 517, 518 and 519 of the Spike glycoprotein of SARS-CoV-2 (SEQ ID NO: 231). In one embodiment, the second antigen binding molecule binds to at least 31, 32, 33, 34 or 35 amino acids selected from the group consisting of residues 368, 369, 370, 371, 372, 374, 375, 376, 377, 378, 379, 380, 381, 382, 383, 384, 385, 386, 388, 389, 390, 392, 408, 411, 412, 413, 414, 427, 428, 429, 430, 515, 516, 517, 518 and 519 of the Spike glycoprotein of SARS-CoV-2 (SEQ ID NO: 231). In one embodiment, the second antigen binding molecule binds to residues 368, 369, 370, 371, 372, 374, 375, 376, 377, 378, 379, 380, 381, 382, 383, 384, 385, 386, 388, 389, 390, 392, 408, 411, 412, 413, 414, 427, 428, 429, 430, 515, 516, 517, 518 and 519 of the Spike glycoprotein of SARS-CoV-2 (SEQ ID NO: 231).

In one embodiment, the first antigen binding molecule that binds to all or part of a first epitope comprised within a SARS-CoV-2 protein and the second antigen binding molecule binds to all or part of a second epitope CoV-2 comprised within a coronavirus protein, wherein the first and second epitopes are substantially non-overlapping. The SARS-CoV-2 protein may be the Spike glycoprotein [SEQ ID NO: 231], optionally the Receptor Binding Domain (RBD) on the S1 subunit the Spike Protein.

The linker of the invention links the two antigen binding molecules in an optimal spatial arrangement to facilitate exceptional properties when bound to the RBD binding domain of the spike protein of SARS-CoV-2. The linker permits the first and second antigen binding molecules to be positioned in an angular arrangement such that they can bind the first and second epitope respectively. When a coronavirus binding molecule of the invention is bound to its two target epitopes, the N′ terminal to C′ terminal distance of the linker is approximately to 70 ångströms, optionally 50 to 65 ångströms. In one embodiment, the distance is approximately 50 to 55 ångströms. In one embodiment, the distance is approximately 55 to 60 ↑ngströms. In one embodiment, the distance is approximately 60 to 65 ångströms. The specified distance is the span of the linker itself, including any additional spacer(s) if present. The coronavirus binding molecule of the invention use Ubiquitin (Ub) or ubiquitin-like proteins in their natural folded state as a linker, therefore the distances specified herein refer to Ubiquitin (Ub) or ubiquitin-like protein in their natural 3D conformation, having a ubiquitin fold.

In one embodiment, the coronavirus molecule comprises an optional spacer of 4 to 50 amino acids joined to the n-terminal or c-terminal of the ubiquitin or ubiquitin-like protein, wherein the spacer comprises:
a. one or more GlySer repeats; and/or
b. one or more polyA stretches.

In one aspect, the coronavirus binding molecule further comprises additional antigen binding molecules. In one embodiment, the coronavirus binding molecule comprises a third and optionally a fourth or fifth antigen binding molecule that binds to all or part of a third, fourth or fifth epitope comprised within the coronavirus protein, optionally a SARS-CoV-2 protein, optionally wherein the third, fourth or fifth antigen binding molecules are linked to the first and second antigen binding molecules via one of more linkers and the linkage is either directly to the first or the second antigen binding molecules or to the ubiquitin or a ubiquitin-like protein or one of the further optional spacers. In one embodiment, the coronavirus protein is the spike protein, optionally the Receptor Binding Domain (RBD) of the Spike Protein.

The coronavirus binding molecules of the invention can comprise specific single domain antibodies, as defined herein. In one embodiment, the antigen binding molecules forming the bidentate molecules of the present invention may comprise one or more than one of the specific amino acid sequences disclosed herein. The bidentate molecule may be formed by providing a first antigen binding molecule comprising a first amino acid sequence, as specified herein, and a second antigen binding molecules, containing a second amino acid sequence, as specified herein. Antigen binding molecules or single domain antibodies forming the bidentate molecules of the invention can comprise one or more modifications, as detailed herein, and will retain binding affinity for a coronavirus peptide, preferably the receptor binding domain of the S protein of SARS-CoV-2.

In one embodiment, a coronavirus binding molecule is provided comprising a first antigen binding molecule having an amino acid or polynucleotide sequence based on C5, H3, H11_D4, H11_H4, H11_A10, H11_B5 or VHH_H6 and/or a second antigen binding molecule having an amino acid or polynucleotide sequence based on A8, F2, VHH72, CR3022, EY6A, C1 or B12. The tables below illustrate the various combinations of antigen binding molecules that may form a bidentate polypeptide of the invention.

In a preferred embodiment, a coronavirus binding molecule is provided comprising a first single domain antibody comprising an amino acid or polynucleotide sequence based on C5 and a second single domain antibody having an amino acid or polynucleotide sequence based on A8, F2, VHH72, C1 or B12. In a preferred embodiment, a coronavirus binding molecule is provided comprising a first single domain antibody comprising an amino acid or polynucleotide sequence based on C5, H3, H11_D4, H11_H4, H11_A10, H11_B5 or VHH_H6 and a second single domain antibody having an amino acid or polynucleotide sequence based on A8. In a most preferred embodiment, a coronavirus binding molecule is provided comprising a first single domain antibody comprising an amino acid or polynucleotide sequence based on C5 and a second single domain antibody having an amino acid or polynucleotide sequence based on A8.

As detailed throughout, the amino acid or polynucleotide sequence can comprise the CDR3, CDR2 and/or CDR1 or a variant thereof of the specified single domain antibodies, comprise the amino acid sequence or a variant thereof of the specified single domain antibodies, or comprise the polynucleotide sequence of variant thereof of the specified single domain antibodies.

| | EPITOPE 1 | | | | | | | |
|---|---|---|---|---|---|---|---|---|
| EPITOPE 2 | C5 | H11-H4 | H11-D4 | H3 | H11-A10 | H11-B5 | H11-H6 | VHH_H6 |
| F2 | C5/F2 | H11-H4/ | H11-D4/ | H3/F2 | H11-A10/ | H11-B5/ | H11-H6/ | VHH_H6/ |
| | | F2 | F2 | | F2 | F2 | F2 | F2 |
| VHH72 | C5/ | H11-H4/ | H11-D4/ | H3/ | H11-A10/ | H11-B5/ | H11-H6/ | VHH_H6/ |
| | VHH72 | VHH72 | VHH72 | VHH72 | VHH72 | VHH72 | VHH72 | VHH72 |
| CR3022 | C5/CR | H11-H4/ | H11-D4/ | H3/ | H11-A10/ | H11-B5/ | H11-6/ | VHH_H6/ |
| | 3022 | CR3022 | CR3022 | CR3022 | CR3022 | CR3022 | CR3022 | CR3022 |
| EY6A | C5/ | H11-H4/ | H11-D4/ | H3/ | H11-A10/ | H11-B5/ | H11-H6/ | VHH_H6/ |
| | EY6A | EY6A | EY6A | EY6A | EY6A | EY6A | EY6A | EY6A |
| C1 | C5/C1 | H11-H4/ | H11-D4/ | H3/C1 | H11-A10/ | H11-B5/ | H11-H6/ | VHH_H6/ |
| | | C1 | C1 | | C1 | C1 | C1 | C1 |
| B12 | C5/B12 | H11-H4/ | H11-D4/ | H3/B12 | H11-A10/ | H11-B5/ | H11-H6/ | VHH_H6/ |
| | | B12 | B12 | | B12 | B12 | B12 | B12 |
| A8 | C5/A8 | H11-H4/ | H11-D4/ | H3A8 | H11-A10/ | BH11-5/ | H11-H6/ | VHH_H6/ |
| | | A8 | A8 | | A8 | A8 | A8 | A8 |

| | EPITOPE 2 | | | | | |
|---|---|---|---|---|---|---|
| EPITOPE 1 | F2 | VHH72 | CR3022 | EY6A | C1 | B12 |
| C5 | F2/C5 | VHH72/C5 | CR3022/C5 | EY6A/C5 | C1/C5 | B12/C5 |
| H11-H4 | F2/H4 | VHH72/H11- | CR3022/ | EY6A/H11- | C1/H11- | B12/H11- |
| | | H4 | H11-H4 | H4 | H4 | H4 |
| H11-D4 | F2/D4 | VHH72/H11- | CR3022/ | EY6A/H11- | C1/H11- | B12/H11- |
| | | D4 | H11-D4 | D4 | D4 | D4 |
| H3 | F2/H3 | VHH72/H3 | CR3022/H3 | EY6A/H3 | C1/H3 | B12/H3 |
| H11-A10 | F2/A10 | VHH72/H11- | CR3022/ | EY6A/H11- | C1/H11- | B12/H11- |
| | | A10 | H11-A10 | A10 | A10 | A10 |
| H11-B5 | F2/B5 | VHH72H11-/ | CR3022/ | EY6A/H11- | C1/H11- | B12/H11- |
| | | B5 | H11-B5 | B5 | B5 | B5 |
| H11-H6 | F2/H6 | VHH72/H11- | CR3022/ | EY6A/H11- | C1/H11- | B12/H11- |
| | | H6 | H11-H6 | H6 | H6 | H6 |
| VHH_H6 | F2/VH_ | VHH72/VH_ | CR3022/ | EY6A/ | C1/VH_ | B12/VH_ |
| | H6 | H6 | VH_H6 | VH_H6 | H6 | H6 |

In one embodiment, the first antigen binding molecule of the invention is a single domain antibody comprising a complementary determining region 3 (CDR3) selected from the group consisting of SEQ ID NOs: 12, 15, 72, 73, 74, 75, 76 or 237 wherein the amino acid sequences comprise between 0 and 7 amino acid modifications, optionally 0 and 6, 0 and 5, 0 and 4, 0 and 3, 0 and 2 and 0 and 1 amino acid modifications. In a preferred embodiment, the complementary determining region 3 (CDR3) is SEQ ID NO: 12. In one embodiment, the complementary determining region 3 (CDR3) is SEQ ID NO: 15. In one embodiment, the complementary determining region 3 (CDR3) is SEQ ID NO: 72. In one embodiment, the complementary determining region 3 (CDR3) is SEQ ID NO: 73. In one embodiment, the complementary determining region 3 (CDR3) is SEQ ID NO: 74. In one embodiment, the complementary determining region 3 (CDR3) is SEQ ID NO: 75. In one embodiment, the complementary determining region 3 (CDR3) is SEQ ID NO: 76. In a preferred embodiment, the complementary determining region 3 (CDR3) is SEQ ID NO: 237.

In one embodiment, the first antigen binding molecule of the invention comprises a complementary determining region 3 (CDR3) selected from the group consisting of SEQ ID NOs: 12, 15, 72, 73, 74, 75, 76 and 237, wherein the amino acid sequences of SEQ ID NO: comprise between 0 and 7 amino acid modifications, optionally between 0 and 5 or 0 and 2 modifications; and wherein the CDR3 regions of SEQ ID NO: 12 comprises between 0 and 5 amino acid modifications, optionally between 0 and 2 amino acid modifications.

In one embodiment, the second antigen binding molecule of the invention is a single domain antibody comprising a complementary determining region 3 (CDR3) selected from the group consisting of SEQ ID NOs: 198, 3, 6 and 9, wherein the amino acid sequences comprise between 0 and 7 amino acid modifications, optionally 0 and 6, 0 and 5, 0 and 4, 0 and 3, 0 and 2 and 0 and 1 amino acid modifications. In one embodiment, the complementary determining region 3 (CDR3) is SEQ ID NO: 3. In one embodiment, the complementary determining region 3 (CDR3) is SEQ ID NO: 6. In one embodiment, the complementary determining region 3 (CDR3) is SEQ ID NO: 9. In a preferred embodiment, the complementary determining region 3 (CDR3) is SEQ ID NO: 198.

In one embodiment, the second antigen binding molecule of the invention comprises a complementary determining region 3 (CDR3) selected from the group consisting of SEQ ID NOs: 3, 6 and 9, wherein the amino acid sequences of SEQ ID NOs: 6 and 9 comprise between 0 and 7 amino acid modifications, optionally between 0 and 2 modifications; and wherein the CDR3 regions of amino acid sequences of SEQ ID NO: 3 comprises between 0 and 5 amino acid modifications, optionally between 0 and 2 amino acid modifications.

In one embodiment the antigen binding molecules of the invention may further comprise a CDR2 region. The CDR2 region may be defined according to a SEQ ID NO disclosed herein. In each embodiment, the antigen binding molecule may further comprise four framework regions (FR1, FR2, FR3 and FR4).

In one embodiment, the first antigen binding molecule comprises
(a) a CDR2 comprising SEQ ID NO:11 and a CDR3 comprising SEQ ID NO:12;
(b) a CDR2 comprising SEQ ID NO:34 and a CDR3 comprising SEQ ID NO:15;
(c) a CDR2 comprising SEQ ID NO:34 and a CDR3 comprising SEQ ID NO:72;
(d) a CDR2 comprising SEQ ID NO:34 and a CDR3 comprising SEQ ID NO:73;
(e) a CDR2 comprising SEQ ID NO:34 and a CDR3 comprising SEQ ID NO:74;
(f) a CDR2 comprising SEQ ID NO:34 and a CDR3 comprising SEQ ID NO:75;
(g) a CDR2 comprising SEQ ID NO:34 and a CDR3 comprising SEQ ID NO:76; or
(h) a CDR2 comprising SEQ ID NO:236 and a CDR3 comprising SEQ ID NO:237;
   wherein the amino acid sequence of CDR3 comprises between 0 and 7 amino acid modifications and wherein the amino acid sequence of CDR2 comprises between 0 and 4 amino acid modifications. In one embodiment the CDR3 regions comprise between 0 and 6, 0 and 5, 0 and 4, 0 and 3, 0 and 2 or 0 and 1 amino acid modifications. In one embodiment the CDR2 regions comprise between 0 and 3, 0 and 2, 0 and 4, 0 and 1 amino acid modifications.

In one embodiment, the second antigen binding molecule comprises
(a) a CDR2 comprising SEQ ID NO:197 and a CDR3 comprising SEQ ID NO:198;
(b) a CDR2 comprising SEQ ID NO:2 and a CDR3 comprising SEQ ID NO:3;
(c) a CDR2 comprising SEQ ID NO:5 and a CDR3 comprising SEQ ID NO:6; or
(d) a CDR2 comprising SEQ ID NO:8 and a CDR3 comprising SEQ ID NO:9;
   wherein the amino acid sequence of CDR3 comprises between 0 and 7 amino acid modifications and wherein the amino acid sequence of CDR2 comprises between 0 and 4 amino acid modifications. In one embodiment the CDR3 regions comprise between 0 and 6, 0 and 5, 0 and 4, 0 and 3, 0 and 2 or 0 and 1 amino acid modifications. In one embodiment the CDR2 regions comprise between 0 and 3, 0 and 2, 0 and 4, 0 and 1 amino acid modifications.

In one embodiment, the antigen binding molecule of the invention may further comprise a CDR1 region and CDR2 region. The CDR1 region and the CDR2 region may be defined according to a SEQ ID NO disclosed herein. In each embodiment, the single domain antibody may further comprise four framework regions (FR1, FR2, FR3 and FR4).

In one embodiment the, the first antigen binding molecule comprises
(a) a CDR1 comprising SEQ ID NO:10, a CDR2 comprising SEQ ID NO:11 and a CDR3 comprising SEQ ID NO:12;
(b) a CDR1 comprising SEQ ID NO:13, a CDR2 comprising SEQ ID NO:14 and a CDR3 comprising SEQ ID NO:15;
(c) CDR1 comprising SEQ ID NO:33, a CDR2 comprising SEQ ID NO:34 and a CDR3 comprising SEQ ID NO:72;
(d) CDR1 comprising SEQ ID NO:33, a CDR2 comprising SEQ ID NO:34 and a CDR3 comprising SEQ ID NO:73;
(e) CDR1 comprising SEQ ID NO:33, a CDR2 comprising SEQ ID NO:34 and a CDR3 comprising SEQ ID NO:74;
(f) CDR1 comprising SEQ ID NO:33, a CDR2 comprising SEQ ID NO:34 and a CDR3 comprising SEQ ID NO:75;
(g) CDR1 comprising SEQ ID NO:33, a CDR2 comprising SEQ ID NO:34 and a CDR3 comprising SEQ ID NO:76; or
(h) CDR1 comprising SEQ ID NO:235, a CDR2 comprising SEQ ID NO:236 and a CDR3 comprising SEQ ID NO:237;
   wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications. In one embodiment the CDR3 regions comprise between 0 and 6, 0 and 5, 0 and 4, 0 and 3, 0 and 2 or 0 and 1 amino acid modifications. In one embodiment the CDR2 regions comprise between 0 and 3, 0 and 2, 0 and 4, 0 and 1 amino acid modifications. In one embodiment the CDR1 regions comprise between 0 and 3, 0 and 2, 0 and 4, 0 and 1 amino acid modifications.

In one embodiment, the second antigen binding molecule comprises
(a) CDR1 comprising SEQ ID NO:196, a CDR2 comprising SEQ ID NO:197 and a CDR3 comprising SEQ ID NO:198;
(b) CDR1 comprising SEQ ID NO:1, a CDR2 comprising SEQ ID NO:2 and a CDR3 comprising SEQ ID NO:3;
(b) a CDR1 comprising SEQ ID NO:4, a CDR2 comprising SEQ ID NO:5 and a CDR3 comprising SEQ ID NO:6; or
(c) a CDR1 comprising SEQ ID NO:7, a CDR2 comprising SEQ ID NO:8 and a CDR3 comprising SEQ ID NO:9;
   wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications. In one embodiment the CDR3 regions comprise between 0 and 6, 0 and 5, 0 and 4, 0 and 3, 0 and 2 or 0 and 1 amino acid modifications. In one embodiment the CDR2 regions comprise between 0 and 3, 0 and 2, 0 and 4, 0 and 1 amino acid modifications. In one embodiment the CDR1 regions comprise between 0 and 3, 0 and 2, 0 and 4, 0 and 1 amino acid modifications.

In one embodiment, the second antigen binding molecule comprises the known antibody CR3022 or variant or fragment thereof. The known antibody CR3022 may comprise a heavy chain having a sequence selected from SEQ ID NO: 26 or 27 and/or a light chain having a sequence selected from SEQ ID NO: 28 or 29.

In one embodiment, the second antigen binding molecule comprises the known antibody EY6A or variant or fragment thereof. The known antibody EY6A may comprise a heavy chain having the sequence SEQ ID NO: 30 and/or comprise a light chain having a sequence SEQ ID NO: 31.

In one embodiment, the second antigen binding molecule comprises the known single domain antibody (nanobody) VHH 72 or variant or fragment thereof. VHH 72 may comprise the sequence SEQ ID NO: 32.

The heavy and/or the light chain of the known antibody may comprise one or more additional modifications, for example between 0 and 10, 0 and 9, 0 and 8, 0 and 7, 0 and 6, 0 and 5, 0 and 4, 0 and 3, 0 and 2 or 0 and 1 amino acid modifications. The modifications can be substitutions, deletions or insertions. In one embodiment, the modifications are substitutions.

In one aspect, a coronavirus binding molecule is provided comprising:
(a) a first antigen binding molecule that competes with C5, H3, H11-D4, H11-H4, H11-A10, H11-B5, H11-H6 or VHH_H6 for binding to the SARS-CoV-2 receptor-binding domain;
(b) a second antigen binding molecule that competes with A8, CR3022, VHH72 or EY6A, F2, C1 or B12 for binding to the SARS-CoV-2 receptor-binding domain; and
(c) a linker,
   wherein the linker comprises:
      (a) a ubiquitin or a ubiquitin-like protein;
      (b) further optional spacer of between 4 and 50 amino acids joined to the n-terminal of the ubiquitin or ubiquitin-like protein; and
      (c) further optional spacer of between 4 and 50 amino acids joined to the c-terminal of the ubiquitin or ubiquitin-like protein;
   and wherein the linker joins the first antigen binding molecule to the second antigen binding molecule,
   and optionally wherein the first and second epitopes are substantially non-overlapping.

In one embodiment, a coronavirus binding molecule is provided comprising:
(a) a first antigen binding molecule that competes with C5 for binding to the SARS-CoV-2 receptor-binding domain;
(b) a second antigen binding molecule that competes with CR3022 for binding to the SARS-CoV-2 receptor-binding domain; and
(c) a linker,
wherein the linker comprises:
   (a) a ubiquitin or a ubiquitin-like protein;
   (b) further optional spacer of between 4 and 50 amino acids joined to the n-terminal of the ubiquitin or ubiquitin-like protein; and
   (c) further optional spacer of between 4 and 50 amino acids joined to the c-terminal of the ubiquitin or ubiquitin-like protein;
and wherein the linker joins the first antigen binding molecule to the second antigen binding molecule,
and optionally wherein the first and second epitopes are substantially non-overlapping.

In one embodiment, a coronavirus binding molecule is provided comprising:
(a) a first antigen binding molecule that competes with CR3022 for binding to the SARS-CoV-2 receptor-binding domain;
(b) a second antigen binding molecule that competes with H11-H4 (SEQ ID NO: 120) for binding to the SARS-CoV-2 receptor-binding domain; and
(c) a linker,
wherein the linker comprises:
   (a) a ubiquitin or a ubiquitin-like protein;
   (b) further optional spacer of between 4 and 50 amino acids joined to the n-terminal of the ubiquitin or ubiquitin-like protein; and
   (c) further optional spacer of between 4 and 50 amino acids joined to the c-terminal of the ubiquitin or ubiquitin-like protein;
and wherein the linker joins the first antigen binding molecule to the second antigen binding molecule,
and optionally wherein the first and second epitopes are substantially non-overlapping.

In one embodiment, coronavirus binding molecule is provided comprising:
(a) a first antigen binding molecule comprising an amino acid sequence as disclosed herein;
(b) a second antigen binding molecule that binds to epitope 2;
(c) a linker,
wherein the linker comprises:
   (i) a ubiquitin or a ubiquitin-like protein;
   (ii) further optional spacer of between 4 and 50 amino acids joined to the n-terminal of the ubiquitin or ubiquitin-like protein; and
   (iii) further optional spacer of between 4 and 50 amino acids joined to the c-terminal of the ubiquitin or ubiquitin-like protein;
and wherein the linker joins the first antigen binding molecule to the second antigen binding molecule, and optionally wherein the first and second epitopes are substantially non-overlapping.

Additionally, coronavirus binding molecules are provided comprising:
(a) a first antigen binding molecule that binds to epitope 1;
(b) a second antigen binding molecule comprising an amino acid sequence as disclosed herein;
(c) a linker,
wherein the linker comprises:
   (i) a ubiquitin or a ubiquitin-like protein;
   (ii) further optional spacer of between 4 and 50 amino acids joined to the n-terminal of the ubiquitin or ubiquitin-like protein; and
   (iii) further optional spacer of between 4 and 50 amino acids joined to the c-terminal of the ubiquitin or ubiquitin-like protein;
and wherein the linker joins the first antigen binding molecule to the second antigen binding molecule, and optionally wherein the first and second epitopes are substantially non-overlapping.

Additionally, coronavirus binding molecules are provided comprising:
(a) a first antigen binding molecule comprising an amino acid sequence as disclosed herein;
(b) a second antigen binding molecule comprising an amino acid sequence as disclosed herein;
(c) a linker,
wherein the linker comprises:
   (i) a ubiquitin or a ubiquitin-like protein;
   (ii) further optional spacer of between 4 and 50 amino acids joined to the n-terminal of the ubiquitin or ubiquitin-like protein; and
   (iii) further optional spacer of between 4 and 50 amino acids joined to the c-terminal of the ubiquitin or ubiquitin-like protein;
and wherein the linker joins the first antigen binding molecule to the second antigen binding molecule, and optionally wherein the first and second epitopes are substantially non-overlapping.

In one embodiment, a coronavirus binding molecule is provided comprising:
(a) a first antigen binding molecule comprising a complementary determining region 3 (CDR3) selected from the group consisting of SEQ ID NOs: 12, 15, 72, 73, 74, 75, 76 and 237 wherein the CDR3 comprises between 0 and 7 amino acid modifications;
(b) a second antigen binding molecule comprising a complementary determining region 3 (CDR3) selected from the group consisting of SEQ ID NOs: 198, 3, 6 and 9, wherein the CDR3 comprises between 0 and 7 amino acid modifications; and
(c) a linker,
wherein the linker comprises:
   (i) a ubiquitin or a ubiquitin-like protein;
   (ii) further optional spacer of between 4 and 50 amino acids joined to the n-terminal of the ubiquitin or ubiquitin-like protein; and
   (iii) further optional spacer of between 4 and 50 amino acids joined to the c-terminal of the ubiquitin or ubiquitin-like protein;
and wherein the linker joins the first antigen binding molecule to the second antigen binding molecule.

In one embodiment, a coronavirus binding molecule is provided comprising:
(a) a first antigen binding molecule comprising
   (i) a CDR2 comprising SEQ ID NO:11 and a CDR3 comprising SEQ ID NO:12;
   (ii) a CDR2 comprising SEQ ID NO:14 and a CDR3 comprising SEQ ID NO:15;
   (iii) a CDR2 comprising SEQ ID NO:34 and a CDR3 comprising SEQ ID NO:72;
   (iv) a CDR2 comprising SEQ ID NO:34 and a CDR3 comprising SEQ ID NO:73;
   (v) a CDR2 comprising SEQ ID NO:34 and a CDR3 comprising SEQ ID NO:74;
   (vi) a CDR2 comprising SEQ ID NO:34 and a CDR3 comprising SEQ ID NO:75;
   (vii) a CDR2 comprising SEQ ID NO:34 and a CDR3 comprising SEQ ID NO:76; or
   (viii) a CDR2 comprising SEQ ID NO:236 and a CDR3 comprising SEQ ID NO:237;
wherein the amino acid sequence of CDR3 comprises between 0 and 7 amino acid modifications and wherein the amino acid sequence of CDR2 comprises between 0 and 4 amino acid modifications;
(b) a second binding molecule comprising
   (i) a CDR2 comprising SEQ ID NO:197 and a CDR3 comprising SEQ ID NO:198;
   (ii) a CDR2 comprising SEQ ID NO:2 and a CDR3 comprising SEQ ID NO:3;
   (iii) a CDR2 comprising SEQ ID NO:5 and a CDR3 comprising SEQ ID NO:6; or
   (iv) a CDR2 comprising SEQ ID NO:8 and a CDR3 comprising SEQ ID NO:9;
wherein the amino acid sequence of CDR3 comprises between 0 and 7 amino acid modifications and wherein the amino acid sequence of CDR2 comprises between 0 and 4 amino acid modifications; and
(c) a linker,
wherein the linker comprises:
   (i) a ubiquitin or a ubiquitin-like protein;
   (ii) further optional spacer of between 4 and 50 amino acids joined to the n-terminal of the ubiquitin or ubiquitin-like protein; and
   (iii) further optional spacer of between 4 and 50 amino acids joined to the c-terminal of the ubiquitin or ubiquitin-like protein;
and wherein the linker joins the first antigen binding molecule to the second antigen binding molecule.

In one embodiment, a coronavirus binding molecule is provided comprising:
(a) a first antigen binding molecule comprising
   (i) a CDR1 comprising SEQ ID NO:10, a CDR2 comprising SEQ ID NO:11 and a CDR3 comprising SEQ ID NO:12;
   (ii) a CDR1 comprising SEQ ID NO:13, a CDR2 comprising SEQ ID NO:14 and a CDR3 comprising SEQ ID NO:15;
   (iii) CDR1 comprising SEQ ID NO:33, a CDR2 comprising SEQ ID NO:34 and a CDR3 comprising SEQ ID NO:72;
   (iv) CDR1 comprising SEQ ID NO:33, a CDR2 comprising SEQ ID NO:34 and a CDR3 comprising SEQ ID NO:73;
   (v) CDR1 comprising SEQ ID NO:33, a CDR2 comprising SEQ ID NO:34 and a CDR3 comprising SEQ ID NO:74;
   (vi) CDR1 comprising SEQ ID NO:33, a CDR2 comprising SEQ ID NO:34 and a CDR3 comprising SEQ ID NO:75;
   (vii) CDR1 comprising SEQ ID NO:33, a CDR2 comprising SEQ ID NO:34 and a CDR3 comprising SEQ ID NO:76; or
   (viii) CDR1 comprising SEQ ID NO:235, a CDR2 comprising SEQ ID NO:236 and a CDR3 comprising SEQ ID NO:237;
wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications; and
(b) a second antigen binding molecule comprising
   (i) CDR1 comprising SEQ ID NO:196, a CDR2 comprising SEQ ID NO:197 and a CDR3 comprising SEQ ID NO:198;
   (ii) CDR1 comprising SEQ ID NO:1, a CDR2 comprising SEQ ID NO:2 and a CDR3 comprising SEQ ID NO:3;
   (iii) a CDR1 comprising SEQ ID NO:4, a CDR2 comprising SEQ ID NO:5 and a CDR3 comprising SEQ ID NO:6; CDR2 comprising SEQ ID NO:5 and a CDR3 comprising SEQ ID NO:6; or
   (iv) a CDR1 comprising SEQ ID NO:7, a CDR2 comprising SEQ ID NO:8 and a CDR3 comprising SEQ ID NO:9;
wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications; and
(c) a linker,
wherein the linker comprises:
   (i) a ubiquitin or a ubiquitin-like protein;
   (ii) further optional spacer of between 4 and 50 amino acids joined to the n-terminal of the ubiquitin or ubiquitin-like protein; and
   (iii) further optional spacer of between 4 and 50 amino acids joined to the c-terminal of the ubiquitin or ubiquitin-like protein;
and wherein the linker joins the first antigen binding molecule to the second antigen binding molecule.

In one embodiment, a coronavirus binding molecule is provided comprising:
(a) a first antigen binding molecule comprising a complementary determining region 3 (CDR3) selected from the group consisting of SEQ ID NOs: 12, 15, 72, 73, 74, 75, 76 and 237 wherein the CDR3 comprises between 0 and 7 amino acid modifications;
(b) a second antigen binding molecule comprising a complementary determining region 3 (CDR3) selected from the group consisting of SEQ ID NOs: 198, 3, 6 and 9, wherein the CDR3 comprises between 0 and 7 amino acid modifications; and
(c) a linker, wherein the linker comprises SUMO; and optionally further comprises a
   (i) spacer of between 4 and 50 amino acids joined to the n-terminal of the ubiquitin or ubiquitin-like protein; and
   (ii) spacer of between 4 and 50 amino acids joined to the c-terminal of the ubiquitin or ubiquitin-like protein;
and wherein the linker joins the first antigen binding molecule to the second antigen binding molecule.

In one embodiment, a coronavirus binding molecule is provided comprising:
(a) a first antigen binding molecule comprising
   (i) a CDR2 comprising SEQ ID NO:11 and a CDR3 comprising SEQ ID NO:12;
   (ii) a CDR2 comprising SEQ ID NO:14 and a CDR3 comprising SEQ ID NO:15;
   (iii) a CDR2 comprising SEQ ID NO:34 and a CDR3 comprising SEQ ID NO:72;
   (iii) a CDR2 comprising SEQ ID NO:34 and a CDR3 comprising SEQ ID NO:73
   (iv) a CDR2 comprising SEQ ID NO:34 and a CDR3 comprising SEQ ID NO:74;
   (v) a CDR2 comprising SEQ ID NO:34 and a CDR3 comprising SEQ ID NO:75;
   (vi) a CDR2 comprising SEQ ID NO:34 and a CDR3 comprising SEQ ID NO:76; or
   (vii) a CDR2 comprising SEQ ID NO:236 and a CDR3 comprising SEQ ID NO:236;
wherein the amino acid sequence of CDR3 comprises between 0 and 7 amino acid modifications and wherein the amino acid sequence of CDR2 comprises between 0 and 4 amino acid modifications;
(b) a second binding molecule comprising
   (i) a CDR2 comprising SEQ ID NO:197 and a CDR3 comprising SEQ ID NO:198;
   (ii) a CDR2 comprising SEQ ID NO:2 and a CDR3 comprising SEQ ID NO:3;
   (iii) a CDR2 comprising SEQ ID NO:5 and a CDR3 comprising SEQ ID NO:6; or
   (iv) a CDR2 comprising SEQ ID NO:8 and a CDR3 comprising SEQ ID NO:9;
wherein the amino acid sequence of CDR3 comprises between 0 and 7 amino acid modifications and wherein the amino acid sequence of CDR2 comprises between 0 and 4 amino acid modifications; and
(c) a linker, wherein the linker comprises SUMO; and optionally further comprises a
   (i) spacer of between 4 and 50 amino acids joined to the n-terminal of the ubiquitin or ubiquitin-like protein; and
   (ii) spacer of between 4 and 50 amino acids joined to the c-terminal of the ubiquitin or ubiquitin-like protein;
and wherein the linker joins the first antigen binding molecule to the second antigen binding molecule.

In one embodiment, a coronavirus binding molecule is provided comprising:
(a) a first antigen binding molecule comprising
   (i) a CDR1 comprising SEQ ID NO:10, a CDR2 comprising SEQ ID NO:11 and a CDR3 comprising SEQ ID NO:12;
   (ii) a CDR1 comprising SEQ ID NO:13, a CDR2 comprising SEQ ID NO:14 and a CDR3 comprising SEQ ID NO:15;
   (iii) CDR1 comprising SEQ ID NO:33, a CDR2 comprising SEQ ID NO:34 and a CDR3 comprising SEQ ID NO:72;
   (iii) CDR1 comprising SEQ ID NO:33, a CDR2 comprising SEQ ID NO:34 and a CDR3 comprising SEQ ID NO:73;
   (iv) CDR1 comprising SEQ ID NO:33, a CDR2 comprising SEQ ID NO:34 and a CDR3 comprising SEQ ID NO:74;
   (v) CDR1 comprising SEQ ID NO:33, a CDR2 comprising SEQ ID NO:34 and a CDR3 comprising SEQ ID NO:75;
   (vi) CDR1 comprising SEQ ID NO:33, a CDR2 comprising SEQ ID NO:34 and a CDR3 comprising SEQ ID NO:76; or
   (vii) CDR1 comprising SEQ ID NO:235, a CDR2 comprising SEQ ID NO:236 and a CDR3 comprising SEQ ID NO:237;
wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications; and
(b) a second antigen binding molecule comprising
   (i) CDR1 comprising SEQ ID NO:196, a CDR2 comprising SEQ ID NO:197 and a CDR3 comprising SEQ ID NO:198;
   (ii) CDR1 comprising SEQ ID NO:1, a CDR2 comprising SEQ ID NO:2 and a CDR3 comprising SEQ ID NO:3;
   (iii) a CDR1 comprising SEQ ID NO:4, a CDR2 comprising SEQ ID NO:5 and a CDR3 comprising SEQ ID NO:6; CDR2 comprising SEQ ID NO:5 and a CDR3 comprising SEQ ID NO:6; or
   (iv) a CDR1 comprising SEQ ID NO:7, a CDR2 comprising SEQ ID NO:8 and a CDR3 comprising SEQ ID NO:9;
wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications; and
(c) a linker, wherein the linker comprises SUMO; and optionally further comprises a
   (i) spacer of between 4 and 50 amino acids joined to the n-terminal of the ubiquitin or ubiquitin-like protein; and
   (ii) spacer of between 4 and 50 amino acids joined to the c-terminal of the ubiquitin or ubiquitin-like protein;
and wherein the linker joins the first antigen binding molecule to the second antigen binding molecule.

In one embodiment, a coronavirus binding molecule comprising:
(a) a first antigen binding molecule comprising a complementary determining region 3 (CDR3) selected from the group consisting of SEQ ID NOs: 12, 15, 72, 73, 74, 75, 76 and 237 wherein the CDR3 comprises between 0 and 7 amino acid modifications;
(b) a second antigen binding molecule comprising a complementary determining region 3 (CDR3) selected from the group consisting of SEQ ID NOs: 198, 3, 6 and 9, wherein the CDR3 comprises between 0 and 7 amino acid modifications; and
(c) a linker, wherein the linker comprises SUMO;
between 4 and 8 amino acids joined to the n-terminal of SUMO and
between 4 and 8 amino acids joined to the c-terminal of SUMO;
and wherein the linker joins the first antigen binding molecule to the second antigen binding molecule is provided.

In one embodiment, a coronavirus binding molecule comprising:
(a) a first antigen binding molecule comprising
   (i) a CDR2 comprising SEQ ID NO:11 and a CDR3 comprising SEQ ID NO:12;
   (ii) a CDR2 comprising SEQ ID NO:14 and a CDR3 comprising SEQ ID NO:15;
   (iii) a CDR2 comprising SEQ ID NO:34 and a CDR3 comprising SEQ ID NO:72;
   (iv) a CDR2 comprising SEQ ID NO:34 and a CDR3 comprising SEQ ID NO:73;
   (v) a CDR2 comprising SEQ ID NO:34 and a CDR3 comprising SEQ ID NO:74;
   (vi) a CDR2 comprising SEQ ID NO:34 and a CDR3 comprising SEQ ID NO:75;
   (vii) a CDR2 comprising SEQ ID NO:34 and a CDR3 comprising SEQ ID NO:76; or
   (viii) a CDR2 comprising SEQ ID NO:236 and a CDR3 comprising SEQ ID NO:237;
wherein the amino acid sequence of CDR3 comprises between 0 and 7 amino acid modifications and wherein the amino acid sequence of CDR2 comprises between 0 and 4 amino acid modifications;
(b) a second antigen binding molecule comprising
   (i) a CDR2 comprising SEQ ID NO:197 and a CDR3 comprising SEQ ID NO:198;
   (ii) a CDR2 comprising SEQ ID NO:2 and a CDR3 comprising SEQ ID NO:3;
   (iii) a CDR2 comprising SEQ ID NO:5 and a CDR3 comprising SEQ ID NO:6; or
   (iv) a CDR2 comprising SEQ ID NO:8 and a CDR3 comprising SEQ ID NO:9;
wherein the amino acid sequence of CDR3 comprises between 0 and 7 amino acid modifications and wherein the amino acid sequence of CDR2 comprises between 0 and 4 amino acid modifications; and
(c) a linker, wherein the linker comprises SUMO;
between 4 and 8 amino acids joined to the n-terminal of SUMO and between 4 and 8 amino acids joined to the c-terminal of SUMO;
and wherein the linker joins the first antigen binding molecule to the second antigen binding molecule is provided.

In one embodiment, a coronavirus binding molecule comprising:
(a) a first antigen binding molecule comprising
   (i) a CDR1 comprising SEQ ID NO:10, a CDR2 comprising SEQ ID NO:11 and a CDR3 comprising SEQ ID NO:12;
   (ii) a CDR1 comprising SEQ ID NO:13, a CDR2 comprising SEQ ID NO:14 and a CDR3 comprising SEQ ID NO:15;
   (iii) CDR1 comprising SEQ ID NO:33, a CDR2 comprising SEQ ID NO:34 and a CDR3 comprising SEQ ID NO:72; or
   (iv) CDR1 comprising SEQ ID NO:33, a CDR2 comprising SEQ ID NO:34 and a CDR3 comprising SEQ ID NO:73;
   (v) CDR1 comprising SEQ ID NO:33, a CDR2 comprising SEQ ID NO:34 and a CDR3 comprising SEQ ID NO:74;
   (vi) CDR1 comprising SEQ ID NO:33, a CDR2 comprising SEQ ID NO:34 and a CDR3 comprising SEQ ID NO:75;
   (vii) CDR1 comprising SEQ ID NO:33, a CDR2 comprising SEQ ID NO:34 and a CDR3 comprising SEQ ID NO:76; or
   (viii) CDR1 comprising SEQ ID NO:235, a CDR2 comprising SEQ ID NO:236 and a CDR3 comprising SEQ ID NO:237;
wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications; and
(b) a second antigen binding molecule comprising
   (i) CDR1 comprising SEQ ID NO:196, a CDR2 comprising SEQ ID NO:197 and a CDR3 comprising SEQ ID NO:198;
   (ii) CDR1 comprising SEQ ID NO:1, a CDR2 comprising SEQ ID NO:2 and a CDR3 comprising SEQ ID NO:3;
   (iii) a CDR1 comprising SEQ ID NO:4, a CDR2 comprising SEQ ID NO:5 and a CDR3 comprising SEQ ID NO:6; CDR2 comprising SEQ ID NO:5 and a CDR3 comprising SEQ ID NO:6; or
   (iv) a CDR1 comprising SEQ ID NO:7, a CDR2 comprising SEQ ID NO:8 and a CDR3 comprising SEQ ID NO:9;
wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications; and
(c) a linker, wherein the linker comprises SUMO;
between 4 and 8 amino acids joined to the n-terminal of SUMO and
between 4 and 8 amino acids joined to the c-terminal of SUMO;
and wherein the linker joins the first antigen binding molecule to the second antigen binding molecule is provided.

In one embodiment, a coronavirus binding molecule comprising:
(a) a first antigen binding molecule comprising a complementary determining region 3 (CDR3) selected from the group consisting of SEQ ID NOs: 12, 15, 72, 73, 74, 75, 76 and 237;
(b) a second antigen binding molecule comprising a complementary determining region 3 (CDR3) selected from the group consisting of SEQ ID NOs: 198, 3, 6 and 9; and
(c) a linker, wherein the linker comprises SUMO;
between 4 and 8 amino acids joined to the n-terminal of SUMO and
between 4 and 8 amino acids joined to the c-terminal of SUMO;
and wherein the linker joins the first antigen binding molecule to the second antigen binding molecule is provided.

In one embodiment, a coronavirus binding molecule comprising:
(a) a first antigen binding molecule comprising
   (i) a CDR2 comprising SEQ ID NO:11 and a CDR3 comprising SEQ ID NO:12;
   (ii) a CDR2 comprising SEQ ID NO:14 and a CDR3 comprising SEQ ID NO:15;
   (iii) a CDR2 comprising SEQ ID NO:34 and a CDR3 comprising SEQ ID NO:72;
   (iv) a CDR2 comprising SEQ ID NO:34 and a CDR3 comprising SEQ ID NO:73;
   (v) a CDR2 comprising SEQ ID NO:34 and a CDR3 comprising SEQ ID NO:74;
   (vi) a CDR2 comprising SEQ ID NO:34 and a CDR3 comprising SEQ ID NO:75; or
   (vii) a CDR2 comprising SEQ ID NO:34 and a CDR3 comprising SEQ ID NO:76;
   (viii) a CDR2 comprising SEQ ID NO:236 and a CDR3 comprising SEQ ID NO:237; and
(b) a second binding molecule comprising
   (i) a CDR2 comprising SEQ ID NO:197 and a CDR3 comprising SEQ ID NO:198;
   (i) a CDR2 comprising SEQ ID NO:2 and a CDR3 comprising SEQ ID NO:3;
   (ii) a CDR2 comprising SEQ ID NO:5 and a CDR3 comprising SEQ ID NO:6; or
   (iii) a CDR2 comprising SEQ ID NO:8 and a CDR3 comprising SEQ ID NO:9; and
(c) a linker, wherein the linker comprises SUMO;
between 4 and 8 amino acids joined to the n-terminal of SUMO and
between 4 and 8 amino acids joined to the c-terminal of SUMO;
and wherein the linker joins the first antigen binding molecule to the second antigen binding molecule is provided.

In one embodiment, a coronavirus binding molecule comprising:
(a) a first antigen binding molecule comprising
   (i) a CDR1 comprising SEQ ID NO:10, a CDR2 comprising SEQ ID NO:11 and a CDR3 comprising SEQ ID NO:12;
   (ii) a CDR1 comprising SEQ ID NO:13, a CDR2 comprising SEQ ID NO:14 and a CDR3 comprising SEQ ID NO:15;
   (iii) CDR1 comprising SEQ ID NO:33, a CDR2 comprising SEQ ID NO:34 and a CDR3 comprising SEQ ID NO:72;
   (iv) CDR1 comprising SEQ ID NO:33, a CDR2 comprising SEQ ID NO:34 and a CDR3 comprising SEQ ID NO:73; and
   (v) CDR1 comprising SEQ ID NO:33, a CDR2 comprising SEQ ID NO:34 and a CDR3 comprising SEQ ID NO:74;
   (vi) CDR1 comprising SEQ ID NO:33, a CDR2 comprising SEQ ID NO:34 and a CDR3 comprising SEQ ID NO:75;
   (vii) CDR1 comprising SEQ ID NO:33, a CDR2 comprising SEQ ID NO:34 and a CDR3 comprising SEQ ID NO:76; or
   (viii) CDR1 comprising SEQ ID NO:235, a CDR2 comprising SEQ ID NO:236 and a CDR3 comprising SEQ ID NO:237;
(b) a second antigen binding molecule comprising
   (i) CDR1 comprising SEQ ID NO:196, a CDR2 comprising SEQ ID NO:197 and a CDR3 comprising SEQ ID NO:198;
   (ii) CDR1 comprising SEQ ID NO:1, a CDR2 comprising SEQ ID NO:2 and a CDR3 comprising SEQ ID NO:3;
   (ii) a CDR1 comprising SEQ ID NO:4, a CDR2 comprising SEQ ID NO:5 and a CDR3 comprising SEQ ID NO:6; CDR2 comprising SEQ ID NO:5 and a CDR3 comprising SEQ ID NO:6; or
   (iii) a CDR1 comprising SEQ ID NO:7, a CDR2 comprising SEQ ID NO:8 and a CDR3 comprising SEQ ID NO:9; and
(c) a linker, wherein the linker comprises SUMO;
between 4 and 8 amino acids joined to the n-terminal of SUMO and
between 4 and 8 amino acids joined to the c-terminal of SUMO;
and wherein the linker joins the first antigen binding molecule to the second antigen binding molecule is provided.

In one aspect, an amino acid sequence is provided comprising the first and second antigen binding molecules of the invention.

In one embodiment, a first antigen binding molecule comprising an amino acid sequence having at least 70% identity to a sequence selected from the group consisting of: SEQ ID NO: 19, 20, 119, 120, 121, 122, 123 and 239 is provided. Each of these sequences comprises three CDR regions (CDR1, CDR2 and CDR3) and four framework regions (FR1, FR2, FR3 and FR4). In one embodiment, the amino acid sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity to a sequence selected from the group consisting of: SEQ ID NO: 19, 20, 119, 120, 121, 122 and 123. In one embodiment, a first antigen binding domain comprising an amino acid sequence selected from the group consisting of SEQ ID NO: 19, 20, 119, 120, 121, 122, 123 and 239 is provided. In one embodiment, a first antigen binding domain consisting or essentially consisting of comprising an amino acid sequence selected from the group consisting of SEQ ID NO: 19, 20, 119, 120, 121, 122 and 123 is provided.

In one embodiment, a second antigen binding molecule comprising an amino acid sequence having at least 70% identity to a sequence selected from the group consisting of: SEQ ID NO: 213, 16, 17 and 18 is provided. Each of these sequences comprises three CDR regions (CDR1, CDR2 and CDR3) and four framework regions (FR1, FR2, FR3 and FR4). In one embodiment, the amino acid sequence has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity to a sequence selected from the group consisting of: SEQ ID NO: 213, 16, 17 and 18. In one embodiment, a second antigen binding molecule comprising a sequence selected from the group consisting of SEQ ID NO: 213, 16, 17 and 18 is provided. In one embodiment, a second antigen binding molecule consisting or essentially consisting a sequence selected from the group consisting of SEQ ID NO: 213, 16, 17 and 18 is provided.

In one embodiment, the first antigen binding molecule comprises a polynucleotide sequence having at least 70% identity to a sequence selected from the group consisting of: SEQ ID NO: 24, 25, 140, 141, 142, 143, 144 and 238 is provided. In one embodiment, the polynucleotide sequence has at least 75%, 80%, 85%, 90%, 91%, 92%, 93%, 94%, 95%, 96%, 97%, 98% or at least 99% identity to a sequence selected from the group consisting of: SEQ ID NO: 24, 25, 140, 141, 142, 143, 144 and 238. In one embodiment, the first antigen binding molecule comprises a sequence selected from the group consisting of SEQ ID NO: 24, 25, 140, 141, 142, 143, 144 and 238. In one embodiment, the first antigen binding molecule consists or essentially consists of a sequence selected from the group consisting of SEQ ID N024, 25, 140, 141, 142, 143, 144 and 238.

In one embodiment, the second antigen binding molecule comprises a polynucleotide sequence having at least 70% identity to a sequence selected from the group consisting of: SEQ ID NO: 212, 21, 22 and 23. In one embodiment, the polynucleotide sequence has at least 75%, 80%, 85%, 90%, 91%, 92%, 93%, 94%, 95%, 96%, 97%, 98% or at least 99% identity to a sequence selected from the group consisting of: SEQ ID NO: 212, 21, 22 and 23. In one embodiment, the second antigen binding molecule comprises a sequence selected from the group consisting of SEQ ID NO: 212, 21, 22 and 23. In one embodiment, the second antigen binding molecule consists or essentially consists of SEQ ID NO: 212, 21, 22 or 23.

In one aspect, a multivalent polypeptide comprising one or optionally two or more of the single domain antibodies of the invention is provided.

In one embodiment, a multivalent polypeptide comprising one or more single domain antibodies of the invention and one or more additional known antibodies or single domain antibodies is provided. In this embodiment, the single domain antibodies of the invention are joined or linked together with additional known antibodies or single domain antibodies to form a multivalent polypeptide. In one embodiment, a first and/or a second antigen binding molecule comprises a known antibody.

In one embodiment, the known antibody is CR3022 or EY6A or a variant thereof. The known antibody CR3022 may comprise a heavy chain having a sequence selected from SEQ ID NO: 26 or 27. The known antibody CR3022 may additionally comprise a light chain having a sequence selected from SEQ ID NO: 28 or 29.

The heavy and/or the light chain of the CR3022 may comprise one or more additional modifications, for example between 0 and 10, 0 and 9, 0 and 8, 0 and 7, 0 and 6, 0 and 5, 0 and 4, 0 and 3, 0 and 2 or 0 and 1 amino acid modifications. The modifications can be substitutions, deletions or insertions. In one embodiment, the modifications are substitutions.

In one embodiment, the known antibody EY6A may comprise a heavy chain having the sequence of SEQ ID NO: 30 or 31.

The heavy and/or the light chain of the EY6A may comprise one or more additional modifications, for example between 0 and 10, 0 and 9, 0 and 8, 0 and 7, 0 and 6, 0 and 5, 0 and 4, 0 and 3, 0 and 2 or 0 and 1 amino acid modifications. The modifications can be substitutions, deletions or insertions. In one embodiment, the modifications are substitutions.

In one embodiment, the known single domain antibody (nanobody) is VHH-72 has the sequence of SEQ ID NO: 32.

The sequence of EY6A may comprise one or more additional modifications, for example between 0 and 10, 0 and 9, 0 and 8, 0 and 7, 0 and 6, 0 and 5, 0 and 4, 0 and 3, 0 and 2 or 0 and 1 amino acid modifications. The modifications can be substitutions, deletions or insertions. In one embodiment, the modifications are substitutions.

In one embodiment, the known antibody is selected from the group consisting of H11, A7, F9, C10, B11, E11, D1, G7, F5, G11, B4, G9 and C7, H11-D4, H11-H4, H11-H6, H11-A10, H11-B5, H11-A7, H11-F7, H11-F6, H11-G8, H11-D1, H11-A9, H11-C6, H11-E3, H11-F4, H11-C5, H11-C2, H11-B11, H11-A3, H11-D12, H11-D6 and H11-F8 (amino acid sequences provided as SEQ ID NOs: 93-105, polynucleotide sequences provided as SEQ ID NOs: 106-118 respectively) or a fragment or variant thereof.

In one embodiment, the known antibody is an affinity matured version of H11 selected from the group consisting of H11-D4, H11-H4, H11-H6, H11-A10, H11-B5, H11-A7, H11-F7, H11-F6, H11-G8, H11-D1, H11-A9, H11-C6, H11-E3, H11-F4, H11-C5, _H11-C2, H11-B11, H11-A3, H11-D12, H11-D6 and H11-F8 (amino acid sequences provided as SEQ ID NOs: 119-139, polynucleotide sequences provided as SEQ ID NOs: 140-160 respectively) or a fragment or variant thereof.

In one embodiment, the known single domain antibody comprises or further comprises a complementary determining region, complementary determining region 3 (CDR3), selected from Table 7 or Table 8. In one embodiment, the known single domain antibody comprises a complementary determining region selected from CDR1, complementary determining region 2 (CDR2) or complementary determining region 3 (CDR3), wherein the CDR1, CDR2 or CDR3 is selected from Table 7 or 8. In one embodiment, the known single domain antibody comprises at least one or at least two complementary determining region(s) selected from CDR1, CDR2 or CDR3 is provided, wherein the CDR1, CDR2 or CDR3 is selected from Table 7 or 8. In one embodiment, the known single domain antibody comprises three complementary determining regions: CDR1, CDR2, and CDR3, wherein the CDR1, CDR2 and CDR3 is selected from Table 7 or 8.

TABLE 7
CDR alignments of primary hits H11, A7, F9,
C10, B11, E11, D1, G7, F5,G11, B4, G9 and
C7, H11-D4, H11-H4, H11-H6, H11-A10, H11-B5,
H11-A7, H11-F7, H11-F6, H11-G8, H11-D1, H11-
A9, H11-C6, H11-E3, H11-F4, H11-C5, H11-C2,
H11-B11, H11-A3, H11-D12, H11-D6 and H11-F8

| | | SEQ | | SEQ | | SEQ |
|---|---|---|---|---|---|---|
| | | ID | | ID | | ID |
| ID | CDR1 | NO | CDR2 | NO | CDR3 | NO |
| H11 | GRTFSTAA | 33 | IRWSGGSA | 34 | AQTRVTRSLLS | 35 |
| | | | | | DYATWPYDY | |
| A7 | GFTFDDYA | 36 | ISWNGGGT | 37 | AKDFHRYGSST | 38 |
| | | | | | LEYDY | |
| F9 | GFTFSSYF | 39 | IGDRGNT | 40 | YSRNRILQHY | 41 |
| C10 | GFAFSSYD | 42 | IDSGGGVT | 43 | NAVDGLGYLEYDY | 44 |
| B11 | GFTFDDYA | 45 | IYSYISTT | 46 | AARRLDTTDYKY | 47 |
| E11 | GFTFSNYA | 48 | IGSDGRHP | 49 | LPGWSTVGTFLDA | 50 |
| D1 | GFTFSSYG | 51 | INSGGGST | 52 | AKDASEDAGRL | 53 |
| | | | | | YWTDY | |
| G7 | GFTFDTYD | 54 | EDSTGNK | 55 | AKGLRSWGA | 56 |
| F5 | GTIFRINA | 57 | ISNGDSGD | 58 | YCNVEASWPHKY | 59 |
| G11 | GFTFDDYG | 60 | VNSGGGT | 61 | AKRDGSWWGYTTDY | 62 |
| B4 | GFTFSSYW | 63 | INTGGDTT | 64 | ARTGVVRGPGDLDA | 65 |
| G9 | GFTFYDYH | 66 | IDTGGGRT | 67 | VRDGDARGPDYDY | 68 |
| C7 | GFTFSSYD | 69 | ISGSDSTT | 70 | ARPLGQYTS | 71 |

+0 ABLE 8
CDR alignments of affinity-matured hits H11-D4,
H11-H4, H11-H6, H11-A10, H11-B5, H11-A7, H11-F7,
H11-F6, H11-G8, H11-D1, H11-A9, H11-C6, H11-E3,
H11-F4, H11-C5, H11-C2, H11-B11, H11-A3, H11-D12,
H11-D6 and H11-F8

| | SEQ | | SEQ | | SEQ | | SEQ |
|---|---|---|---|---|---|---|---|
| Clone_ | ID | | ID | | ID | | ID |
| ID | NO | CDR1 | NO | CDR2 | NO | CD3 | NO |
| H11 | 33 | GRTFSTAA | 33 | IRWSGGSA | 34 | AQTRVTRSLL | 35 |
| (parent) | | | | | | SDYATWPYDY | |
| H11-D4 | 33 | GRTFSTAA | 33 | IRWSGGSA | 34 | ARTENVRSLL | 72 |
| | | | | | | SDYATWPYDY | |
| H11-H4 | 33 | GRTFSTAA | 33 | IRWSGGSA | 34 | AQTHYVSYLL | 73 |
| | | | | | | SDYATWPYDY | |
| H11-H6 | 33 | GRTFSTAA | 33 | IRWSGGSA | 34 | AGSKITRSLL | 74 |
| | | | | | | SDYATWPYDY | |
| H11-A10 | 33 | GRTENTAA | 33 | IMWSGGSA | 34 | AGFSATRSLL | 75 |
| | | | | | | SDYATWPYDY | |
| H11-B5 | 33 | GRTFSTAA | 33 | IRWSGGSA | 34 | ASYQATRSLL | 76 |
| | | | | | | SDYATWPYDY | |
| H11-A7 | 33 | GRTFSTAA | 33 | IRWSGGSA | 34 | AQTDNVRALL | 77 |
| | | | | | | SDYATWPYDY | |
| H11-F7 | 33 | GRTFSTAA | 33 | IRWSGGSA | 34 | AIFSNVRSLL | 78 |
| | | | | | | SDYATWPYDY | |
| H11-F6 | 33 | GRTFSTAA | 33 | IRWSGGSA | 34 | ARTSNVRSLL | 79 |
| | | | | | | SDYATWPYDY | |
| H11-G8 | 33 | GRTFSTAA | 33 | IRWSGGSA | 34 | AGSGVTRSLL | 80 |
| | | | | | | SDYATWPYDY | |
| H11-D1 | 33 | GRTFSTAA | 33 | IRWSGGSA | 34 | AGWRATRSLL | 81 |
| | | | | | | SDYATWPYDY | |
| H11-A9 | 33 | GRTFSTAA | 33 | IRWSGGSA | 34 | APYEATRSLL | 82 |
| | | | | | | SDYATWPYDY | |
| H11-C6 | 33 | GRTESTAA | 33 | IRWSGGSA | 34 | AQTGYTSWLL | 83 |
| | | | | | | SDYATWPYDY | |
| H11-E3 | 33 | GRTFSTAA | 33 | IRWSGGSA | 34 | AVYHATRSLL | 84 |
| | | | | | | SDYATWPYDY | |
| H11-F4 | 33 | GRTFSTAA | 33 | IRWSGGSA | 34 | AESTITRSLL | 85 |
| | | | | | | SDYATWPYDY | |
| H11-C5 | 33 | GRTFSTAA | 33 | IRWSGGSA | 34 | AQTSYVSFLL | 86 |
| | | | | | | SDYATWPYDY | |
| H11-C2 | 33 | GRTFSTAA | 33 | IRWSGGSA | 34 | AGSRATRSLL | 87 |
| | | | | | | SDYATWPYDY | |
| H11-B11 | 33 | GRTFSTAA | 33 | IRWSGGSA | 34 | AETLNTRSLL | 88 |
| | | | | | | SDYATWPYDY | |
| H11-A3 | 33 | GRTFSTAA | 33 | IRWSGGSA | 34 | ARSDNVRSLL | 89 |
| | | | | | | SDYATWPYDY | |
| H11-D12 | 33 | GRTFSTAA | 33 | IRWSGGSA | 34 | ADWGVTRSLL | 90 |
| | | | | | | SDYATWPYDY | |
| H11-D6 | 33 | GRTFSTAA | 33 | IRWSGGSA | 34 | ASSSVTRSLL | 91 |
| | | | | | | SDYATWPYDY | |
| H11-F8 | 33 | GRTFSTAA | 33 | IRWSGGSA | 34 | ADASATRSLL | 92 |
| | | | | | | SDYATWPYDY | |

In one embodiment, the known single domain antibody comprises:
(a) a CDR1 comprising SEQ ID NO:33, a CDR2 comprising SEQ ID NO:34 and a CDR3 comprising SEQ ID NO:35;
(b) a CDR1 comprising SEQ ID NO:36, a CDR2 comprising SEQ ID NO:37 and a CDR3 comprising SEQ ID NO:38;
(c) a CDR1 comprising SEQ ID NO:39, a CDR2 comprising SEQ ID NO:40 and a CDR3 comprising SEQ ID NO:41;
(d) a CDR1 comprising SEQ ID NO:42, a CDR2 comprising SEQ ID NO:43 and a CDR3 comprising SEQ ID NO:44;
(e) a CDR1 comprising SEQ ID NO:45, a CDR2 comprising SEQ ID NO:46 and a CDR3 comprising SEQ ID NO:47;
(f) a CDR1 comprising SEQ ID NO:48, a CDR2 comprising SEQ ID NO:49 and a CDR3 comprising SEQ ID NO:50;
(g) a CDR1 comprising SEQ ID NO:51, a CDR2 comprising SEQ ID NO:52 and a CDR3 comprising SEQ ID NO:53;
(h) a CDR1 comprising SEQ ID NO:54, a CDR2 comprising SEQ ID NO:55 and a CDR3 comprising SEQ ID NO:56;
(i) a CDR1 comprising SEQ ID NO:56, a CDR2 comprising SEQ ID NO:58 and a CDR3 comprising SEQ ID NO:59;
(j) a CDR1 comprising SEQ ID NO:60, a CDR2 comprising SEQ ID NO:61 and a CDR3 comprising SEQ ID NO:62;
(k) a CDR1 comprising SEQ ID NO:63, a CDR2 comprising SEQ ID NO:64 and a CDR3 comprising SEQ ID NO:65;
(l) a CDR1 comprising SEQ ID NO:66, a CDR2 comprising SEQ ID NO:67 and a CDR3 comprising SEQ ID NO:68;
(m) a CDR1 comprising SEQ ID NO:69, a CDR2 comprising SEQ ID NO:70 and a CDR3 comprising SEQ ID NO:71;
(n) CDR1 comprising SEQ ID NO:33, a CDR2 comprising SEQ ID NO:34 and a CDR3 comprising SEQ ID NO:72;
(o) CDR1 comprising SEQ ID NO:33, a CDR2 comprising SEQ ID NO:34 and a CDR3 comprising SEQ ID NO:73;
(p) CDR1 comprising SEQ ID NO:33, a CDR2 comprising SEQ ID NO:34 and a CDR3 comprising SEQ ID NO:74;
(q) CDR1 comprising SEQ ID NO:33, a CDR2 comprising SEQ ID NO:34 and a CDR3 comprising SEQ ID NO:75;
(r) CDR1 comprising SEQ ID NO:33, a CDR2 comprising SEQ ID NO:34 and a CDR3 comprising SEQ ID NO:76;
(s) CDR1 comprising SEQ ID NO:33, a CDR2 comprising SEQ ID NO:34 and a CDR3 comprising SEQ ID NO:77;
(t) CDR1 comprising SEQ ID NO:33, a CDR2 comprising SEQ ID NO:34 and a CDR3 comprising SEQ ID NO:78;
(u) CDR1 comprising SEQ ID NO:33, a CDR2 comprising SEQ ID NO:34 and a CDR3 comprising SEQ ID NO:79;
(v) CDR1 comprising SEQ ID NO:33, a CDR2 comprising SEQ ID NO:34 and a CDR3 comprising SEQ ID NO:80;
(w) CDR1 comprising SEQ ID NO:33, a CDR2 comprising SEQ ID NO:34 and a CDR3 comprising SEQ ID NO:81;
(x) CDR1 comprising SEQ ID NO:33, a CDR2 comprising SEQ ID NO:34 and a CDR3 comprising SEQ ID NO:82;
(y) CDR1 comprising SEQ ID NO:33, a CDR2 comprising SEQ ID NO:34 and a CDR3 comprising SEQ ID NO:83;
(z) CDR1 comprising SEQ ID NO:33, a CDR2 comprising SEQ ID NO:34 and a CDR3 comprising SEQ ID NO:84;
(aa) CDR1 comprising SEQ ID NO:33, a CDR2 comprising SEQ ID NO:34 and a CDR3 comprising SEQ ID NO:85;
(bb) CDR1 comprising SEQ ID NO:33, a CDR2 comprising SEQ ID NO:34 and a CDR3 comprising SEQ ID NO:86;
(cc) CDR1 comprising SEQ ID NO:33, a CDR2 comprising SEQ ID NO:34 and a CDR3 comprising SEQ ID NO:87;
(dd) CDR1 comprising SEQ ID NO:33, a CDR2 comprising SEQ ID NO:34 and a CDR3 comprising SEQ ID NO:56;
(ee) CDR1 comprising SEQ ID NO:33, a CDR2 comprising SEQ ID NO:34 and a CDR3 comprising SEQ ID NO:88;
(ff) CDR1 comprising SEQ ID NO:33, a CDR2 comprising SEQ ID NO:34 and a CDR3 comprising SEQ ID NO:89;
(gg) CDR1 comprising SEQ ID NO:33, a CDR2 comprising SEQ ID NO:34 and a CDR3 comprising SEQ ID NO:90; or
(hh) CDR1 comprising SEQ ID NO:33, a CDR2 comprising SEQ ID NO:34 and a CDR3 comprising SEQ ID NO:91;
   wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications. In a preferred embodiment, the known single domain antibody comprises;
(a) CDR1 comprising SEQ ID NO:33, a CDR2 comprising SEQ ID NO:34 and a CDR3 comprising SEQ ID NO:73; or
(b) CDR1 comprising SEQ ID NO:33, a CDR2 comprising SEQ ID NO:34 and a CDR3 comprising SEQ ID NO:74.

In one embodiment the CDR3 regions comprise between 0 and 6, 0 and 5, 0 and 4, 0 and 3, 0 and 2 or 0 and 1 amino acid modifications. In one embodiment the CDR2 regions comprise between 0 and 3, 0 and 2, 0 and 4, 0 and 1 amino acid modifications. In one embodiment the CDR1 regions comprise between 0 and 3, 0 and 2, 0 and 4, 0 and 1 amino acid modifications.

In one embodiment, the known single domain antibody comprises an amino acid sequence having at least 70% identity to a sequence selected from the group consisting of: SEQ ID NO: 93 to 105. In one embodiment, known single domain antibody comprises an amino acid sequence having at least 70% identity to a sequence selected from the group consisting of: SEQ ID NO: 119 to 139. Each of these sequences comprises three CDR regions (CDR1, CDR2 and CDR3) and four framework regions (FR1, FR2, FR3 and FR4). In one embodiment, the known single domain antibody has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity to a sequence selected from the group consisting of: SEQ ID NO: 93 to 105. In one embodiment, the known single domain antibody has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity to a sequence selected from the group consisting of: SEQ ID NO: 119 to 139. At least herein and throughout means, in some embodiments, the recited percentage up to 100%. For example, at least 75% can mean, in some embodiments, 75% to 100%.

In one embodiment, the known single domain antibody comprises an amino acid sequence selected from the group consisting of SEQ ID NO: 93 to 105. In one embodiment, the known single domain antibody comprises an amino acid sequence selected from the group consisting of SEQ ID NO: 119 to 139. In one embodiment, the known single domain antibody consists or essentially consists of a sequence selected from the group SEQ ID NO: 93 to 105. In one embodiment, the known single domain antibody consists or essentially consists of a sequence selected from the group SEQ ID NO: 119 to 139.

In one embodiment, the known single domain antibody comprises a polynucleotide sequence having at least 70% identity to a sequence selected from the group consisting of: SEQ ID NO: 106 to 118. In one embodiment, known single domain antibody comprises a polynucleotide sequence having at least 70% identity to a sequence selected from the group consisting of: SEQ ID NO: 140 to 160. Each of these sequences comprises three CDR regions (CDR1, CDR2 and CDR3) and four framework regions (FR1, FR2, FR3 and FR4). In one embodiment, the known single domain antibody has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity to a sequence selected from the group consisting of: SEQ ID NO: 106 to 118. In one embodiment, the known single domain antibody has at least 75%, 80%, 85%, 90%, 91%, 92%, 93%, 94%, 95%, 96%, 97%, 98% or at least 99% identity, optionally 75-100%, 80-100%, 85-100%, 90-100%, 91-100%, 92-100%, 93-100%, 94-100%, 95-100%, 96-100%, 97-100%, 98-100% identity to a sequence selected from the group consisting of: SEQ ID NO: 140 to 160.

In one embodiment, the known single domain antibody comprises a polynucleotide sequence selected from the group consisting of SEQ ID NO: 106 to 118. In one embodiment, the known single domain antibody comprises a polynucleotide sequence selected from the group consisting of SEQ ID NO: 140 to 160. In one embodiment, the known single domain antibody consists or essentially consists of a sequence selected from the group SEQ ID NO: 106 to 118. In one embodiment, the known single domain antibody consists or essentially consists of a sequence selected from the group SEQ ID NO: 140 to 160. At least herein and throughout means, in some embodiments, the recited percentage up to 100%. For example, at least 75% can mean, in some embodiments, 75% to 100%.

The multivalent polypeptide may be bivalent, optionally monospecific bivalent or bispecific bivalent. In one embodiment, the bivalent polypeptide may comprise one single domain antibody of the invention and one known antibody or nanobody. In one embodiment, the bispecific bivalent polypeptide may additionally be biparatopic, i.e. it recognizes two distinct non-overlapping epitopes on the same target antigen.

The multivalent polypeptide may be trivalent, optionally monospecific trivalent, bispecific trivalent or trispecific trivalent. In one embodiment, the trivalent polypeptide may comprise two single domain antibodies of the invention and one known antibody or nanobody or, alternatively, comprise one single domain antibody of the invention and two known antibodies or single domain antibodies. The multivalent polypeptide may be tetravalent, optionally monospecific tetravalent, bispecific tetravalent, trispecific tetravalent or tetraspecific tetravalent. In one embodiment, the tetraspecific polypeptide may comprise three single domain antibodies of the invention and one known antibody or nanobody or, alternatively, comprising two single domain antibodies of the invention and two known antibodies or nanobodies or, alternatively, comprising one single domain antibody of the invention and three known antibodies or single domain antibodies. Additional multivalent polypeptides comprising both one or more single domain antibodies of the invention and one or more additional known antibodies or single domain antibodies having a higher valency, for example multivalent polypeptides binding 5, 6, 7, 8, 9 or 10 more antigen binding sites, are also provided. Such multivalent polypeptides can be monospecific, bispecific, trispecific, tetraspecific or multispecific.

The multivalent polypeptide may be a dimer (homodimer or heterodimer), trimer (homotrimer or heterotrimer), tetramer (homotetramer or heterotetramer) or multimer (homomultimer or heteromultimer).

In one aspect, a multivalent polypeptide comprising two or more single domain antibodies as disclosed herein is provided. In this aspect, single domain antibodies of the invention are joined or linked together to form a multivalent polypeptide.

The multivalent polypeptide may be bivalent, optionally monospecific bivalent or bispecific bivalent. In this regard, the bivalent polypeptide may comprise two of the same single domain antibodies of the invention or two different single domain antibodies of the invention. The multivalent polypeptide may be trivalent, optionally monospecific trivalent, bispecific trivalent or trispecific trivalent. In this regard, the trivalent polypeptide may comprise three of the same single domain antibodies of the invention, two same single domain antibodies of the invention and one different single domain antibody of the invention, or three different single domain antibodies of the invention. The multivalent polypeptide may be tetravalent, optionally monospecific tetravalent, bispecific tetravalent, trispecific tetravalent or tetraspecific tetravalent. In this regard, the tetravalent polypeptide may comprise four of the same single domain antibodies of the invention, three of the same single domain antibodies of the invention and one different single domain antibody of the invention, two of the same single domain antibodies of the invention and two further different single domain antibodies of the invention (the further single domain antibodies themselves being either the same or different), or four different single domain antibodies of the invention. Additional multivalent polypeptides comprising one or more single domain antibodies of the invention having a higher valency, for example multivalent polypeptides binding 5, 6, 7, 8, 9 or 10 more antigen binding sites, are also provided. Such multivalent polypeptides can be monospecific, bispecific, trispecific, tetraspecific or multispecific.

The multivalent polypeptide may be a dimer (homodimer or heterodimer), trimer (homotrimer or heterotrimer), tetramer (homotetramer or heterotetramer) or multimer (homomultimer or heteromultimer).

In one embodiment, a bivalent polypeptide is provided comprising a first single domain antibody having an amino acid or polynucleotide sequence based on B12, C1, C5, F2, H3, NbSA_A10, NbSA_D10, A8, 3_05, 8_G11, 12_F11 or VHH_H6 (the first row of the table below) and a second single domain antibody having an amino acid or polynucleotide based on B12, 01, 05, F2, H3, NbSA_A10, NbSA_D10, A8, 3_05, 8_G11, 12_F11 or VHH_H6 (the first column of the table below). The table below illustrates the various combinations of two single domain antibodies of the invention that may form a bivalent polypeptide. In a preferred embodiment, a bivalent polypeptide is provided comprising a first single domain antibody comprising an amino acid or polynucleotide sequence based on C5 or A8 and a second single domain antibody having an amino acid or polynucleotide based on B12, C1, C5, F2, H3, NbSA_A10, NbSA_D10, A8, 3_C5, 8_G11, 12_F11 or VHH_H6 (the fourth and eight columns of the table below). In a preferred embodiment, a bivalent polypeptide is provided comprising a first single domain comprising an amino acid or polynucleotide sequence based on A8 and a second single domain antibody comprising an amino acid or polynucleotide sequence based on A8. In a further preferred embodiment, a bivalent polypeptide is provided comprising a first single domain comprising an amino acid or polynucleotide sequence based on C5 and a second single domain antibody comprising an amino acid or polynucleotide sequence based on C5. As detailed throughout, the amino acid or polynucleotide sequence can comprise the CDR3, CDR2 and/or CDR1 or a variant thereof of the specified single domain antibodies, comprise the amino acid sequence or a variant thereof of the specified single domain antibodies, or comprise the polynucleotide sequence of variant thereof of the specified single domain antibodies.

| | | | | | | NbSA_ | NbSA_ | | | | | VHH_ |
|---|---|---|---|---|---|---|---|---|---|---|---|---|
| | B12 | F2 | C1 | C5 | H3 | A10 | D10 | A8 | 3_C5 | 8_G11 | 12_F11 | H6 |
| B12 | B12/ | F2/B12 | C1/B12 | C5/B12 | H3/B12 | NbSA_ | NbSA_ | 2_A8/ | 3_C5/ | 8_G3/ | 12_F11/ | VHH_ |
| | B12 | | | | | A10/ | D10/ | B12 | B12 | B12 | B12 | H6/ |
| | | | | | | B12 | B12 | | | | | B12 |
| F2 | B12/F2 | F2/F2 | C1/F2 | C5/F2 | H3/F2 | NbSA_ | NbSA_ | 2_A8/ | 3_C5/ | 8_G3/ | 12_F11/ | VHH_ |
| | | | | | | A10/F2 | D10/F2 | F2 | F2 | F2 | F2 | H6/F2 |
| C1 | B12/ | F2/C1 | C1/C1 | C5/C1 | H3/C1 | NbSA_ | NbSA_ | A8/C1 | 3_C5/ | 8_G3/ | 12_F11/ | VHH_ |
| | C1 | | | | | A10/C1 | D10/C1 | | C1 | C1 | C1 | H6/C1 |
| C5 | B12/C5 | F2/C5 | C1/C5 | C5/C5 | H3/C5 | NbSA_ | NbSA_ | A8/C5 | 3_C5/ | 8_G3/ | 12_F11/ | VHH_ |
| | | | | | | A10/C5 | D10/C5 | | C5 | C5 | C5 | H6/C5 |
| H3 | B12/H3 | F2/H3 | C1/H3 | C5/H3 | H3/H3 | NbSA_ | NbSA_ | A8/H3 | 3_C5/ | 8_G3/ | 12_F11/ | VHH_ |
| | | | | | | A10/H3 | D10/H3 | | H3 | H3 | H3 | H6/H3 |
| NbSA_ | B12/ | F2/ | C1/ | C5/ | H3/ | NbSA_ | NbSA_ | A8/ | 3_C5/ | 8_G3 | 12_F11/ | VHH_ |
| A10 | NbSA_ | NbSA_ | NbSA_ | NbSA_ | NbSA_ | A10/ | D10/ | NbSA_ | NbSA_ | NbSA_ | NbSA | H6/ |
| | A10 | A10 | A10 | A10 | A10 | NbSA_ | NbSA_ | A10 | A10 | A10/ | A10 | NbSA_ |
| | | | | | | A10 | A10 | | | | | A10 |
| NbSA_ | B12/ | F2/ | C1/ | C5/ | H3/ | NbSA_ | NbSA_ | A8/ | 3_C5/ | 8_G3/ | 12_F11/ | VHH_ |
| D10 | NbSA_ | NbSA_ | NbSA_ | NbSA_ | NbSA_ | A10/ | D10/ | NbSA_ | NbSA_ | NbSA | NbSA | H6/ |
| | D10 | D10 | D10 | D10 | D10 | NbSA_ | NbSA_ | D10 | D10 | D10 | D10 | NbSA_ |
| | | | | | | D10 | D10 | | | | | D10 |
| A8 | B12/A8 | F2/A8 | C1/A8 | C5/A8 | H3/A8 | NbSA_ | NbSA_ | A8/A8 | 3_C5/ | 8_G3/ | 12_F11/ | VHH_ |
| | | | | | | A10/A8 | D10/A8 | | 2_A8 | 2_A8 | 2_A8 | H6/2_ |
| | | | | | | | | | | | | A8 |
| 3_C5 | B12/3_ | F2/3_ | C1/3_ | C5/3_ | H3/3_ | NbSA_ | NbSA_ | A8/3_ | 3_C5/ | 8_G3/ | 12_F11/ | VHH_ |
| | C5 | C5 | C5 | C5 | C5 | A10/3_ | D10/3_ | C5 | 3_C5 | 3_C5 | 3_C5 | H6/3_ |
| | | | | | | C5 | C5 | | | | | C5 |
| 8_G11 | B12/8_ | F2/8_ | C1/8_ | C5/8_ | H3/8_ | NbSA_ | NbSA_ | A8/8_ | 3_C5/ | 8_G3/ | 12_F11/ | VHH_ |
| | G11 | G11 | G11 | G11 | G11 | A10/8_ | D10/8 | G11 | 8_G11 | 8_G11 | 8_G11 | H6/8_ |
| | | | | | | G11 | G11 | | | | | G11 |
| 12_F11 | B12/ | F2/12_ | C1/12_ | C5/12_ | H3/12_ | NbSA_ | NbSA_ | A8/12_ | 3_C5/ | 8_G3/ | 12_F11/ | VHH_ |
| | 12_F11 | F11 | F11 | F11 | F11 | A10/ | D10/ | F11 | 12_F11 | 12_F11 | 12_F11 | H6/12_ |
| | | | | | | 12_F11 | 12_F11 | | | | | F11 |
| VHH_ | B12/ | F2/ | C1/ | C5/ | H3/ | NbSA_ | NbSA | A8/ | 3_C5/ | 8_G3/ | 12_F11/ | VHH_ |
| H6 | VHH_ | VHH_ | VH_H6 | VH_H6 | VH_H6 | A10/ | D10/ | VHH_ | VHH_ | VHH_ | VHH_ | H6/ |
| | H6 | H6 | | | | VHH_ | VHH_ | H6 | H6 | H6 | H6 | VHH_ |
| | | | | | | H6 | H6 | | | | | H6 |

In one embodiment, a trivalent polypeptide is provided comprising a first single domain amino antibody having an amino acid or polynucleotide sequence based on C5, a second single domain antibody having an amino acid or polynucleotide based on B12, C1, C5, F2, H3, NbSA_A10, NbSA_D10, A8, 3_05, 8_G11, 12_F11 or VHH_H6 and a third single domain antibody having an amino acid or polynucleotide based B12, C1, C5, F2, H3, NbSA_A10, NbSA_D10, A8, 3_05, 8_G11, 12_F11 or VHH_H6. In one embodiment, a trivalent polypeptide is provided comprising two single domain amino antibodies having an amino acid or polynucleotide sequence based on C5, and a third single domain antibody having an amino acid or polynucleotide based on B12, C1, C5, F2, H3, NbSA_A10, NbSA_D10, A8, 3_05, 8_G11, 12_F11 or VHH_H6. In one embodiment, a trivalent polypeptide is provided comprising three single domain amino antibodies having an amino acid or polynucleotide sequence based on C5. In one embodiment, a trivalent polypeptide is provided comprising a first single domain amino antibody having an amino acid or polynucleotide sequence based on C1, a second single domain antibody having an amino acid or polynucleotide based on B12, C1, C5, F2, H3, NbSA_A10, NbSA_D10, A8, 3_C5, 8_G11, 12_F11 or VHH_H6 and a third single domain antibody having an amino acid or polynucleotide based on B12, C1, C5, F2, H3, NbSA_A10, NbSA_D10, A8, 3_05, 8_G11, 12_F11 or VHH_H6. In one embodiment, a trivalent polypeptide is provided comprising two single domain amino antibodies having an amino acid or polynucleotide sequence based on C1, and a third single domain antibody having an amino acid or polynucleotide based on B12, C1, C5, F2, H3, NbSA_A10, NbSA_D10, A8, 3_05, 8_G11, 12_F11 or VHH_H6. In one embodiment, a trivalent polypeptide is provided comprising three single domain amino antibodies having an amino acid or polynucleotide sequence based on C1. In one embodiment, a trivalent polypeptide is provided comprising a first single domain amino antibody having an amino acid or polynucleotide sequence based on A8, a second single domain antibody having an amino acid or polynucleotide based on B12, C1, C5, F2, H3, NbSA_A10, NbSA_D10, A8, 3_05, 8_G11, 12_F11 or VHH_H6 and a third single domain antibody having an amino acid or polynucleotide based on B12, C1, C5, F2, H3, NbSA_A10, NbSA_D10, A8, 3_C5, 8_G11, 12_F11 or VHH_H6. In one embodiment, a trivalent polypeptide is provided comprising two single domain amino antibodies having an amino acid or polynucleotide sequence based on A8, and a third single domain antibody having an amino acid or polynucleotide based on B12, C1, C5, F2, H3, NbSA_A10, NbSA_D10, A8, 3_05, 8_G11, 12_F11 or VHH_H6. In one embodiment, a trivalent polypeptide is provided comprising three single domain amino antibodies having an amino acid or polynucleotide sequence based on 2_A8. As detailed throughout, the amino acid or polynucleotide sequence can comprise the CDR3, CDR2 and/or CDR1 or a variant thereof of the specified single domain antibodies, comprise the amino acid sequence or a variant thereof of the specified single domain antibodies, or comprise the polynucleotide sequence of variant thereof of the specified single domain antibodies.

In one embodiment, a trimer comprising three single domain antibodies is provided, each single domain antibody comprising:
(a) a CDR1 comprising SEQ ID NO:7, a CDR2 comprising SEQ ID NO:8 and a CDR3 comprising SEQ ID NO:9 (C1);
(b) a CDR1 comprising SEQ ID NO:13, a CDR2 comprising SEQ ID NO:14 and a CDR3 comprising SEQ ID NO:15 (H3);
(c) a CDR1 comprising SEQ ID NO:10, a CDR2 comprising SEQ ID NO:11 and a CDR3 comprising SEQ ID NO:12 (C5); or
(d) a CDR1 comprising SEQ ID NO:196, a CDR2 comprising SEQ ID NO:197 and a CDR3 comprising SEQ ID NO:198 (A8);
wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications.

In one embodiment, a trimer comprising three single domain antibodies is provided, each single domain antibody comprising:
(a) a CDR1 comprising SEQ ID NO:7, a CDR2 comprising SEQ ID NO:8 and a CDR3 comprising SEQ ID NO:9 (C1);
(b) a CDR1 comprising SEQ ID NO:13, a CDR2 comprising SEQ ID NO:14 and a CDR3 comprising SEQ ID NO:15 (H3);
(c) a CDR1 comprising SEQ ID NO:10, a CDR2 comprising SEQ ID NO:11 and a CDR3 comprising SEQ ID NO:12 (C5); or
(d) a CDR1 comprising SEQ ID NO:196, a CDR2 comprising SEQ ID NO:197 and a CDR3 comprising SEQ ID NO:198 (A8).

The coronavirus biding molecules or multivalent polypeptides of the invention may comprise a suitable linker, as further described herein. The linker is used to join the one or more single domain antibodies of the invention to one or more known antibodies or single domain antibodies and/or further single domain antibodies of the invention to form a multivalent polypeptide.

In one embodiment, the linker comprises 1 to 50 amino acids. In one embodiment, the linker comprises 5 to 35, optionally 5 to 25, or 5 to 15 amino acids. In one embodiment, the linker comprises 4 to 8 amino acids, optionally 4, 5, 6, 7 or 8 amino acids. In one embodiment, the linker comprises one or more amino acids, for example two or more amino acids, three or more amino acids, four or more amino acids, five or more amino acids, six or more amino acids, seven or more amino acids, eight or more amino acids, nine or more amino acids, or ten or more amino acids. The linker can comprise any known amino acid residues, however may preferably comprise glycine and serine residues. In one embodiment, the linker comprises one or more glycine residues and/or one or more serine residues. In one embodiment, the linker comprises at least 1, at least 5, at least 10, or at least 20 glycine and/or serine residues. In one embodiment, the linker comprises 5 to 35, optionally 5 to 25, or 5 to 15 glycine and/or serine residues. In one embodiment, the linker comprises two glycine-serine repeats (GSGS). In one embodiment, the linker comprises three glycine-serine repeats (GSGSGS). In one embodiment, the linker comprises four glycine-serine repeats (GSGSGSGS). In one embodiment, the linker comprises multiple glycine-serine repeats, represented by the general formula (GS)n, wherein n is the number of GS repeats present, for example n is 1, 2, 3, 4, 5, 6, 7, 8, 9, 10, 11, 12, 13, 14, 15, 16, 17, 18, 19 or 20. The linker may be joined to either the n-terminal, the c-terminal, or in the case that the multivalent polypeptides comprise multiple linkers, the linker may be joined at both the n- and the c-terminal of the single domain antibodies of the invention

In one embodiment, the linker comprises a protein from the ubiquitin-like protein superfamily, either ubiquitin (Ub) itself or a ubiquitin-like protein (ULP). Such proteins have been extensively characterised and are well known to the skilled person. Such proteins comprise a ubiquitin-like folding motif. The linker of the invention can be varied in composition and length to tailor the design to the optimal arrangement for linking the first and second antigen binding molecules or single domain antibodies of the invention and positioning them in the optimal spatial arrangement for targeting the first and second epitope. The linker can further be optimised in composition and length to optimise binding characteristics of the coronavirus binding molecule or single domain antibodies, for example Kd, as described herein.

In one embodiment, the linker comprises a protein selected from the group consisting of ubiquitin, Small Ubiquitin-like Modifier 1 (SUMO-1, also known in humans as Smt3c, PIC1, GMP1, sentrin and UbI1), Small Ubiquitin-like Modifier 2 (SUMO-2, also known in humans as Smt3a and Sentrin3), Small Ubiquitin-like Modifier 3 (SUMO-3, also known as Smt3b and Sentrin2), Small Ubiquitin-like Modifier 4 (SUMO-4), FAU, NEDD-8, UBL-1, and GDX, Rub1, APG8, ISG15, URM 1, HUB1, elonginB, or PLIC2. In one embodiment, the protein is ubiquitin. In a preferred embodiment, the protein is Small Ubiquitin-like Modifier (SUMO). In one embodiment, the linker comprises two or more ubiquitins (Ub) or a ubiquitin-like proteins (ULP), optionally 2, 3, 4 or 5 ubiquitins (Ub) or a ubiquitin-like proteins (ULP). The linker may be extended to additionally comprise amino acids at both the n-terminal and the c-terminal ends of the ubiquitin (Ub) or ubiquitin-like protein linker. In one embodiment, additional amino acids are joined to the n-terminal end of the ubiquitin (Ub) or ubiquitin-like protein linker. In one embodiment, additional amino acids are joined to the c-terminal end of the ubiquitin (Ub) or ubiquitin-like protein linker. In one embodiment, additional amino acids are joined to the c-terminal and the n-terminal end of the ubiquitin (Ub) or ubiquitin-like protein linker. The amino acids joined to either the n-terminal, the c-terminal, or both the n- and the c-terminal of the ubiquitin (Ub) or ubiquitin-like protein linker can comprise one or more additional amino acids. In one embodiment, 5 to 50 amino acids may be joined to either the n-terminal, the c-terminal, or both the n- and the c-terminal of the ubiquitin (Ub) or ubiquitin-like protein linker. In one embodiment, 4 to 8 amino acids may be joined to either the n-terminal, the c-terminal, or both the n- and the c-terminal of the ubiquitin (Ub) or ubiquitin-like protein linker, optionally 4, 5, 6, 7 or 8 amino acids. In one embodiment, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids may be joined to either the n-terminal, the c-terminal, or both the n- and the c-terminal of the ubiquitin (Ub) or ubiquitin-like protein linker. In one embodiment, 1 to 10, 1 to 8, 1 to 6, 1 to 4 or 1 to 2 amino acids may be joined to either the n-terminal, the c-terminal, or both the n- and the c-terminal of the ubiquitin (Ub) or ubiquitin-like protein linker. In a preferred embodiment, 6 amino acid residues may be joined to either the n-terminal, the c-terminal, or both the n- and the c-terminal of the ubiquitin (Ub) or ubiquitin-like protein linker.

In the case that amino acids are joined to both the n-terminal and the c-terminal of the ubiquitin (Ub) or ubiquitin-like protein linker, the number of amino acids at each terminal may be the same or different, i.e. the extensions at either side may be of the same length, or the extensions at each terminal may be of differing lengths.

The amino acids joined to either the n-terminal, the c-terminal, or both the n- and the c-terminal of either the single domain antibody or the ubiquitin (Ub) or ubiquitin-like protein linker can comprise any amino acid. In a preferred embodiment, the one or more amino acids are a glycine-serine (GS) linker. The glycine serine linker can be repeated to increase the chain length, for example two glycine serine linkers (GSGS), three glycine-serine linkers (GSGSGS), four glycine-serine linkers (GSGSGSGS) or five three glycine-serine linkers (GSGSGSGS) may be joined to either the n-terminal, the c-terminal, or both the n- and the c-terminal of the ubiquitin (Ub) or ubiquitin-like protein linker. In a preferred embodiment, the amino acids joined to either the n-terminal, the c-terminal, or both the n- and the c-terminal of the ubiquitin (Ub) or ubiquitin-like protein linker are GSGSGS. In one embodiment, the linker comprises GSGSGS at both the c-terminal and the n-terminal end of the ubiquitin (Ub) or ubiquitin-like protein linker. In one embodiment, the linker comprises a poly-A tail.

In a preferred embodiment, the linker comprises SUMO and an extension of three glycine-serine linkers (GSGSGS) at the n-terminal end of the SUMO and an extension of three glycine-serine linkers (GSGSGS) at the c-terminal end.

In one embodiment, SUMO-1 comprises SEQ ID NO: 240 or 241 or a variant thereof (https://www.uniprot.org/uniprot/P63165). In one embodiment, SUMO-2 comprises SEQ ID NO: 242 or 243 or a variant thereof (https://www.uniprot.org/uniprot/P61956; https://www.uniprot.org/uniprot/P16956). In one embodiment, SUMO-3 comprises SEQ ID NO: 244 or 245 or a variant thereof (https://www.uniprot.org/uniprot/P55854 https://www.uniprot.org/uniprot/P55854). In one embodiment, SUMO-4 comprises SEQ ID NO: 246 or a variant thereof (https://www.uniprot.org/uniprot/Q6EEV6).

The linker optionally further comprises a spacer joined to the n-terminal and/or c-terminal ends of the ubiquitin (Ub) or ubiquitin-like protein linker. In one embodiment, a spacer is joined to the n-terminal end of the ubiquitin (Ub) or ubiquitin-like protein linker. In one embodiment, a spacer is joined to the c-terminal end of the ubiquitin (Ub) or ubiquitin-like protein linker. In one embodiment, a spacer is joined to the c-terminal and the n-terminal end of the ubiquitin (Ub) or ubiquitin-like protein linker.

The linker, including any optional spacers attached the n and/or c terminal of the ubiquitin (Ub) or ubiquitin-like protein, may be used to link the c-terminal of the first antigen binding molecule to the n-terminal of the second antigen binding molecule, or alternatively may be used to link the c-terminal of the second antigen binding molecule to the n-terminal of the first antigen binding molecule.

The spacer may comprise one or more amino acids, preferably between 4 and 50 amino acids. In one embodiment, 4 to 50 amino acids may be joined to either the n-terminal, the c-terminal, or both the n- and the c-terminal of the ubiquitin (Ub) or ubiquitin-like protein linker. In one embodiment, 4 to 8 amino acids may be joined to either the n-terminal, the c-terminal, or both the n- and the c-terminal of the ubiquitin (Ub) or ubiquitin-like protein linker, optionally 4, 5, 6, 7 or 8 amino acids. In one embodiment, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids may be joined to either the n-terminal, the c-terminal, or both the n- and the c-terminal of the ubiquitin (Ub) or ubiquitin-like protein linker. In one embodiment, 1 to 10, 1 to 8, 1 to 6, 1 to 4 or 1 to 2 amino acids may be joined to either the n-terminal, the c-terminal, or both the n- and the c-terminal of the ubiquitin (Ub) or ubiquitin-like protein linker. In a preferred embodiment, 6 amino acid residues may be joined to either the n-terminal, the c-terminal, or both the n- and the c-terminal of the ubiquitin (Ub) or ubiquitin-like protein linker.

In one embodiment, a multivalent polypeptide is provided comprising two single domain antibodies selected from the group consisting of:
i. two single domain antibodies each comprising SEQ ID NO: 12 (C5 CDR3);
ii. two single domain antibodies each comprising SEQ ID NO: 198 (A8 CDR3);
iii. two single domain antibodies each comprising SEQ ID NO: 9 (C1 CDR3);
iv. two single domain antibodies each comprising SEQ ID NO: 3 (B12 CDR3);
v. two single domain antibodies each comprising SEQ ID NO: 6 (F2 CDR3);
vi. two single domain antibodies each comprising SEQ ID NO: 15 (H3 CDR3);
vii. two single domain antibodies each comprising SEQ ID NO: 192 (NbSA_A10 CDR3);
viii. two single domain antibodies each comprising SEQ ID NO: 195 (NbSA_D10 CDR3);
ix. two single domain antibodies each comprising SEQ ID NO: 201 (3_C5 CDR3); x. two single domain antibodies each comprising SEQ ID NO: 204 (8_G11 CDR3); and
xi. two single domain antibodies each comprising SEQ ID NO: 207 (12_F11 CDR3); and
xiii. two single domain antibodies each comprising SEQ ID NO: 237 (VHH_H6 CDR3) wherein the amino acid sequence of CDR3 comprises between 0 and 7 amino acid modifications. In one embodiment, the multivalent polypeptide is a monospecific bivalent polypeptide. In a preferred embodiment, the multivalent polypeptide comprises two single domain antibodies each comprising SEQ ID NO: 198 (2_A8 CDR3), wherein the amino acid sequence of CDR3 comprises between 0 and 7 amino acid modifications. In a preferred embodiment, the multivalent polypeptide comprises two single domain antibodies each comprising SEQ ID NO: 12 (C5 CDR3), wherein the amino acid sequence of CDR3 comprises between 0 and 7 amino acid modifications.

In one embodiment, a multivalent polypeptide is provided comprising two single domain antibodies selected from the group consisting of:
i. two single domain antibodies each comprising a CDR2 comprising SEQ ID NO:11 and a CDR3 comprising SEQ ID NO:12 (C5);
ii. two single domain antibodies each comprising a CDR2 comprising SEQ ID NO:197 and a CDR3 comprising SEQ ID NO:198 (A8)
iii. two single domain antibodies each comprising a CDR2 comprising SEQ ID NO:8 and a CDR3 comprising SEQ ID NO:9 (C1);
iv. two single domain antibodies each comprising a CDR2 comprising SEQ ID NO:2 and a CDR3 comprising SEQ ID NO:3 (B12);
v. two single domain antibodies each comprising a CDR2 comprising SEQ ID NO:5 and a CDR3 comprising SEQ ID NO:6 (F2); and
vi. two single domain antibodies each comprising a CDR2 comprising SEQ ID NO:14 and a CDR3 comprising SEQ ID NO:15 (H3);
vii. two single domain antibodies each comprising a CDR2 comprising SEQ ID NO:191 and a CDR3 comprising SEQ ID NO:192
viii. two single domain antibodies each comprising a CDR2 comprising SEQ ID NO:194 and a CDR3 comprising SEQ ID NO:195
ix. two single domain antibodies each comprising a CDR2 comprising SEQ ID NO:200 and a CDR3 comprising SEQ ID NO:201;
x. two single domain antibodies each comprising a CDR2 comprising SEQ ID NO:203 and a CDR3 comprising SEQ ID NO:204;
xi. two single domain antibodies each comprising a CDR2 comprising SEQ ID NO:206; and a CDR3 comprising SEQ ID NO:207; and
xii. two single domain antibodies each comprising a CDR2 comprising SEQ ID NO:236; and a CDR3 comprising SEQ ID NO:237;
wherein the amino acid sequence of CDR3 comprises between 0 and 7 amino acid modifications and wherein the amino acid sequence of CDR2 comprises between 0 and 4 amino acid modifications. In one embodiment, the multivalent polypeptide is a monospecific bivalent polypeptide. In a preferred embodiment, the multivalent polypeptide comprises two single domain antibodies each comprising a CDR2 comprising SEQ ID NO:197 and a CDR3 comprising SEQ ID NO:198 (2_A8), wherein the amino acid sequence of CDR3 comprises between 0 and 7 amino acid modifications and wherein the amino acid sequence of CDR2 comprises between 0 and 4 amino acid modifications. In a preferred embodiment, the multivalent polypeptide comprises two single domain antibodies each comprising a CDR2 comprising SEQ ID NO:11 and a CDR3 comprising SEQ ID NO:12 (C5), wherein the amino acid sequence of CDR3 comprises between 0 and 7 amino acid modifications and wherein the amino acid sequence of CDR2 comprises between 0 and 4 amino acid modifications.

In one embodiment, a multivalent polypeptide is provided comprising two single domain antibodies selected from the group consisting of:
i. two single domain antibodies each comprising a CDR1 comprising SEQ ID NO:10, a CDR2 comprising SEQ ID NO:11 and a CDR3 comprising SEQ ID NO:12 (C5);
ii. two single domain antibodies each comprising a CDR1 comprising SEQ ID NO:196, a CDR2 comprising SEQ ID NO:197 and a CDR3 comprising SEQ ID NO:198 (A8);
iii. two single domain antibodies each comprising a CDR1 comprising SEQ ID NO:7, a CDR2 comprising SEQ ID NO:8 and a CDR3 comprising SEQ ID NO:9 (C1);
iv. two single domain antibodies each comprising CDR1 comprising SEQ ID NO:1, a CDR2 comprising SEQ ID NO:2 and a CDR3 comprising SEQ ID NO:3 (B12);
v. two single domain antibodies each comprising a CDR1 comprising SEQ ID NO:4, a CDR2 comprising SEQ ID NO:5 and a CDR3 comprising SEQ ID NO:6 (F2); and
vi. two single domain antibodies each comprising a CDR1 comprising SEQ ID NO:13, a CDR2 comprising SEQ ID NO:14 and a CDR3 comprising SEQ ID NO:15 (H3);
vii. two single domain antibodies each comprising a CDR1 comprising SEQ ID NO:190, a CDR2 comprising SEQ ID NO:191 and a CDR3 comprising SEQ ID NO:192;
viii. two single domain antibodies each comprising a CDR1 comprising SEQ ID NO:193, a CDR2 comprising SEQ ID NO:194 and a CDR3 comprising SEQ ID NO:195;
ix. two single domain antibodies each comprising a CDR1 comprising SEQ ID NO:199, a CDR2 comprising SEQ ID NO:200 and a CDR3 comprising SEQ ID NO:201;
x. two single domain antibodies each comprising a CDR1 comprising SEQ ID NO:194, a CDR2 comprising SEQ ID NO:203 and a CDR3 comprising SEQ ID NO:204;
xi. two single domain antibodies each comprising a CDR1 comprising SEQ ID NO:205, a CDR2 comprising SEQ ID NO:206 and a CDR3 comprising SEQ ID NO:207; and
xii. two single domain antibodies each comprising a CDR1 comprising SEQ ID NO:235, a CDR2 comprising SEQ ID NO:236 and a CDR3 comprising SEQ ID NO:237;
wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications. In one embodiment, the multivalent polypeptide is a monospecific bivalent polypeptide. i In a preferred embodiment, the multivalent polypeptide comprises two single domain antibodies each comprising a CDR1 comprising SEQ ID NO:7, a CDR2 comprising SEQ ID NO:8 and a CDR3 comprising SEQ ID NO:9 (A8), wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications. In a preferred embodiment, the multivalent polypeptide comprises two single domain antibodies each comprising two single domain antibodies each comprising a CDR1 comprising SEQ ID NO:10, a CDR2 comprising SEQ ID NO:11 and a CDR3 comprising SEQ ID NO:12 (C5), wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications.

In one embodiment, a trivalent polypeptide is provided comprising three single domain antibodies selected from the group consisting of:
i. three single domain antibodies each comprising SEQ ID NO: 12 (C5 CDR3);
ii. three single domain antibodies each comprising SEQ ID NO: 198 (A8);
iii. three single domain antibodies each comprising SEQ ID NO: 9 (C1 CDR3);
iv. three single domain antibodies each comprising SEQ ID NO: 3 (B12 CDR3);
v. three single domain antibodies each comprising SEQ ID NO: 6 (F2 CDR3);
vi. three single domain antibodies each comprising SEQ ID NO: 15 (H3 CDR3);
vii. three single domain antibodies each comprising SEQ ID NO: 192;
viii. three single domain antibodies each comprising SEQ ID NO: 195;
ix. three single domain antibodies each comprising SEQ ID NO: 201;
x. three single domain antibodies each comprising SEQ ID NO: 204;
xi. three single domain antibodies each comprising SEQ ID NO: 207; and
xii. three single domain antibodies each comprising SEQ ID NO: 237;
wherein the amino acid sequence of CDR3 comprises between 0 and 7 amino acid modifications. In one embodiment, the trivalent polypeptide is a monospecific trivalent polypeptide. In a preferred embodiment, the trivalent polypeptide comprises three single domain antibodies each comprising SEQ ID NO: 9 (C1 CDR3), wherein the amino acid sequence of CDR3 comprises between 0 and 7 amino acid modifications. In a preferred embodiment, the trivalent polypeptide comprises three single domain antibodies each comprising SEQ ID NO: 198 (A8), wherein the amino acid sequence of CDR3 comprises between 0 and 7 amino acid modifications. In a most preferred embodiment, the trivalent polypeptide comprises three single domain antibodies each comprising SEQ ID NO: 12 (C5 CDR3), wherein the amino acid sequence of CDR3 comprises between 0 and 7 amino acid modifications.

In one embodiment, a trivalent polypeptide is provided comprising three single domain antibodies selected from the group consisting of:
i. three single domain antibodies each comprising a CDR2 comprising SEQ ID NO:11 and a CDR3 comprising SEQ ID NO:12 (C5);
ii. three single domain antibodies each comprising a CDR2 comprising SEQ ID NO:197 and a CDR3 comprising SEQ ID NO:198;
iii. three single domain antibodies each comprising a CDR2 comprising SEQ ID NO:8 and a CDR3 comprising SEQ ID NO:9 (C1);
iv. three single domain antibodies each comprising a CDR2 comprising SEQ ID NO:2 and a CDR3 comprising SEQ ID NO:3 (B12);
v. three single domain antibodies each comprising a CDR2 comprising SEQ ID NO:5 and a CDR3 comprising SEQ ID NO:6 (F2); and
vi. three single domain antibodies each comprising a CDR2 comprising SEQ ID NO:14 and a CDR3 comprising SEQ ID NO:15 (H3);
vii. three single domain antibodies each comprising a CDR2 comprising SEQ ID NO:191 and a CDR3 comprising SEQ ID NO:192;
viii. three single domain antibodies each comprising a CDR2 comprising SEQ ID NO:194 and a CDR3 comprising SEQ ID NO:195;
ix. three single domain antibodies each comprising a CDR2 comprising SEQ ID NO:200 and a CDR3 comprising SEQ ID NO:201;
x. three single domain antibodies each comprising a CDR2 comprising SEQ ID NO:203 and a CDR3 comprising SEQ ID NO:204;
xi. three single domain antibodies each comprising a CDR2 comprising SEQ ID NO:206 and a CDR3 comprising SEQ ID NO:207; and
xii. three single domain antibodies each comprising a CDR2 comprising SEQ ID NO:236 and a CDR3 comprising SEQ ID NO:237;
wherein the amino acid sequence of CDR3 comprises between 0 and 7 amino acid modifications and wherein the amino acid sequence of CDR2 comprises between 0 and 4 amino acid modifications. In one embodiment, the trivalent polypeptide is a monospecific trivalent polypeptide. In a preferred embodiment, the trivalent polypeptide comprises three single domain antibodies each comprising a CDR2 comprising SEQ ID NO:197 and a CDR3 comprising SEQ ID NO:198 (A8), wherein the amino acid sequence of CDR3 comprises between 0 and 7 amino acid modifications and wherein the amino acid sequence of CDR2 comprises between 0 and 4 amino acid modifications. In a preferred embodiment, the trivalent polypeptide comprises three single domain antibodies each comprising a CDR2 comprising SEQ ID NO:11 and a CDR3 comprising SEQ ID NO:12 (C5), wherein the amino acid sequence of CDR3 comprises between 0 and 7 amino acid modifications and wherein the amino acid sequence of CDR2 comprises between 0 and 4 amino acid modifications.

In one embodiment, a trivalent polypeptide is provided comprising three single domain antibodies selected from the group consisting of:
i. three single domain antibodies each comprising a CDR1 comprising SEQ ID NO:10, a CDR2 comprising SEQ ID NO:11 and a CDR3 comprising SEQ ID NO:12 (C5);
ii. three single domain antibodies each comprising a CDR1 comprising SEQ ID NO:196, a CDR2 comprising SEQ ID NO:197 and a CDR3 comprising SEQ ID NO:198 (A8);
iii. three single domain antibodies each comprising a CDR1 comprising SEQ ID NO:7, a CDR2 comprising SEQ ID NO:8 and a CDR3 comprising SEQ ID NO:9 (C1);
iv. three single domain antibodies each comprising CDR1 comprising SEQ ID NO:1, a CDR2 comprising SEQ ID NO:2 and a CDR3 comprising SEQ ID NO:3 (B12);
v. three single domain antibodies each comprising a CDR1 comprising SEQ ID NO:4, a CDR2 comprising SEQ ID NO:5 and a CDR3 comprising SEQ ID NO:6 (F2); and
vi. three single domain antibodies each comprising a CDR1 comprising SEQ ID NO:13, a CDR2 comprising SEQ ID NO:14 and a CDR3 comprising SEQ ID NO:15 (H3);
vii. three single domain antibodies each comprising a CDR1 comprising SEQ ID NO:190, a CDR2 comprising SEQ ID NO:191 and a CDR3 comprising SEQ ID NO:192;
viii. three single domain antibodies each comprising a CDR1 comprising SEQ ID NO:193, a CDR2 comprising SEQ ID NO:194 and a CDR3 comprising SEQ ID NO:195;
ix. three single domain antibodies each comprising a CDR1 comprising SEQ ID NO:199, a CDR2 comprising SEQ ID NO:200 and a CDR3 comprising SEQ ID NO:201;
x. three single domain antibodies each comprising a CDR1 comprising SEQ ID NO:202, a CDR2 comprising SEQ ID NO:203 and a CDR3 comprising SEQ ID NO:204;
xi. three single domain antibodies each comprising a CDR1 comprising SEQ ID NO:205, a CDR2 comprising SEQ ID NO:206 and a CDR3 comprising SEQ ID NO:207; and
xii. three single domain antibodies each comprising a CDR1 comprising SEQ ID NO:235, a CDR2 comprising SEQ ID NO:236 and a CDR3 comprising SEQ ID NO:237;
wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications. In a preferred embodiment, the trivalent polypeptide comprises three single domain antibodies each comprising a CDR1 comprising SEQ ID NO:196, a CDR2 comprising SEQ ID NO:197 and a CDR3 comprising SEQ ID NO:198 (A8), wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications. In a preferred embodiment, the trivalent polypeptide comprises three single domain antibodies each comprising two single domain antibodies each comprising a CDR1 comprising SEQ ID NO:10, a CDR2 comprising SEQ ID NO:11 and a CDR3 comprising SEQ ID NO:12 (C5), wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications.

In one embodiment, a multivalent polypeptide is provided comprising three or more single domain antibodies (sdAB) of the invention, represented by Formula I or II:

(sdAB)_{N}-(Linker)_{N-1} Formula I:

(sdAB)_{N}-(Linker)_{N} Formula II:

wherein the number N is the number of single domain antibodies (sdAb) and wherein the linker(s) can be the same or different and may include one of more of: polyA linkers; GS linkers and/or ubiquitin or ubiquitin-like linkers, optionally SUMO or SUMO-like linkers. In one embodiment n is selected from the group consisting of 3, 4, 5, 6, 7, 8, 9 and 10. In one embodiment n is 3. In one embodiment n is 4. In one embodiment n is 5.

In one embodiment, a multivalent polypeptide is provided comprising three or more single domain antibodies (sdAB), represented by Formula I or II:

(sdAB)_{N}-(Linker)_{N-1} Formula I:

(sdAB)_{N}-(Linker)_{N} Formula II:

and wherein each single domain antibody comprises SEQ ID NO: 12 (C5 CDR3) and wherein the amino acid sequence of CDR3 comprises between 0 and 7 amino acid modifications, and wherein the number N is the number of single domain antibodies (sdAb) and wherein the linker(s) can be the same or different and may include one of more of: polyA linkers; GS linkers and/or ubiquitin or ubiquitin-like linkers, optionally SUMO or SUMO-like linkers. In one embodiment n is selected from the group consisting of 3, 4, 5, 6, 7, 8, 9 and 10. In one embodiment n is 3. In one embodiment n is 4. In one embodiment n is 5.

In one embodiment, a multivalent polypeptide is provided comprising three or more single domain antibodies (sdAB), represented by Formula I or II:

(sdAB)_{N}-(Linker)_{N-1} Formula I:

(sdAB)_{N}-(Linker)_{N} Formula II:

and wherein each single domain antibody comprises a CDR2 comprising SEQ ID NO:11 and a CDR3 comprising SEQ ID NO:12 (C5), and wherein the amino acid sequence of CDR3 comprises between 0 and 7 amino acid modifications and wherein the amino acid sequence of CDR2 comprises between 0 and 4 amino acid modifications, wherein the number N is the number of single domain antibodies (sdAb) and wherein the linker(s) can be the same or different and may include one of more of: polyA linkers; GS linkers and/or ubiquitin or ubiquitin-like linkers, optionally SUMO or SUMO-like linkers. In one embodiment n is selected from the group consisting of 3, 4, 5, 6, 7, 8, 9 and 10. In one embodiment n is 3. In one embodiment n is 4. In one embodiment n is 5.

In one embodiment, a multivalent polypeptide is provided comprising three or more single domain antibodies (sdAB), represented by Formula I or II:

(sdAB)_{N}-(Linker)_{N-1} Formula I:

(sdAB)_{N}-(Linker)_{N} Formula II:
and wherein each single domain antibody comprises a CDR1 comprising SEQ ID NO:10, a CDR2 comprising SEQ ID NO:11 and a CDR3 comprising SEQ ID NO:12 (C5), and wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications,
and wherein the number N is the number of single domain antibodies (sdAb) and wherein the linker(s) can be the same or different and may include one of more of: polyA linkers; GS linkers and/or ubiquitin or ubiquitin-like linkers, optionally SUMO or SUMO-like linkers. In one embodiment n is selected from the group consisting of 3, 4, 5, 6, 7, 8, 9 and 10. In one embodiment n is 3. In one embodiment n is 4. In one embodiment n is 5.

In one embodiment, a multivalent polypeptide is provided comprising three or more single domain antibodies (sdAB), represented by Formula I or II:

(sdAB)_{N}-(Linker)_{N-1} Formula I:

(sdAB)_{N}-(Linker)_{N} Formula II:
and wherein each single domain antibody comprises a CDR1 comprising SEQ ID NO:196, a CDR2 comprising SEQ ID NO:197 and a CDR3 comprising SEQ ID NO:198 (2_A8), and wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications,
and wherein the number N is the number of single domain antibodies (sdAb) and wherein the linker(s) can be the same or different and may include one of more of: polyA linkers; GS linkers and/or ubiquitin or ubiquitin-like linkers, optionally SUMO or SUMO-like linkers. In one embodiment n is selected from the group consisting of 3, 4, 5, 6, 7, 8, 9 and 10. In one embodiment n is 3. In one embodiment n is 4. In one embodiment n is 5.

In one embodiment, a bivalent polypeptide (C5-AAA-C5) is provided having the nucleotide sequence SEQ ID NO: 162 or 181, or variant thereof. In one embodiment, a bivalent polypeptide (C5-AAA-C5) is provided having the amino acid sequence SEQ ID NO: 171 or 182, or a variant thereof. In one embodiment, a bivalent polypeptide (C5-9GS-C5) is provided having the nucleotide sequence SEQ ID NO: 163 or SEQ ID NO: 183, or a variant thereof. In one embodiment, a bivalent polypeptide (C5-9GS-C5) is provided having the amino acid sequence SEQ ID NO: 172 or SEQ ID NO: 184, or a variant thereof. In one embodiment, a bivalent polypeptide (C5-GSGSGS-SUMO-GSGSGS-C5) is provided having the nucleotide sequence SEQ ID NO: 164 or SEQ ID NO: 185, or a variant thereof. In one embodiment, a bivalent polypeptide (C5-GSGSGS-SUMO-GSGSGS-C5) is provided having the amino acid sequence SEQ ID NO: 173 or SEQ ID NO: 186, or a variant thereof.

In one embodiment, a bidentate polypeptide (C5-GSGSGS-SUMO-GSGSGS-C5) is provided having the nucleotide sequence SEQ ID NO: 165, or a variant thereof.

In one embodiment, a bidentate polypeptide (C5-GSGSGS-SUMO-GSGSGS-F2) is provided having the amino acid sequence SEQ ID NO: 174, or a variant thereof. In one embodiment, a bidentate polypeptide (F2-GSGSGS-SUMO-GSGSGS-C5) is provided having the nucleotide sequence SEQ ID NO: 166, or a variant thereof.

In one embodiment, a bidentate polypeptide (F2-GSGSGS-SUMO-GSGSGS-C5) is provided having the amino acid sequence SEQ ID NO: 175, or a variant thereof. In one embodiment, a bidentate polypeptide (C1-GSGSGS-SUMO-GSGSGS-C5) is provided having the nucleotide sequence SEQ ID NO: 167, or a variant thereof.

In one embodiment, a bidentate polypeptide (C1-GSGSGS-SUMO-GSGSGS-C5) is provided having the amino acid sequence SEQ ID NO: 176, or a variant thereof. In one embodiment, a bidentate polypeptide (C1-GSGSGS-SUMO-GSGSGS-H3) is provided having the nucleotide sequence SEQ ID NO: 168, or a variant thereof. In one embodiment, a bidentate polypeptide (C1-GSGSGS-SUMO-GSGSGS-H3) is provided having the amino acid sequence SEQ ID NO: 177, or a variant thereof. In one embodiment, a bidentate polypeptide (C1-GSGSGS-SUMO-GSGSGS-H11-H4) is provided having the nucleotide sequence SEQ ID NO: 178, or a variant thereof. In one embodiment, a bidentate polypeptide (C1-GSGSGS-SUMO-GSGSGS-H11-H4) is provided having the nucleotide sequence SEQ ID NO:179, or a variant thereof:

In one embodiment, a bidentate polypeptide (F2-GSGSGS-SUMO-GSGSGS-VHH_H6) is provided having the amino acid sequence SEQ ID NO: 247, or a variant thereof. In one embodiment, a bidentate polypeptide (F2-GSGSGS-SUMO-GSGSGS-VHH_H6) is provided having the amino acid sequence SEQ ID NO: 248, or a variant thereof. In one embodiment, a trivalent polypeptide (C5-6GS-C5-6GS-C5) is provided having the nucleotide sequence SEQ ID NO: 187, or a variant thereof. In one embodiment, a trivalent polypeptide (C5-6GS-C5-6GS-C5) is provided having the amino acid sequence SEQ ID NO: 188 or SEQ ID NO: 189, or a variant thereof. In one embodiment, a trivalent polypeptide (A8) is provided having the nucleotide sequence SEQ ID NO: 230, or a variant thereof.

In one embodiment, a trivalent polypeptide (A8) is provided having the amino acid sequence SEQ ID NO: 221 or SEQ ID NO: 222, or a variant thereof. In one embodiment, a trivalent polypeptide (ci) is provided having the nucleotide sequence SEQ ID NO: 223, or a variant thereof. In one embodiment, a trivalent polypeptide (ci) is provided having the amino acid sequence SEQ ID NO: 224 or SEQ ID NO: 225, or a variant thereof.

In one embodiment, a trivalent polypeptide (F2) is provided having the nucleotide sequence SEQ ID NO: 226, or a variant thereof. In one embodiment, a trivalent polypeptide (F2) is provided having the amino acid sequence SEQ ID NO: 227, or a variant thereof. In one embodiment, a trivalent polypeptide (H3) is provided having the nucleotide sequence SEQ ID NO: 228, or a variant thereof. In one embodiment, a trivalent polypeptide (H3) is provided having the amino acid sequence SEQ ID NO: 229, or a variant thereof. In one embodiment, a bidentate polypeptide (VHH_72-SUMO-C5) is provided having the amino acid sequence SEQ ID NO: 234, or a variant thereof.

Variants of the specified sequences may comprise one or more modifications (amino acid or nucleotide substitutions, deletions or insertions), two or more, three or more modifications, four or more, five or more, six or more, seven or more, eight or more, nine or more of 10 or more modifications. In one embodiment, a variant comprises 1, 2, 3, 4, 5, 6, 7, 8, 9 or 10 modifications. Variants comprising one or more modifications, as detailed herein, will retain binding affinity for a coronavirus peptide, preferably the receptor binding domain of the S protein of SARS-CoV-2.

In one embodiment, a single domain antibody, multivalent polypeptide or antigen binding molecule of the invention is fused to or conjugated to an Fc domain, such as a human Fc, to create a fusion protein. In one embodiment, a bidentate polypeptide of the invention is fused to a Fc domain. In one embodiment, a first antigen binding molecule of the invention is fused to or conjugated to an Fc domain, such as a human Fc, to create a fusion protein. In one embodiment, a second antigen binding molecule of the invention is fused to or conjugated to an Fc domain, such as a human Fc, to create a fusion protein.

The IgG Fc region is comprised of two halves, each half comprising a CH2 and CH3, and wherein each half is joined by a hinge region. In one embodiment, the single domain antibodies, multivalent polypeptides or antigen binding molecules of the invention may be fused to an Fc fragment comprising CH2 and CH3 (i.e. one half of the Fc region), optionally wherein the Fc fragment comprises the hinge region. In one embodiment, the single domain antibodies, multivalent polypeptides or antigen binding molecules of the invention may be fused to an Fc domain comprising two halves, each half comprising a CH2 and CH3, and wherein each half is joined by a hinge region.

In one embodiment, the Fc domain is an IgG Fc domain, optionally selected from the group consisting of the Fc domain if IgG1, IgG2, IgG3 and IgG4. In one embodiment, single domain antibody, multivalent polypeptide or antigen binding molecule of the invention is fused to or conjugated to the Fc domain of a human IgG1. In one embodiment, single domain antibody, multivalent polypeptide or antigen binding molecule of the invention is fused to or conjugated to the Fc domain of a human IgG4.

In one embodiment, the IgG1 Fc domain comprises SEQ ID NO: 169 or a variant thereof. In one embodiment, the IgG1 Fc domain SEQ ID NO: 180 or a variant thereof.

A variant of the IgG1 Fc domain may comprise one or more modifications (amino acid or nucleotide substitutions, deletions or insertions), two or more modifications, three or more modifications. In one embodiment, the IgG1 Fc domain comprises 1, 2, 3, 4, 5, 6, 7, 8, 9 or 10 modifications.

The Fc domain may be joined to the c-terminal or the n-terminal of the single domain antibodies, multivalent polypeptides or antigen binding molecules bind of the invention. In a preferred embodiment, the Fc domain is joined to the c-terminal of the single domain antibodies or multivalent polypeptides of the invention.

Modifications of Fc regions, for example amino acid modifications (substitutions, deletions, insertions), are well known to the skilled person and single domain antibodies, multivalent polypeptides or antigen binding molecules of the invention may be fused or conjugated to an FC domain comprising one or more modifications, for example an Fc domain comprising modifications to reduce or enhance effector mediated functions such as antibody-dependent cellular cytotoxicity (ADCC) or cell-mediated cytotoxicity (CDC), to reduce or enhance binding to a receptor such as the Fc receptor or the C1q receptor. In one embodiment a single domain antibody, multivalent polypeptide or antigen binding molecule of the invention is fused to or conjugated to an IgG1 Fc mutant, wherein the Fc mutant is selected from the group consisting of S267E/H268F/S324T/S239D/I332E, H268F/S324T/S239D/I332E, S267E/H268F/S324T/G236A/I332E, S267E/H268F/S324T, H268F/S324T, G236A/I332E, F243L/R292P/Y300L/V3051/P396L, S239D/I332E, S239D/1332E/A330L, S298A/E333A/K334A, G236A/S239D/I332E, K326W/E333S, S267E/H268F/S324T, E345R/E430G/S440Y, N297A, N297Q, N297G, L235E, L234A/L235A, A330S/P331S, M252Y/S254T/T256E, M428L/N434S, S267E/L328F and N325S/L328F. In one embodiment a single domain antibody. multivalent polypeptide or antigen binding molecule of the invention is fused to or conjugated to an IgG2 Fc mutant, wherein the Fc mutant is selected from the group consisting of H268Q/V309L/A330S/P331S and V234A/G237A/P238S/H268A/V309L/A330S/P331S. In one embodiment a single domain antibody, multivalent polypeptide or antigen binding molecule of the invention is fused to or conjugated to an IgG4 Fc mutant, wherein the Fc mutant is F234A/L235A. In one embodiment a single domain antibody, multivalent polypeptide or antigen binding molecule of the invention is fused to or conjugated to an IgG4 Fc domain wherein the domain is S228P. In one embodiment a single domain antibody, multivalent polypeptide or antigen binding molecules of the invention is fused to or conjugated to an IgG1 Fc domain wherein the domain is L235A.

Multimers, for examples dimers, trimers and tetramers, can be formed when a single domain antibody of the invention is covalently linked to one or more additional single domain antibodies. In one embodiment, a single polypeptide chain is provided comprising two or more single domain antibodies of the invention, as defined herein. In one embodiment, a single polypeptide chain is provided comprising three or more single domain antibodies of the invention, as defined herein. In one embodiment, a single polynucleotide chain is provided encoding two or more single domain antibodies of the invention, as defined herein. In one embodiment, a single polynucleotide chain is provided encoding three or more single domain antibodies of the invention, as defined herein. In one embodiment, a single polypeptide chain is provided comprising one or more single domain antibodies of the invention and one or more known single domain antibodies, as defined herein. In one embodiment, a single polypeptide chain is provided comprising two or more single domain antibodies of the invention and one or more known single domain antibodies, as defined herein. In one embodiment, a single polynucleotide chain is provided encoding one or more single domain antibodies of the invention and one or more known single domain antibodies, as defined herein. In one embodiment, a single polynucleotide chain is provided encoding two or more single domain antibodies of the invention and one or more known single domain antibodies, as defined herein. The multimers may or may not comprise a linker sequence, for example a linker as defined herein.

Dimers can be formed when a single domain antibody of the invention fused to an Fc domain dimerizes with a second single domain antibody or of the invention fused to an Fc domain. Dimerization occurs between the Fc portions via a combination of covalent and non-covalent interactions. Each half of the dimer comprises a single domain antibody of the invention. In one embodiment, the dimer comprises two identical single domain antibodies covalently bound together via the Fc domain (a homodimer). In one embodiment, the dimer comprises two different single domain antibodies covalently bound together via the Fc domain (a heterodimer). In one embodiment, a dimer is provided comprising a first bivalent, optionally bidentate, polypeptide fused to an Fc domain and a second bivalent, optionally bidentate, polypeptide fused to an Fc domain. In this instance, the resulting dimer is tetravalent.

Dimers can also be formed when a coronavirus binding molecule comprising an antigen binding molecule fused to an Fc domain dimerizes with a second coronavirus binding molecule comprising an antigen binding molecule fused to an Fc domain. Dimerization occurs between the Fc portions via a combination of covalent and non-covalent interactions. Each half of the dimer comprises a bidentate coronavirus binding molecule of the invention. The resulting dimer is tetravalent.

Multimers, for examples dimers, trimers and tetramers, can also be formed when a single domain antibody of the invention non-covalently links other single domain antibodies, for example known single domain antibodies or additional single domain antibodies of the invention. In one embodiment, a dimer is provided wherein the dimer comprises two single domain antibodies of the invention, wherein the single domain antibodies are non-covalently linked via a dimerization domain. In one embodiment, a dimer is provided wherein the dimer comprises a single domain antibody of the invention and a known single domain antibody, wherein the single domain antibodies are non-covalently linked via a dimerization domain. In one embodiment, a trimer is provided wherein the trimer comprises three single domain antibodies of the invention, wherein the single domain antibodies are non-covalently linked via a trimerization domain. In one embodiment, a trimer is provided wherein the trimer comprises two single domain antibodies of the invention and one known single domain antibody, wherein the single domain antibodies are non-covalently linked via a trimerization domain. In one embodiment, a trimer is provided wherein the trimer comprises one single domain antibody of the invention and two known single domain antibodies, wherein the single domain antibodies are non-covalently linked via a trimerization domain.

In one embodiment a dimer is provided, wherein a single domain antibody of the invention fused to a Fc domain is dimerized with a further single domain antibody of the invention fused to a Fc. The Fc domain itself causes dimerization. In one embodiment, a dimer is provided comprising:
a) a first single domain antibody having an amino acid or polynucleotide sequence based on C5, B12, F2, C1 or H3, wherein the first single domain antibody is fused to a Fc domain; and
b) a second single domain antibody having an amino acid or polynucleotide based on C5, B12, F2, C1 or H3, wherein the second single domain antibody is fused to a Fc domain; or
a) a first single domain antibody having an amino acid or polynucleotide sequence based on C5, A8, B12, F2, C1, H3, NbSA_A10, NbSA_D10, 3_05, 8_G11, 12_F11 and VHH_H6 wherein the first single domain antibody is fused to a Fc domain; and
b) a second single domain antibody having an amino acid or polynucleotide based on C5, A8, B12, F2, C1, H3, NbSA_A10, NbSA_D10, 3_05, 8_G11, 12_F11 and VHH_H6, wherein the second single domain antibody is fused to a Fc domain.

As detailed throughout, the amino acid or polynucleotide sequence can comprise the CDR3, CDR2 and/or CDR1 or a variant thereof of the specified single domain antibodies, comprise the amino acid sequence or a variant thereof of the specified single domain antibodies, or comprise the polynucleotide sequence of variant thereof of the specified single domain antibodies.

In one embodiment, an anti-SARS-CoV-2 dimer ((C5-Fc)2) is provided comprising:
a) a first single domain antibody comprising a CDR1 comprising SEQ ID NO:10, a CDR2 comprising SEQ ID NO:11 and a CDR3 comprising SEQ ID NO:12, and wherein a Fc domain is fused to the c-terminal of the single domain antibody;
b) a second single domain antibody comprising a CDR1 comprising SEQ ID NO:10, a CDR2 comprising SEQ ID NO:11 and a CDR3 comprising SEQ ID NO:12, and wherein a Fc domain is fused to the c-terminal of the single domain antibody, wherein the amino acid sequence of each CDR3 comprises between 0 and 7 amino acid modifications, wherein the amino acid sequence of each CDR2 comprises between 0 and 4 amino acid modifications and wherein the amino acid sequence of each CDR1 comprises between 0 and 4 amino acid modifications. In a preferred embodiment, the Fc domain is human IgG1 or variant thereof.

In one embodiment, an anti-SARS-CoV-2 dimer ((A8-Fc)2) is provided comprising:
a) a first single domain antibody comprising a CDR1 comprising SEQ ID NO:196, a CDR2 comprising SEQ ID NO:197 and a CDR3 comprising SEQ ID NO:198 and wherein a Fc domain is fused to the c-terminal of the single domain antibody;
b) a second single domain antibody comprising a CDR1 comprising SEQ ID NO:196, a CDR2 comprising SEQ ID NO:197 and a CDR3 comprising SEQ ID NO:198, and wherein a Fc domain is fused to the c-terminal of the single domain antibody,
wherein the amino acid sequence of each CDR3 comprises between 0 and 7 amino acid modifications, wherein the amino acid sequence of each CDR2 comprises between 0 and 4 amino acid modifications and wherein the amino acid sequence of each CDR1 comprises between 0 and 4 amino acid modifications. In a preferred embodiment, the Fc domain is human IgG1 or variant thereof.

In one embodiment, a dimer is provided comprising a first single domain antibody having an amino acid or polynucleotide sequence based on B12, C1, C5, F2, H3, NbSA_A10, NbSA_D10, A8, 3_05, 8_G11, 12_F11 or VHH_H6 (the first row of the table below) fused to a Fc domain, and a second single domain antibody having an amino acid or polynucleotide based on B12, C1, C5, F2, H3, NbSA_A10, NbSA_D10, A8, 3_05, 8_G11, 12_F11 or VHH_H6 (the first column of the table below) fused to a Fc domain. In one embodiment, a dimer is provided comprising a first single domain antibody having an amino acid or polynucleotide sequence based on B12, C1, C5, F2, H3, NbSA_A10, NbSA_D10, A8, 3_05, 8_G11, 12_F11 or VHH_H6 (the first row of the table below) covalently linked to a second single domain antibody having an amino acid or polynucleotide based on B12, C1, C5, F2, H3, NbSA_A10, NbSA_D10, A8, 3_C5, 8_G11, 12_F11 or VHH_H6 (the first column of the table below). In one embodiment, a dimer is provided comprising a first single domain antibody having an amino acid or polynucleotide sequence based on B12, C1, C5, F2, H3, NbSA_A10, NbSA_D10, A8, 3_C5, 8_G11, 12_F11 or VHH_H6 (the first row of the table below) non-covalently linked, optionally via a dimerization domain, to a second single domain antibody having an amino acid or polynucleotide based on B12, C1, C5, F2, H3, NbSA_A10, NbSA_D10, A8, 3_C5, 8_G11, 12_F11 or VHH_H6 (the first column of the table below). The table below illustrates the various combinations of two single domain antibodies of the invention that may form a dimer. In a preferred embodiment, a dimer is provided comprising a first single domain antibody comprising an amino acid or polynucleotide sequence based on 2_A8 or C5 fused to a Fc domain, and a second single domain antibody having an amino acid or polynucleotide based on B12, C1, C5, F2, H3, NbSA_A10, NbSA_D10, A8, 3_C5, 8_G11, 12_F11 or VHH_H6 (the first column of the table below) fused to a Fc domain. In a preferred embodiment, a dimer is provided comprising a first single domain antibody comprising an amino acid or polynucleotide sequence based on 2_A8 or C5 covalently linked to a second single domain antibody having an amino acid or polynucleotide based on B12, C1, C5, F2, H3, NbSA_A10, NbSA_D10, A8, 3_C5, 8_G11, 12_F11 or VHH_H6 (the first column of the table below). In a preferred embodiment, a dimer is provided comprising a first single domain antibody comprising an amino acid or polynucleotide sequence based on 2_A8 or C5 non-covalently linked, optionally via a dimerization domain, to a second single domain antibody having an amino acid or polynucleotide based on B12, C1, C5, F2, H3, NbSA_A10, NbSA_D10, A8, 3_05, 8_G11, 12_F11 or VHH_H6 (the first column of the table below). In a further preferred embodiment, a dimer is provided comprising a first single domain comprising an amino acid or polynucleotide sequence based on A8 or C5 fused to a Fc domain and a second single domain antibody comprising an amino acid or polynucleotide sequence based on A8 or C5 fused to a Fc domain. In a further preferred embodiment, a dimer is provided comprising a first single domain comprising an amino acid or polynucleotide sequence based on A8 or C5 covalently linked to a second single domain antibody comprising an amino acid or polynucleotide sequence based on A8 or C5. In a further preferred embodiment, a dimer is provided comprising a first single domain comprising an amino acid or polynucleotide sequence based on 2_A8 or C5 non-covalently linked, optionally via a dimerization domain, to a second single domain antibody comprising an amino acid or polynucleotide sequence based on A8 or C5. As detailed throughout, the amino acid or polynucleotide sequence can comprise the CDR3, CDR2 and/or CDR1 or a variant thereof of the specified single domain antibodies, comprise the amino acid sequence or a variant thereof of the specified single domain antibodies, or comprise the polynucleotide sequence of variant thereof of the specified single domain antibodies.

| | | | | | | NbSA_ | NbSA_ | | | | | VHH_ |
|---|---|---|---|---|---|---|---|---|---|---|---|---|
| | B12 | F2 | C1 | C5 | H3 | A10 | D10 | A8 | 3_C5 | 8_G11 | 12_F11 | H6 |
| B12 | B12/ | F2/B12 | C1/B12 | C5/B12 | H3/B12 | NbSA_ | NbSA_ | A8/B12 | 3_C5/ | 8_G3/ | 12_F11/ | VHH_ |
| | B12 | | | | | A10/ | D10/ | | B12 | B12 | B12 | H6/B12 |
| | | | | | | B12 | B12 | | | | | |
| F2 | B12/F2 | F2/F2 | C1/F2 | C5/F2 | H3/F2 | NbSA_ | NbSA_ | A8/F2 | 3_C5 | 8_G3/ | 12_F11/ | VHH_ |
| | | | | | | A10/F2 | D10/F2 | | /F2 | F2 | F2 | H6/F2 |
| C1 | B12/C1 | F2/C1 | C1/C1 | C5/C1 | H3/C1 | NbSA_ | NbSA_ | A8/C1 | 3_C5/ | 8_G3/ | 12_F11/ | VHH_ |
| | | | | | | A10/C1 | D10/C1 | | C1 | C1 | C1 | H6/C1 |
| C5 | B12/C5 | F2/C5 | C1/C5 | C5/C5 | H3/C5 | NbSA_ | NbSA_ | A8/C5 | 3_C5/ | 8_G3/ | 12_F11/ | VHH_ |
| | | | | | | A10/C5 | D10/C5 | | C5 | C5 | C5 | H6/C5 |
| H3 | B12/H3 | F2/H3 | C1/H3 | C5/H3 | H3/H3 | NbSA_ | NbSA_ | A8/H3 | 3_C5/ | 8_G3/ | 12_F11/ | VHH_ |
| | | | | | | A10/H3 | D10/H3 | | H3 | H3 | H3 | H6/H3 |
| NbSA_ | B12/ | F2/ | C1/ | C5/ | H3/ | NbSA_ | NbSA_ | A8/ | 3_C5/ | 8_G3 | 12 F11/ | VHH_ |
| A10 | NbSA_ | NbSA_ | NbSA_ | NbSA_ | NbSA_ | A10/ | D10/ | NbSA_ | NbSA_ | NbSA_ | NbSA_ | H6/ |
| | A10 | A10 | A10 | A10 | A10 | NbSA_ | NbSA_ | A10 | A10 | A10/ | A10 | NbSA_ |
| | | | | | | A10 | A10 | | | | | A10 |
| NbSA_ | B12/ | F2/ | C1/ | C5/ | H3/ | NbSA_ | NbSA_ | A8/ | 3_C5/ | 8_G3/ | 12_F11/ | VHH_ |
| D10 | NbSA_ | NbSA_ | NbSA_ | NbSA_ | NbSA_ | A10/ | D10/ | NbSA_ | NbSA_ | NbSA_ | NbSA_ | H6/ |
| | D10 | D10 | D10 | D10 | D10 | NbSA_ | NbSA_ | D10 | D10 | D10 | D10 | NbSA_ |
| | | | | | | D10 | D10 | | | | | D10 |
| A8 | B12A8 | F2/A8 | C1A8 | C5A8 | H3A8 | NbSA_ | NbSA_ | A8/A8 | 3_C5/ | 8_G3/ | 12_F11/ | VHH_ |
| | | | | | | A10/A8 | D10/A8 | | A8 | A8 | A8 | H6 /A8 |
| 3_C5 | B12/ | F2/3_ | C1/3_ | C5/3_ | H3/3_ | NbSA_ | NbSA_ | A8/3_ | 3_C5/ | 8_G3/ | 12_F11/ | VHH_ |
| | 3_C5 | C5 | C5 | C5 | C5 | A10/3_ | D10/3_ | C5 | 3_C5 | 3_C5 | 3_C5 | H6/3_ |
| | | | | | | C5 | C5 | | | | | C5 |
| 8_G11 | B12/ | F2/8_ | C1/8_ | C5/8_ | H3/8_ | NbSA_ | NbSA_ | A8/8_ | 3_C5/ | 8_G3/ | 12_F11/ | VHH_ |
| | 8_G11 | G11 | G11 | G11 | G11 | A10/8_ | D10/8_ | G11 | 8_G11 | 8_G11 | 8_G11 | H6/8_ |
| | | | | | | G11 | G11 | | | | | G11 |
| 12_F11 | B12/ | F2/12_ | C1/12_ | C5/12_ | H3/12_ | NbSA_ | NbSA_ | A8/12_ | 3_C5/ | 8_G3/ | 12_F11/ | VHH_ |
| | 12_F11 | F11 | F11 | F11 | F11 | A10/ | D10/ | F11 | 12_ | 12_ | 12_F11 | H6/12_ |
| | | | | | | 12_F11 | 12_F11 | | F11 | F11 | | F11 |
| VHH_ | B12/H6 | F2/H6 | C1/H6 | C5/H6 | H3/H6 | NbSA_ | NbSA_ | A8/H6 | 3_C5/ | 8_G3/ | 12_F11/ | VHH_ |
| H6 | | | | | | A10/H6 | D10/H6 | | H6 | H6 | H6 | H6/ |
| | | | | | | | | | | | | VHH_ |
| | | | | | | | | | | | | H6 |

In one embodiment, a fusion protein (C5-Fc) is provided comprising a single domain antibody (C5) fused to the Fc region of hIgG1 having the following sequence, or a variant thereof:

SEQ ID NO: 161
CAGGTGCAGCTGGTGGAGTCTGGGGGAGGCTCGGTGCAGG
CTGGGGGGTCTCTGACACTCTCCTGTGTCGCCTCTGGAGT
CACTTTGGGACGTCATGCCATAGGCTGGTTCCGCCAGGCC
CCCGGGAAGGAGCGTGAGAGAGTCTCGTGTATTAGAACAT
TTGATGGCATCACAAGTTATGTAGAGTCCACGAAGGGCCG
ATTCACCATCTCCAGTAACAATGCCATGAACACGGTGTAT
CTGCAAATGAATAGCCTCAAACCTGAAGACACGGCCGTTT
ATTTCTGTGCACTGGGAGTGACTGCAGCCTGTTCAGATAA
TCCCTACTTCTGGGGCCAGGGGACCCAGGTCACCGTCTCC
TCAGCGTCGACCGAGCCCAAATCTTGTGACAAAACTCACA
CATGCCCACCGTGCCCAGCACCTGAACTCCTGGGGGGACC
GTCAGTCTTCCTCTTCCCCCCAAAACCCAAGGACACCCTC
ATGATCTCCCGGACCCCTGAGGTCACATGCGTGGTGGTGG
ACGTGAGCCACGAAGACCCTGAGGTCAAGTTCAACTGGTA
CGTGGACGGCGTGGAGGTGCATAATGCCAAGACAAAGCCG
CGGGAGGAGCAGTACAACAGCACGTACCGTGTGGTCAGCG
TCCTCACCGTCCTGCACCAGGACTGGCTGAATGGCAAGGA
GTACAAGTGCAAGGTCTCCAACAAAGCCCTCCCAGCCCCC
ATCGAGAAAACCATCTCCAAAGCCAAAGGGCAGCCCCGAG
AACCACAGGTGTACACCCTGCCCCCATCCCGGGAGGAGAT
GACCAAGAACCAGGTCAGCCTGACCTGCCTGGTCAAAGGC
TTCTATCCCAGCGACATCGCCGTGGAGTGGGAGAGCAATG
GGCAGCCGGAGAACAACTACAAGACCACGCCTCCCGTGCT
GGACTCCGACGGCTCCTTCTTCCTCTATAGCAAGCTCACC
GTGGACAAGAGCAGGTGGCAGCAGGGGAACGTCTTCTCAT
GCTCCGTGATGCATGAGGCTCTGCACAACCACTACACGCA
GAAGAGCCTCTCCCTGTCCCCGGGTAAA
SEQ ID NO: 170
QVQLVESGGGSVQAGGSLTLSCVASGVTLGRHAIGWFRQA
PGKERERVSCIRTFDGITSYVESTKGRFTISSNNAMNTVY
LQMNSLKPEDTAVYFCALGVTAACSDNPYFWGQGTQVTVS
SASTEPKSCDKTHTCPPCPAPELLGGPSVFLFPPKPKDTL
MISRTPEVTCVVVDVSHEDPEVKFNWYVDGVEVHNAKTKP
REEQYNSTYRVVSVLTVLHQDWLNGKEYKCKVSNKALPAP
IEKTISKAKGQPREPQVYTLPPSREEMTKNQVSLTCLVKG
FYPSDIAVEWESNGQPENNYKTTPPVLDSDGSFFLYSKLT
VDKSRWQQGNVFSCSVMHEALHNHYTQKSLSLSPGK

In some embodiments, the one or more amino acid modifications are in the CDR region or regions. In some embodiments, the one or more amino acid modifications are in the framework regions, i.e. not in the CDR region or regions. In some embodiments, the one or more polynucleotide modifications are in the CDR region or regions. In some embodiments, the one or more polynucleotide modifications are in the framework regions, i.e. not in the CDR region or regions. In some embodiments, the one or more amino acid modifications are in the CDR region or regions and the framework regions. In some embodiments, the one or more polynucleotide modifications are in the CDR region or regions and the framework regions.

In one embodiment the CDR3 regions comprise between 0 and 7, 0 and 6, 0 and 5, 0 and 4, 0 and 3, 0 and 2 or 0 and 1 amino acid modifications. In one embodiment the CDR2 regions comprise 0 and 4, 0 and 3, 0 and 2 or 0 and 1 amino acid modifications. In one embodiment the CDR1 regions comprise 0 and 4, 0 and 3, 0 and 2 or 0 and 1 amino acid modifications. The modifications can be substitutions, deletions or insertions. In one embodiment, the modifications are substitutions.

In one embodiment, a single domain antibody or antigen binding molecule of the invention comprising one or more modifications has a binding affinity for the receptor binding domain of the SARS-CoV-2 S-protein that is substantially equal to, or better than (for example, a lower Kd value) than the specified sequence without any modifications.

The single domain antibodies or coronavirus binding molecules of the invention bind to the receptor binding domain of the SARS-CoV-2 S-protein. In one embodiment, the single domain antibodies or coronavirus binding molecules of the invention block or modulate the binding between the receptor binding domain of a coronavirus, in particular the SARS-CoV-2 spike (S) protein, and the angiotensin converting enzyme 2 receptor (ACE2 receptor). In one embodiment, the single domain antibodies or coronavirus binding molecules of the invention inhibit binding of the receptor binding domain of the SARS-CoV-2 spike (S) protein to the ACE2 receptor, wherein binding of the receptor binding domain of the SARS-CoV-2 spike (S) protein to the ACE2 receptor is inhibited by at least 10%, optionally at least 20%, at least 30%, at least 40%, at least 50%, at least 60%, at least 70%, at least 80%, at least 90%, at least 95% or 100%. Percentage inhibition of binding to the ACE2 receptor can be measured in numerous ways, as well understood by the skilled person, including but not limited to surface plasmon resonance.

In one embodiment, a coronavirus binding molecule is provided wherein the first antigen binding molecule, when bound to the first epitope blocks the RBD of the coronavirus from binding to the human ACE2 protein.

By interfering with the interaction between a coronavirus spike protein and its target, the single domain antibodies, multivalent polypeptides, fusion proteins or coronavirus binding molecules of the invention can neutralize coronavirus infection. In one embodiment the single domain antibodies, multivalent polypeptides, fusion proteins or coronavirus binding molecules of the invention can neutralize SARS-CoV-2 infection. In one embodiment, the single domain antibodies, multivalent polypeptides, fusion proteins or coronavirus binding molecules have an ND50 value (ND50=concentration of antibody that reduces the number of infected cells by 50%) of less than 100 pM, less than 10 μM, less than 5 μM, less than 1 μM, less than 0.5 μM, less than 0.1 μM or less than 0.01 μM. In one embodiment, the single domain antibodies have an ND50 value of less than 100 nM less than 10 nM, less than 5 nM, less than 1 nM, less than 0.5 nM or less than 0.1 nM. In one embodiment, the single domain antibodies, multivalent polypeptides, fusion proteins or coronavirus binding molecules have an ND50 value of less than 0.1 nM less than 10 pM, less than 5 pM, less than 1 pM, less than 0.5 pM or less than 0.1 pM. The ND50 value can be determined using any standard neutralization assay, including that disclosed herein.

In some embodiments, administration of the single domain antibodies, multivalent polypeptides, fusion proteins or coronavirus binding molecules of the present invention prevents or substantially reduces non-neutralised virus from replicating and/or spreading. In some embodiments, single domain antibodies, multivalent polypeptides, fusion proteins or coronavirus binding molecules of the present invention are capable of forming plaques that are 5% smaller than in the presence of a positive control, for example CR3022. In some embodiments, the plaques are 10% smaller than in the presence of a positive control (for example CR3022); in some embodiments, plaques are 15%, 20%, 25%, 30%, 35%, 40%, 45%, 50%, 55%, 60%, 65%, 70%, 75%, 80%, 85%, 90%, or 95% smaller than in the presence of a positive control (for example CR3022).

In one embodiment, the single domain antibodies, multivalent polypeptides, fusion proteins or coronavirus binding molecules of the invention have a Kd value for SARS-CoV-2 spike protein of less than 500 pM, less than 400 pM, less than 200 pM, less than 100 pM, less than 75 pM, less than 50 μm, less than 25 pM, less than 10 pM, less than 5 pM, less than 1 pM or less than 0.1 pM. Binding affinity can be measured according to several standard well-known techniques, including for example surface plasma resonance. In one embodiment, a single domain antibody, multivalent polypeptide, fusion protein or coronavirus binding molecule of the invention having one or more modifications as specified herein has a binding affinity value that is within 20% (i.e. within the range of 20% below or 20% above the binding affinity value of the corresponding single domain antibody without one or more modifications) of the binding affinity value of the corresponding single domain antibody, multivalent polypeptide, fusion protein or coronavirus binding molecule without one or more modifications. In one embodiment, the binding affinity value of a single domain antibody, multivalent polypeptide, fusion protein or coronavirus binding molecule of the invention having one or more modifications as specified herein is within 10%, optionally 5%, 4%, 3%, 2% or 1% of the binding affinity value of the corresponding single domain antibody, multivalent polypeptide, fusion protein or coronavirus binding molecule without one or more modifications.

Furthermore, the single domain antibodies, multivalent polypeptides, fusion proteins or coronavirus binding molecules of the invention can modulate, reduce or prevent coronavirus infectivity. The single domain antibodies, multivalent polypeptides or fusion proteins of the invention can modulate, block or inhibit the fusion of a coronavirus to a target host cell. The single domain antibodies, multivalent polypeptides, fusion proteins or coronavirus binding molecules of the invention can modulate, block or inhibit entry of coronavirus into a target host cell.

In one aspect, an affinity matured mutant of a single domain antibody of the invention is provided. In one embodiment, the CDR1 of the single domain antibody of the invention is affinity matured. In one embodiment, the CDR2 of the single domain antibody of the invention is affinity matured. In one embodiment, the CDR3 of the single domain antibody of the invention is affinity matured. In one embodiment, CDR3 is affinity matured and either CDR1 or CDR2 are also affinity matured. In one embodiment, CDR3 is affinity matured and CDR2 is also affinity matured. In one embodiment, CDR3 is affinity matured and CDR1 is also affinity matured. In one embodiment, each of CDR1, CDR2 and CDR3 are affinity matured. In one embodiment, at least one, at least two, at least three or all four of the framework regions (FR1, FR2, FR3 and FR4) are affinity matured. In one embodiment, each of CDR1, CDR2, CDR3, FR1, FR2, FR3 and FR4 are affinity matured. In one embodiment, the affinity of the affinity matured mutant of a single domain antibody of the invention has a higher affinity for SARS-CoV-2 receptor binding domain (RBD) than the parental antibody from which it was derived.

In one aspect, a humanized single domain antibody of the invention is provided. Humanization requires the modification of the amino acid sequence of the antibody. Methods to reduce the immunogenicity of the single domain antibodies of the invention include CDR grafting on to a suitable antibody framework scaffold or remodelling variable surface residues, e.g. by site-directed mutagenesis. Methods of humanization of Nanobodies® are known to the skilled person, see for example Vincke et al., 2009. In one embodiment, the CDR1 of the single domain antibody of the invention is humanized. In one embodiment, the CDR2 of the single domain antibody of the invention is humanized. In one embodiment, the CDR3 of the single domain antibody of the invention is humanized. In one embodiment, at least one or at least two of the CDR1, CDR2 and CDR3 are humanized. In one embodiment, each of CDR1, CDR2 and CDR3 are humanized. In one embodiment, at least one, at least two, at least three or all four of the framework regions (FR1, FR2, FR3 and FR4) are humanized. In one embodiment, each of CDR1, CDR2, CDR3, FR1, FR2, FR3 and FR4 are humanized. In some embodiments, the single domain antibodies are conservatively humanised, for example to retain better antigen binding.

In one embodiment, a vector suitable for expressing a single domain antibody, multivalent polypeptide or fusion protein sequence of the invention is provided. The vector may be a plasmid, viral vector, cosmid, phage or artificial chromosome. In one aspect, a host cell comprising an expression vector or plasmid, wherein the expression vector or plasmid comprises a polynucleotide of the invention is provided. In one embodiment, the host cell comprises a polynucleotide of the invention integrated within the genome of the host cell. In one embodiment, the host cell is a prokaryotic cell, for example a bacterial cell, or a eukaryotic cell, for example a yeast cell or mammalian cell. In one embodiment, the host cell is Escherichia coli or CHO cells.

In one aspect, a method for producing a single domain antibody, multivalent polypeptide or fusion protein of the invention is provided comprising the steps of (a) culturing a host cell as provided herein under conditions suitable for producing a single domain antibody, multivalent polypeptide or fusion protein to obtain a culture containing single domain antibodies, multivalent polypeptides or fusion proteins and (b) isolating said single domain antibodies from the culture.

One aspect of the invention provides single domain antibodies, multivalent polypeptides, fusion proteins or coronavirus binding molecules as defined above in a composition or pharmaceutical composition. The compositions may comprise, consist essentially of or consist of the single domain antibodies, multivalent polypeptides, fusion proteins or coronavirus binding molecules of the invention.

In one embodiment, a pharmaceutical composition comprising single domain antibodies, multivalent polypeptides, fusion proteins or coronavirus binding molecules of the invention is provided. The pharmaceutical compositions may be for human or animal usage in human and veterinary medicine. The pharmaceutical composition may be formulated according to route of administration. In one embodiment, the pharmaceutical composition is formulated for oral, nasal, ocular, buccal, vaginal, rectal, transdermal, intravenous, intramuscular or subcutaneous administration. In a preferred route of administration, the pharmaceutical composition is formulated for administration by inhalation, optionally nasal and or oral inhalation. Pharmaceutical compositions in this form may include aerosols, fine particles or dust.

In one embodiment, the composition or pharmaceutical composition optionally comprises one or more pharmaceutically acceptable excipients. In one embodiment, the composition or pharmaceutical composition optionally comprises one or more pharmaceutically acceptable adjuvants. In one embodiment, the composition or pharmaceutical composition is optionally admixed with one or more pharmaceutically acceptable diluents, excipients or carriers. Examples of such suitable excipients for the different forms of pharmaceutical compositions described herein may be found in the “Handbook of Pharmaceutical Excipients, 2^{nd} Edition, (1994), Edited by A Wade and PJ Weller.

The composition or pharmaceutical composition may comprise one or more additional components. In one embodiment, the composition or pharmaceutical composition additionally comprises a pharmaceutically acceptable carrier. In one embodiment, the carrier is suitable for pulmonary delivery. In one embodiment, the composition or pharmaceutical composition additionally comprises a therapeutically active agent.

In one embodiment, the composition or pharmaceutical composition may be joined or conjugated to a protein or biologically active molecule. In one embodiment, the composition or pharmaceutical composition is part of a fusion protein and fused to one or more proteins or biologically active molecules. The protein or biologically active molecule may be a fluorescent protein, a bioluminescent protein, a split fluorescent protein (i.e. split into two or more parts that will join together in the presence of drug), a split bioluminescent protein, a biosensor, a fluorescent biosensor or a split or hinged biosensor.

In one embodiment, a vaccine comprising single domain antibodies, multivalent polypeptides, fusion proteins or coronavirus binding molecules of the invention is provided. In one embodiment, the vaccine comprises a polynucleotide encoding a single domain antibody, multivalent polypeptide, fusion protein or coronavirus binding molecule of the invention is provided.

The compositions, pharmaceutical compositions and vaccines of the invention can elicit an immune response in a subject, preferably an immune response to SARS-CoV-2. In some embodiments, the immune response is a protective immune response. In some embodiments, the immune response that reduces the symptoms or severity of SARS-CoV-2 in a subject.

A pharmaceutical device, for example an inhaler or nebulizer, suitable to administer the pharmaceutical compositions of the invention is also provided. In one embodiment, the pharmaceutical device, for example an inhaler or nebulizer, comprises a single domain antibody, multivalent polypeptide, fusion protein or coronavirus binding molecule of the invention.

A kit providing single domain antibodies, multivalent polypeptides, fusion proteins or coronavirus binding molecules of the invention is also provided. Such kits may include instructions for use and/or additional pharmaceutically active components. The single domain antibodies, multivalent polypeptides, fusion proteins or coronavirus binding molecules and the additional pharmaceutically active components may be formulated together, or alternatively in some embodiments, the single domain antibodies, multivalent polypeptides, fusion proteins or coronavirus binding molecules and the additional pharmaceutically active components may be present separately in the kit.

In one aspect, there is provided a single domain antibody, multivalent polypeptide, fusion protein or coronavirus binding molecule of the invention or a pharmaceutical composition of the invention for use in medicine. The single domain antibodies, multivalent polypeptides, fusion proteins, coronavirus binding molecules or pharmaceutical compositions of the invention can be used to treat a coronavirus, optionally Middle Eastern respiratory syndrome (MERS-CoV) or severe acute respiratory syndrome coronavirus 1 (SARS-CoV-1), preferably COVID-19. The single domain antibodies, multivalent polypeptides, fusion proteins, coronavirus binding molecules or pharmaceutical compositions of the invention can be used to treat specific strains of COVID-19, including the prototypical Wuhan/Victoria strain, the B.1.1.7 variant (UK or Kent variant/Alpha), the B.1.351 variant (South African variant/Beta), the P.1 variant (Brazilian/Gamma), the B.1.617.2 variant (Indian variant/Delta), the B.1.427/B.1.429 variant (Epsilon), the P.2 variant (Zeta), The B.1.525 variant (Eta), the P.3 variant (Theta), the B.1.526 variant (Iota) and the B.1.617.1 variant (Kappa). The single domain antibodies, multivalent polypeptides, fusion proteins, coronavirus binding molecules or pharmaceutical compositions of the invention can be used to block or modify the interaction of the spike protein of a coronavirus, in particular SARS-CoV-2, with its target, angiotensin converting enzyme 2 receptor. In one embodiment, the single domain antibodies, multivalent polypeptides, fusion proteins, coronavirus binding molecules or pharmaceutical compositions of the invention block, reduce or inhibit binding of the spike protein of a coronavirus, in particular SARS-CoV-2, with its target, angiotensin converting enzyme 2 (ACE2) receptor. By interfering with the interaction between the spike protein and its target, the single domain antibodies, multivalent polypeptides, fusion proteins or pharmaceutical compositions of the invention can neutralize coronavirus and/or can modulate, reduce or prevent coronavirus infectivity. The single domain antibodies, multivalent polypeptides, fusion proteins, coronavirus binding molecules or pharmaceutical compositions of the invention can modulate, block or inhibit the fusion of coronavirus to a target host cell. The single domain antibodies, multivalent polypeptides, fusion proteins, coronavirus binding molecules or pharmaceutical compositions of the invention can modulate, block or inhibit entry of coronavirus into a target host cell.

The single domain antibodies, multivalent polypeptides, fusion proteins, coronavirus binding molecules or pharmaceutical compositions of the invention can be used for the treatment or prophylaxis of coronavirus infection, in particular COVID-19. In one embodiment, there is provided a single domain antibody, multivalent polypeptide, fusion protein, coronavirus binding molecule or pharmaceutical composition of the invention for use in the treatment or prophylaxis of a coronavirus infection optionally Middle Eastern respiratory syndrome (MERS-CoV) or severe acute respiratory syndrome coronavirus 1 (SARS-CoV-1), preferably COVID-19. In one embodiment, there is provided a single domain antibody, multivalent polypeptide, fusion protein, coronavirus binding molecule or pharmaceutical composition of the invention for use in the treatment or prophylaxis of COVID-19.

In one aspect, a method for the treatment of a coronavirus in a subject is provided, comprising administering to a subject a therapeutically active amount of a single domain antibody, multivalent polypeptide, fusion protein, coronavirus binding molecule or pharmaceutical composition of the invention. In one embodiment the subject is a mammal, preferably a human.

In one aspect, the use of a single domain antibody, multivalent polypeptide, fusion protein, coronavirus binding molecule or pharmaceutical composition of the invention in the manufacture of a medicament for use in the treatment and/or prevention of a coronavirus is provided. In one embodiment, the use of a single domain antibody, multivalent polypeptide, fusion protein, coronavirus binding molecule or pharmaceutical composition of the invention in the manufacture of a medicament for use in the treatment of a coronavirus is provided.

In one embodiment, the coronavirus is selected from the group consisting of MERS-CoV, SARS-CoV-1 and COVID-19. In one embodiment, the coronavirus is COVID-19. In one embodiment, the coronavirus is a COVID-19 strain selected from the prototypical Wuhan/Victoria strain, the B.1.1.7 variant (UK or Kent variant/Alpha), the B.1.351 variant (South African variant/Beta), the P.1 variant (Brazilian/Gamma), the B.1.617.2 variant (Indian variant/Delta), the B.1.427/B.1.429 variant (Epsilon), the P.2 variant (Zeta), The B.1.525 variant (Eta), the P.3 variant (Theta), the B.1.526 variant (Iota) and the B.1.617.1 variant (Kappa). The invention may relate to treating a subject displaying severe symptoms of COVID-19 or alternatively to treating a subject showing milder symptoms of COVID-19 or alternatively to treating a subject who has tested positive for COVID-19 but is asymptomatic for the disease. In some embodiments, the single domain antibodies, multivalent polypeptides, fusion proteins, coronavirus binding molecules or pharmaceutical compositions of the invention are useful for treating a cytokine storm associated with a coronavirus infection.

In one embodiment, a trivalent polypeptide of the invention comprising three single domain amino antibodies having an amino acid or polynucleotide sequence based on C1 is provided for use in treating COVID-19 (Wuhan/Victoria strain), the B.1.1.7 COVID_19 variant (UK or Kent variant/Alpha) and/or the B.1.351 COVID-19 variant (South African variant/Beta), In one embodiment, a trivalent polypeptide of the invention comprising three single domain amino antibodies having an amino acid or polynucleotide sequence based on A8 is provided for use in treating COVID-19 (Wuhan/Victoria strain), the B.1.1.7 COVID_19 variant (UK or Kent variant/Alpha) and/or the B.1.351 COVID-19 variant (South African variant/Beta), In one embodiment, a trivalent polypeptide of the invention comprising three single domain amino antibodies having an amino acid or polynucleotide sequence based on H3 is provided for use in treating COVID-19 (Wuhan/Victoria strain) and/or the B.1.1.7 COVID_19 variant (UK or Kent variant/Alpha). In one embodiment, a trivalent polypeptide of the invention comprising three single domain amino antibodies having an amino acid or polynucleotide sequence based on C5 is provided for use in treating COVID-19 (Wuhan/Victoria strain) and/or the B.1.1.7 COVID_19 variant (UK or Kent variant/Alpha).

In one embodiment, methods for the detection of a coronavirus protein, such as MERS-CoV, SARS-CoV-1 and SARS-CoV-2 are provided. In a preferred embodiment, a method for the detection of a SARS-CoV-2 protein is provided. In one embodiment, a method for detecting the presence of a coronavirus S-protein is provided. In one embodiment, a method for a method for detecting the presence of a SARS-CoV-2 S-protein is provided.

In one embodiment, a method for detecting a coronavirus protein in a sample is provided, wherein the method comprises the steps of (a) contacting a sample with the single domain antibodies, multivalent polypeptides, fusion proteins or coronavirus binding molecules of the invention and (b) detecting the antibody-antigen complex, wherein the presence of the complex indicates the presence of coronavirus protein. In step (a) of the method the sample is contacted with the single domain antibodies, multivalent polypeptides, fusion proteins or coronavirus binding molecules under suitable conditions for an antibody-antigen complex to form. The antigen is the coronavirus protein. In one embodiment, a method for detecting the presence of a coronavirus S-protein is provided. In one embodiment, a method for a method for detecting the presence of a SARS-CoV-2 S-protein, optionally the receptor binding domain of the S-protein, is provided.

The sample can be a biological sample, optionally a bodily fluid such as blood, serum, nasal secretions, sputum, plasma, urine or spinal fluid. In one embodiment the biological sample is bodily fluid obtained using a throat or nasal swab. In one embodiment, the biological sample is a tissue sample. The sample can be obtained from or isolated from a mammal, preferably a human. In one embodiment, the sample is obtained from or isolated from a subject who is suspected to have coronavirus.

Detecting the presence of coronavirus protein in a sample from a subject provides a positive indication that the subject is infected with coronavirus. In one embodiment, the results of the method of detection are used to diagnose a subject in relation to coronavirus. The presence of coronavirus protein in the method of detection would provide a positive diagnosis for coronavirus. The method of detection may also be used to provide a prediction of outcome in relation to infection of coronavirus infection.

In one embodiment, a method for detecting coronavirus protein in a subject is provided, wherein the method comprises the steps of (a) administering to a subject a single domain antibody, multivalent polypeptide, fusion protein or coronavirus binding molecule of the invention and (b) detecting the presence of an antibody-antigen complex, wherein the presence of the complex indicates the presence of coronavirus protein in the subject. In one embodiment, a method for detecting coronavirus protein in a subject is provided, wherein the method comprises the steps of (a) administering to a subject a single domain antibody of the invention, (b) obtaining a sample from a subject and contacting the sample with a single domain antibody of the invention and (c) detecting the antibody-antigen complex, wherein the presence of the complex indicates the presence of coronavirus protein in the subject. The antigen is the coronavirus protein. In one embodiment, a method for detecting coronavirus protein in a subject is provided, wherein the method comprises the steps of (a) obtaining a sample from a subject, (b) contacting a sample from the subject with a single domain antibody of the invention and (c) detecting the antibody-antigen complex, wherein the presence of the complex indicates the presence of coronavirus protein in the subject. The sample may be an isolated sample (i.e. previously obtained from a subject).

In one aspect, a method for diagnosing coronavirus infection in a subject is provided, wherein the method comprises the steps of (a) administering to a subject a single domain antibody of the invention and (b) detecting the presence of an antibody-antigen complex, wherein the presence of the complex provides a positive diagnosis of coronavirus in the subject. In one embodiment, a method for diagnosing coronavirus infection in a subject is provided, wherein the method comprises the steps of (a) administering to a subject a single domain antibody of the invention, (b) obtaining a sample from a subject and contacting the sample with a single domain antibody of the invention and (c) detecting the antibody-antigen complex, wherein the presence of the complex provides a positive diagnosis of coronavirus in the subject. In one embodiment, a method for diagnosing coronavirus infection in a subject is provided, wherein the method comprises the steps of (a) obtaining a sample from a subject, (b) contacting a sample from the subject with a single domain antibody of the invention and (c) detecting the antibody-antigen complex, wherein the presence of the complex provides a positive diagnosis of coronavirus in the subject. In one embodiment, a method for diagnosing coronavirus infection in a subject, the method comprising (a) contacting a sample with a single domain antibody of the invention, (b) detecting the number of antibody-polypeptide complexes and (c) detecting the presence of coronavirus in the sample, wherein the presence of the complex provides a positive diagnosis of coronavirus in the subject. The sample may be an isolated sample (i.e. previously obtained from a subject).

In one embodiment, the method comprises the step of comparing the sample with reference sample values for levels of the antibody-antigen complex. An antigen-antibody complex value above that of the reference sample value can provide a positive indication of coronavirus infection. The sample may be an isolated sample (i.e. previously obtained from a subject).

In some embodiments the single domain antibody of the invention, may further comprise a marker such as a radiolabelled marker, imaging marker, MRI-marker, fluorescent marker or other detectable marker. Such antibodies can be used in each of the detection or diagnosis methods described herein to enable the detection of the antibody in the subject in real time. Such antibodies can also be used in each of the detection or diagnosis methods described herein to enable the detection of the antibody in a sample, such as a tissue or blood sample, isolated or obtained from a subject.

In one embodiment, an assay to detect a coronavirus is provided, wherein the assay comprises (a) contacting a sample obtained from a patient with a single domain antibody of the invention, wherein the single domain antibody comprises a detectable label or reporter molecule to selectively isolate the coronavirus in the patient sample. In one embodiment, an assay to detect coronavirus is provided, wherein the assay comprises (a) contacting a sample obtained from a patient with a fusion protein comprising a single domain antibody of the invention and a biosensor, optionally a fluorescent or hinged biosensor, The assay may for example be an enzyme-linked immunosorbent assay (ELISA), an immunofluorescence assay, a radioimmunoassay (RIA) or a fluorescence-activated cell sorting (FACS). The detectable label or reporter molecule can be a fluorescent or chemical molecule (e.g. fluorescein isothiocyanate, or rhodamine), a biosensor, a radioisotope or enzyme (e.g. alkaline phosphatase, β-galactosidase, horseradish peroxidase or luciferase).

In one embodiment a kit is provided, wherein the kit comprises (a) a detectable marker (b) a single domain antibody of the invention. The detectable label or reporter molecule can be a fluorescent or chemical molecule (e.g. fluorescein isothiocyanate, or rhodamine), a biosensor, optionally a fluorescent or hinged biosensor or a radioisotope or enzyme (e.g. alkaline phosphatase, β-galactosidase, horseradish peroxidase or luciferase).

The methods described herein can be in vitro or ex vivo. The methods described herein can also be performed in vivo.

The invention is described by reference to the following non-limiting Examples. Apart from where specified otherwise, the SARS-CoV-2 strain used in the below examples is prototypical Wuhan/Victoria variant (‘WT’).

### Example 1

The amino sequences of single-domain antibodies (or nanobodies) that specifically bind protein to the Spike (S) protein of SARS-CoV-2 are described. The expressed proteins bind to the receptor-binding domain (RBD) of the virus with high, picomolar affinity.

Antibodies to the receptor-binding domain of SARS-CoV-2 were raised in a llama by primary immunisation with a combination of purified RBD alone and fused to human IgG1, followed by a single boost with purified S protein mixed with RBD. A phage display VHH library was constructed from the cDNA of peripheral blood mononuclear cells, and two rounds of bio-panning selected RBD binders. The phage clones with the highest affinity for RBD were identified by an inhibition ELISA and classified by sequencing into unique complementary determining region 3 (CDR3). Previously, a series of anti-RBD VHHs had been isolated from a non-immunised llama VHH library, among which the strongest binder was designated H11-H4 with a KD of 5 nM (Huo, Le Bas et al. 2020). Competition with H11-H4-Fc for binding to RBD in an ELISA format was used to identify not only new binders from the immunised library that bound to the same epitope as H11-H4 but with higher affinity but also ones that recognised different epitopes. From these analyses five VHHs were selected for production and further characterization:
(1) B12—SEQ ID NO: 16
(2) F2—SEQ ID NO: 17
(3) C1—SEQ ID NO: 18
(4) C5—SEQ ID NO: 19
(5) H3—SEQ ID NO: 20

Immunisation and construction of VHH library Prototypical (Wuhan/Victoria) SARS-CoV-2 receptor-binding domain (amino acids 330-532), SARS-CoV-2 receptor-binding domain fused to hIgG1 Fc (RBD-Fc) and trimeric Spike protein (amino acids 1-1208) were produced as described by Huo et al 2020. Antibodies were raised in a llama by intramuscular immunization with 200 pg of recombinant RBD and 200 pg of RBD-Fc on day 0, and then 200 pg RBD and 200 pg S protein on day 28. The adjuvant used was Gerbu LQ #3000. Blood (150 ml) was collected on day 38. Immunizations and handling of the llama were performed under the authority of the project license PA1FB163A. Peripheral blood mononuclear cells were prepared using Ficoll-Paque PLUS according to the manufacturers protocol; total RNA extracted using TRIzol™; reverse transcription and PCR was carried using SuperScript IV Reverse Transcriptase using reverse transcription primer:

CALL_GSP
(5′-CCTGCGGCTCCCGGGTCTGCCCTTTGGCC-3′)

The pool of VHH encoding sequences were amplified by PCR using primers as given in Table 1.

+0 ABLE 1
Primers used for PCR amplification
of VHH encoding sequences.

| Primer | Sequence |
|---|---|
| Round One | |
| CALL_001 | 5′-GTCCTGGCTGCTCTTCTACAAGG-3′ |
| CALL_002 | 5′-GGTACGTGCTGTTGAACTGTTCC-3′ |
| Round_Two | |
| VHH_For | 5′-GTTATTACTCGCGGCCCAGCCGGCC |
| | GGCCGATGTGCAGCTGCAGGAGTCTGG |
| | ATRGGAGG-3′ |
| VHH_Rev_Ig | 5′- |
| G2 | GGTGATGGTGTTGGCCTCCCGGGCCGGC |
| | CGCTGGTTGTGGTTTTGGTGTCTT-3′ |
| VHH_Rev_Ig | 5′- |
| G3 | GGTGATGGTGTTGGCCTCCCGGGCCGGC |
| | CGCGGAGCTGGGGTCTTCGCTGTG-3′ |

Following purification by agarose gel electrophoresis, the VHH cDNAs were cloned into the Sfil sites of the phagemid vector pADL-23c. In this vector, the VHH encoding sequence is preceded by a pelB leader sequence followed by a linker, His6 and cMyc tag (GPGGQHHHHHHGAEQKLISEEDLS). Electro-competent E. coli TG1 cells were transformed with the recombinant pAD-23c vector resulting in a VHH library of about 4×10⁹ independent transformants. The resulting TG1 library stock was then infected with M13K07 helper phage to obtain a library of VHH-presenting phages.

Isolation of VHHs

Phages displaying VHHs specific for the RBD of SARS-CoV-2 were enriched after two rounds of bio-panning on 50 nM and 2 nM of biotinylated RBD respectively, through capturing with Dynabeads™ M-280 (Thermo Fisher Scientific). Enrichment after each round of panning was determined by plating the cell culture with 10-fold serial dilutions. After the second round of panning, 93 individual phagemid clones were picked, VHH displaying phages were recovered by infection with M13K07 helper phage and tested for binding to RBD by a combination of competition and inhibition ELISAs. In these assays, RBD was immobilized on a 96-well plate and binding of phage clones was measured in the presence of excess soluble RBD (inhibition ELISA) or the RBD-binding H11-H4-Fc (Huo, Le Bas et al. 2020) (competition ELISA). Phage binders were ranked according to the inhibition assay and then classified as either competitive with H11-H4 (i.e. sharing the same epitope) or non-competitive (i.e. binding to a different epitope on RBD). Clones were sequenced and grouped according to CDR3 sequence identity.

Protein Production

The monovalent VHH were cloned into the vector pOPINO (Bird, Rada et al. 2014) containing an OmpA leader sequence and C-terminal His6 tag through Infusion reaction, the pOPINO vector having been digested with KpnI and PmeI. The resulting vectors were transformed into the WK6 E. coli strain and protein expression induced by 1 mM IPTG grown overnight at 20° C. Periplasmic extracts were prepared by osmotic shock and VHH proteins purified by immobilised metal affinity using an automated protocol implemented on an AKTAxpress followed by a Hiload 16/60 Superdex 75 or a Superdex 75 10/300GL column, using phosphate-buffered saline (PBS) pH 7.4 buffer.

Bivalent polypeptides were also produced by fusion of VHHs to IgG Fc, for instance C5-Fc (SEQ ID NO: 170) which dimerises to bivalent (C5-Fc)2. To produce these bivalent Fc fusions, VHHs were cloned into the vector AbVec-hIgG1 and digested with AgeI and SalI. AbVec-hIgG1 contains a murine heavy chain leader sequence and a human IgG1 Fc. Other Fcs are likewise suitable, including, for example, IgG4 with or without stabilising and/or humanising modifications, such as S288P and those modifications taught in, for example, Atyeo et al. 2020, Suzuki et al. 2018, and Dumet et al 2019. Following transient expression in expi293 cells, the Fc fusion proteins were purified by affinity chromatography on Protein A-sepharose (Huo, Le Bas et al. 2020).

Binding Activity

The RBD binding kinetics of the five selected single-domain antibodies were measured by surface plasmon resonance (SPR) and the calculated KD values showed that affinities were in the picomolar range (Table 2).

TABLE 2
SPR RBD binding kinetics of isolated VHHs produced
in E. coli (data from repeat assays with multiple
injections of analyte of C1, H3, F2, and C5)

| Nanobody VHH | Ka (1/Ms) | Kd (1/s) | K_{D} (pM) | T_{1/2} (min) |
|---|---|---|---|---|
| B12 | 3.6E+06 | 2.7E−04 | 77 | 42 |
| C1 | 9.3E+05 | 5.7E−04 | 615 | 20 |
| C5 | 9.8E+06 | 9.8E−04 | 99 | 12 |
| F2 | 4.7E+06 | 1.9E−04 | 40 | 61 |
| H3 | 1.3E+07 | 3.3E−04 | 25 | 35 |

Competition binding experiments were carried out by SPR to investigate whether the VHHs blocked the binding of RBD to ACE2 and the overlap with the epitope recognized by the human monoclonal antibody CR3022 (Jan ter Meulen Edward N. van den Brink Leo L. M. Poon 2006) and or H11-H4 (Huo, Le Bas et al. 2020). The results showed that C1 and C5 completely blocked ACE-2 binding whereas F2 was partially inhibitory and B12 failed to block binding (Table 3). This is consistent with the observation that both F2 and B12 competed with CR3022 that binds to a region of RBD that is on the opposite of the domain from the ACE-2 binding site (Huo, Zhao et al. 2020). The results, summarized in Table 3, showed that C5 and C1 were able to block ACE2 binding completely. C5 but not C1 competed with H11-H4 for binding to RBD whereas C1 but not C5 competed with CR3022 binding to RBD. This is consistent with the observation that CR3022 and H11-H4 bind to opposite sites of the ACE2 binding region By contrast, both B12 and F2 competed with CR3022 but either did not compete or only partially competed with ACE2 binding, respectively.

TABLE 3
Summary of the nature of isolated VHH interaction
with characterized RBD antibodies.

| | ACE2-Fc | H11-H4-Fc | CR3022-Fc |
|---|---|---|---|
| B12 | Non-competitive | Non-competitive | Competitive |
| C1 | Competitive | Non-competitive | Competitive |
| C5 | Competitive | Competitive | Non-competitive |
| F2 | Non-competitive | Non-competitive | Competitive |
| H3 | Competitive | Competitive | Non-competitive |

C5 and H3 would be expected to target a similar epitope to that of H11-H4, human monoclonal antibodies and other nanobodies that neutralise SARS-CoV-2 by competing directly with the interaction between the spike protein and the ACE2 receptor (cluster 2 antibodies (Kuan-Ying et al 2021)). C1 and F2 belong to the group of antibodies (cluster 1 antibodies (Kuan-Ying et al 2021) including CR302221 and EY-6A24 that bind to a region distinct from the ACE2 receptor binding interface. These two antibodies have been reported to destabilize the trimeric spike protein and by this mechanism prevent receptor engagement (Huo, J et al 2020; Zhou, D et al 2020) thereby neutralizing the virus.

The surface plasmon resonance experiments were performed using a Biacore T200 (GE Healthcare). All assays were performed using a Sensor Chip Protein A (GE Healthcare), with a running buffer of PBS pH 7.4 supplemented with 0.005% v/v Surfactant P20 (GE Healthcare) at 25° C. To determine the binding kinetics between the RBD of SARS-CoV-2 and the isolated VHHs, RBD-Fc was immobilized onto the sensor chip. In the reciprocal assay, Fc-fusion of the antibodies were immobilised and RBD was injected over the sensor chip. All data were fitted to a 1:1 binding model using the Biacore T200 Evaluation Software 3.1. In the competition assays, ˜1,000 RU CR3022-Fc, ACE2-Fc, or H11-H4-Fc were immobilized as the ligand, isolated VHHs incubated with RBD were used as analyte. Competition assays were performed with a Sensor Chip Protein A (Cytiva).

Neutralisation Activity

The virus neutralizing activity of C1 and C5 were tested in a micro-titre neutralisation assay (MN) (Amanat, White et al. 2020). VHH-Fc fusions were serially diluted into Dulbecco's Modified Eagles Medium (DMEM) containing 1% (w/v) foetal bovine serum (FBS) in a 96-well plate. SARS-CoV-2 Victoria strain passage 4 (vero 76) [9×10⁴ pfu/ml] diluted 1:5 in DMEM-FBS was added to each well, with media only as negative controls. After incubation for 30 min at 37° C. Vero cells (100 μl) were added to each well and the plates incubated for 2 h at 37° C. Carboxymethyl cellulose (100 μl of 1.5% v/v) was then added to each well and the plates incubated for a further 18-20 h at 37° C. Cells were fixed with paraformaldehyde (100 μl/well 4% v/v) for 30 min at room temperature and then stained for SARS-CoV-2 nucleoprotein using a human monoclonal antibody (EY2A). Bound antibody was detected by incubation with a goat anti-human IgG HRP conjugate and following substrate addition imaged using an ELISPOT reader. The neutralization titre was defined as the titre of VHH-Fc that reduced the Foci forming unit (FFU) by 50% compared to the control wells.

The virus neutralizing activity of C1-Fc and C5-Fc showed 100% inhibition of virus infection of cells (FIG. 8A), with NT50s of 402 pM and 26.5 pM respectively (mean of n=2 experiments). Monovalent VHH C5 neutralises SARS-CoV2 with an IC50 of 32 nM (FIG. 8B).

X-Ray Crystallography and Structural Analysis

The crystal structures of the C5-RBD complex (FIG. 1A) and the F2-RBD complex (FIG. 1B) were solved by X-ray crystallography. Purified VHH_C5 or VHH_C1 was mixed with deglycosylated RBD at molar ratio of 1:1, and the complex purified by size exclusion chromatography as described by Huo et al. 2020.

Crystals were grown at 20° C. by sitting drop vapour diffusion, and diffraction data collected and processed at the Diamond Light Source. The structure was solved by molecular replacement using standard methods and PDB id 6YZ5 as the search model and has been refined to high resolution.

The C5-RBD structure (FIG. 1A) showed that the epitope recognized by C5 overlaps very substantially overlapping the RBD binding site for ACE2 (FIG. 3A). Thus it is clear why C5 prevents ACE2 binding and is potently neutralising. The C5 epitope partly overlaps with the H11 class of nanobody epitope (e.g. H11-H4) that explains why C5 also competes with H11-H4 for binding to RBD. The literature has identified another epitope (the CR3022 epitope), which is located in a different region of the RBD (FIG. 3B). The structure of F2 and, separately, C1 bound to the RBD show that both F2 and C1 overlap with this CR3022 epitope.

Cryo-EM Protein Purification and Data Collection

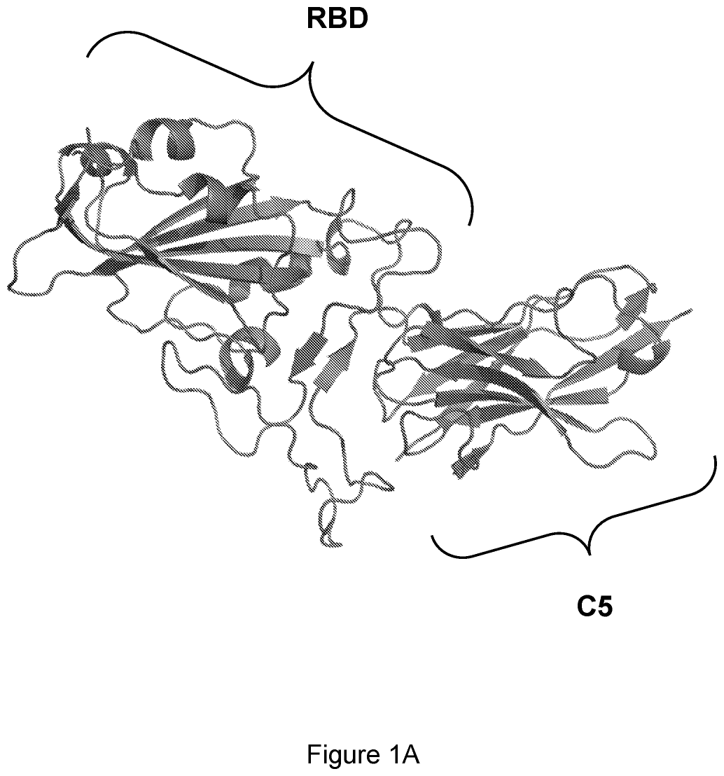

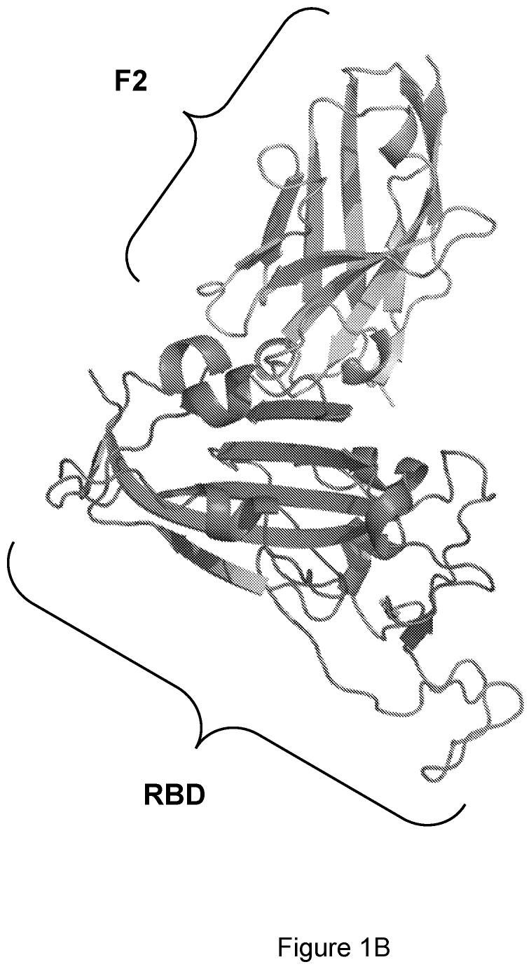

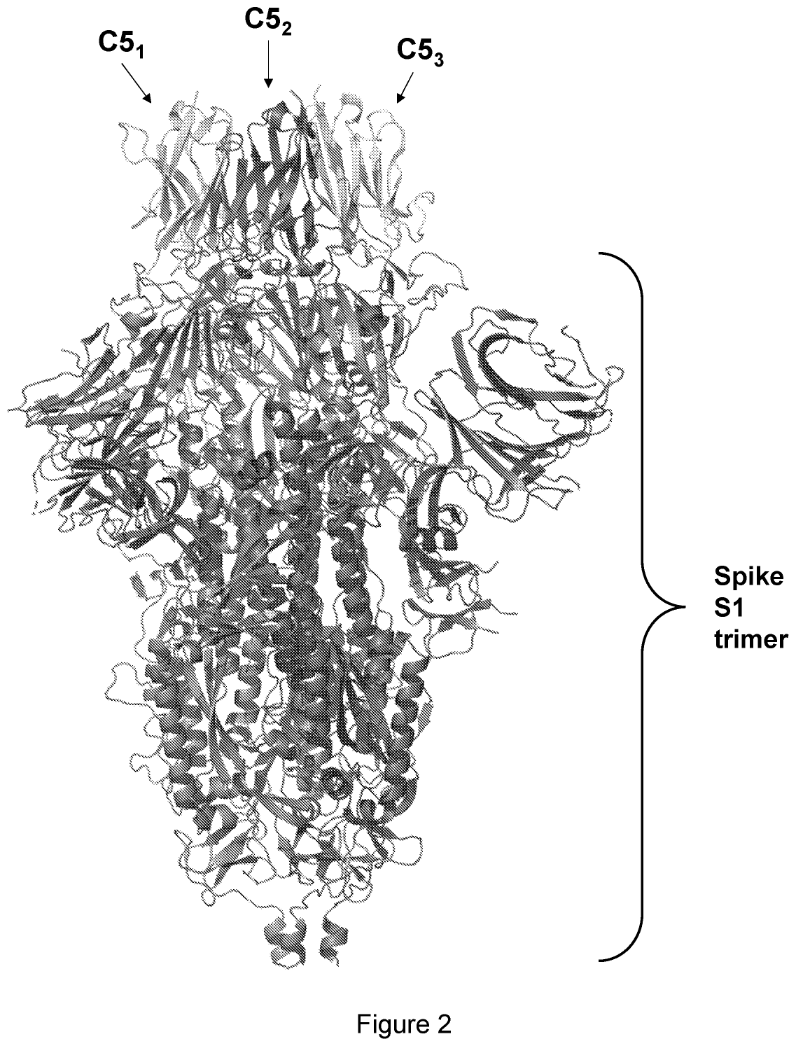

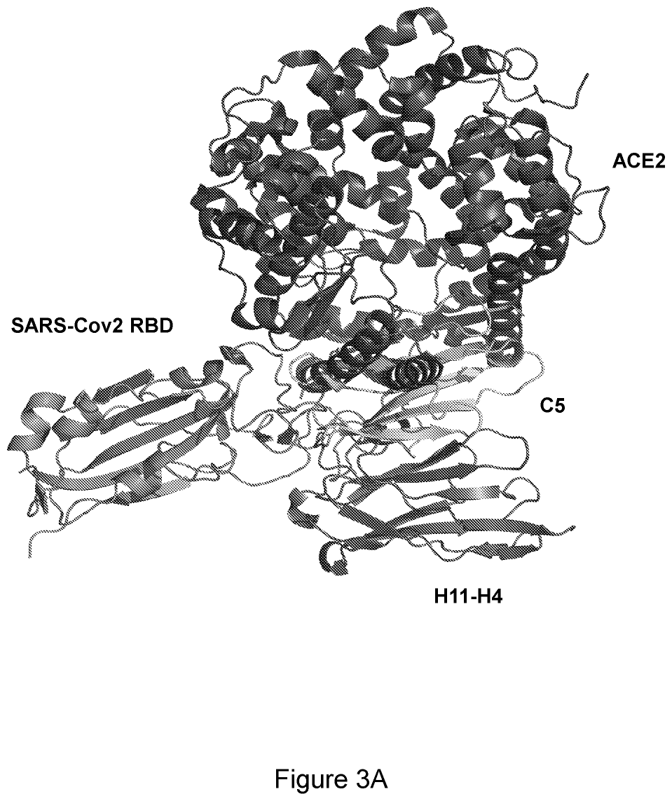

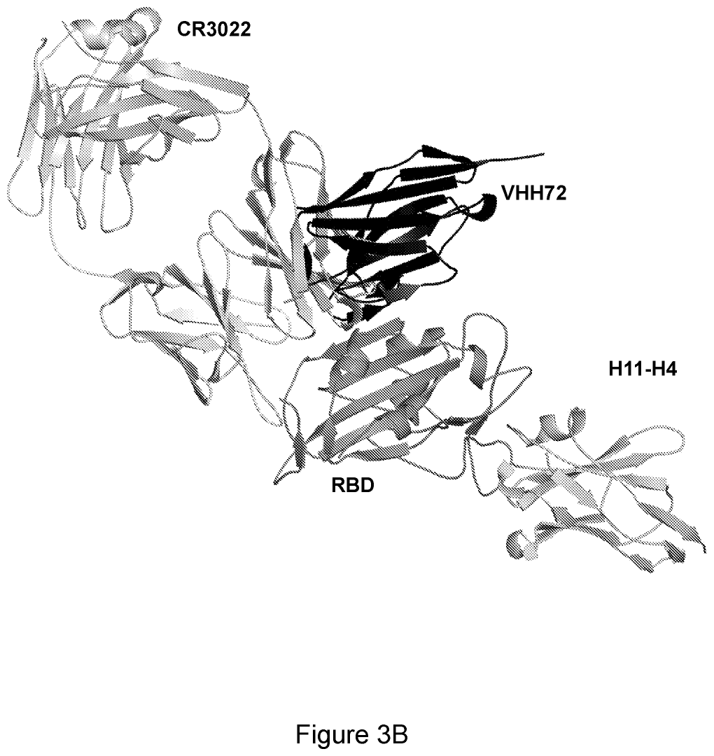

The structure of Spike-C5 trimer was determined through single particle electron microscopy (‘EM’) (FIG. 2). Purified spike protein in 10 mM Hepes, pH 8, 150 mM NaCl, was incubated with C5 purified in 50 mM Tris, pH 7, 150 mM NaCl, at a molar ratio of 1:3.6 (Spike trimer:nanobody) at 16° C. Structure determination and refinement was performed according to the methodology of Huo et al (2020) Nat Struct Mol Biol.

This showed that C5 nanobodies bound to the “3 down” (inactive) form of the spike trimer (FIG. 2) suggesting that the nanobody drives the spike protein into this conformation of the spike protein. C5 (unlike H11-H4) cannot bind to the “1 up 2 down” active form due to steric clashes. Incubation of C1 or F2 with the trimeric spike protein led to ill-defined aggregates on EM grids, indicating they destabilise the trimer, which would disrupt ACE2 engagement.

Construction of Receptor Binding Domain Variants

The RBD-WT was used a as template to generate the Alpha RBD variant, by amplifying two fragments with pairs of primers (1) TTGneo_RBD_F and N501Y_R and (2) TTGneo_RBD_R and N501Y_F which were then joined together by PCR using primer TTGneo_RBD_F and TTGneo_RBD_R (see Table 4). The Alpha RBD gene product was then cloned into the pOPINTTGneo vector by Infusion® cloning. The Alpha RBD was used as a template to generate the Beta RBD by amplifying two fragments with primers of (1) TTGneo_RBD_F and E484K_R and (2) TTGneo_RBD_R and E484K_F; the two fragments were then joined together by PCR primers TTGneo_RBD_F and TTGneo_RBD_R. The gene product was then cloned into the pOPINTTGneo vector by Infusion® cloning to create an intermediate vector which was then used as template to amplify two fragments with pairs of primers of (1) TTGneo_RBD_F and K417V_R and (2) TTGneo_RBD_R and K417V_F; the two fragments were then joined together with a PCR reaction using primer TTGneo_RBD_F and TTGneo_RBD_R. The final Beta RBD gene product was then cloned into the pOPINTTGneo vector by Infusion® cloning. To generate the hulgG1 Fc-fusion versions of RBDs, the RBD genes from the pOPINTTGneo vector were amplified by a pair of primers TTGneo_RBD_F and RBD_Fc_R, followed by being cloned into the pOPINTTGneo-Fc vector by Infusion® cloning. The pOPINTTGneo-Fc contains a mu-phosphatase leader sequence, a hulgG1 Fc and C-terminal His6 tag44. Delta RBD was amplified by PCR from a plasmid containing the full length Delta spike DNA and cloned into pOPINTTGneo by Infusion cloning.

TABLE 4
Primers for RBD variant construction

| | |
|---|---|
| TTGneo_RBD_F | gcgtagctgaaaccggcccg |
| | aatatcacaaatctttgt |
| TGneo_RBD_R | GTGATGGTGATGTTTATTTG |
| | TACTTTTTTTCGGTCCGCAC |
| | AC |
| 501Y_F | Cagtcatacggatttcaacc |
| | aacttacggggttggctatc |
| | agccgtaccgc |
| 501Y_R | gcggtacggctgatagccaa |
| | ccccgtaagttggttgaaat |
| | ccgtatgactG |
| 417V_F | CAGATCGCGCCGGGCCAAAC |
| | GGGCGTGATAGCTGACTATA |
| | ATTATAAG |
| 417V_R | CTTATAATTATAGTCAGCTA |
| | TCACGCCCGTTTGGCCCGGC |
| | GCGATCTG |
| 484K_F | ggttcaaccccctgcaatgg |
| | cgtcAAGGGTTTTAACTGTT |
| | ACTTCCCAC |
| 484K_R | GTGGGAAGTAACAGTTAAAA |
| | CCCTTgacgccattgcaggg |
| | ggttgaacc |
| BD_FC_R | 5′-CAGAACTTCCAGTTTAT |
| | TTGTACTTTTTTTCGGTCCG |
| | C-3′ |

Binding to SARS-CoV-2 RBD Variants

The binding of the VHHs (C1, C5, H3 and F2) to RBDs of SARS-CoV-2 variants was measured by SPR. As expected from structural analyses neither C5 or H3 bound to the Beta variant (lineage B.1.351) originally identified in South Africa due to the charge change mutation of residue 484 from Glu to Lys that forms a critical salt bridge interaction with C5 and H3 in the WT RBD complex. Binding of C5 to the Alpha variant was significantly reduced compared to the RBD-WT probably due to the mutation of N501Y in the Alpha RBD that is involved in the interaction with C5. Binding of H3 which does to interact with N501 was only marginally reduced. By contrast, binding of C1 and F2 that recognise a different region of the RBD was not affected by the either the N501Y or E484K mutations.

TABLE 5
Binding affinities to the RBDS of SARS-
CoV-2 variants determined by SPR

| | | Kₐ | K_{d} | K_{D} | T_{1/2} |
|---|---|---|---|---|---|
| Analyte | Ligand | (1/Ms) | (1/s) | (pM) | (min) |
| C1 | Bio-RBD-WT | 9.3E+05 | 5.7E−04 | 615 | 20 |
| C1 | Bio-RBD-Alpha | 7.5E+05 | 5.4E−04 | 725 | 21 |
| C1 | Bio-RBD-Beta | 9.2E+05 | 6.0E−04 | 648 | 19 |
| C1 | Bio-RBD-Delta | 5.8E+05 | 6.2E−04 | 1065 | 19 |
| C5 | Bio-RBD-WT | 9.8E+06 | 9.8E−04 | 99 | 12 |
| C5 | Bio-RBD-Alpha | 6.8E+06 | 1.7E−02 | 2523 | 1 |
| C5 | Bio-RBD-Delta | 2.7E+06 | 4.2E−03 | 1600 | 3 |
| H3 | Bio-RBD-WT | 1.3E+07 | 3.3E−04 | 25 | 35 |
| H3 | Bio-RBD-Alpha | 1.2E+07 | 1.2E−03 | 102 | 10 |
| H3 | Bio-RBD-Delta | 1.6E+06 | 1.9E−03 | 1200 | 6 |
| F2 | Bio-RBD-WT | 4.7E+06 | 1.9E−04 | 40 | 61 |
| F2 | Bio-RBD-Alpha | 4.8E+06 | 2.3E−04 | 47 | 51 |
| F2 | Bio-RBD-Beta | 5.9E+06 | 2.2E−04 | 38 | 52 |
| F2 | Bio-RBD-Delta | 3.9E+06 | 1.9E−04 | 49 | 61 |

### Example 2

Two strategies were used to obtain new VHH binders to the SARS-CoV-2 variants and in particular the Beta strain (lineage B.1.351) that was originally detected in South Africa. In the first approach, the library of Example 1 generated from a llama immunised with the prototypical (Wuhan/Victoria) spike protein (200 pg of recombinant RBD and 200 pg of RBD-Fc on day 0, 200 pg RBD and 200 pg S protein on day 28, 200 pg RBD and 200 pg S protein on day 56, blood (150 ml) was collected on day 65) was re-screened with the RBD of the Beta strain containing the mutations E484K and N501Y. In order to enrich the library for binders that would at the RBD-ACE-2 interface (C5 epitope), it was pre-incubated with the C1 nanobody previously isolated and which binds to an epitope distal from this interface. However, this approach did not result in identification of any binders to the C5 epitope. Unexpectedly, two new nanobodies—NbSA_A10 and NbSA_D10—were isolated that bound to new epitope(s) with KDs of 30 pM and 936 pM respectively (FIG. 12a&b). These nanobodies did not block ACE2 or CR3022 binding (FIG. 12c&d). NbSA_A10 and NbSA_D10 were isolated and produced according to methodology set out in Example 1.

The library was pre-incubated with NbSA_A10 to remove VHH binders that bind to the same or similar epitope and then re-screened with SA RBD. From this iterative screen, a number of new nanobodies were isolated and their binding kinetics measured by SPR (Table 6). Of these, A8 had the highest binding affinity for RBD (KD of 35 pM) (FIG. 13a) and was shown to compete with ACE-2 and CR3022 for binding to SA RBD (FIG. 13b).

TABLE 6
SPR RBD binding kinetics of nanobody analytes.

| | | Kₐ | K_{d} | K_{D} | T_{1/2} |
|---|---|---|---|---|---|
| Analyte | Ligand | (1/Ms) | (1/s) | (pM) | (min) |
| A8 | Beta RBD-Fc | 5.00E+06 | 1.80E−04 | 35 | 65 |
| 3_C5 | Beta RBD-Fc | 9.00E+05 | 8.00E−04 | 895 | 14 |
| 8_G11 | Beta RBD-Fc | 1.30E+06 | 5.70E−04 | 443 | 20 |
| 12_F11 | Beta RBD-Fc | 4.10E+06 | 9.60E−04 | 236 | 12 |
| C1 | Beta-RBD-Biotin | 9.20E+05 | 6.00E−04 | 648 | 19 |

The structure of A8 in complex with RBD was determined by X-ray crystallography confirming that it bound in the same region of the RBD as CR3022 (FIG. 21)

In the second approach for isolating potential new VHH binders to the Beta SARS-CoV-2 a llama was immunised with a combination of Beta RBD and full-length spike protein of the Beta variant as described in Example 1. The VHH library constructed from the PBMCs of the immunised llama, was screened with the Beta-RBD and a VHH binder obtained (SEQ ID NO: 238/239), VHH_H6, that showed sub-nanomolar binding not only to Beta-RBD but also Kappa and the prototypical Victoria/Wuhan sequence (Table 17). Interestingly the binding affinity for RBD-Beta showed a tenfold increase for the bivalent Fc fusion version of the VHH_H6 but no difference in binding to the RBD-Victoria or RBD-Delta.

VHH_H6 competed with ACE-2 binding but not CR3022 (FIG. 19) placing its binding site at or close to the ACE-2 interaction surface and therefore likely to block virus infection of cells in a neutralisation assay. The epitope recognized by H6 was mapped by determining the structure of the VHH_H6-RBD complex by X-ray crystallography identifying a novel antibody epitope on the surface of the RBD distinct from that of C5 and H3 (FIG. 20). Of particular note is that residue 484 in the RBD that is mutated in the Beta (E484K), Gamma(E484K) and Kappa (E484Q) variants disrupting the binding by some human monoclonal antibodies (Zhou et al 2021), is not involved in the binding site.

TABLE 17
Binding kinetics determined by SPR

| | | Ka | Kd | KD | T½ |
|---|---|---|---|---|---|
| Analyte | Ligand | (1/Ms) | (1/s) | (pM) | (min) |
| VHH_H6 | RBD-Kappa-Fc | 6.2E+05 | 4.1E−04 | 666 | 28 |
| VHH_H6 | RBD-Beta-Fc | 7.8E+05 | 4.4E−04 | 564 | 26 |
| VHH_H6 | RBD-Victoria-Fc | 7.9E+05 | 3.7E−04 | 472 | 31 |
| RBD-Delta | VHH_H6-Fc | 1.2E+05 | 7.0E−04 | 592 | 28 |
| RBD-Beta | VHH_H6-Fc | 3.9E+06 | 1.2E−04 | 30 | 99 |
| RBD-Victoria | VHH_H6-Fc | 1.6E+06 | 4.2E−04 | 261 | 28 |

### Example 3

Joining two or more of the same VHHs together creates a monospecific bivalent polypeptide that is predicted to bind with higher affinity than the monovalent VHH due to the effect of avidity.

To test this, three bivalent versions of VHH C5 were designed as single polypeptides and constructed by PCR.
(1) C5-AAA-C5—residues 22 to 266 of SEQ ID NO: 171 or 182
(2) C5-GGGGSGGGS-C5—residues 22 to 272 of SEQ ID NO: 172 or 184
(3) C5-GSGSGS-SUMO-GSGSGS-C5—residues 22 to 369 SEQ ID NO: 173 or 186

Protein Production

Bivalent polypeptides were cloned into the vector pOPINO (Bird, Rada et al. 2014) containing an OmpA leader sequence and C-terminal His6 tag through Infusion reaction, the pOPINO vector having been digested with KpnI and PmeI. The resulting vectors were transformed into the WK6 E. coli strain and protein expression induced by 1 mM IPTG grown overnight at 20° C. Periplasmic extracts were prepared by osmotic shock and VHH proteins purified by immobilised metal affinity using an automated protocol implemented on an ÄKTAxpress followed by a Hiload 16/60 superdex 75 or a Superdex 75 10/300GL column, using phosphate-buffered saline (PBS) pH 7.4 buffer.

Binding Activity

Binding activity was measured by SPR according to the methodology provided in Example 1. All curves were plotted using GraphPad Prism 8. SPR kinetic analysis of binding of C5 polypeptides to the trimeric spike protein revealed astonishingly tight binding of mono-specific bivalent C5 polypeptides (Table 4; FIG. 7). The single-figure dissociation constants achieved with the C5 VHHs joined by the linkers AAA and 6GS-SUMO-6GS are very low and comparable to the results for the bivalent C5-Fc described in Example 1.

All monospecific bivalent C5 structures tested perform equivalently in an SPR biophysical assay of binding to trimeric spike protein (Table 4; FIG. 7). The on rates (denoted by the initial steep, positive gradient in FIG. 7) are comparable for all ligands. The downward slope is the off rate. The monospecific bivalent molecules all have slow off rates (almost no wash off) and are exquisitely tight binders (as shown by the single figure pM dissociation constants).

| Nanobody | Ka (1/Ms) | Kd (1/s) | K_{D} (pM) | T_{1/2} (h) |
|---|---|---|---|---|
| C5 | 1.4E+07 | 1.9E−03 | 134 | 0.1 |
| C5-Fc | 1.6E+07 | 9.0E−05 | 6 | 2.1 |
| C5-AAA-C5 | 1.4E+07 | 7.6E−05 | 5 | 2.5 |
| C5-9GS-C5 | 1.0E+06 | 4.2E−05 | 11 | 1.7 |
| C5-6GS- | 8.6E+06 | 4.2E−05 | 5 | 4.6 |
| SUMO-6GS-C5 | | | | |

Table 18: SPR Trimeric spike binding kinetics of C5 and C5 bivalent polypeptides; C5-Fc was produced in mammalian cells and C5, C5-AAA-C5, C5-9GS-C5, and 05-6GS-SUMO-6GS-C5 E. coli.

### Example 4

Joining two or more different VHHs together creates a bispecific bivalent polypeptide, also known as bidentate. These bidentate polypeptides are predicted to bind with higher affinity than the monovalent VHHs due to the effect of avidity. Further, having two or more joined different VHHs is predicted to confer further functional benefit and utility. Given that F2 competes with CR3022 but not H11-H4 it seems likely that this VHH shares an epitope with CR3022. Careful modelling shows that the epitopes of C5 and F2 can be bridged by a linker which spans the surface of RBD. The C-terminus of F2 and N-terminus of C5 are approximately 52 Å apart. Examination of the three dimensional arrangement of the epitopes, suggested that a linker consisting of short span of glycine and serine residues (GS) followed by the sequence of the protein SUMO (Small Ubiquitin-like Modifier) and then another GS sequence could be used to connect the two VHHs bound to their respective epitopes. SUMOs are a family of small (˜11.6 KDa) proteins involved in the post-translational modification of proteins and play a role in variety of cellular processes including cell cycle regulation and subcellular transport (Hay 2005). The design of the bidentate molecule followed this logic (N′-VHH to epitope 1-(GS)6-SUMO-(GS)6 VHH to epitope 2-C′) or vice versa. SUMO is a rigid molecule and will thus promote rigidity, minimising entropic penalty. The distance end to end of SUMO is 32 Å. Thus at least a further 20 Å separation are needed (around 6 to 8 residues as an elongated strand). The GS residues were introduced to allow both nanobodies to bind their respective epitopes thus gaining enthalpy. They introduce an entropic penalty. This is explicitly part of the design, adding or removing residues (including Pro and the other 17 natural residues) within the GS region to optimise the balance between maximising enthalpic gain and minimising entropic penalty.

Thus, the first construct was made:
F2-SUMO−C5 (SEQ ID NO: 175)

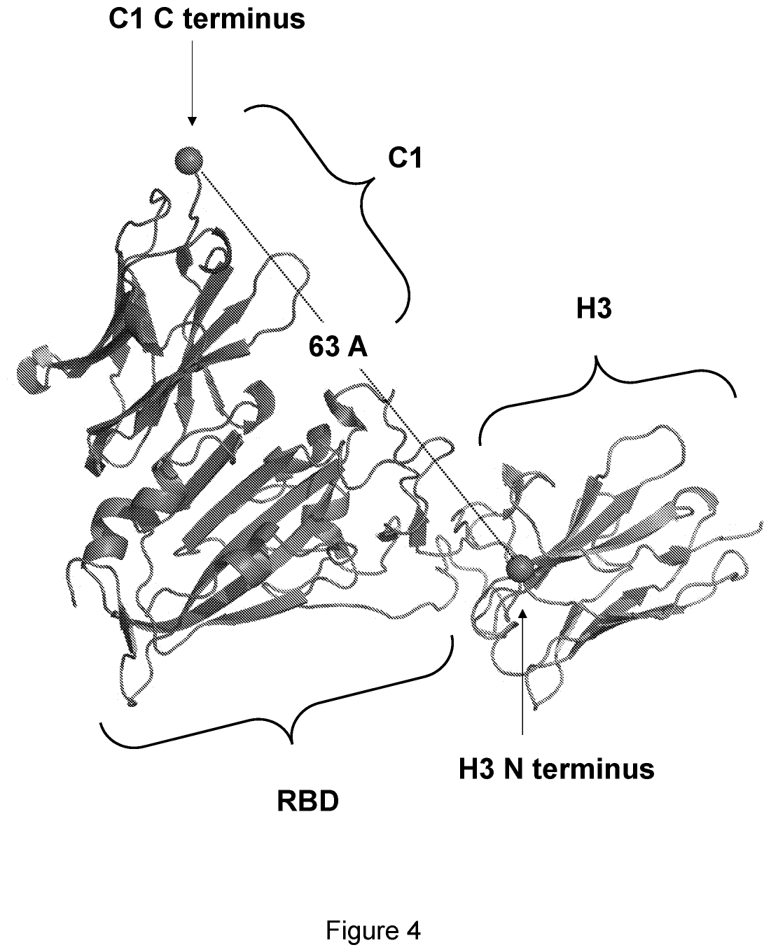

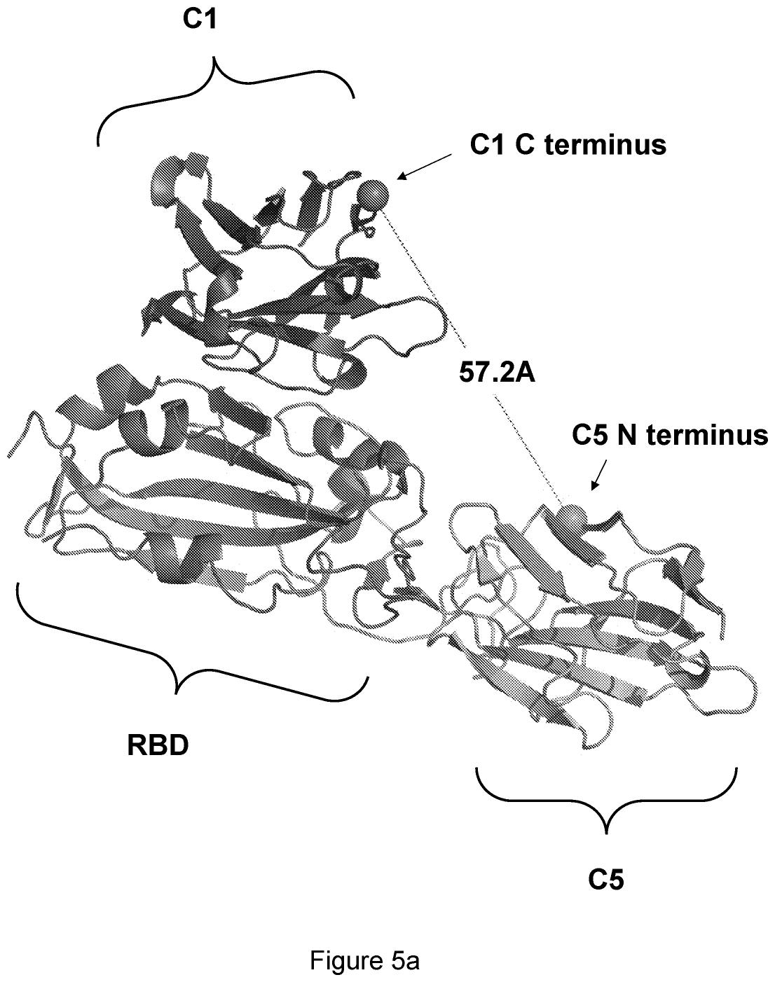

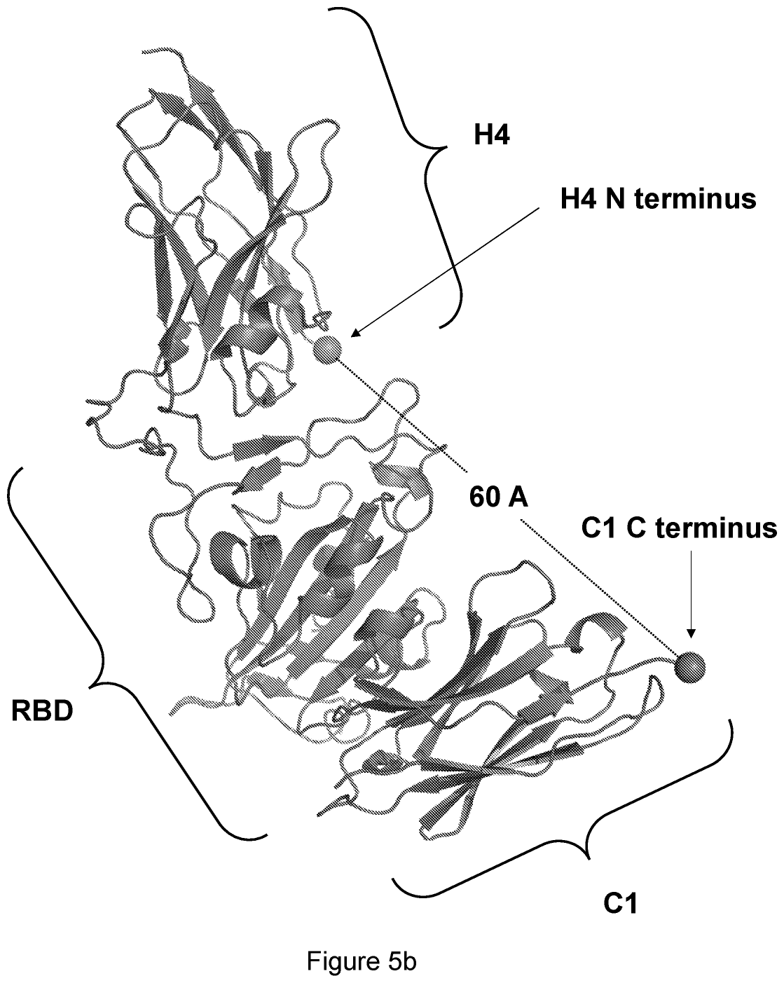

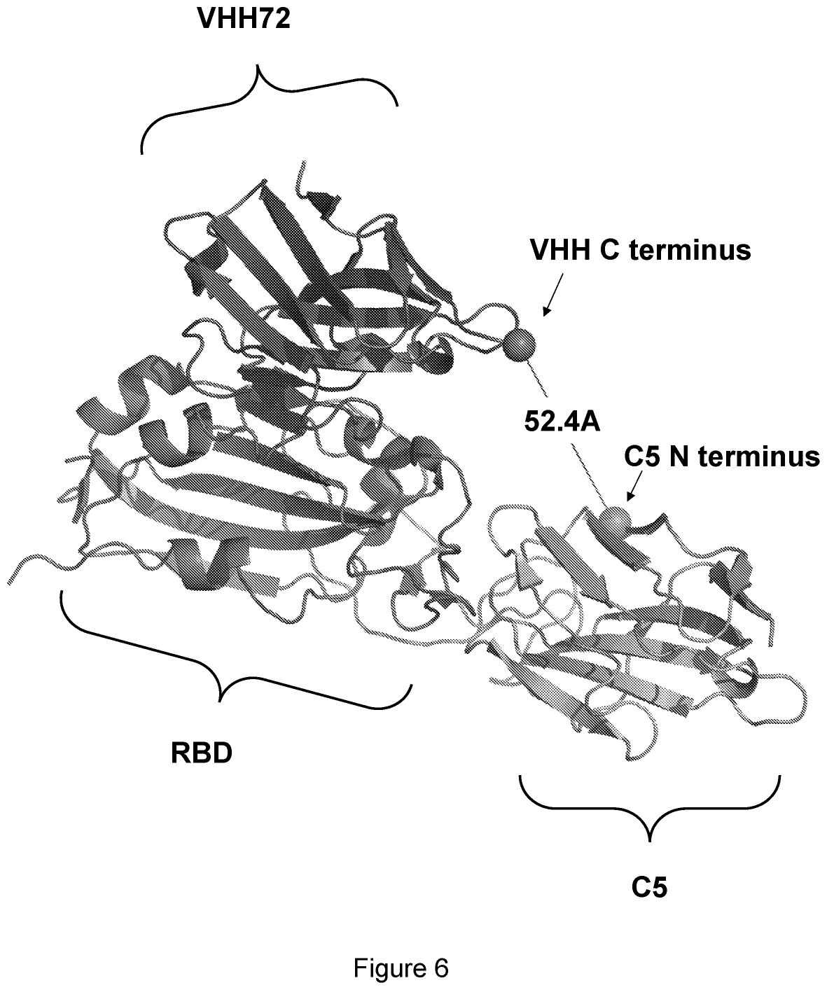

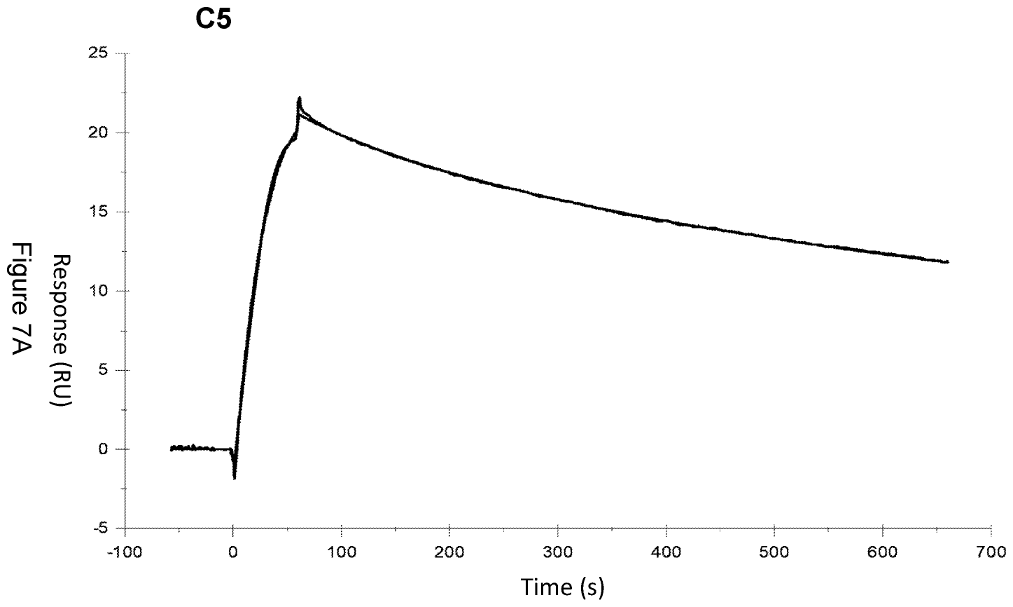

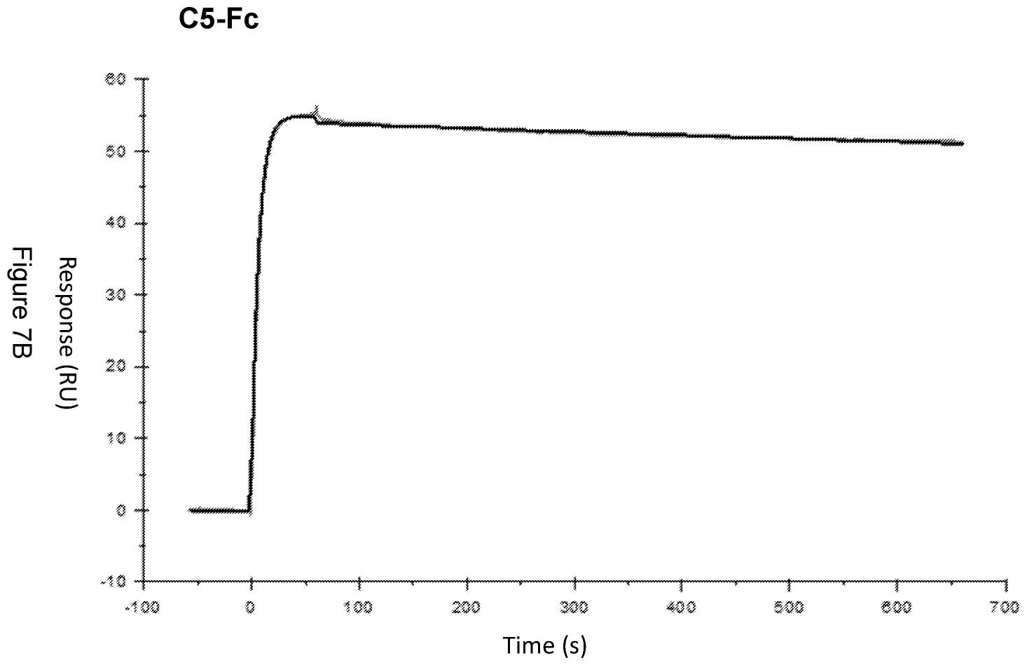

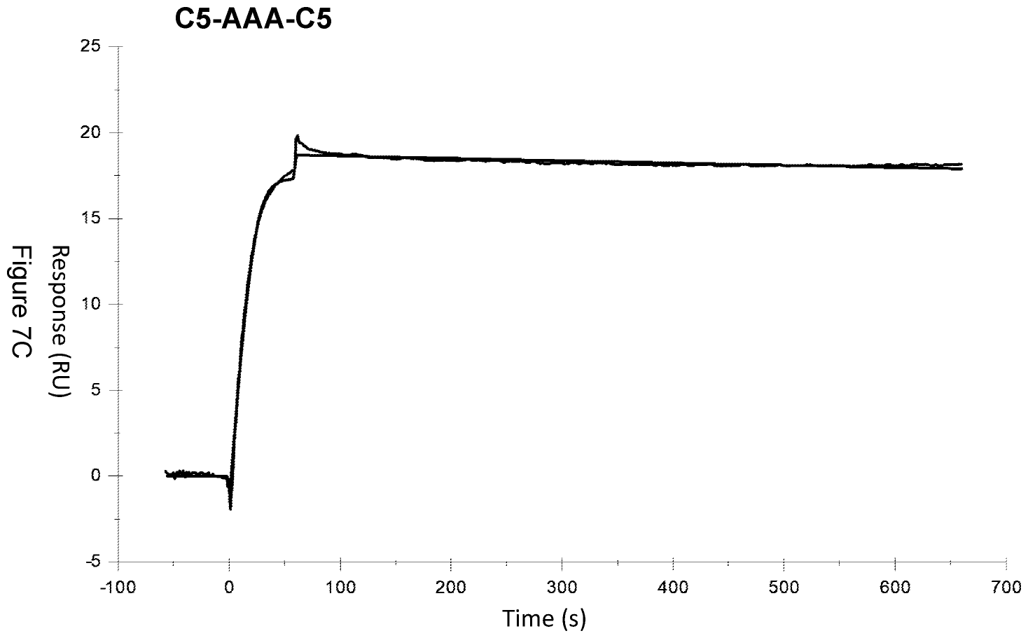

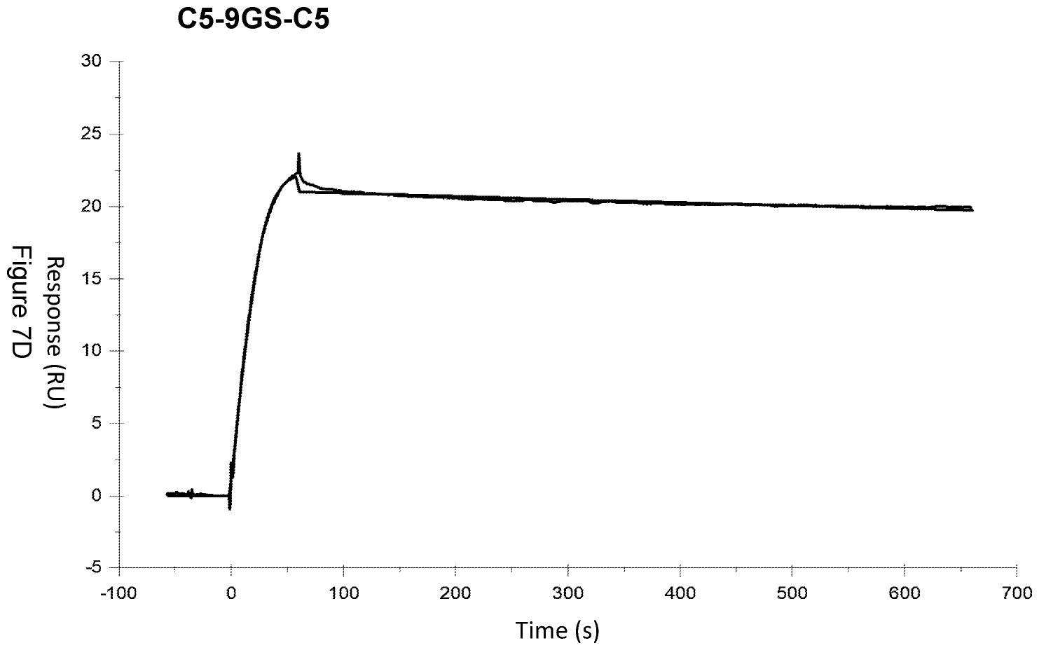

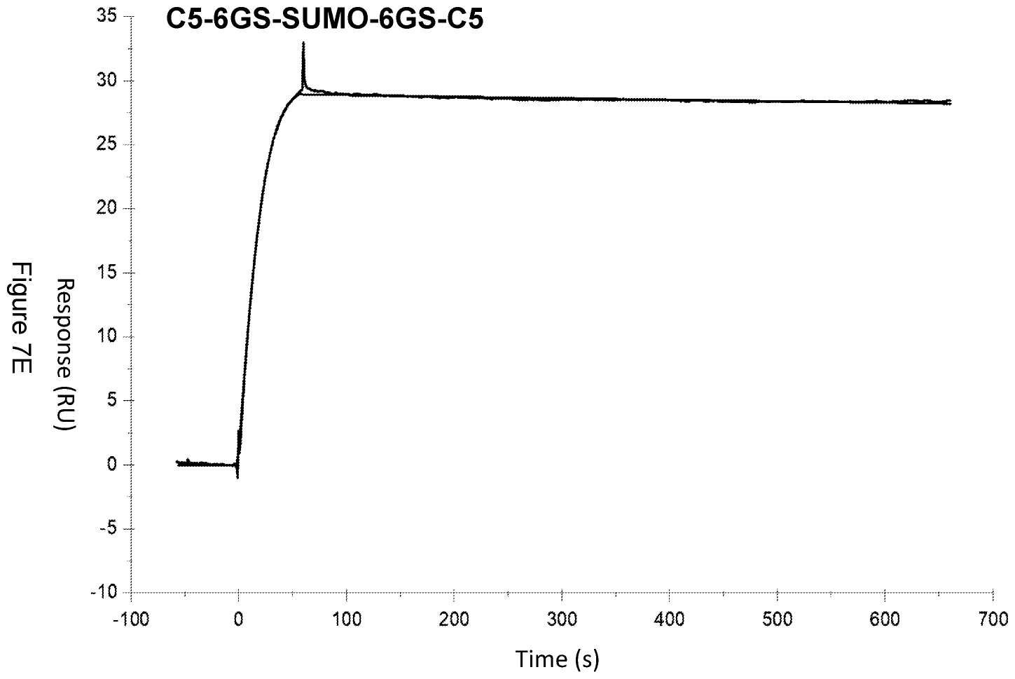

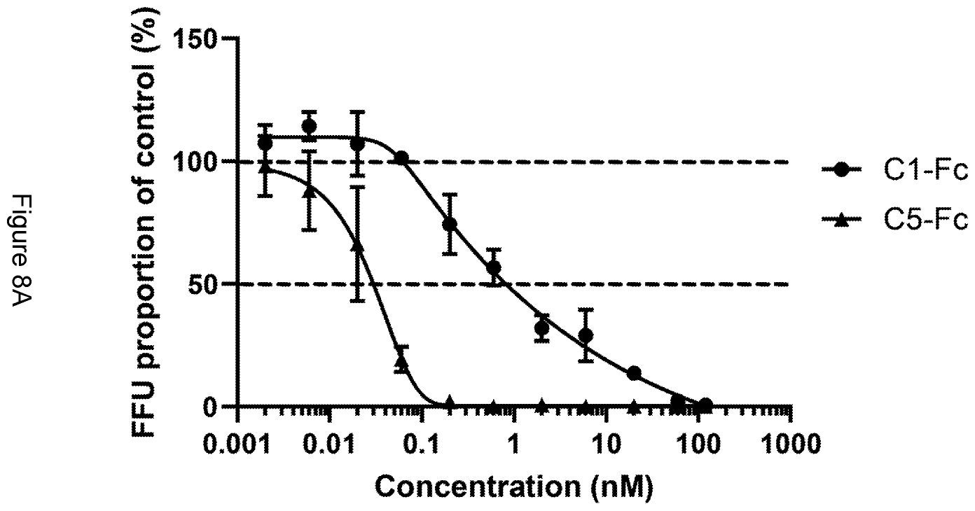

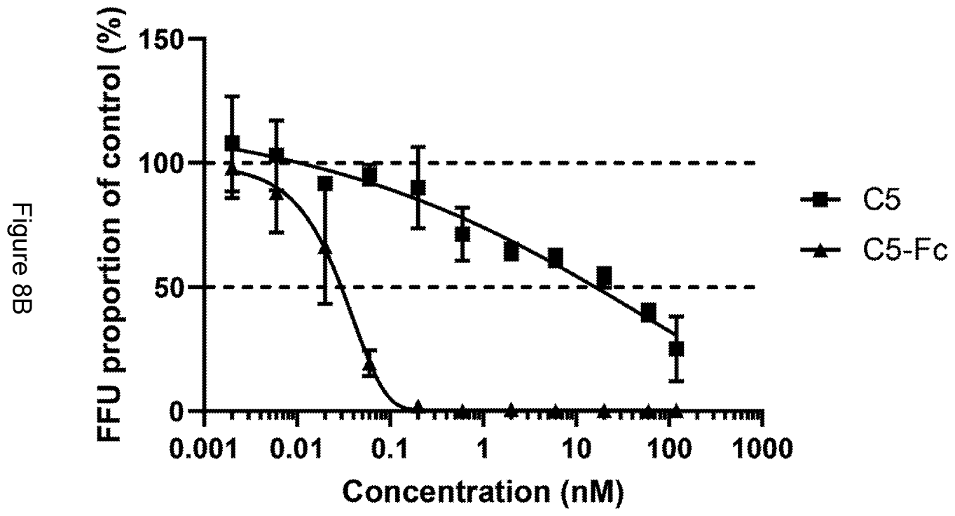

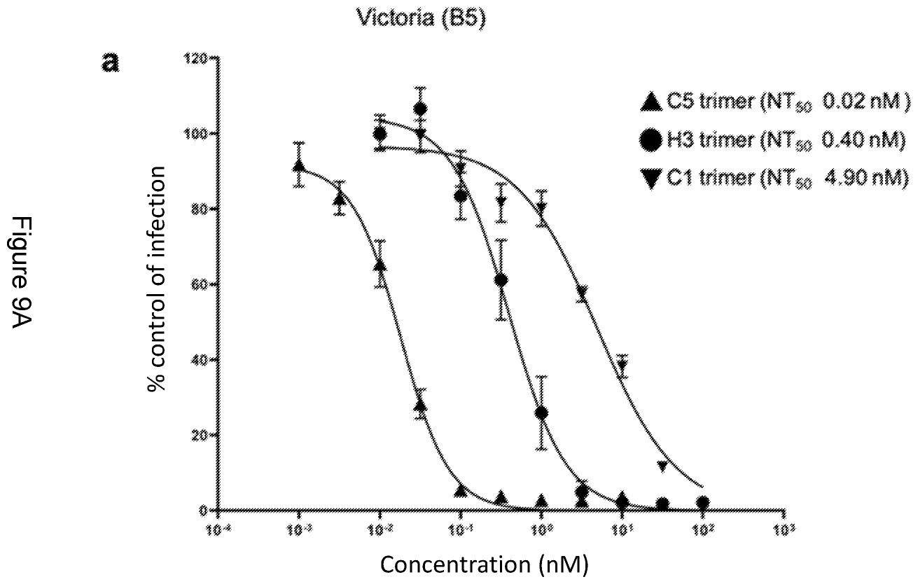

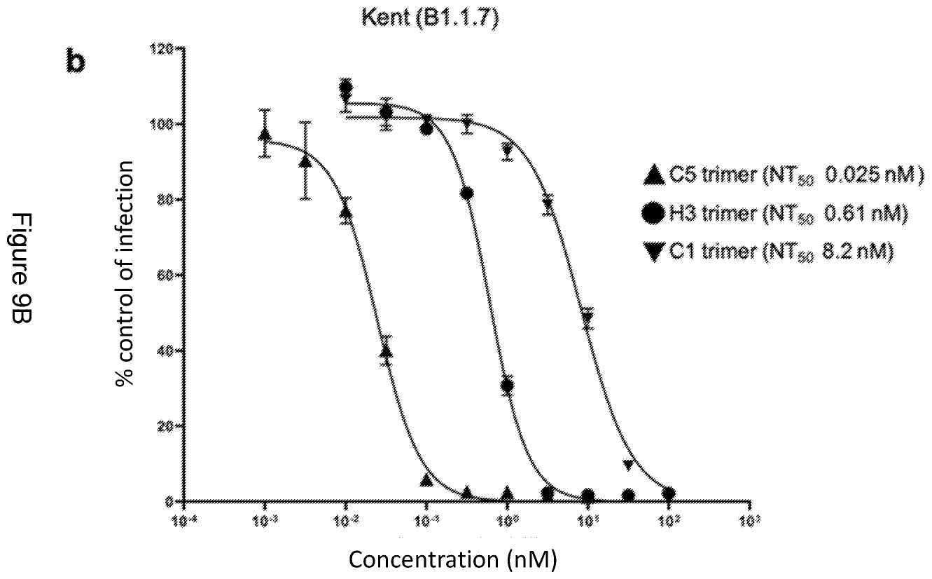

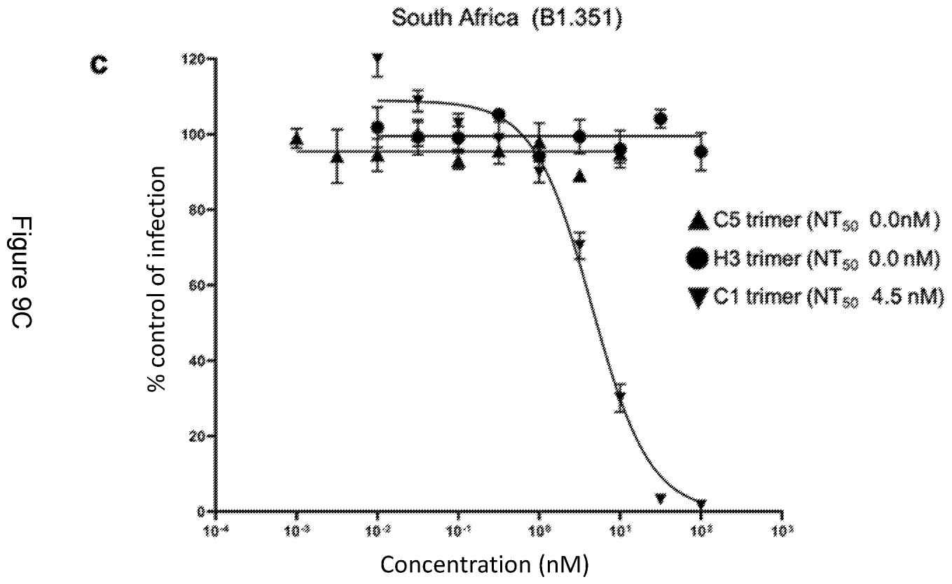

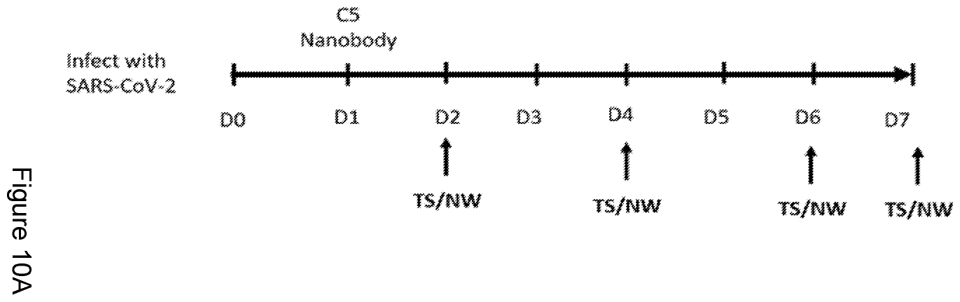

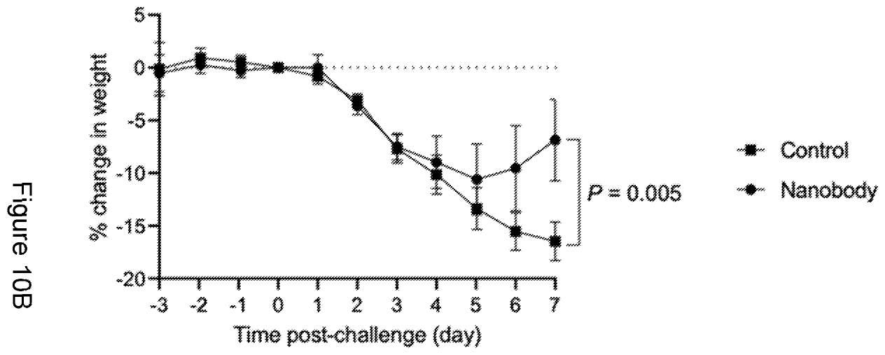

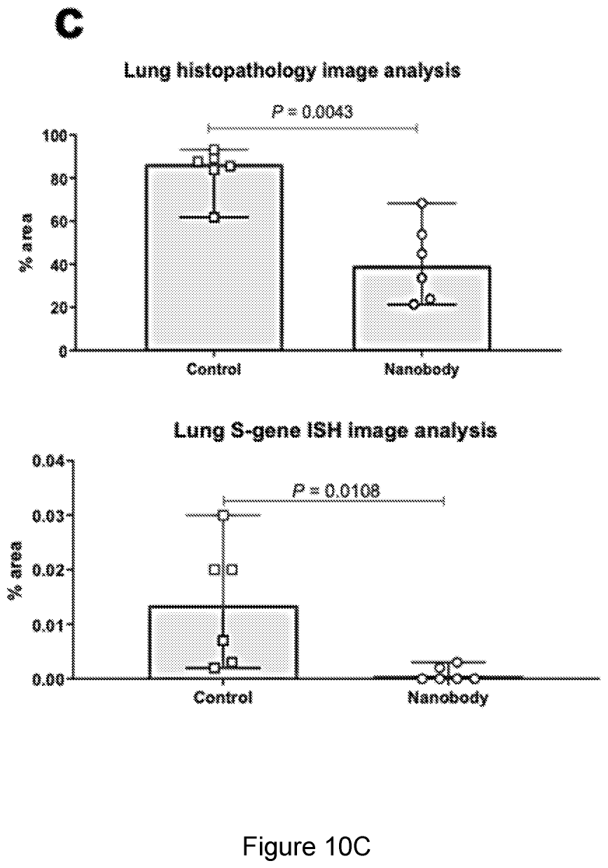

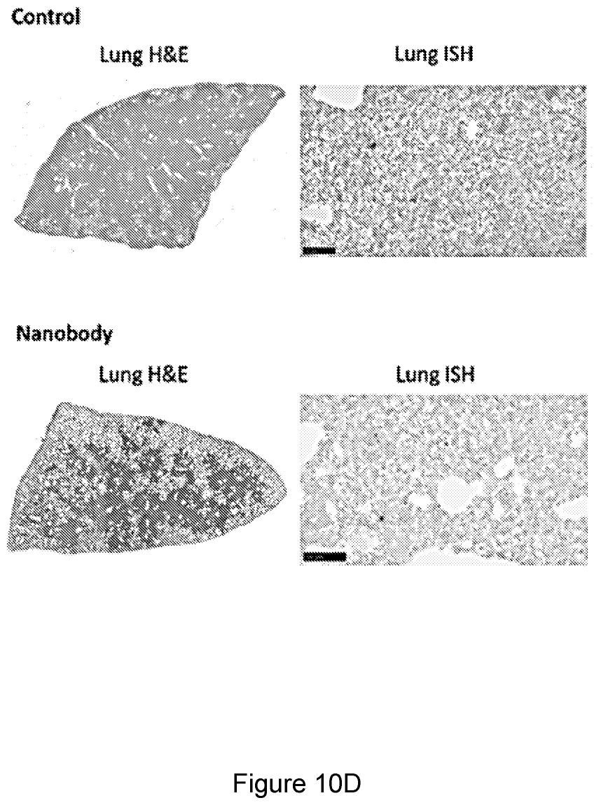

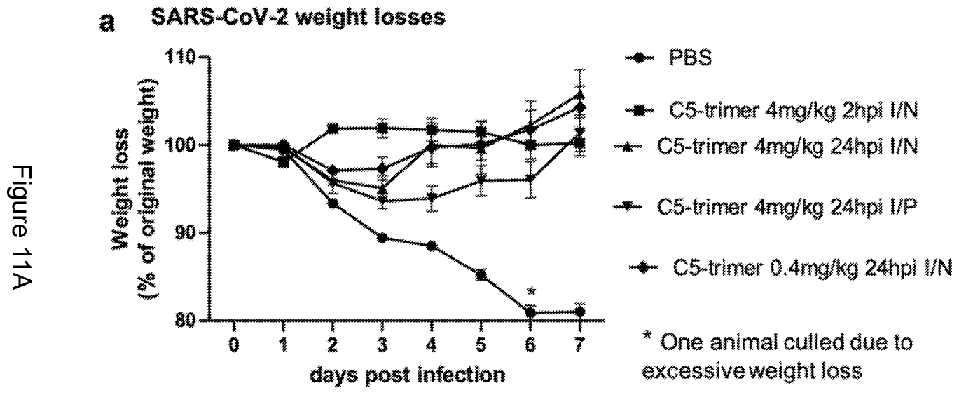

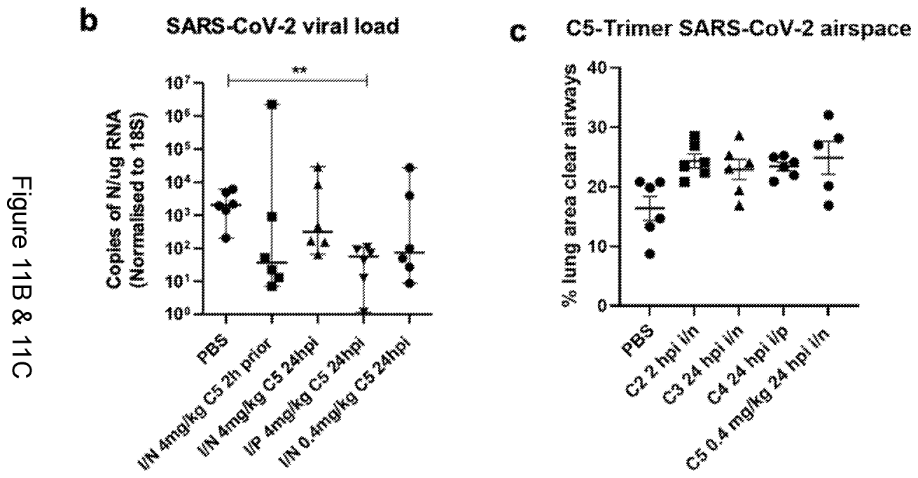

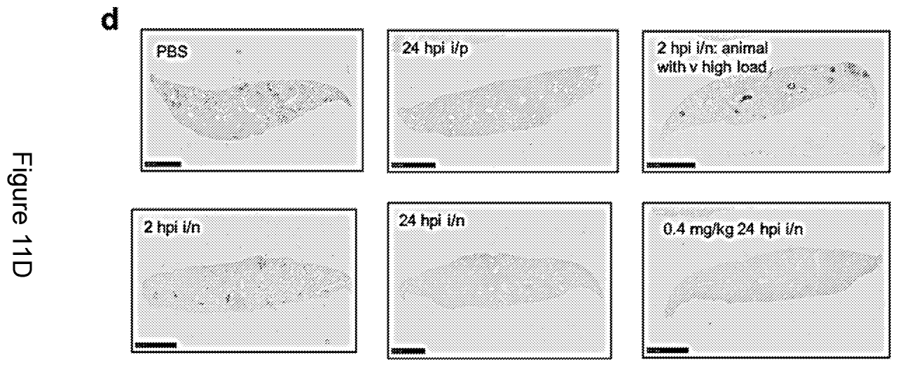

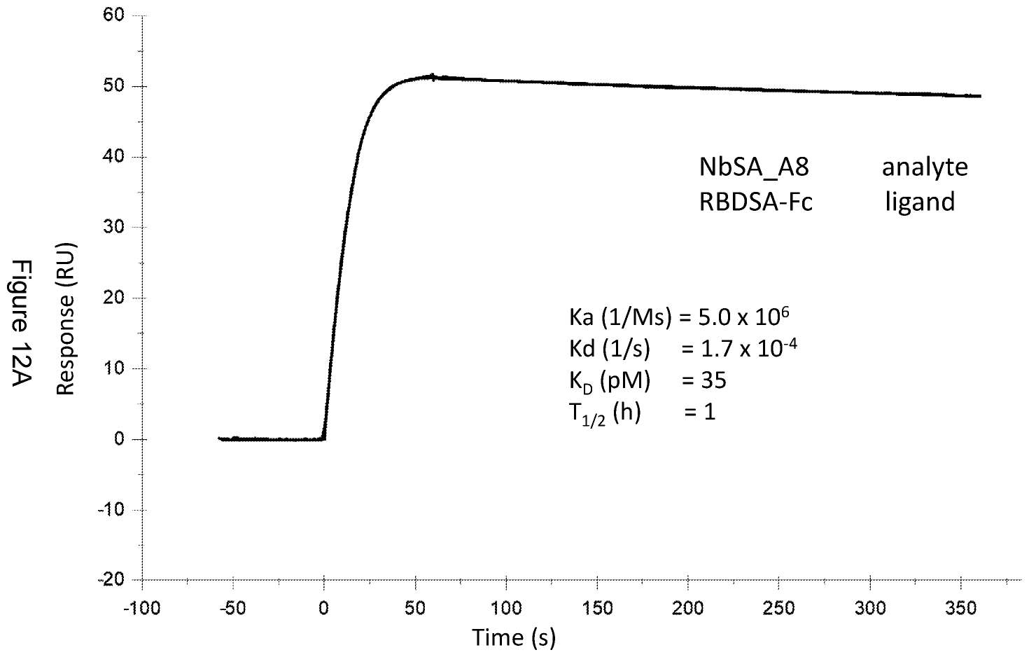

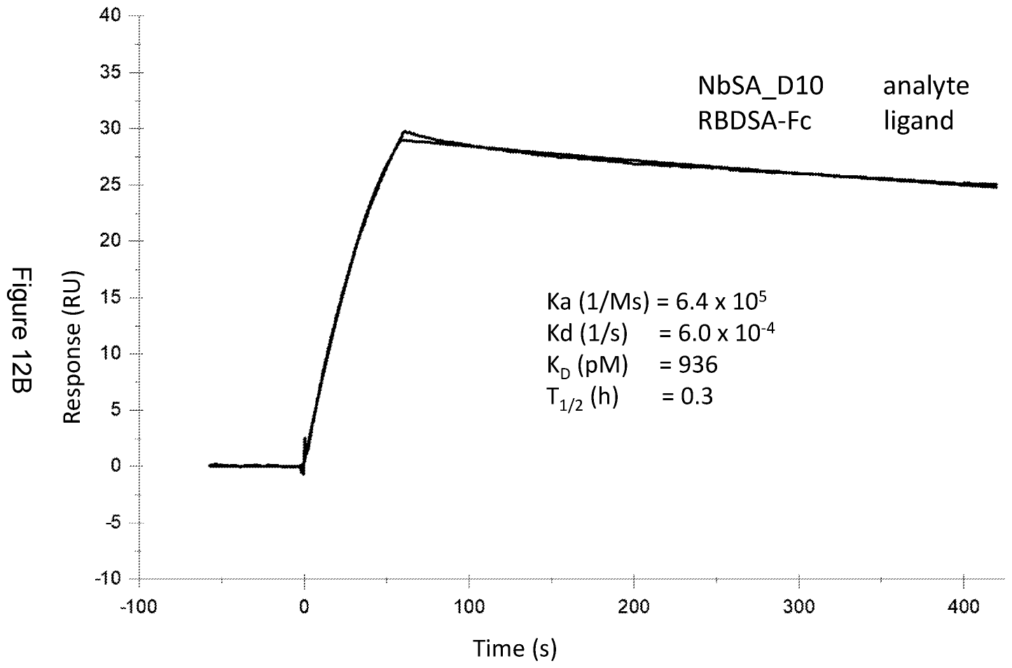

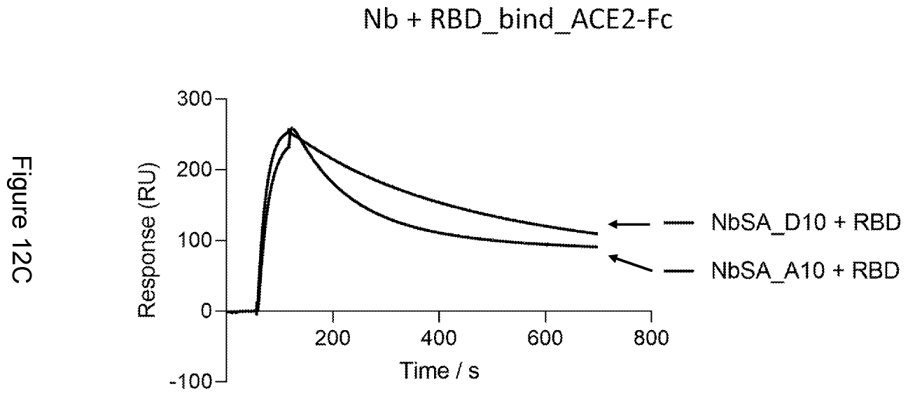

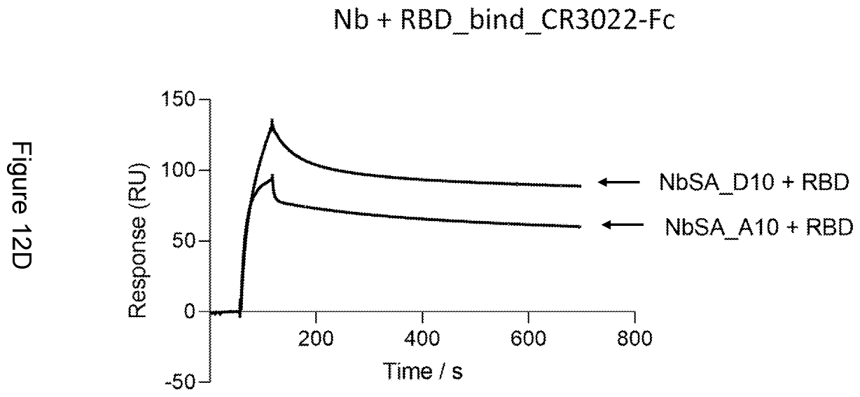

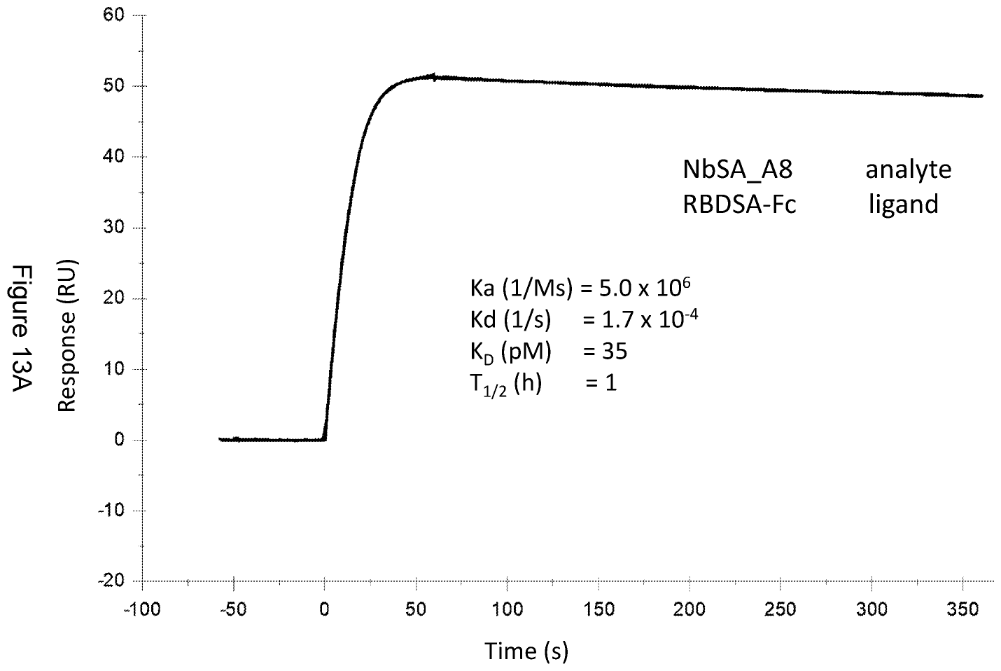

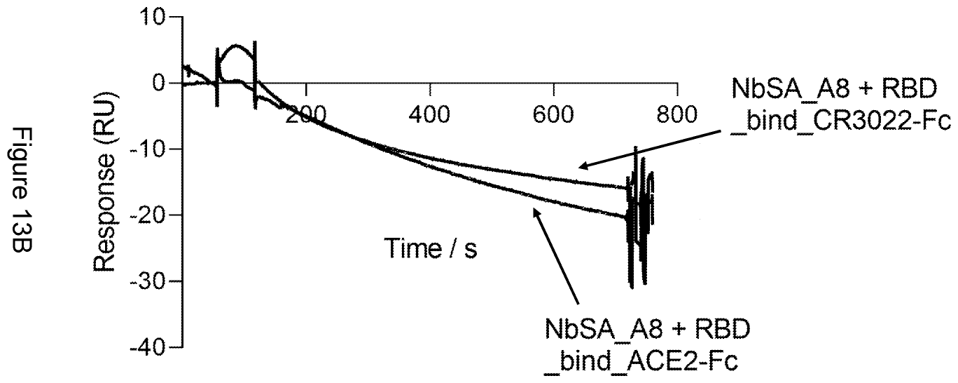

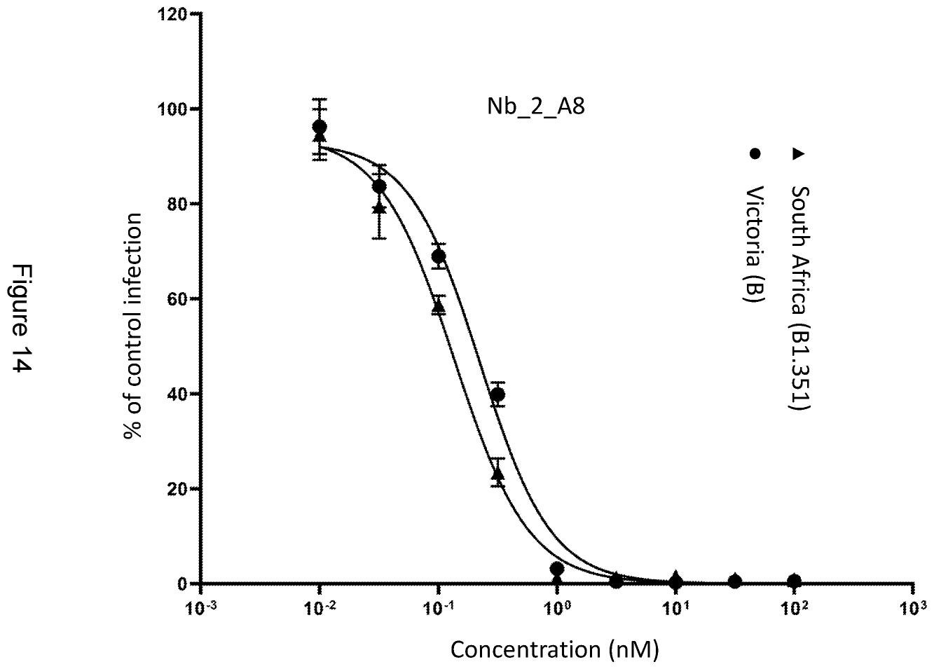

This same design rationale was applied to careful modelling of C1 and C5 (with a distance of approximately 57 Å between the C-terminus of C1 and the N-terminus of C5; FIG. 5a), C1 and H3 (with a distance of approximately 63 Å between the C-terminus of C1 and the N-terminus of H3; FIG. 4), C1 and H4 (SEQ ID NO: 120) (with a distance of approximately 60 Å between the C-terminus of C1 and the N-terminus of H4; FIG. 5b), and VHH_72 (SEQ ID NO: 32) and C5 (with a distance of approximately 54 Å between the C-terminus of VHH_72 and the N-terminus of C5; FIG. 6).

Thus, five additional constructs were created:
C1-SUMO-C5 (FIG. 5a; SEQ ID NO: 176)
C1-SUMO-H3 (FIG. 4; SEQ ID NO: 177)
C1-SUMO-H11-H4 (FIG. 5b; SEQ ID NO: 179)
VHH72-SUMO-C5 (FIG. 6; SEQ ID NO: 234)
F2-SUMO—VHH_H6 (SEQ ID 248)
C1, F2 and VHH_H6 bind to a different epitope (the same as CR3022) to that recognised by both H3 and H11-H4. C5 binds to the same epitope recognised by H3 and H11-H4 (Table 3).

Protein Production

Bidentate antibody polypeptides described here consists of three parts: the N-terminal VHH, the SUMO in the middle and the C-terminal VHH. The genes encoding each of these components were amplified by PCR and joined together by strand overlap PCR with primers AbVec_NSN_F1 and AbVec_NSN_R3 (Table 5). The resulting PCR fragment was then cloned into an engineered AbVec-hIgG1 vector digested with AgeI and SalI The AbVec-hIgG1 vector contains a murine heavy chain leader sequence and a human IgG1 fusion. Other Fcs are likewise suitable, including, for example, IgG4 with or without stabilising and/or humanising modifications, such as S288P and those modifications taught in, for example, Atyeo et al. 2020, Suzuki et al. 2018, and Dumet et al 2019. Following transient expression in expi293 cells, the proteins were purified by affinity chromatography on Protein A-sepharose (Huo, Le Bas et al. 2020).

TABLE 9
Primers used for construction
of bidentate polypeptides

| | |
|---|---|
| Primer | Sequence |

N-terminal Nb domain

| | |
|---|---|
| AbVec NSN | 5′-CTAGTAGCAACTGCAACCGGTGTT |
| F1 (Fc- | CACTCTCAGGTGCAGCTGGTGGAGTCT |
| fusion) | GG-3′ |
| NSN_R1 | 5′-CTGGTTCACTTCGCTATCGGAGCC |
| | AGAACCGCTCCCTGAGGAGACGGTGAC |
| | CTGGGTCCC-3′ |

SUMO

| | |
|---|---|
| NSN_F2 | 5′-GGGACCCAGGTCACCGTCTCCTCA |
| | GGGAGCGGTTCTGGCTCCGATAGCGAA |
| | GTGAACCAG-3′ |
| NSN_R2 | 5′-CCAGACTCCACCAGCTGCACCTGCG |
| | ACCCGCTACCTGAGCCGATCTGTTCGCG |
| | ATGCGC-3′ |

C-terminal Nb domain

| | |
|---|---|
| NSN_F3 | 5′-GCGCATCGCGAACAGATCGGCTCAG |
| | TGAGCGGGTCGCAGGTGCAGCTGGTGGA |
| | GTCTGG-3′ |
| AbVec NSN | 5′-GATTTGGGCTCGGTCGACGCTGAGG |
| R3 (Fc- | AGACGGTGACCTGGGTCCC-3′ |
| fusion) | |

Binding Activity

Binding activity was measured by SPR according to the methodology provided in Example 1. It was found that combining VHH_C1 with either VHH_H3 or VHH_H11-H4 results in an approximately 40-fold increase in affinity (Table 10). Combining C1 with C5 results in an approximately 240-fold increase in affinity. Combining F2 with C5 results in an approximately 85-fold increase in affinity.

The F2-SUMO-VHH_H6-Fc F2-SUMO-C5-Fc, and C1-SUMO-C5-Fc biparatopic bivalent polypeptides, in particular, display remarkably high binding affinities, with dissociation constants of 0.6, 0.75 and 1.63 pM respectively.

TABLE 10
SPR RBD binding kinetics of biparatopic VHH polypeptides.

| Nanobody | Ka (1/Ms) | Kd (1/s) | K_{D} (pM) | T_{1/2} (h) |
|---|---|---|---|---|
| F2-SUMO- | 1.3E+07 | 7.6E−06 | 0.6 | 25.4 |
| VHH_H6_Fc | | | | |
| F2-SUMO-C5-Fc | 2.3E+07 | 1.7E−05 | 0.75 | 11.0 |
| C1-SUMO-C5-Fc | 3.0E+07 | 4.9E−05 | 1.63 | 246 |
| C1-SUMO-H3-Fc | 4.3E+06 | 4.7E−05 | 11 | 4.1 |
| C1-SUMO-H11- | 5.1E+06 | 6.8E−05 | 13 | 2.8 |
| H4-Fc | | | | |

Neutralisation Activity

Neutralisation activity was measured by micro-titre neutralisation (‘MN’) assay according to the methodology provided in Example 1. Fc fusions were serially diluted into Dulbecco's Modified Eagles Medium (DMEM) containing 1% (w/v) foetal bovine serum (FBS) in a 96-well plate. SARS-CoV-2 Victoria strain passage 4 (vero 76) [9×10⁴ pfu/ml] diluted 1:5 in DMEM-FBS was added to each well, with media only as negative controls. After incubation for 30 min at 37° C. Vero cells (100 μl) were added to each well and the plates incubated for 2 h at 37° C. Carboxymethyl cellulose (100 μl of 1.5% v/v) was then added to each well and the plates incubated for a further 18-20 h at 37° C. Cells were fixed with paraformaldehyde (100 μl/well 4% v/v) for 30 min at room temperature and then stained for SARS-CoV-2 nucleoprotein using a human monoclonal antibody (EY2A). Bound antibody was detected by incubation with a goat anti-human IgG HRP conjugate and following substrate addition imaged using an ELISPOT reader. The neutralization titre was defined as the titre of VHH-Fc that reduced the Foci forming unit (FFU) by 50% compared to the control wells.

The F2-SUMO-C5, C1-SUMO-H3, C1-SUMO-H11-H4 were tested in the SARS-CoV2 microneutralisation assay and showed sub-nanomolar neutralisation activities.

### Example 5

Trivalent versions of the four nanobodies, C5 (SEQ ID NO: 189), C1 (SEQ ID NO: 225) H3 (SEQ ID NO: 229) and A8 (SEQ ID NO: 221) were constructed by joining the VHH domains with a glycine-serine flexible linker, (GS)6. To generate the trimeric VHHs, the C1, C5, H3 and A8 gene fragments were used as templates to amplify three fragments by PCR with the following pairs of primers: TriNb_Neo_F1 and TriNb_R1; TriNb_F2 and TriNb_R2; TriNb_F3 and TriNb_Neo_R1 (Table C); the three fragments were then joined together with a PCR reaction using primers TriNb_Neo_F2 and TriNb_Neo_R2 (Table C). The trimeric gene product was then inserted into the pOPINTTGneo vector by Infusion® cloning. pOPINTTG contains a mu-phosphatase leader sequence and C-terminal His6 tag44. The nanobody homo-trimers (C5, C1, A8 and H3) were produced by transient expression in expi293 cells and purified by metal chelate affinity chromatography and size exclusion.

TABLE 11
Primers for trivalent VHH construction

| | |
|---|---|
| TriNb_Neo_F1 | 5′-GCGTAGCTGAAACCGGCCAGGT |
| | GCAGCTGGTGGAGTCTGGG-3′ |
| TriNb_R1 | 5′-GACTCCACCAGCTGCACCTG |
| | GGAGCCAGAACCGCTCCCTGAGG |
| | AGACGGTGACCTGG-3′ |
| TriNb_F2 | 5′-CCCAGGTCACCGTCTCCTCAG |
| | GGAGCGGTTCTGGCTCCCAGGTGC |
| | AGCTGGTGGAG-3′ |
| TriNb_R2 | 5′-GACTCCACCAGCTGCACCTGC |
| | GACCCGCTACCTGAGCCTGAGGAG |
| | ACGGTGACCTGG-3′ |
| TriNb_F3 | 5′-CCCAGGTCACCGTCTCCTCAG |
| | GCTCAGGTAGCGGGTCGCAGGTGC |
| | AGCTGGTGGAG-3′ |
| TriNb_Neo_R1 | 5′-GTGATGGTGATGTTTTGAGG |
| | AGACGGTGACCTGGGTCCC-3′ |
| TriNb_Neo_F2 | 5′ -GCGTAGCTGAAACCGGCCAG-3′ |
| TriNb_Neo_R2 | 5′ -GTGATGGTGATGTTTTGAGG-3′ |

Potent Neutralisation of SARS-CoV2 Variants In Vitro by Trimeric Nanobodies

The nanobody trimers C5 (SEQ ID NO: 189), C1 (SEQ ID NO: 225), H3 (SEQ ID NO: 229) A8 (SEQ ID NO: 221) were produced by transient expression in expi293 cells and purified by metal chelate affinity chromatography and size exclusion. Binding of the trimeric nanobodies binding to the RBD was measured by SPR as per Example 1, and an approximate 10 to 100-fold enhancement in K_{D} was observed compared to the monomers (Table 12). Notably, the H3 trimer was shown to have a sub-picomolar KD for the RBD-WT (where WT is the Wuhan/Victoria strain), with an off rate of approximately 6 hours. Binding of C5 trimer to the RBDs from both the SARS-CoV-2 Alpha (lineage B.1.1.7) and Delta variants (lineage B.1.167.2) was similar to RBD-WT whilst binding of C5 monomer was ˜25-fold and ˜100 fold weaker respectively (Table 5). Only C1 and A8 showed binding to the RBD (South Africa) from the SARS-CoV-2 Beta variant (Lineage B.1.351 (Table 12).

TABLE 12
SPR RBD binding kinetics of nanobody trimers.

| | | Kₐ | K_{d} | K_{D} | T_{1/2} |
|---|---|---|---|---|---|
| Analyte | Ligand | (1/Ms) | (1/s) | (pM) | (min) |
| C5 trimer | RBD-WT-Fc | 7.1E+06 | 1.2E−04 | 18 | 92 |
| C5 trimer | Bio-RBD-Alpha | 9.9E+06 | 2.8E−04 | 29 | 41 |
| C5 trimer | Bio-RBD-Delta | 1.0E+7 | 1.4E−04 | 14 | 82 |
| H3 trimer | RBD-WT-Fc | 1.2E+08 | 3.3E−05 | 0.3 | 349 |
| H3 trimer | Bio-RBD-Alpha | 1.8E+07 | 1.2E−04 | 6 | 98 |
| C1 trimer | RBD-WT-Fc | 9.0E+05 | 4.8E−05 | 53 | 242 |
| C1 trimer | Bio-RBD-Alpha | 1.0E+06 | 7.4E−05 | 73 | 154 |
| C1 trimer | Bio-RBD-Beta | 8.2E+05 | 6.2E−05 | 75 | 186 |
| A8 trimer | RBD-WT-Fc | 9.1E+06 | 4.1E−0.5 | 5 | 278 |
| A8 trimer | Bio-RBD-Beta | 9.4E+06 | 4.6E−0.5 | 5 | 251 |
| A8 trimer | Bio-RBD-Delta | 9.2E+06 | 5.2E−0.5 | 6 | 222 |

Micro-neutralisation assays were carried out (as per Example 1) to test the effectiveness of the three nanobody trimers to block infection of Vero E6 cells by either Victoria (VVT), Alpha, Beta and Delta strains of the virus. All nanobodies potently neutralized some if not all the strains. As anticipated from the in vitro binding data, only A8 and C1 were active against the Beta strain. Although H3 bound more tightly than C5 to the RBDs in vitro, it was less potent than C5 against both WT and Alpha strains. Crucially, C5 was equipotent in neutralising the Victoria (WT), Alpha and Delta viruses whereas A8 and C1 neutralised all four strains (Table 13)

TABLE 13
Neutralisation of live viruses in the MN assay
ND 50 MN assays

| | Variant | | | | |
|---|---|---|---|---|---|
| | | | Alpha | Beta (South | |
| | B | | (Kent | Africa | Delta |
| Nanobody | (Victoria) | | B.1.1.7) | B.1.351) | (B.1.617.2) |
| C1 trimer | 4.9 | nM | 8.2 nM | 4.5 nM | 3.1 nM |
| C5 trimer | 0.02 | nM | 0.25 nM | 0 | 0.02 nM |
| H3 trimer | 0.4 | nM | 0.6 nM | 0 | 0.3 nM |
| A8 trimer | 0.09 | nM | 0.1 nM | 0.2 nM | 0.1 nM |

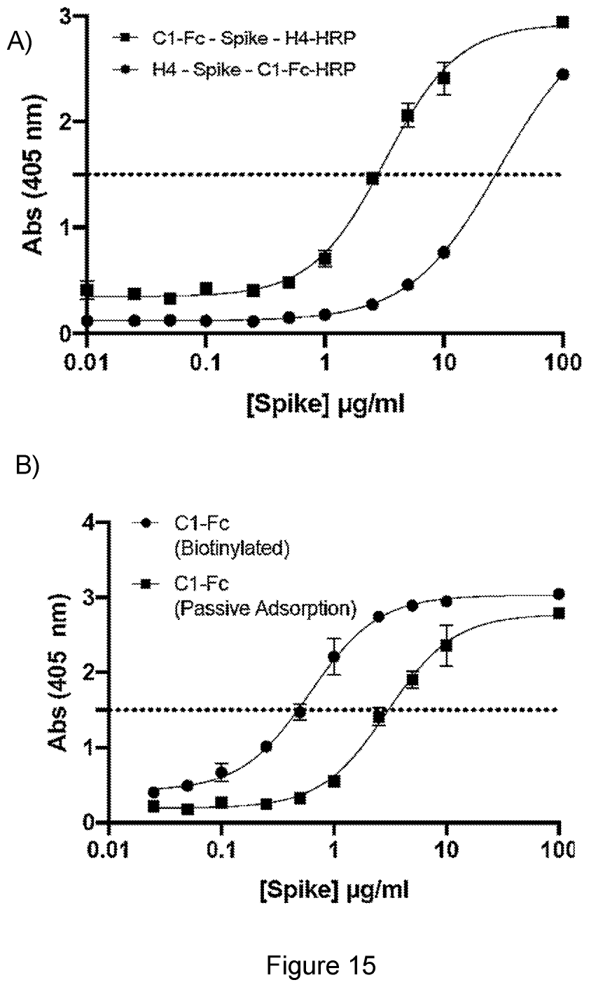

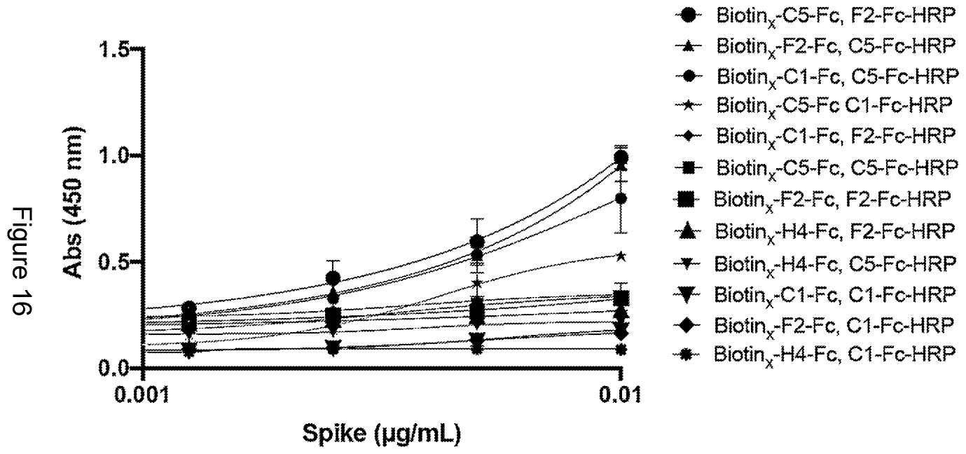

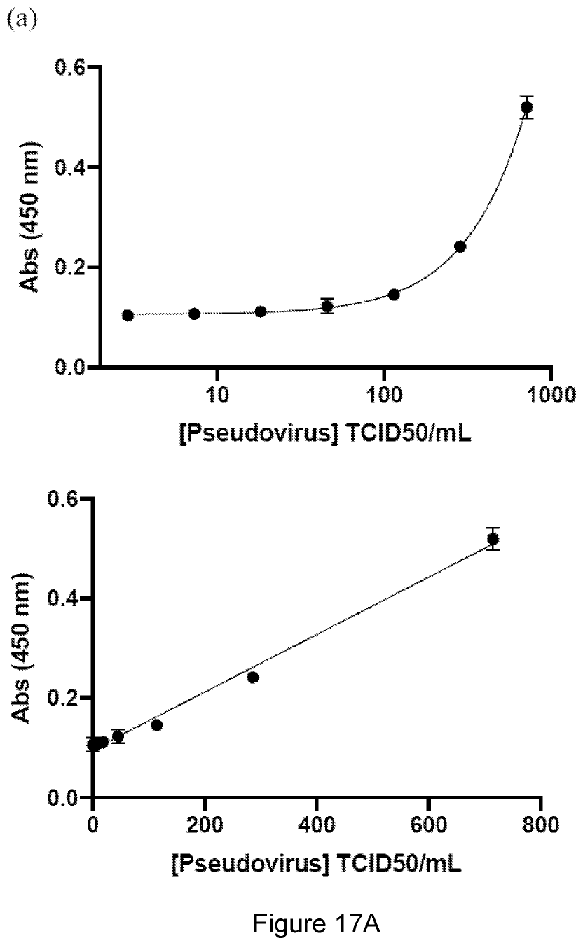

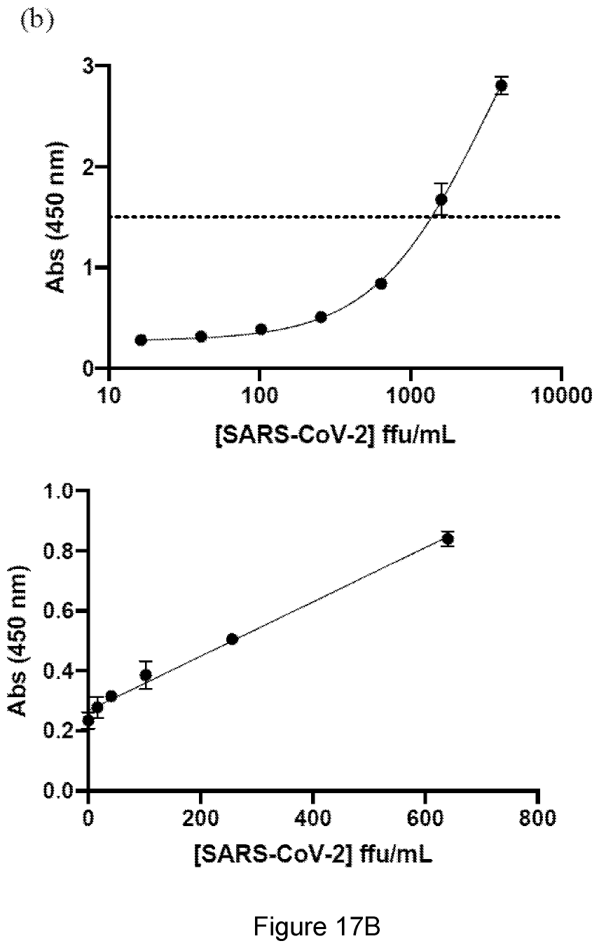

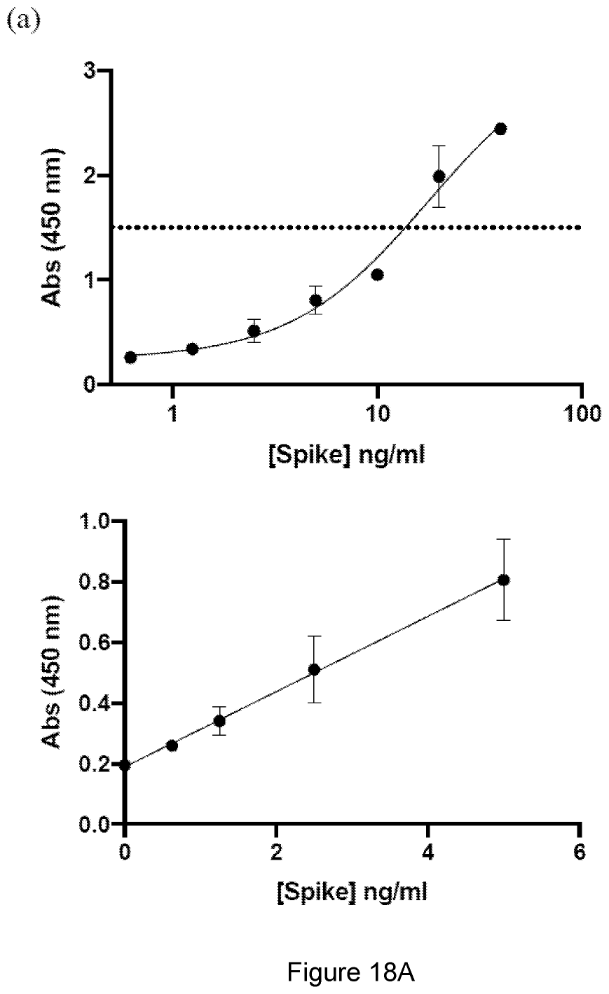

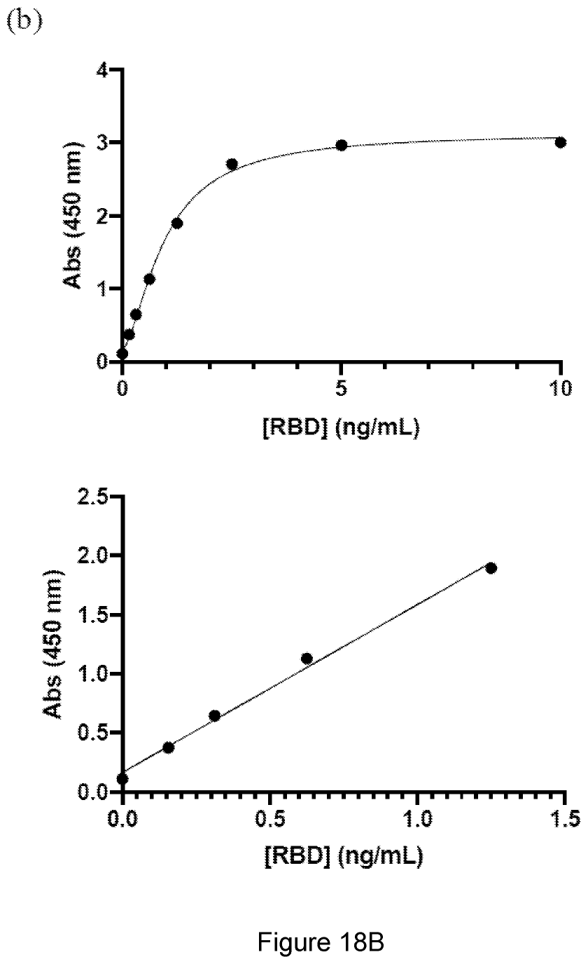

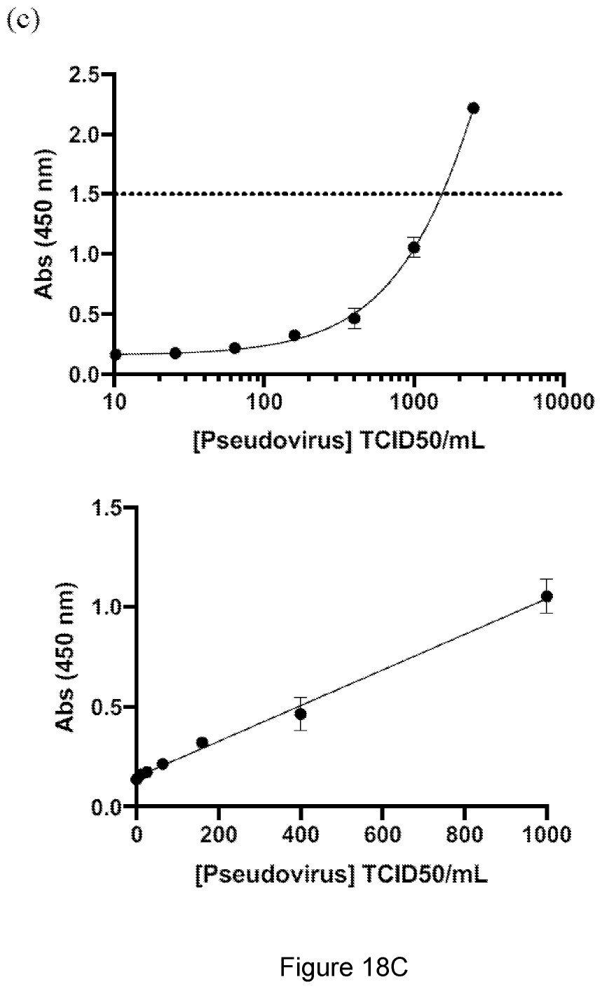

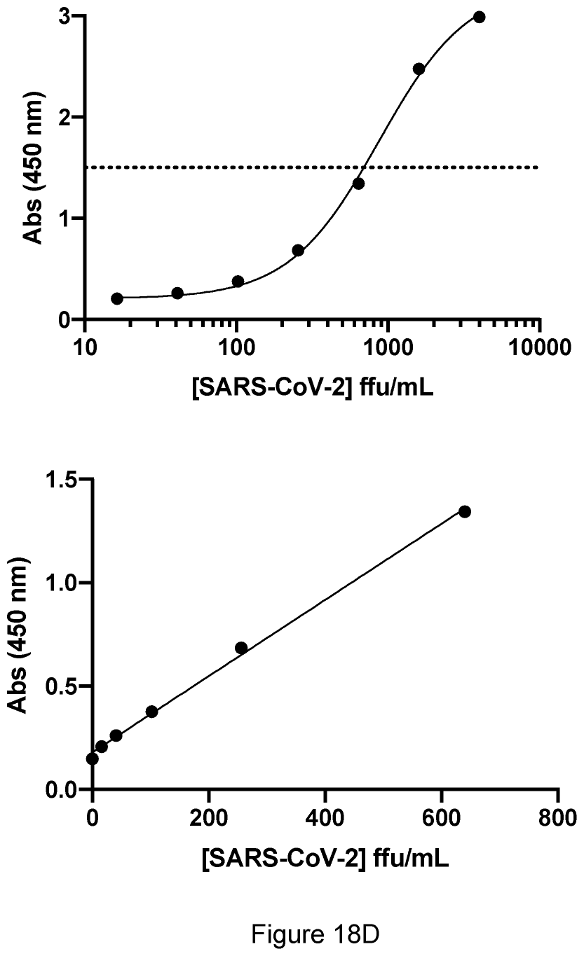

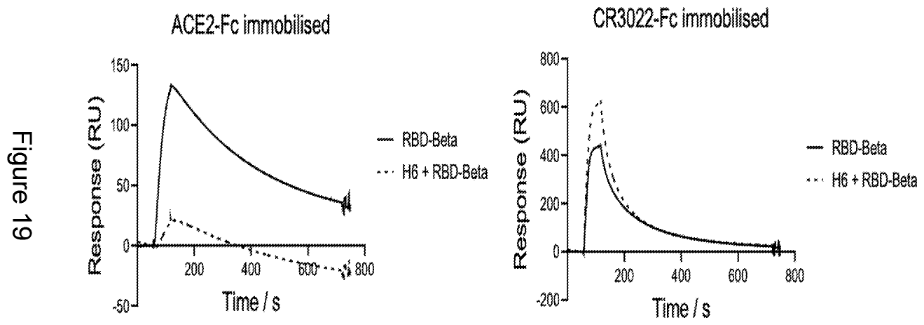

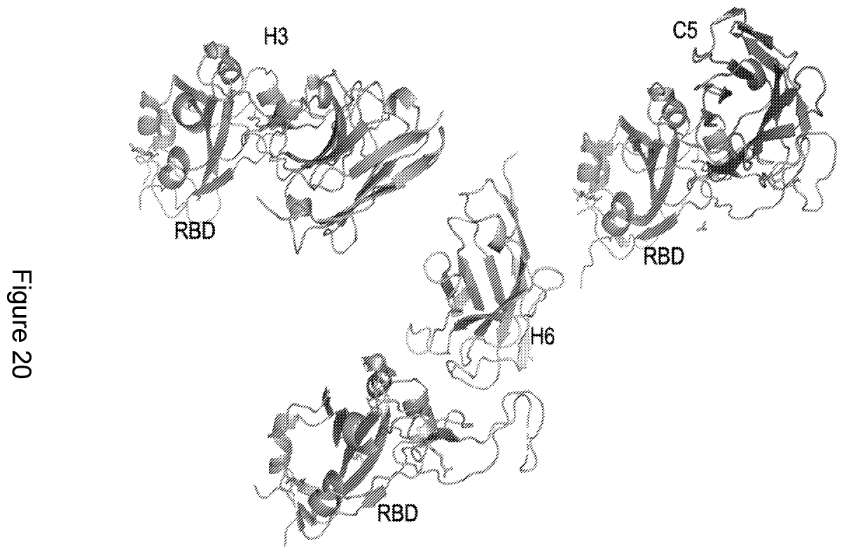

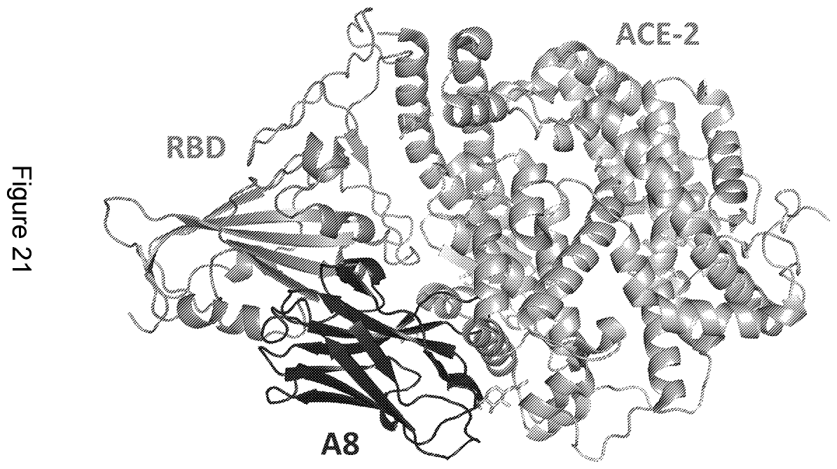

The neutralization potency of the C5 trimer was confirmed in the Gold Standard Plaque Reduction Neutralisation Test (PRNT) against the BVIC01 strain which gave an ND50 of 3 pM (data FIG. 9).

Plaque reduction neutralization tests (PRNT) were carried out at Public Health England using SARS-CoV-2 (hCoV-19/Australia/VIC01/2020) (GISAID accession number EPUSL_406844) generously provided by The Doherty Institute, Melbourne, Australia at P1 and passaged twice in Vero/hSLAM cells [ECACC 04091501]. Virus was diluted to a concentration of 933 p.f.u. ml-1 (70 p.f.u./75 μl) and mixed 50:50 in minimal essential medium (MEM; Life Technologies) containing 1% FBS (Life Technologies) and 25 mM HEPES buffer (Sigma) with doubling antibody dilutions in a 96-well V-bottomed plate. The plate was incubated at 37° C. in a humidified box for 1 h to allow neutralization to take place. Afterwards, the virus-antibody mixture was transferred into the wells of a twice Dulbecco's PBS-washed 24-well plate containing confluent monolayers of Vero E6 cells (ECACC 85020206, PHE) that had been cultured in MEM containing 10% (v/v) FBS. Virus was allowed to adsorb onto cells at 37° C. for a further hour in a humidified box, then the cells were overlaid with MEM containing 1.5% carboxymethyl cellulose (Sigma), 4% (v/v) FBS and 25 mM HEPES buffer. After five days incubation at 37° C. in a humidified box, the plates were fixed overnight with 20% formalin/PBS (v/v), washed with tap water and then stained with 0.2% crystal violet solution (Sigma) and plaques were counted. A mid-point probit analysis (written in R programming language for statistical computing and graphics) was used to determine the dilution of antibody required to reduce SARS-CoV-2 viral plaques by 50% (ND50) compared with the virus-only control (n=5). The script used in R was based on a previously reported source script (Nettleship, J E et al 2009). Antibody dilutions were run in duplicate and an internal positive control for the PRNT assay was also run in duplicate using a sample of heat-inactivated (56° C. for 30 min) human MERS convalescent serum pH 7.4, 137 mM NaCl, 1 mM CaCl) and 1 mg ml-1 trypsin (Sigma-Aldrich) to neutralize SARS-CoV-2 (National Institute for Biological Standards and Control, UK).

C5-Fc Fusion Shows Therapeutic Efficacy In Vivo

To probe neutralization in vivo, we tested the C5-Fc fusion in the Syrian Hamster model of COVID-19 (Chan, J. F. et al 2020; Imai, M. et al. 2020; Sia, S. F. et al 2020), which has demonstrated with SARS-CoV to show clinical disease (loss of weight and clinical signs), viral replication restricted to respiratory tissues and shedding of virus in nasal secretions. The RBD binding affinity (KD 37 pM) and virus neutralisation potency (ND50 of 2 pM; 180 pg/ml) of C5-Fc was similar to the trivalent protein, confirming the importance of multivalency (Table 18). The study consisted of an experimental and control group each of six animals. Animals in both groups were challenged intranasally with SARS-CoV-2 Victoria (5×10⁴ pfu) and then one group treated 24 h later with a single dose of C5-Fc (4 mg/kg) administered intraperitoneal (IP′) whilst the control were injected with PBS only. As a measure of disease progression, the animals were weighed each day over 7 days and nasal swabs taken every other day. On day 7 the animals were culled and viral load in lung, trachea and duodenum measured by ISH and sg-PCR. Vital organs were formalin-fixed for histopathology and antibody staining with anti-SARS Cov2 nucleoprotein (NP) to detect presence of the virus. The control group of six animals that received vehicle (PBS) only showed 16% weight loss by day 7 whereas the treated group after an initial weight loss recovered to a 5% weight loss relative to pre-challenged weights with a trend towards the same weight as un-challenged sentinel group (FIG. 10). Histopathology and RNAScope ISH technique were used to compare the pathology and the presence of viral RNA in tissues from nanobody-treated and untreated control hamsters, combining a semiquantitative scoring system and digital image analysis to calculate the area of lung with pneumonia and the quantity of virus. Lesions consistent with infection with SARS-CoV-2 were observed only in the lungs and nasal cavity. No lesions were observed in any other organ studied. Virus RNA was only observed in the lung and nasal cavity. The lung lesions consisted of a bronchointerstitial pneumonia showing areas of lung consolidation and were characterized by infiltration of macrophages and neutrophils, but also some lymphocytes and plasma cells. The area with pneumonia was significantly reduced in the nanobody-treated hamsters, together with a marked reduction of histopathology scores in the nasal cavity (Statistically significant differences were also found for the presence of virus RNA in the lung or the nasal cavity (FIG. 10)). Together, these results showed that a single therapeutic dose of C5-Fc administered IP reached the site of action in the lungs and nasal cavity and reduced viral load and associated pathology. Therefore, based on these positive results we undertook a larger study to evaluate the C5 trimer in the Syrian hamster model.

Trimeric C5 Nanobody Shows Topical Therapeutic Efficacy

The smaller molecular size of the C5-trimer (40 kDa) compared to the C5-Fc (80 kDa plus 2N-linked glycans) makes this suitable for topical administration directly to the airways. Therefore, in the second animal study, the efficacy of the trimeric version of C5 was evaluated in the COVID-19 hamster model by administration using both IP and intranasal routes. The study consisted of five groups of six animals that were challenged with SARS-CoV-2 Victoria (1×10⁴ pfu) on day 1 and weight changes followed over 7 days. To compare to the results obtained with the C5-Fc, the trimer was administered IP at 4 mg/kg and the same dose delivered directly to the airways via the nasal installation. A tenfold lower intranasal dose of 0.4 mg/kg of C5-timer was also tested. As in the first study, animals in the untreated group showed a significant and progressive weight loss (20% by day 7), whereas all animals treated therapeutically, 24 h after viral challenge, showed only an initial weight loss and from day 2 recovered to pre-challenged weights (FIG. 11). The animals pre-treated prophylactically 2 h prior to virus through the nasal route showed no change in weight indicating that the infection had been effectively blocked at day 1. Analysis of viral load in the lung by qPCR of nucleoprotein (NP) RNA showed a significant decrease in the median value in treated compared to the untreated control animals, though two animals still had relatively high levels of NP RNA (FIG. 11).

Collectively the animal studies have established that a multivalent nanobody (Fc fusion or trimer) targeted to the RBD of SARS-CoV-2 S protein delivered either systemically or topically has a therapeutic effect in disease model of COVID-19. In particular, efficacy was observed with a single intranasal dose of 0.4 mg/kg (equating to approximately 40 ug/animal) of the C5-trimer demonstrating the high potency of this biological agent. A further dose ranging study will establish the minimum amount of the nanobody required to be therapeutically effective in the hamster disease model.

### Example 6

A quantitative ELISA has been developed for measuring the amount of SARS-CoV2 spike protein, with application for in-process monitoring of spike production in the manufacture of antibody testing kits. Knowledge of the epitopes recognised by the SARS-Cov2 specific VHH enables the selection of the appropriate pairs of VHHs/antibodies in the design of a sandwich ELISA. The quantitative ELISA uses a combination of VHHs and/or antibodies that bind to spatially distinct, spatially separated epitopes on the RBD.

The results of an ELISA assay in which biotinylated VHH_C1-Fc is coated onto 96-well ELISA plates and captured spike protein is detected by HRP (Horse Radish Peroxide)-conjugated VHH_H11-H4 are representative of input levels of spike protein and sensitive to 100 ng/ml.

Chemicals

PNGase F was purchased from NEB and mTGase was sourced form Zedira (T001). Amino-PEG3-biotin was purchased form ThermoFisher. MES buffer and SDS PAGE gels were purchased from Invitrogen. Vivaspin filter membranes were purchased from Sartorius and the membranes were washed with milliQ water and PBS before use. Other chemicals were purchased from SigmaAldrich unless otherwise stated.

Protein Production

Purified H4, H4-Fc, C5-Fc, C1-Fc, F2-Fc, RBD and SARS-CoV-2 Spike were prepared as previously described. HRP-nanobody conjugates were prepared according to method supplied with Abcam Lightning Link HRP conjugation kit (www.abcam.com) and used without any further treatment. The extent of conjugation was estimated by analysing the bands of conjugated nanobody in SDS-PAGE gel electrophoresis. The addition of biotin non-specifically to lysine residues of the nanobodies was achieved using Thermo Fisher EZ-Link Sulfo-NHS-Biotinylation Kit or EZ-Link Sulfo-NHS-LC-Biotinylation Kit (www.thermo.com). The kits were used as per the provided instructions. Once complete, the reaction solutions were dialysed in PBS to remove any unreacted biotin reagent, and concentration determined by nanodrop. The extent of biotinylation was established with Thermo Scientific Fluorescence Biotin Quantification Kit.

Site Specific Labelling N-linked glycans on 500 ug C5-Fc (1 mg/mL in PBS) were removed by incubation at 37° C. for 18 h with 5 uL of 5×PBS buffer and 10 uL of PNGaseF enzyme. The completion of the reaction was determined by gel electrophoresis. Deglycosylated C5-Fc (dgC5-Fc) was purified on a protein A affinity column (GE lifesciences) and eluted with citrate buffer (pH 3) and neutralized using 1 M Tris (pH 9). The purified dgC5-Fc was buffer exchanged into PBS using a 10 kDa Vivaspin filter membrane. 100 pg of dgC5-Fc (1 mg/mL) was then incubated at 37° C. with amino-peg3-Biotin (80 equivalents) and mTGase enzyme (6 U/mL) (modifies PREEQYNST). 1 uL aliquots of the reaction were collected and analysed by reducing LCMS (concentration of 0.002 mg/mL protein in water and reduced by 20 mM DTT). The modified nanobody was analysed on a ProSwift TM RP-4H HPLC column using 0.1% aqueous formic acid and 95% acetonitrile as mobile phases. The data was deconvoluted and analysed using MassLynx v4 software. We consistently observed another product in our reactions which showed a loss of mass of 15 Da. We assigned this to formation of an internal bond with lysine in a competing reaction that results in the loss of mass. Since any such product will not bind to the streptavidin-coated ELISA plates thus have no influence we continued with purification of the reaction mixture by protein A affinity chromatography. The purified product, which had a single biotin moiety per C5-Fc monomer was used in our ELISA assays.

SARS-CoV-2 WT (Heat/Empigen and 4% FA) and Pseudovirus

Infectious SARS-CoV-2 virus was grown in Vero CCL81 or Vero E6-TMPRSS2. Propagation was performed in T175 tissue culture flasks. When cells reached approximately 70% confluence, all medium was removed and 1 mL (DMEM, 1% FBS, 1% P/S) of virus-containing medium was added to each flask (MOI ˜0.001). This was left to incubate for 10 mins at room temperature before topping up with a further 19 mL of medium.

Flasks were left to incubate for 48-72 hours post-infection until significant cytopathic effect was visible in the culture. Harvesting consisted of pooling the media, pelleting cell debris (5 mins, 500 RCF), and subsequent aliquoting of the supernatant.

Virus titre was determined by focus-forming assay. Vero CCL81 cell suspension was added to serially diluted virus stock and incubated for 2 hours. A viscous carboxymethylcellulose (CMC) overlay was added and plates incubated a further 22 hours. Medium was then removed, monolayers fixed with 4% formaldehyde (30 mins), and cells stained for SARS-CoV-2 nucleocapsid by a standard primary/secondary-HRP procedure. Titre was measured as focus-forming units per mL (ffu/mL) (Skelly et al. 2021—https://doi.org/10.21203/rs.3.rs-226857/v2). Pseudovirus was produced by co-transfection of HEK-293T cells with a pNL4-3 (ΔEnv, luciferase) lentiviral plasmid and a plasmid encoding the SARS-CoV-2 spike gene.

Transfection was with polyethylenimine (PEI) for 4 hours. After which, cells were washed, fresh media (DMEM, 10% FBS, 1% P/S) applied, and virus production left to proceed for 48 hours. Virus was harvested by pooling supernatant and concentration by polyethylene glycol (PEG) precipitation.

Titration of pseudovirus was performed by infecting cells overexpressing ACE2 (MDCK-ACE2) for 48 hours with a limiting dilution of virus stock. Readout was by standard luciferase assay of cell lysate, with each well classified as positive or negative for infection. Titre was measured in units of TCID50/mL.

Inactivation of infectious virus stocks was carried out either by formaldehyde fixation or combined heat/detergent inactivation. For formaldehyde fixation: 32% stock solution was mixed with virus sample to achieve a final 4% formaldehyde concentration and incubated for 30 mins before removal from the CL3 facility.

For heat/detergent inactivation: Empigen detergent was diluted in MilliQ water to a 5% solution. This was mixed 1:10 with the virus sample (final Empigen concentration 0.5%). Sample was then heated for 30 mins at 56° C. ELISA

For passive absorption, high binding Nunc 96-well microplates from Sigma were first rinsed with phosphate buffered saline (PBS, 3×200 pL). To each well we added 100 pL of purified protein (C1-Fc or H4) at 5 pg/mL and incubated for 3 hours at 37° C. The plate was then washed with PBS and blocked by addition of 3% milk/PBS (200 pL/well) at 4° C. overnight. After a further wash with PBS, the recombinant SARS-CoV-2 spike protein was added in serial dilutions ranging from 100 pg/mL to 0.01 pg/mL and incubated for 90 minutes at 37° C. with PBS as a negative control. The wells were washed again with PBS and 100 pL/well of HRP-conjugated probe molecules diluted (from 0.5 mg/mL) in PBS (1:3000) and added to each well and incubated at room temperature for 2 hours. The wells were washed again with PBS, before a freshly prepared solution of developer was added.

For biotinylated nanobodies, streptavidin coated high capacity 96-well microplates from Sigma were washed with Tris-HCl 50 mM, NaCl 150 mM, 0.1% BSA and 0.05% Tween-20 (EWB). A 100 pL solution of biotinylated nanobodies previously diluted to 0.5 pg/mL in EWB was added to each well and incubated at room temperature for 2 hours. The plate was washed with EWB and 100 μL of a serial dilution of spike protein (typically 10-0.0001 pg/mL) were arrayed in the wells followed by 30 minute incubation at room temperature. Following a subsequent wash step with EWB, 100 pL/HRP-Fc-conjugated nanobodies (0.5 mg/mL) were diluted in EWB (1:1000) was added to each well and left for 30 minutes at room temperature.

Two developers were used, firstly 100 μL ABTS (0.3 mg/mL) in a peroxide solution (0.01%) was added and the plate was shielded from light and left for 20 minutes, at which point the absorbance at 405 and 410 nm was read by SpectraMax M3 microplate reader (Molecular Devices). For the second developer, 100 μL 3,3′, 5,5′-tetramethylbenzidine (TMB, 0.2 mg/mL) in a peroxide solution (0.01%), after which the plate was shielded from light for 20 minutes. H2SO4 (2M, 100 μL/well) was added to each well the OD at 450 nm was recorded using the same SpectraMax M3 microplate reader as above.

ELISA with Virus

C5-Fc was specifically biotinylated as above and the probe nanobody chosen was F2-Fc conjugated to HRP as described above. Streptavidin coated plates and the TMB development protocols described above were used. Three different means of inactivation of SARS-CoV-2 were employed addition of detergent (Empigen) followed heating (60 C, 30 minutes); addition of 4% formaldehyde (FA) and exposure to 12.2 kGy of X-ray irradiation. A pseudotyped lentivirus NL4.3 expressing SARS-CoV-2 spike protein was also evaluated and was supplied as live virus in PBS. SARS-CoV-2 virus samples were tested in serial dilutions ranging from 4000 ffu/mL to 16.384 ffu/mL. Pseudovirus dilution series ranged from 2500 TCID50/mL to 10.24 TCI D50/m L.

Limit of Detection

The sensitivity of ELISA is largely judged by the limit of detection (LOD); the lowest detectable level of analyte that can reliably be distinguished from background. We used LOD=(3.3*Sy)/k, where Sy is the standard deviation of blank replicate samples, and k is the gradient of slope from linear regression analysis. As standard deviation can vary significantly, k can be a helpful way to gauge sensitivity.

Results:

Optimising the Capture Component

We started with the pM binder C1-Fc (VHH conjugated to human IgG1 Fc (bivalent and glycosylated)) and nM binder H4 (VHH domain only) {Huo et al., 2020, #67790}. Using C1-Fc as the capture agent, with H4-HRP as the probe gave a limit of detection of ˜1.85 pg/mL of Spike protein (estimated >3 times the standard deviation of blank samples). When H4 was adsorbed onto the plate and the HRPconjugates of C1-Fc were used as the probe, the detection limit increased to 20 pg/mL of Spike protein. We concluded that the VHH domain on its own was either not readily adsorbed onto the plate or when absorbed onto the plate its binding site was obscured. We then investigated whether biotinylated nanobodies and binding to streptavidin treated plates improved the sensitivity of the assay. High binding Nunc plates were treated with unmodified C1-Fc and separately a streptavidin coated plate were treated with biotinx-C1-Fc prepared with ThermoFisher EZ-Link Sulfo-NHS-Biotin. This reagent biotinylates lysine residue side chains in a non-specific stochastic manner. Both plates were treated with recombinant S glycoprotein ranging from 100 pM to 0.25 pM, and then probed with H4-HRP. Where C1-Fc was passively absorbed the limit of detection of S protein was calculated as 1.3 pg/mL essentially the same result as a standard plate (FIG. 15). Using the biotinx-C1-Fc with streptavidin plates lowered the limit of detection to 0.1 pg/m L.

Evaluating Nanobody Pairs

We continued with streptavidin coated plates using the capture nanobody in the form biotinx-Nb-Fc (0.5 pg/mL) and the probe nanobody HRP-Nb (1:1000 dilution from 0.5 mg/mL stock). We switched to use ABTS as the substrate of HRP, since this commercially available molecule has higher sensitivity and faster colour development than TM B {Hosoda et al., 1986, #32295}. We found that H4-Fc-HRP when used as the probe gave unreliable detection results. Dot blotting of H4-Fc-HRP with other nanobodies suggested it may have non-specific interactions, the other nanobodies did not show this behaviour. The results from the various remaining combinations are given in Table 14 and FIG. 16. The estimated limit of detection varied between 4 pg/mL (biotinx-H4-Fc, C1-Fc-HRP) to less than 1 pg/mL (Table A). The biotinx-C5-Fc, F2-Fc-HRP combination gave a regression gradient (79 ng/mL), strong absorbance and a limit of sensitivity of 0.5 pg/mL which we judged to be optimal performance. As a negative control we repeated the ELISA with nanobody pairings with overlapping epitope combinations. These were as expected much less sensitive and when plotted gave lines with significantly less steep slopes, thus do not respond as strongly to changes in concentration.

TABLE 14
The estimated limit of detection in ng/ml

| | | Probe | | | | | |
|---|---|---|---|---|---|---|---|
| Capture | | C1-Fc-HRP | | F2-Fc-HRP | | C5-Fc-HRP | |
| | biotinₓ-C1-Fc | — | | 9 | (12) | 0.1 | (64) |
| | biotinₓ-F2-Fc | 1 | (9) | — | | 0.4 | (79) |
| | biotinₓ-C5-Fc | 0.2 | (47) | 0.51 | (79) | — | |
| | biotinₓ-H4-Fc | 5 | (2) | 3 | (8) | 3 | (6.5) |

*In parenthesis is the slop (×1000) of the line. In bold is the combination selected.

Testing Against Virus

We were unable to test live virus due to safety restrictions, instead we instead tested intact pseudotyped NL4.3 HIV-1 backbone virus displaying surface SARS-CoV-2 S glycoproteins. Our lower limit of detection was 21 TCID50/mL of infectious pseudotyped virus (FIG. 17a). We were able to test inactivated VVT SARS-CoV-2 virus. Three different methods of inactivation were chosen: a) 4% formaldehyde b) x-ray irradiation (12.19 kGy) c) empigen (0.05%) and heat (60° C., 30 minutes). We were unable to detect formaldehyde inactivated SARS-CoV-2 but detected X-ray inactivated virus at 305 pfu/mL and heat/empigen inactivated virus at 103 ffu/mL (FIG. 17b).

TABLE 15
Parameters of FIG. 17.

| | Slope | St. | | | |
|---|---|---|---|---|---|
| | (×10⁴) | dev. | LOD | | r² |
| Pseudovirus | 7.0 | 0.00442 | 21 | TCID50/mL | 0.996 |
| SARS-CoV-2 (Empigen) | 8.9 | 0.0277 | 103 | ffu/mL | 0.980 |

TABLE 16
Parameters of FIG. 18.

| | | St. | | | |
|---|---|---|---|---|---|
| | Slope | dev | LOD | | r² |
| Spike | 0.1241 | 0.00552 | 147 | pg/mL | 0.911 |
| RBD | 1.421 | 0.0143 | 33 | pg/mL | 0.993 |
| Pseudovirus | 14.7 × 10⁻⁴ | 0.00711 | 15.8 | TCID50/mL | 0.993 |
| SARS-CoV-2 | 18.4 × 10⁻⁴ | 0.00889 | 15.9 | ffu/mL | 0.997 |
| (empigen) | | | | | |

Site Selective Biotinylation

We evaluated whether site specific regioselectively controlled biotinylation of the capture agents would improve the sensitivity of the assay. A streptavidin coated 96-well plate was treated with biotinx-C5-Fc-biotin and site specific biotinylated C5-Fc (biotin-SS-C5-Fc-biotin) at the same concentration. We used recombinant SARS-CoV-2 S glycoprotein, RBD (FIG. 18b), pseudovirus (FIG. 18c), and heat-empigen inactivated WT SARS-CoV-2 (FIG. 18d) as substrates. The plates were probed with F2-Fc-HRP. Using biotin-SS-C5-Fc-biotin as the capture showed an increased sensitivity compared to the multi-biotinylated form for purified Spike protein (147 pg/mL vs 514 pg/mL) and for purified RBD (33 pg/mL vs 85 pg/mL) consistent with the lower molecular weight of the isolated domain. Increased sensitivity was also observed in viral samples: for pseudovirus (16 TCID50/mL vs 21 TCID50/mL) with heat empigen inactivated SARS-CoV-2 (16 ffu/mL vs 103 ffu/mL) and X-ray inactivated virus (286 vs 305 pfu/ml). With purified RBD the lower limit of detection was found to be 46 pg/mL, consistent with the lower molecular weight of the isolated domain.

Consistent with other reports using nanobodies in sandwich ELISA, the direct adsorption of nanobodies onto simple plates gave an ELISA that was less successful than biotinylated protein and streptavidin coated plates. The use of Fc fusion simplifies the biotinylation strategy since the Fc portion has many lysine residues and means a standard protocol can be employed. We identified the optimal combination to consist of biotinx-C5-Fc as the capture agent with F2-Fc-HRP as the probe agent. This combination gave an ELISA that had a limit of detection of between 514 pg/mL (FIG. 16). The ELISA showed linear response indicating it would be suitable for reliably quantitating Spike. Other combinations also gave a limit of detection below 1 ng/mL. By using site selective biotinylation the limit of detection was reduced to 147 ng/ml for Spike protein. The use of the nanobody pairs thus give an ELISA that is simple to use as a laboratory tool to monitor the heterologous production of Spike protein. As new variants of the Spike protein become important, for example carrying the E484K mutant, the probe nanobody, C5, will need to be changed. The advantage of nanobodies is that they are relatively straightforward to raise against new antigens in comparison to their human counterparts. This technology thus offers a robust assay platform for process monitoring. We verified that the ELISA was also compatible with the Spike protein when presented as an intact viral particle by using pseudotype virus confirming that the nanobody can recognise natively folded protein as part of an infectious virus. We also obtained evidence that the assay was sensitive to the “quality” of protein, virus inactivated by formaldehyde (which is expected to chemically alter the binding epitopes) was not detected. SARS-CoV-2 virus inactivated with the mild detergent empigen was detected with a sensitivity of 103 ffu/mL (140 TCID50/mL). We excluded the possibility that empigen was responsible for the gain by showing it had no effect on the detection of Spike protein. Using the reported estimate of 1000 virons in a ffu, 25 spikes in each virion and 600 kDa as the weight of the Spike trimer, this corresponds to around pg of Spike per mL. We hypothesised this 20 fold gain arose from the polyvalent presentation of the Spike protein as part of the viral membrane (avidity effect). Pseudovirus, where the protein is presented on the viral membrane, was detected with comparably high sensitivity 26 TCID50/mL consistent with an avidity effect in the sensitivity of the ELISA. Finally X-ray inactivated (Leung et al., 2020; Afrough et al., 2020) SARS-CoV-2 prepared in a different lab using a different protocol was detected 305 pfu/mL (436 TCID50/mL), corresponding to 90 pg/mL of Spike, once again consistent with an avidity effect. We attribute the difference between ionising radiation and empigen sample to result from differences in protocols and possible damage to the Spike.

Site specific biotinylation of the C1-Fc capture agent resulted in an improvement in ELISA for all test samples when compared to non-specific biotinylation. Notably, the same avidity effect was observed with these agents. In absolute terms the biotin-SS-C5-Fc and F2-Fc-HRP combination was able to detect <2 ffu of empigen inactivated virus.

### REFERENCES

Amanat, F., K. M. White, L. Miorin, S. Strohmeier, M. McMahon, P. Meade, W. C. Liu, R. A. Albrecht, V. Simon, L. Martinez-Sobrido, T. Moran, A. Garcia-Sastre and F. Krammer (2020). “An In Vitro Microneutralization Assay for SARS-CoV-2 Serology and Drug Screening.” Curr Protoc Microbiol 58(1): e108.
Atyeo, C. Slein, M. D. Fischinger, S. Burke, J. Schafer, A. Leist, S. R. Honko, A. Johnson, R. Storm, N. Bernett, M. Linde, C. Suscovich, T. Griffiths, A. Desjarlais, J. R. Juelg, B. D. Goutsmit, J. Baris, R., Alter, G. (2020) “Therapeutic potential of SARS-CoV-2-specific monoclonal antibody CR3022.” Cell Host and Microbe, Pre-Print SSRN: ssrn.com/abstract=3612156
Bird, L. E., H. Rada, J. Flanagan, J. M. Diprose, R. J. Gilbert and R. J. Owens (2014). “Application of In-Fusion cloning for the parallel construction of E. coli expression vectors.” Methods Mol Biol 1116: 209-234.
Compte, M.; Harwood, S. L.; Munoz, I. G.; Navarro, R.; Zonca, M.; Perez-Chacon, G.; Erce-Llamazares, A.; Merino, N.; Tapia-Galisteo, A.; Cuesta, A. M.; et al. A tumor-targeted trimeric 4-1BB-agonistic antibody induces potent anti-tumor immunity without systemic toxicity. Nat. Commun. 2018, 9, 4809
Detalle, L., T. Stohr, C. Palomo, P. A. Piedra, B. E. Gilbert, V. Mas, A. Millar, U. F. Power, C. Stortelers, K. Allosery, J. A. Melero and E. Depla (2016). “Generation and Characterization of ALX-C171, a Potent Novel Therapeutic Nanobody for the Treatment of Respiratory Syncytial Virus Infection.” Antimicrob Agents Chemother 60(1): 6-13.
Dumet, C. Pottier, J. Gouilleux-Gruart, V. Watier, H. (2019) “Insights into the IgG heavy chain engineering patent landscape as applied to IgG4 antibody development.” MABS 11(8): 1341-1350
Els Conrath, K., M. Lauwereys, L. Wyns and S. Muyldermans (2001). “Camel single-domain antibodies as modular building units in bispecific and bivalent antibody constructs.” J Biol Chem 276(10): 7346-7350.
Hay, R. T. (2005). “SUMO: a history of modification.” Mol Cell 18(1): 1-12.
Huo, J., A. Le Bas, R. R. Ruza, H. M. E. Duyvesteyn, H. Mikolajek, T. Malinauskas, T. K. Tan, P. Rijal, M. Dumoux, P. N. Ward, J. Ren, D. Zhou, P. J. Harrison, M. Weckener, D. K. Clare, V. K. Vogirala, J. Radecke, L. Moynie, Y. Zhao, J. Gilbert-Jaramillo, M. L. Knight, J. A. Tree, K. R. Buttigieg, N. Coombes, M. J. Elmore, M. W. Carroll, L. Carrique, P. N. M. Shah, W. James, A. R. Townsend, D. I. Stuart, R. J. Owens and J. H. Naismith (2020). “Neutralizing nanobodies bind SARS-CoV-2 spike RBD and block interaction with ACE2.” Nat Struct Mol Biol. https://doi.org/10.1038/s41594-C20-C469-6.
Huo, J., Y. Zhao, J. Ren, D. Zhou, H. M. E. Duyvesteyn, H. M. Ginn, L. Carrique, T. Malinauskas, R. R. Ruza, P. N. M. Shah, T. K. Tan, P. Rijal, N. Coombes, K. R. Bewley, J. A. Tree, J. Radecke, N. G. Paterson, P. Supasa, J. Mongkolsapaya, G. R. Screaton, M. Carroll, A. Townsend, E. E. Fry, R. J. Owens and D. I. Stuart (2020). “Neutralization of SARS-CoV-2 by Destruction of the Prefusion Spike.” Cell Host Microbe. https://doi.org/10.1016/j.chom.2020.06.010
Jan ter Meulen Edward N. van den Brink Leo L. M. Poon, W. E. M., Cynthia S. W. Leung Freek Cox, Chung Y. Cheung, Arjen Q. Bakker, Johannes A. Bogaards, Els van Deventer, Wolfgang Preiser, Hans Wilhelm Doerr, Vincent T. Chow4 John de Kruif, Joseph S. M. Peiris, Jaap Goudsmit (2006). “Human Monoclonal Antibody Combinationagainst SARS Coronavirus: Synergy and Coverage of Escape Mutants.” PLoS MEDICINE 3(7): e237.
Rogers, T. F., F. Zhao, D. Huang, N. Beutler, A. Burns, W. T. He, O. Limbo, C. Smith, G. Song, J. Woehl, L. Yang, R. K. Abbott, S. Callaghan, E. Garcia, J. Hurtado, M. Parren, L. Peng, S. Ramirez, J. Ricketts, M. J. Ricciardi, S. A.
Rawlings, N. C. Wu, M. Yuan, D. M. Smith, D. Nemazee, J. R. Teijaro, J. E. Voss, I. A. Wilson, R. Andrabi, B. Briney, E. Landais, D. Sok, J. G. Jardine and D. R. Burton (2020). “Isolation of potent SARS-CoV-2 neutralizing antibodies and protection from disease in a small animal model.” Science 15 Jun. 2020: eabc7520 DOI: 10.1126/science.abc7520
Schepens, X. Saelens and J. S. McLellan (2020). “Structural Basis for Potent Neutralization of Betacoronaviruses by Single-Domain Camelid Antibodies.” Cell 181(6): 1436-1441.
Suzuki, S. Annaka, H. Konno, S. Kumagai, I. Asano, R (2018). “Engineering the hinge region of human IfF1 Fc-fused bispecific antibodies to improve fragmentation resistance.” Nature Scientific Reports 8:17253 DOI:10.1038/541598-C18-35489-y
von Krbek, L. K., A. J. Achazi, S. Schoder, M. Gaedke, T. Biberger, B. Paulus and C. A. Schalley (2017). “The Delicate Balance of Preorganisation and Adaptability in Multiply Bonded Host-Guest Complexes.” Chemistry 23(12): 2877-2883.
Wrapp, D., D. De Vlieger, K. S. Corbett, G. M. Torres, N. Wang, W. Van Breedam, K. Roose, L. van Schie, V.-C. C.-R. Team, M. Hoffmann, S. Pohlmann, B. S. Graham, N. Callewaert, B.
Zivanov, J. et al. New tools for automated high-resolution cryo-EM structure determination in RELION-3. Elife 7, (2018).
Punjani, A., Rubinstein, J. L., Fleet, D. J. & Brubaker, M. A. cryoSPARC: algorithms for rapid unsupervised cryo-EM structure determination. Nat Methods 14, 290-296 (2017).
Asarnow, D., Palovcak, E. & Cheng, Y. UCSF pyem v0.5. Zenodo. https://doi.org/10.5281/zenodo.3576630 (2019).
Pettersen, E. F. et al. UCSF chimera—A visualization system for exploratory research and analysis. J. Comp.l Chem, 25, 1605-1612 (2004).
Emsley, P. & Cowtan, K. Coot: model-building tools for molecular graphics. Acta Crystallogr D Struct Biol 60, 2126-2132 (2004).
Liebschner, D. et al. Macromolecular structure determination using X-rays, neutrons and electrons: recent developments in Phenix. Acta Crystallogr D Struct Biol 75, 861-877 (2019).
Walls, A. C. et al. Structure, Function, and Antigenicity of the SARS-CoV-2 Spike Glycoprotein. Cell 181, 281-292.e6 (2020).
Murshudov, G. N. et al. REFMACS for the refinement of macromolecular crystal structures. Acta Crystallogr D Biol Crystallogr 67, 355-367 (2011).
Burnley, T., Palmer, C. M. & Winn, M. Recent developments in the CCP-EM software suite. Acta Crystallogr D Struct Biol 73, 469-477 (2017).
Wagner, T. et al. SPHIRE-crYOLO is a fast and accurate fully automated particle picker for cryo-EM. Commun Biol 2, 218 (2019).
Huo, J. et al. Neutralization of SARS-CoV-2 by Destruction of the Prefusion Spike.
Cell Host Microbe, doi:10.1016/j.chom.2020.06.010 (2020).
Kuan-Ying A. Huangl, T. K. T., Ting-Hua Chenl, Chung-Guei Huangl, Ruth Harvey, Saira Hussain, Cheng-Pin Chenl, Adam Hardingl, Javier Gilbert-Jaramillol, Xu Liul, Michael Knightl, Lisa Schimanskil, Shin-Ru Shihl, Yi-Chun Lin, Chien-Yu Chengl, Shu-Hsing Chengl, Yhu-Chering Huang, Tzou-Yien, Linl Jia-Tsrong Jan, Che Ma, William Jamesl, Rodney S. Danielsl, John W. McCauleyl, Pramila Rijall, Alain R. Townsend. Breadth and function of antibody response to acute SARSCoV-2 infection in humans. Plos pathogens 17, e1009352 (2021).
Zhou, D. et al. Structural basis for the neutralization of SARS-CoV-2 by an antibody from a convalescent patient. Nat Struct Mol Biol 27, 950-958, doi:10.1038/541594-C20-C480-y (2020).
Nettleship, J. E., Rahman-Huq, N. & Owens, R. J. The production of glycoproteins by transient expression in Mammalian cells. Methods Mol Biol 498, 245-263 doi:10.1007/978-1-59745-196-3_16 (2009).
Chan, J. F. et al. Simulation of the Clinical and Pathological Manifestations of Coronavirus Disease 2019 (COVID-19) in a Golden Syrian Hamster Model: Implications for Disease Pathogenesis and Transmissibility. Clin Infect Dis 71, 2428-2446, doi:10.1093/cid/ciaa325 (2020).
Imai, M. et al. Syrian hamsters as a small animal model for SARS-CoV-2 infection and countermeasure development. Proc Natl Acad Sci USA 117, 16587-16595, doi:10.1073/pnas.2009799117 (2020).
Sia, S. F. et al. Pathogenesis and transmission of SARS-CoV-2 in golden hamsters. Nature 583, 834-838, doi:10.1038/s41586-C20-2342-5 (2020).
Zhou D et al Evidence of escape of SARS-CoV-2 variant B.1.351 from natural and vaccine-induced sera Cell 2021 Apr 29; 184(9):2348-2361
Leung, B., Tran, K., Audet, J. et al. In Vitro inactivation of SARS-CoV-2 using gamma radiation. Applied Biosafety 25(3) (2020). https://doi.org/10.1177/1535676020934242
Afrough, B., Eakins, J., Durley-White, S. et al. X-ray inactivation of RNA viruses without loss of biological characteristics. Sci Rep 10, 21431 (2020).

1-32. (canceled)

33. A composition of matter, wherein the composition of matter comprises:
(I) an anti-SARS-CoV-2 single domain antibody comprising a complementary determining region 3 (CDR3) selected from the group consisting of:
(a) SEQ ID NO: 12 (C5 CDR3);
(b) SEQ ID NO: 198 (2_A8);
(c) SEQ ID NO: 9 (C1 CDR3);
(d) SEQ ID NO: 3 (B12 CDR3);
(e) SEQ ID NO: 6 (F2 CDR3);
(f) SEQ ID NO: 15 (H3 CDR3);
(g) SEQ ID NO: 192 (NbSA_A10);
(h) SEQ ID NO: 195 (NbSA_D10);
(i) SEQ ID NO: 201 (3_C5);
(j) SEQ ID NO: 204 (8_G11);
(k) SEQ ID NO: 207 (12_F11); and
(l) SEQ ID NO: 237 (VHH_H6)
wherein the amino acid sequence of CDR3 comprises between 0 and 7 amino acid modifications; or
the single antibody domain comprises
(a) a CDR2 comprising SEQ ID NO:11 and a CDR3 comprising SEQ ID NO:12 (C5);
(b) a CDR2 comprising SEQ ID NO:197 and a CDR3 comprising SEQ ID NO:198 (2_A8);
(c) a CDR2 comprising SEQ ID NO:8 and a CDR3 comprising SEQ ID NO:9 (C1);
(d) a CDR2 comprising SEQ ID NO:2 and a CDR3 comprising SEQ ID NO:3 (B12);
(e) a CDR2 comprising SEQ ID NO:5 and a CDR3 comprising SEQ ID NO:6 (F2);
(f) a CDR2 comprising SEQ ID NO:14 and a CDR3 comprising SEQ ID NO:15 (H3);
(g) a CDR2 comprising SEQ ID NO:191 and a CDR3 comprising SEQ ID NO:192;
(h) a CDR2 comprising SEQ ID NO:194 and a CDR3 comprising SEQ ID NO:195;
(i) a CDR2 comprising SEQ ID NO:200 and a CDR3 comprising SEQ ID NO:201;
(j) a CDR2 comprising SEQ ID NO:203 and a CDR3 comprising SEQ ID NO:204;
(k) a CDR2 comprising SEQ ID NO:206 and a CDR3 comprising SEQ ID NO:207; or
(l) a CDR2 comprising SEQ ID NO:236 and a CDR3 comprising SEQ ID NO:237 wherein the amino acid sequence of CDR3 comprises between 0 and 7 amino acid modifications and wherein the amino acid sequence of CDR2 comprises between 0 and 4 amino acid modifications; or
(II) a multivalent polypeptide comprising one or optionally two or more single domain antibodies of (I); or
(III) a coronavirus binding molecule comprising:
(a) a first antigen binding molecule that binds to all or part of a first epitope comprised within a coronavirus protein;
(b) a second antigen binding molecule that binds to all or part of a second epitope comprised within a coronavirus protein; and
(c) a linker,
wherein the linker comprises:
(i) a ubiquitin or a ubiquitin-like protein;
(ii) a further optional spacer of between 4 and 50 amino acids joined to the n-terminal of the ubiquitin or ubiquitin-like protein; and
(iii) further optional spacer of between 4 and 50 amino acids joined to the c-terminal of the ubiquitin or ubiquitin-like protein;
and wherein the linker joins the first antigen binding molecule to the second antigen binding molecule, and optionally wherein the first and second epitopes are substantially non-overlapping; or
(IV) a coronavirus binding molecule comprising:
(a) a first antigen binding molecule that binds to all or part of a first epitope comprised within SEQ ID NO: 232;
(b) a second antigen binding molecule that binds to all or part of a second epitope comprised within SEQ ID NO: 233; and
(c) a linker,
wherein the linker comprises:
(i) a ubiquitin or a ubiquitin-like protein;
(ii) further optional spacer of between 4 and 50 amino acids joined to the n-terminal of the ubiquitin or ubiquitin-like protein; and
(iii) further optional spacer of between 4 and 50 amino acids joined to the c-terminal of the ubiquitin or ubiquitin-like protein;
and wherein the linker joins the first antigen binding molecule to the second antigen binding molecule,
and optionally wherein the first and second epitopes are substantially non-overlapping; or
(V) a polynucleotide encoding an antibody or multivalent polypeptide of (I) or (II); or
(VI) a pharmaceutical composition comprising antibody, multivalent polypeptide or coronavirus binding molecule of (I)-(IV); or
(VII) an expression vector comprising the polynucleotide of (V); or
(VIII) a host cell or cell line comprising the vector of (VII); or
(IX) a pharmaceutical device comprising a antibody, multivalent polypeptide or coronavirus binding molecule of (I)-(IV), wherein the pharmaceutical device is suitable for delivery of the antibody or multivalent polypeptide via inhalation or nebulisation; or
(X) a kit for the detection of a coronavirus, wherein the kit comprises:
(a) a detectable marker
(b) an antibody, multivalent polypeptide or coronavirus binding molecule of any one of (I)-(IV).

34. A composition of matter comprising a anti-SARS-CoV-2 single domain antibody according to claim 33(I), wherein the single antibody domain comprises
(a) a CDR1 comprising SEQ ID NO:10, a CDR2 comprising SEQ ID NO:11 and a CDR3 comprising SEQ ID NO:12 (C5);
(b) a CDR1 comprising SEQ ID NO:196, a CDR2 comprising SEQ ID NO:197 and a CDR3 comprising SEQ ID NO:198;
(c) a CDR1 comprising SEQ ID NO:7, a CDR2 comprising SEQ ID NO:8 and a CDR3 comprising SEQ ID NO:9 (C1);
(d) CDR1 comprising SEQ ID NO:1, a CDR2 comprising SEQ ID NO:2 and a CDR3 comprising SEQ ID NO:3 (B12);
(e) a CDR1 comprising SEQ ID NO:4, a CDR2 comprising SEQ ID NO:5 and a CDR3 comprising SEQ ID NO:6 (F2);
(f) a CDR1 comprising SEQ ID NO:13, a CDR2 comprising SEQ ID NO:14 and a CDR3 comprising SEQ ID NO:15 (H3);
(g) a CDR1 comprising SEQ ID NO:190, a CDR2 comprising SEQ ID NO:191 and a CDR3 comprising SEQ ID NO:192;
(h) a CDR1 comprising SEQ ID NO:193, a CDR2 comprising SEQ ID NO:194 and a CDR3 comprising SEQ ID NO:195;
(i) a CDR1 comprising SEQ ID NO:199, a CDR2 comprising SEQ ID NO:200 and a CDR3 comprising SEQ ID NO:201;
(j) a CDR1 comprising SEQ ID NO:202, a CDR2 comprising SEQ ID NO:203 and a CDR3 comprising SEQ ID NO:204;
(k) a CDR1 comprising SEQ ID NO:205, a CDR2 comprising SEQ ID NO:206 and a CDR3 comprising SEQ ID NO:207; or
(l) a CDR1 comprising SEQ ID NO:235, a CDR2 comprising SEQ ID NO:236 and a CDR3 comprising SEQ ID NO:237
wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications;

35. A composition of matter comprising a multivalent polypeptide according to claim 33(II), wherein the one or the two or more single domain antibodies are joined by one or more linkers, optionally as a single fusion protein, and optionally wherein the linker(s) can be the same or different and may include one of more of: polyA linkers; GS linkers and/or ubiquitin or ubiquitin-like
linkers, optionally SUMO or SUMO-like linkers.

36. A composition of matter comprising a multivalent polypeptide according to claim 33(II), which is a bivalent antibody, comprising two single domain antibodies according to claim 33(I), optionally:
a) one of the group consisting of:
i) two single domain antibodies each comprising SEQ ID NO: 12 (C5 CDR3);
ii. two single domain antibodies each comprising SEQ ID NO: 198 (2_A8);
iii) two single domain antibodies each comprising SEQ ID NO: 9 (C1 CDR3);
iv) two single domain antibodies each comprising SEQ ID NO: 3 (B12 CDR3);
v) two single domain antibodies each comprising SEQ ID NO: 6 (F2 CDR3);
vi) two single domain antibodies each comprising SEQ ID NO: 15 (H3 CDR3);
vii. two single domain antibodies each comprising SEQ ID NO: 192 (NbSA_A10);
viii. two single domain antibodies each comprising SEQ ID NO: 195 (NbSA_D10);
ix. two single domain antibodies each comprising SEQ ID NO: 201 (3_C5);
x. two single domain antibodies each comprising SEQ ID NO: 204 (8_G11) and
xi two single domain antibodies each comprising SEQ ID NO: 207 (12_F11)
xii. two single domain antibodies each comprising SEQ ID NO: 237 (VHH_H6) or
b) two single domain antibodies selected from the group consisting of single domain antibodies comprising: (a) a CDR2 comprising SEQ ID NO:11 and a CDR3 comprising SEQ ID NO:12 (C5); (b) a CDR2 comprising SEQ ID NO:197 and a CDR3 comprising SEQ ID NO:198 (2_A8); (c) a CDR2 comprising SEQ ID NO:8 and a CDR3 comprising SEQ ID NO:9 (C1); (d) a CDR2 comprising SEQ ID NO:2 and a CDR3 comprising SEQ ID NO:3 (B12); or (e) a CDR2 comprising SEQ ID NO:5 and a CDR3 comprising SEQ ID NO:6 (F2); or
c) two single domain antibodies selected from the group consisting of single domain antibodies comprising: (a) a CDR1 comprising SEQ ID NO:10, a CDR2 comprising SEQ ID NO:11 and a CDR3 comprising SEQ ID NO:12 (C5); (b) a CDR1 comprising SEQ ID NO:196, a CDR2 comprising SEQ ID NO:197 and a CDR3 comprising SEQ ID NO:198; (c) a CDR1 comprising SEQ ID NO:7, a CDR2 comprising SEQ ID NO:8 and a CDR3 comprising SEQ ID NO:9 (C1); (d) CDR1 comprising SEQ ID NO:1, a CDR2 comprising SEQ ID NO:2 and a CDR3 comprising SEQ ID NO:3 (B12); or (e) a CDR1 comprising SEQ ID NO:4, a CDR2 comprising SEQ ID NO:5 and a CDR3 comprising SEQ ID NO:6 (F2).

37. A composition of matter comprising a multivalent polypeptide according to claim 33(II), comprising three or more single domain antibodies according to claim 33(I), optionally joined by a linker,
optionally represented by Formula I or II:
(sdAB)_{N}-(Linker)_{N-1} Formula I:
(sdAB)_{N}-(Linker)_{N} Formula II:
wherein the number N is the number of single domain antibodies (sdAb) and optionally a
wherein the linker(s) can be the same or different and may include one of more of: polyA linkers; GS linkers and/or ubiquitin or ubiquitin-like linkers, optionally SUMO or SUMO-like linkers.

38. A composition of matter comprising a multivalent polypeptide according to claim 33(II),
wherein the single domain antibody comprises:
a. at least SEQ ID NO: 12 (C5 CDR3), and optionally also SEQ ID NO:10 (C5 CDR1) and/or SEQ ID NO:11 (C5 CDR2);
or
b. at least SEQ ID NO: 198 (2_A8 CDR3), and optionally also SEQ ID NO:196 (2_A8 CDR1) and/or SEQ ID NO:197 (2_A8 CDR2).

39. A composition of matter comprising a multivalent polypeptide according to claim 33(II), optionally comprising two or more single domain antibodies, with at least one of said single domain antibodies being a different single domain antibody from the other single domain antibodies.

40. A composition of matter comprising a multivalent polypeptide according to claim 39, which is optionally a bidentate antibody, comprising first and second single domain antibodies, which are different and are each according to claim 33(I).

41. A composition of matter comprising a multivalent polypeptide according to claim 33(II), wherein the polypeptide is a trimer comprising three single domain antibodies, each single domain antibody comprising:
(a) a CDR1 comprising SEQ ID NO:7, a CDR2 comprising SEQ ID NO:8 and a CDR3 comprising SEQ ID NO:9 (C1);
(b) a CDR1 comprising SEQ ID NO:13, a CDR2 comprising SEQ ID NO:14 and a CDR3 comprising SEQ ID NO:15 (H3);
(c) a CDR1 comprising SEQ ID NO:10, a CDR2 comprising SEQ ID NO:11 and a CDR3 comprising SEQ ID NO:12 (C5); or
(d) a CDR1 comprising SEQ ID NO:196, a CDR2 comprising SEQ ID NO:197 and a CDR3 comprising SEQ ID NO:198 (2_A8);
wherein the amino acid sequence of CDR3 comprises between 0 and 7 amino acid modifications, wherein the amino acid sequence of CDR2 comprises between 0 and 4 amino acid modifications and wherein the amino acid sequence of CDR1 comprises between 0 and 4 amino acid modifications.

42. A composition of matter comprising an antibody or multivalent polypeptide according to claim 33(I) or (II), wherein the amino acid modification is a substitution, insertion or deletion.

43. A composition of matter comprising an antibody or multivalent polypeptide according to claim 33(I) or (II), comprising an amino acid sequence having at least 70% identity to a sequence selected from the group consisting of: SEQ ID NO: 213 (2_A8), 19 (C5), 16 (B12), 17 (F2), 18 (C1), 20 (H3), 209 (NbSA_A10), 211 (NbSA_D10), 215 (3_C5), 217 (8_G11), 219 (12_F11), 220 (C1 (N76Q)) and 239 (VHH_H6).

44. A method for the production of an antibody or multivalent polypeptide, comprising culturing a host cell according to claim 33(VIII) in a culture medium under conditions to express the polynucleotide sequence of the expression vector.

45. A method for the treatment or prevention of coronavirus, said method comprising administering to a subject a therapeutically active amount of a composition of matter comprising an antibody, multivalent polypeptide or coronavirus binding molecule or pharmaceutical composition according to claim 33(I), (II), (III), (IV) or (VI).

46. A method for treatment or prevention of coronavirus according to claim 45, wherein the coronavirus is selected from the group consisting of Middle Eastern respiratory syndrome (MERS-CoV), severe acute respiratory syndrome coronavirus 1 (SARS-CoV-1) and COVID-19.

47. A method or assay for detecting coronavirus, wherein the method or assay is:
(I) a method for diagnosing coronavirus infection in a subject, the method comprising:
(a) contacting an isolated sample with a composition of matter comprising an antibody, multivalent polypeptide or coronavirus binding molecule according to claim 33(I), (II), (III) or (IV),
(b) detecting the number of antibody-antigen complexes,
(c) detecting the presence of coronavirus in the sample,
wherein the presence of the complex provides a positive diagnosis of coronavirus in the subject; or
a method for detecting coronavirus protein in a sample from a patient, wherein the method comprises the steps of:
(a) contacting a sample from the subject with a composition of matter comprising an antibody, multivalent polypeptide or coronavirus binding molecule according to claim 33(I), (II), (III), or (IV), and
(b) detecting the antibody-antigen complex,
wherein the presence of the complex indicates the presence of coronavirus protein in the subject; or
(II) an assay for the detection of a coronavirus, wherein the assay comprises contacting a sample obtained from a patient with a composition of matter comprising an antibody, multivalent polypeptide or coronavirus binding molecule according to claim 33(I), (II), (III), or (IV), wherein the single domain antibody comprises a detectable label or reporter molecule to selectively isolate the coronavirus in the patient sample, optionally wherein the assay is an enzyme-linked immunosorbent assay (ELISA), a radioimmunoassay (RIA) or a fluorescence-activated cell sorting (FACS).

48. The method of claim 47(I), wherein the coronavirus is selected from the group consisting of Middle Eastern respiratory syndrome (MERS-CoV), severe acute respiratory syndrome coronavirus 1 (SARS-CoV-1) and severe acute respiratory syndrome coronavirus 1 (SARS-CoV-2).


SEQUENCE LISTING (248 sequences)

SEQ ID NO: 1 - protein, 7 aa, Artificial Sequence
  B12 CDR1
GGTFSTY

SEQ ID NO: 2 - protein, 7 aa, Artificial Sequence
  B12 CDR2
IRRSGST

SEQ ID NO: 3 - protein, 11 aa, Artificial Sequence
  B12 CDR3
AARRAGREYEY

SEQ ID NO: 4 - protein, 8 aa, Artificial Sequence
  F2 CDR1
GRTFHSYV

SEQ ID NO: 5 - protein, 8 aa, Artificial Sequence
  F2 CDR2
ISWSSTPT

SEQ ID NO: 6 - protein, 18 aa, Artificial Sequence
  F2 CDR3
AADRGESYYYTRPTEYEF

SEQ ID NO: 7 - protein, 8 aa, Artificial Sequence
  C1 CDR1
GFTNDFYS

SEQ ID NO: 8 - protein, 8 aa, Artificial Sequence
  C1 CDR2
LSVSDNTP

SEQ ID NO: 9 - protein, 17 aa, Artificial Sequence
  C1 CDR3
AAGRFAGRDTWPSSYDY

SEQ ID NO: 10 - protein, 8 aa, Artificial Sequence
  C5 CDR1
GVTLGRHA

SEQ ID NO: 11 - protein, 8 aa, Artificial Sequence
  C5 CDR2
IRTFDGIT

SEQ ID NO: 12 - protein, 14 aa, Artificial Sequence
  C5 CDR3
ALGVTAACSDNPYF

SEQ ID NO: 13 - protein, 8 aa, Artificial Sequence
  H3 CDR1
GRTFSTYS

SEQ ID NO: 14 - protein, 8 aa, Artificial Sequence
  H3 CDR2
MRWTGSST

SEQ ID NO: 15 - protein, 20 aa, Artificial Sequence
  H3 CDR3
AITTIVRAYYTEYTEADFGS

SEQ ID NO: 16 - protein, 117 aa, Artificial Sequence
  B12
QVQLVESGGGFVQPGGSLRLSCAVSGGTFSTYGMGWFRQAPGKEREIVAAIRRSGSTYYADSVRGRFTISRDNAKNSVLLQMNSLKPEDTAVYYCAARRAGREYEYWGQGTQVTVSS

SEQ ID NO: 17 - protein, 125 aa, Artificial Sequence
  F2
QVQLVESGGGLVQAGGSLRLACIASGRTFHSYVMAWFRQAPGKEREFVAAISWSSTPTYYGESVKGRFTISRDNAKNTVYLQMNRLKPEDTAVYFCAADRGESYYYTRPTEYEFWGQGTQVTVSS

SEQ ID NO: 18 - protein, 124 aa, Artificial Sequence
  C1
QVQLVESGGGLVQPGGSLRLSCAASGFTNDFYSIAWFRQAPGKEREGVSWLSVSDNTPTYVDSVKDRFTISRHNANNTVYLQMNMLKPEDTAIYYCAAGRFAGRDTWPSSYDYWGQGTQVTVSS

SEQ ID NO: 19 - protein, 121 aa, Artificial Sequence
  C5
QVQLVESGGGSVQAGGSLTLSCVASGVTLGRHAIGWFRQAPGKERERVSCIRTFDGITSYVESTKGRFTISSNNAMNTVYLQMNSLKPEDTAVYFCALGVTAACSDNPYFWGQGTQVTVSS

SEQ ID NO: 20 - protein, 127 aa, Artificial Sequence
  H3
QVQLVESGGGLVKTGGSLRLSCAASGRTFSTYSMGWFRQAPGKEREFVAGMRWTGSSTFYSDSVKGRFTVSRNNAKDTVYLHMNSLKPEDTAVYYCAITTIVRAYYTEYTEADFGSWGQGTQVTVSS

SEQ ID NO: 21 - DNA, 351 bp, Artificial Sequence
  B12
caggtgcagctggtggagtctggaggaggcttcgtgcagcctgggggctctctgagactctcctgcgccgtttctggaggcaccttcagtacctatggcatgggctggttccgccaggctccagggaaggagcgtgagattgtagcagcgattagacggagtggtagcacatactatgcagactccgtgaggggccgattcaccatctccagagacaacgccaagaactcggtgctcctgcaaatgaacagcctgaaacctgaggacacggccgtttattactgtgcagccaggagagcgggtagggagtatgagtactggggccaggggacccaggtcaccgtctcctca

SEQ ID NO: 22 - DNA, 375 bp, Artificial Sequence
  F2
caggtgcagctggtggagtctgggggaggattggtgcaggctgggggctctctaagactcgcttgtatagcctctggacgcaccttccatagctatgtcatggcctggttccgccaggctccagggaaggagcgtgagtttgtagcagctattagttggagtagtacaccgacatactatggagaatccgtgaagggccgattcaccatctccagagacaacgccaagaacacggtgtatctgcaaatgaaccgcctgaaacctgaggacacggccgtttatttctgtgcagcagaccggggtgaaagttactactacactcgacccaccgagtatgaattctggggccaggggacccaggtcaccgtctcctca

SEQ ID NO: 23 - DNA, 372 bp, Artificial Sequence
  C1
caggtgcagctggtggagtctgggggaggcttggtgcagcctgggggctctctgagactctcctgtgcagcctctggattcactaatgatttttatagcatcgcgtggttccgccaggccccaggaaaggagcgtgagggggtctcatggcttagtgtcagtgataataccccaacctacgtagactccgtgaaggaccggttcaccatctccagacacaacgccaacaacaccgtgtacctgcaaatgaacatgctgaaacctgaggacacggccatttactattgtgcagcaggacgcttcgcgggaagggatacttggccctcgtcctatgattactggggccaggggacccaggtcaccgtctcctca

SEQ ID NO: 24 - DNA, 363 bp, Artificial Sequence
  C5
caggtgcagctggtggagtctgggggaggctcggtgcaggctggggggtctctgacactctcctgtgtcgcctctggagtcactttgggacgtcatgccataggctggttccgccaggcccccgggaaggagcgtgagagagtctcgtgtattagaacatttgatggcatcacaagttatgtagagtccacgaagggccgattcaccatctccagtaacaatgccatgaacacggtgtatctgcaaatgaatagcctcaaacctgaagacacggccgtttatttctgtgcactgggagtgactgcagcctgttcagataatccctacttctggggccaggggacccaggtcaccgtctcctca

SEQ ID NO: 25 - DNA, 381 bp, Artificial Sequence
  H3
caggtgcagctggtggagtctgggggaggattggtgaagactgggggctctctgagactctcctgtgcagcctctggccgcaccttcagtacctacagcatgggctggttccgccaggctccagggaaggagcgtgagtttgtagcaggtatgcgctggacgggtagtagtacattctactcagactccgtgaagggccgattcaccgtctccagaaacaacgccaaggacacggtgtatctgcacatgaacagcctgaaacctgaggacacggccgtttattactgtgcaatcacgactatcgtaagagcttactatactgagtataccgaagctgactttggttcctggggccaggggacccaggtcaccgtctcctca

SEQ ID NO: 26 - protein, 229 aa, Artificial Sequence
  CR3022 H-chain (RCSB.org structures)
TQMQLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKHHHHHH

SEQ ID NO: 27 - protein, 222 aa, Artificial Sequence
  CR3022 H-chain (RCSB.org structures) #2
QMQLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 28 - protein, 220 aa, Artificial Sequence
  CR3022 L-chain (RCSB.org structures) #1
DIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 221 aa, Artificial Sequence
  CR3022 L-chain (RCSB.org structures) #2
DIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECS

SEQ ID NO: 30 - protein, 226 aa, Artificial Sequence
  EY6A H-chain (RCSB.org structures)
EVQLVESGGGVVQPGRSLRLSCAASAFTFSSYDMHWVRQAPGKGLEWVAVISYDGSNKYYADSVKGRFTISRDNSKNTLYLQMNSLRAEDTAVYYCAKDGGKLWVYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 31 - protein, 215 aa, Artificial Sequence
  EY6A L-chain (RCSB.org structures)
DIQMTQSPSSLSASVGDRVTITCRASQSISSYLNWYQQKPGKAPKLLIYAASSLQSGVPSRFSGSGSGTDFTLTISSLQPEDFATYYCQQSYSTLALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 127 aa, Artificial Sequence
  VHH-72 nanobody (RCSB.org structures https//www.rcsb.org/structure/6waq)
QVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSSGS

SEQ ID NO: 33 - protein, 8 aa, Artificial Sequence
  H11 CDR1
GRTFSTAA

SEQ ID NO: 34 - protein, 8 aa, Artificial Sequence
  H11 CDR2
IRWSGGSA

SEQ ID NO: 35 - protein, 20 aa, Artificial Sequence
  H11 CDR3
AQTRVTRSLLSDYATWPYDY

SEQ ID NO: 36 - protein, 8 aa, Artificial Sequence
  A7 CDR1
GFTFDDYA

SEQ ID NO: 37 - protein, 8 aa, Artificial Sequence
  A7 CDR2
ISWNGGGT

SEQ ID NO: 38 - protein, 16 aa, Artificial Sequence
  A7 CDR3
AKDFHRYGSSTLEYDY

SEQ ID NO: 39 - protein, 8 aa, Artificial Sequence
  F9 CDR1
GFTFSSYF

SEQ ID NO: 40 - protein, 7 aa, Artificial Sequence
  F9 CDR2
IGDRGNT

SEQ ID NO: 41 - protein, 10 aa, Artificial Sequence
  F9 CDR3
YSRNRILQHY

SEQ ID NO: 42 - protein, 8 aa, Artificial Sequence
  C10 CDR1
GFAFSSYD

SEQ ID NO: 43 - protein, 8 aa, Artificial Sequence
  C10 CDR2
IDSGGGVT

SEQ ID NO: 44 - protein, 13 aa, Artificial Sequence
  C10 CDR3
NAVDGLGYLEYDY

SEQ ID NO: 45 - protein, 8 aa, Artificial Sequence
  B1 CDR1
GFTFDDYA

SEQ ID NO: 46 - protein, 8 aa, Artificial Sequence
  B11 CDR2
IYSYISTT

SEQ ID NO: 47 - protein, 12 aa, Artificial Sequence
  B11 CDR3
AARRLDTTDYKY

SEQ ID NO: 48 - protein, 8 aa, Artificial Sequence
  E11 CDR1
GFTFSNYA

SEQ ID NO: 49 - protein, 8 aa, Artificial Sequence
  E11 CDR2
IGSDGRHP

SEQ ID NO: 50 - protein, 13 aa, Artificial Sequence
  E11 CDR3
LPGWSTVGTFLDA

SEQ ID NO: 51 - protein, 8 aa, Artificial Sequence
  D1 CDR1
GFTFSSYG

SEQ ID NO: 52 - protein, 8 aa, Artificial Sequence
  D1 CDR2
INSGGGST

SEQ ID NO: 53 - protein, 16 aa, Artificial Sequence
  D1 CDR3
AKDASEDAGRLYWTDY

SEQ ID NO: 54 - protein, 8 aa, Artificial Sequence
  G7 CDR1
GFTFDTYD

SEQ ID NO: 55 - protein, 7 aa, Artificial Sequence
  G7 CDR2
EDSTGNK

SEQ ID NO: 56 - protein, 9 aa, Artificial Sequence
  G7 CDR3
AKGLRSWGA

SEQ ID NO: 57 - protein, 8 aa, Artificial Sequence
  F5 CDR1
GTIFRINA

SEQ ID NO: 58 - protein, 8 aa, Artificial Sequence
  F5 CDR2
ISNGDSGD

SEQ ID NO: 59 - protein, 12 aa, Artificial Sequence
  F5 CDR3
YCNVEASWPHKY

SEQ ID NO: 60 - protein, 8 aa, Artificial Sequence
  G11 CDR1
GFTFDDYG

SEQ ID NO: 61 - protein, 7 aa, Artificial Sequence
  G11 CDR2
VNSGGGT

SEQ ID NO: 62 - protein, 14 aa, Artificial Sequence
  G11 CDR3
AKRDGSWWGYTTDY

SEQ ID NO: 63 - protein, 8 aa, Artificial Sequence
  B4 CDR1
GFTFSSYW

SEQ ID NO: 64 - protein, 8 aa, Artificial Sequence
  B4 CDR2
INTGGDTT

SEQ ID NO: 65 - protein, 14 aa, Artificial Sequence
  B4 CDR3
ARTGVVRGPGDLDA

SEQ ID NO: 66 - protein, 8 aa, Artificial Sequence
  G9 CDR1
GFTFYDYH

SEQ ID NO: 67 - protein, 8 aa, Artificial Sequence
  G9 CDR2
IDTGGGRT

SEQ ID NO: 68 - protein, 13 aa, Artificial Sequence
  G9 CDR3
VRDGDARGPDYDY

SEQ ID NO: 69 - protein, 8 aa, Artificial Sequence
  C7 CDR1
GFTFSSYD

SEQ ID NO: 70 - protein, 8 aa, Artificial Sequence
  C7 CDR2
ISGSDSTT

SEQ ID NO: 71 - protein, 9 aa, Artificial Sequence
  C7 CDR3
ARPLGQYTS

SEQ ID NO: 72 - protein, 20 aa, Artificial Sequence
  H11-D4 CDR3
ARTENVRSLLSDYATWPYDY

SEQ ID NO: 73 - protein, 20 aa, Artificial Sequence
  H11-H4 CDR3
AQTHYVSYLLSDYATWPYDY

SEQ ID NO: 74 - protein, 20 aa, Artificial Sequence
  H11-H6 CDR3
AGSKITRSLLSDYATWPYDY

SEQ ID NO: 75 - protein, 20 aa, Artificial Sequence
  H11-A10 CDR3
AGFSATRSLLSDYATWPYDY

SEQ ID NO: 76 - protein, 20 aa, Artificial Sequence
  H11_B5 CDR3
ASYQATRSLLSDYATWPYDY

SEQ ID NO: 77 - protein, 20 aa, Artificial Sequence
  H11-A7 CDR3
AQTDNVRALLSDYATWPYDY

SEQ ID NO: 78 - protein, 20 aa, Artificial Sequence
  H11-F7 CDR3
AIFSNVRSLLSDYATWPYDY

SEQ ID NO: 79 - protein, 20 aa, Artificial Sequence
  H11-F6 CDR3
ARTSNVRSLLSDYATWPYDY

SEQ ID NO: 80 - protein, 20 aa, Artificial Sequence
  H11-G8 CDR3
AGSGVTRSLLSDYATWPYDY

SEQ ID NO: 81 - protein, 20 aa, Artificial Sequence
  H11-D1 CDR3
AGWRATRSLLSDYATWPYDY

SEQ ID NO: 82 - protein, 20 aa, Artificial Sequence
  H11-A9 CDR3
APYEATRSLLSDYATWPYDY

SEQ ID NO: 83 - protein, 20 aa, Artificial Sequence
  H11-C6 CDR3
AQTGYTSWLLSDYATWPYDY

SEQ ID NO: 84 - protein, 20 aa, Artificial Sequence
  H11-E3 CDR3
AVYHATRSLLSDYATWPYDY

SEQ ID NO: 85 - protein, 20 aa, Artificial Sequence
  H11-F4 CDR3
AESTITRSLLSDYATWPYDY

SEQ ID NO: 86 - protein, 20 aa, Artificial Sequence
  H11-C5 CDR3
AQTSYVSFLLSDYATWPYDY

SEQ ID NO: 87 - protein, 20 aa, Artificial Sequence
  H11-C2 CDR3
AGSRATRSLLSDYATWPYDY

SEQ ID NO: 88 - protein, 20 aa, Artificial Sequence
  H11-B11 CDR3
AETLNTRSLLSDYATWPYDY

SEQ ID NO: 89 - protein, 20 aa, Artificial Sequence
  H11-A3 CDR3
ARSDNVRSLLSDYATWPYDY

SEQ ID NO: 90 - protein, 20 aa, Artificial Sequence
  H11-D12 CDR3
ADWGVTRSLLSDYATWPYDY

SEQ ID NO: 91 - protein, 20 aa, Artificial Sequence
  H11-D6 CDR3
ASSSVTRSLLSDYATWPYDY

SEQ ID NO: 92 - protein, 20 aa, Artificial Sequence
  H11-F8 CDR3
ADASATRSLLSDYATWPYDY

SEQ ID NO: 93 - protein, 127 aa, Artificial Sequence
  H11 amino acid
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTRVTRSLLSDYATWPYDYWGQGTQVTVSS

SEQ ID NO: 94 - protein, 123 aa, Artificial Sequence
  A7 amino acid
QVQLVESGGGLVQPGGSLRLSCAASGFTFDDYAMSWVRQAPGKGLEWVSAISWNGGGTYYAESMKGRFTISRDNAKNTLYLQMNSLKSEDTAVYYCAKDFHRYGSSTLEYDYWGQGTQVTVSS

SEQ ID NO: 95 - protein, 116 aa, Artificial Sequence
  F9 amino acid
QVQLVESGGGLVQPGGSLRVSCAASGFTFSSYFMSWVRQAPGKQRELVATIGDRGNTNYADSVKGRFTISRDNAKNTVYLQMNSLKPEDTAVYYCYSRNRILQHYWGQGTQVTVSS

SEQ ID NO: 96 - protein, 120 aa, Artificial Sequence
  C10 amino acid
QVQLVESGGGLVQPGGSLRLSCAASGFAFSSYDMSWVRQAPGKGLEWVSAIDSGGGVTYYADSMKGRFTISRDNAKNTVYLQMNSLKPEDTAVYYCNAVDGLGYLEYDYWGQGTQVTVSS

SEQ ID NO: 97 - protein, 119 aa, Artificial Sequence
  B11 amino acid
QVQLVESGGGLVQPGGSLRLSCAASGFTFDDYAMSWVRQAPGKGLEWVSNIYSYISTTHYADSVKGRFTISTDNAKNTLYLQMNSLKPEDTAVYYCAARRLDTTDYKYWGQGTQVTVSS

SEQ ID NO: 98 - protein, 120 aa, Artificial Sequence
  E11 amino acid
QVQLVESGGGLVQPGGSLRLSCAASGFTFSNYAMTWVRQAPGKGLEWISDIGSDGRHPAYADSVKGRYTISRDNAKNTLYLQMNSLKPEDTAMYYCLPGWSTVGTFLDAWGQGTQVTVSS

SEQ ID NO: 99 - protein, 123 aa, Artificial Sequence
  D1 amino acid
QVQLVESGGGLVQPGGSLRLSCAASGFTFSSYGMSWVRQAPGKGLEWVSGINSGGGSTSYADSVKGRFTISRDNAKNTLYLQMNSLKSEDTAVYYCAKDASEDAGRLYWTDYWGQGTQVTVSS

SEQ ID NO: 100 - protein, 115 aa, Artificial Sequence
  G7 amino acid
QVQLVESGGGLVQPGGSLRLSCATSGFTFDTYDMSWVRQAPGNGPEWVSAEDSTGNKFYADSVKGRFAISRDNARNTLYLQMNSLTSEDTAVYYCAKGLRSWGAWGQGTQVTVSS

SEQ ID NO: 101 - protein, 119 aa, Artificial Sequence
  F5 amino acid
QVQLVESGGGLVQAGGSLRLSCAASGTIFRINAMAWYRQAPGKQRELVAIISNGDSGDSTNYADSVKGRFTISRDSAKNTVYLQMNSLKPEDTAVYYCNVEASWPHKYWGQGTQVTVSS

SEQ ID NO: 102 - protein, 120 aa, Artificial Sequence
  G11 amino acid
QVHLVESGGGLVQPGGSLRLSCAASGFTFDDYGMTWVRQAPGKGLEWVSAVNSGGGTSYADSVKGRFTISRDNAKNTLYLQMNSLKPEDTAVYYCAKRDGSWWGYTTDYWGKGTQVTVSS

SEQ ID NO: 103 - protein, 121 aa, Artificial Sequence
  B4 amino acid
QVQLVESGGGLVQPGGSLRLSCAASGFTFSSYWMYWVRQAPGKGLEWVSVINTGGDTTYYADSVKGRFTISRDNDENTLSLQMNSLKPEDTALYYCARTGVVRGPGDLDAWGQGTQVTVSS

SEQ ID NO: 104 - protein, 120 aa, Artificial Sequence
  G9 amino acid
QVQLVESGGGLVQPGGSLRLSCAASGFTFYDYHMSWVRQAPGKGLEWVSGIDTGGGRTYAADSVKGRFTISRDNAKNTLYLQMNSLKSEDTALYYCVRDGDARGPDYDYWGQGTMVTVSS

SEQ ID NO: 105 - protein, 116 aa, Artificial Sequence
  C7
QVQLVESGGGLVQPGGSLRLSCAASGFTFSSYDMSWVRQAPGKGLEWVSGISGSDSTTVYSDSVKDRFTISRDNAKNTLYLQMNKLKPEDTALYYCARPLGQYTSWGQGTQVTVSS

SEQ ID NO: 106 - DNA, 381 bp, Artificial Sequence
  H11
caggtgcagctggtggagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcacaaacgcgggtcacgcggtccctccttagcgactatgccacttggccttatgactactggggccaggggacccaggtcaccgtctcctca

SEQ ID NO: 107 - DNA, 369 bp, Artificial Sequence
  A7
caggtgcagctggtagagtctgggggaggcttggtgcagcctggggggtctctgagactctcctgtgcagcctctggattcacttttgatgattatgccatgagctgggtccgacaggctccagggaaggggctggagtgggtctcagctattagctggaatggtggtggcacatactatgcagaatccatgaagggccgattcaccatctccagagacaacgccaagaacacgctgtatctgcaaatgaacagtctgaaatctgaggacacggccgtgtattactgtgcaaaagattttcatcgatacggtagtagcactttggagtatgactactggggccaggggacccaggtcaccgtctcctca

SEQ ID NO: 108 - DNA, 348 bp, Artificial Sequence
  F9
caggtgcagctggtagagtctgggggaggcttggtgcagcctggggggtctctgagagtctcctgtgcagcctctggattcaccttcagtagctacttcatgagctgggtccgccaggctccagggaagcagcgcgagttggtcgcgactattggtgatcgtggtaacacaaactatgcagactccgtgaagggccgattcaccatctccagagacaacgccaagaacacagtgtatctgcaaatgaacagcctgaaacctgaggacacggccgtctattactgttattcaagaaaccgtatcttgcaacactactggggccaggggacacaggtcaccgtctcctca

SEQ ID NO: 109 - DNA, 360 bp, Artificial Sequence
  C10
caggtgcagctggtagagtctgggggaggcttggtgcagcctggggggtctctgagactctcctgtgcagcctctggattcgccttcagtagctatgacatgagctgggtccgccaggctccagggaagggactcgaatgggtctcagcgattgatagtggtggtggtgtcacatactatgcagactccatgaagggccgattcaccatctccagagacaacgccaagaacacggtgtatctgcaaatgaacagcctgaaacctgaggacacggccgtgtattactgtaatgcagtagacggcttggggtacttagagtatgactactggggccaggggacccaggtcaccgtctcctca

SEQ ID NO: 110 - DNA, 357 bp, Artificial Sequence
  B11
caggtgcagctggtagagtctgggggaggcttggtacagcctggggggtctctgagactctcctgtgcagcctctggattcacttttgatgattatgccatgagctgggtccgacaggctccagggaaggggctggagtgggtgtccaatatttatagttacattagtaccacacactatgcagactccgtgaagggccgattcaccatctccacagacaacgccaagaacacactgtatctgcaaatgaacagcctgaaacctgaggacacggccgtttattactgtgcagcccgaagacttgacacgaccgactataaatactggggccaggggacacaggtcaccgtctcctca

SEQ ID NO: 111 - DNA, 360 bp, Artificial Sequence
  E11
caggtgcagctggtagagtctgggggaggcttggtgcagcctggggggtctctgagactctcctgtgcagcctctggattcaccttcagtaactatgccatgacctgggtccgccaggctccaggaaaggggctcgagtggatctcagatattggtagtgatggtaggcacccagcctatgcagactccgtgaagggccgatacaccatctccagagacaacgccaagaacacgctatatctgcaaatgaacagcctgaaacctgaggacacggccatgtattactgtctcccgggttggagtaccgtcgggacttttttggacgcatggggccaggggacccaggtcaccgtctcctca

SEQ ID NO: 112 - DNA, 369 bp, Artificial Sequence
  D1
caggtgcagctggtagagtctgggggaggcttggtgcagcctggggggtctctgagactctcctgtgcagcctctggattcaccttcagtagctatggcatgagctgggtccgccaggctccaggaaaggggctcgagtgggtctcaggtattaatagtggtggtggtagcacaagctatgcagactccgtgaagggccgattcaccatctccagagacaacgccaagaacacgctgtatctgcaaatgaacagtctgaaatctgaggacacggccgtgtattactgtgcaaaagacgctagtgaagatgcggggcgactatactggaccgactactggggccaggggacccaggtcaccgtctcctca

SEQ ID NO: 113 - DNA, 345 bp, Artificial Sequence
  G7
caggtgcagctggtggagtctgggggaggcttggtgcagcctgggggttctctgagactctcctgtgcaacctctggattcaccttcgatacgtatgacatgagctgggtccgccaggctccaggaaacgggcccgagtgggtctcagcggaggatagtactgggaacaaattctatgcagactctgtgaagggccgattcgccatctccagagacaacgccaggaacacactatatctgcaaatgaacagtctgacatctgaagacacggccgtgtattactgtgcaaaaggtctacgatcctggggtgcctggggccaggggacccaggtcaccgtctcctca

SEQ ID NO: 114 - DNA, 357 bp, Artificial Sequence
  F5
caggtgcagctggtagagtctgggggaggcttggtgcaggctggggggtctctgagactctcctgtgcagcctctggaaccatcttcagaatcaatgccatggcctggtaccgccaggctccagggaagcagcgcgagttggtcgcaattatttctaatggtgatagtggtgatagtacaaactatgcagactccgtgaagggccgattcaccatctccagagacagcgccaagaacacggtgtatctgcaaatgaacagcctgaaacctgaggacacagccgtctattactgtaatgtagaagcgtcctggccccataagtactggggccaggggacccaggtcaccgtctcctca

SEQ ID NO: 115 - DNA, 360 bp, Artificial Sequence
  G11
caggtgcacctggtagagtctgggggaggcttggtgcagcctggggggtctctgagactctcctgtgcagcctctggattcacttttgatgattatggcatgacctgggtccgccaggctccaggaaaggggctcgagtgggtctcagcggttaatagtggtggtggtacaagctatgcagactccgtgaagggccgattcaccatctccagagacaacgccaagaacacgctgtatctgcaaatgaacagcctgaaacctgaggacacggccgtgtattactgtgcaaaaagagatggtagttggtggggttataccacggactactggggcaaagggacccaggtcaccgtctcctca

SEQ ID NO: 116 - DNA, 363 bp, Artificial Sequence
  B4
caggtgcagctggtggagtctgggggaggcttggtgcagcctggggggtctctgagactctcctgtgcagcctctggattcaccttcagtagctactggatgtattgggtccgtcaggctccagggaaggggctcgagtgggtctcagtaattaatactggtggtgatactacatactatgcagactccgtgaagggccgattcaccatctccagagacaacgacgagaacacgctgtctctgcaaatgaacagcctgaaacctgaggacacggccctgtattactgtgcgagaacaggggtagtacgtggtccgggcgatttggacgcatggggccaggggacccaggtcaccgtctcctca

SEQ ID NO: 117 - DNA, 360 bp, Artificial Sequence
  G9
caggtgcagctggtggagtctgggggaggcttggtgcagcctggggggtctctgagactctcctgtgcagcctctggattcactttttatgattatcacatgagctgggtccgccaggctccagggaaggggctcgagtgggtctcaggtatcgatactggtggtgggcgcacatacgctgcagactccgtgaagggccgattcaccatctccagagacaacgccaagaacacactgtatctgcaaatgaacagcctgaaatctgaggacacggccctgtattattgtgtgagagatggagatgcacgggggcccgactatgactactggggccaggggaccatggtcaccgtctcctca

SEQ ID NO: 118 - DNA, 348 bp, Artificial Sequence
  C7
caggtgcagctggtagagtctgggggaggcttggtgcagcctggggggtctctgagactctcctgtgcagcctctggattcaccttcagtagctatgacatgagctgggtccgccaggctccaggaaaggggctcgagtgggtctcaggtattagcggaagtgatagtactacagtctattcagactccgtgaaggaccgattcaccatctccagagacaacgccaagaatacgttgtatctgcaaatgaacaaactgaaacctgaggacacggccctgtattactgtgcaagaccattgggacaatacacgagttggggccaggggacccaggtcaccgtctcctca

SEQ ID NO: 119 - protein, 127 aa, Artificial Sequence
  H11_D4 amino acid
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARTENVRSLLSDYATWPYDYWGQGTQVTVSS

SEQ ID NO: 120 - protein, 127 aa, Artificial Sequence
  H11_H4 amino acid
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTHYVSYLLSDYATWPYDYWGQGTQVTVSS

SEQ ID NO: 121 - protein, 127 aa, Artificial Sequence
  H11_H6 amino acid
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAGSKITRSLLSDYATWPYDYWGQGTQVTVSS

SEQ ID NO: 122 - protein, 127 aa, Artificial Sequence
  H11_A10 maino acid
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAGFSATRSLLSDYATWPYDYWGQGTQVTVSS

SEQ ID NO: 123 - protein, 127 aa, Artificial Sequence
  H11_B5 amino acid
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCASYQATRSLLSDYATWPYDYWGQGTQVTVSS

SEQ ID NO: 124 - protein, 127 aa, Artificial Sequence
  H11_A7 amino acid
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTDNVRALLSDYATWPYDYWGQGTQVTVSS

SEQ ID NO: 125 - protein, 127 aa, Artificial Sequence
  H11_F7
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAIFSNVRSLLSDYATWPYDYWGQGTQVTVSS

SEQ ID NO: 126 - protein, 127 aa, Artificial Sequence
  H11_F6 amino acid
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARTSNVRSLLSDYATWPYDYWGQGTQVTVSS

SEQ ID NO: 127 - protein, 127 aa, Artificial Sequence
  H11_G8 amino acid
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAGSGVTRSLLSDYATWPYDYWGQGTQVTVSS

SEQ ID NO: 128 - protein, 127 aa, Artificial Sequence
  H11_D1 amino acid
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAGWRATRSLLSDYATWPYDYWGQGTQVTVSS

SEQ ID NO: 129 - protein, 127 aa, Artificial Sequence
  H11_A9 amino acid
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAPYEATRSLLSDYATWPYDYWGQGTQVTVSS

SEQ ID NO: 130 - protein, 127 aa, Artificial Sequence
  H11_C6 amino acid
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTGYTSWLLSDYATWPYDYWGQGTQVTVSS

SEQ ID NO: 131 - protein, 127 aa, Artificial Sequence
  H11_E3 amino acid
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAVYHATRSLLSDYATWPYDYWGQGTQVTVSS

SEQ ID NO: 132 - protein, 127 aa, Artificial Sequence
  H11_F4 amino acid
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAESTITRSLLSDYATWPYDYWGQGTQVTVSS

SEQ ID NO: 133 - protein, 127 aa, Artificial Sequence
  H11_C5 amino acid
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTSYVSFLLSDYATWPYDYWGQGTQVTVSS

SEQ ID NO: 134 - protein, 127 aa, Artificial Sequence
  H11_C2 amino acid
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAGSRATRSLLSDYATWPYDYWGQGTQVTVSS

SEQ ID NO: 135 - protein, 127 aa, Artificial Sequence
  H11_B11 amino acid
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAETLNTRSLLSDYATWPYDYWGQGTQVTVSS

SEQ ID NO: 136 - protein, 127 aa, Artificial Sequence
  H11_A3 amino acid
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARSDNVRSLLSDYATWPYDYWGQGTQVTVSS

SEQ ID NO: 137 - protein, 127 aa, Artificial Sequence
  H11_D12 amino acid
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCADWGVTRSLLSDYATWPYDYWGQGTQVTVSS

SEQ ID NO: 138 - protein, 127 aa, Artificial Sequence
  H11_D6 amino acid
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCASSSVTRSLLSDYATWPYDYWGQGTQVTVSS

SEQ ID NO: 139 - protein, 127 aa, Artificial Sequence
  H11_F8 amino acid
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCADASATRSLLSDYATWPYDYWGQGTQVTVSS

SEQ ID NO: 140 - DNA, 381 bp, Artificial Sequence
  H11_D4
caggtgcagctggtggagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcacggacggagaatgttcggtccctccttagcgactatgccacttggccttatgactactggggccaggggacccaggtcaccgtctcctca

SEQ ID NO: 141 - DNA, 381 bp, Artificial Sequence
  H11_H4
caggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcacaaacgcattatgtttcttatctccttagcgactatgccacttggccttatgactactggggccaggggacccaggtcaccgtctcctca

SEQ ID NO: 142 - DNA, 381 bp, Artificial Sequence
  H11_H6
caggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcaggttctaagattacgcggtccctccttagcgactatgccacttggccttatgactactggggccaggggacccaggtcaccgtctcctca

SEQ ID NO: 143 - DNA, 381 bp, Artificial Sequence
  H11_A10
caggtgcagctggtggagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcaggttttagtgcgactcggtccctccttagcgactatgccacttggccttatgactactggggccaggggacccaggtcaccgtctcctca

SEQ ID NO: 144 - DNA, 381 bp, Artificial Sequence
  H11_B5
caggtgcagctggtggagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcaagttatcaggcgactcggtccctccttagcgactatgccacttggccttatgactactggggccaggggacccaggtcaccgtctcctca

SEQ ID NO: 145 - DNA, 381 bp, Artificial Sequence
  H11_A7
caggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcacaaacggataatgtgcgggcgctccttagcgactatgccacttggccttatgactactggggccaggggacccaggtcaccgtctcctca

SEQ ID NO: 146 - DNA, 381 bp, Artificial Sequence
  H11_F7
caggtgcagctggtggagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcaattttttctaatgtgcggtccctccttagcgactatgccacttggccttatgactactggggccaggggacccaggtcaccgtctcctca

SEQ ID NO: 147 - DNA, 381 bp, Artificial Sequence
  H11_F6
caggtgcagctggtggagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcacggacgtcgaatgtgcggtccctccttagcgactatgccacttggccttatgactactggggccaggggacccaggtcaccgtctcctca

SEQ ID NO: 148 - DNA, 381 bp, Artificial Sequence
  H11_G8
caggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcagggtctggtgtgacgcggtccctccttagcgactatgccacttggccttatgactactggggccaggggacccaggtcaccgtctcctca

SEQ ID NO: 149 - DNA, 381 bp, Artificial Sequence
  H11_D1
caggtgcagctggtggagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcagggtggcgtgcgacgcggtccctccttagcgactatgccacttggccttatgactactggggccaggggacccaggtcaccgtctcctca

SEQ ID NO: 150 - DNA, 381 bp, Artificial Sequence
  H11_A9
caggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcaccgtatgaggctacgcggtccctccttagcgactatgccacttggccttatgactactggggccaggggacccaggtcaccgtctcctca

SEQ ID NO: 151 - DNA, 381 bp, Artificial Sequence
  H11_C6
caggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcacaaacgggttatacgtcttggctccttagcgactatgccacttggccttatgactactggggccaggggacccaggtcaccgtctcctca

SEQ ID NO: 152 - DNA, 381 bp, Artificial Sequence
  H11_E3
caggtgcagctggtggagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcagtgtatcatgctactcggtccctccttagcgactatgccacttggccttatgactactggggccaggggacccaggtcaccgtctcctca

SEQ ID NO: 153 - DNA, 381 bp, Artificial Sequence
  H11_F4
caggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcagagagtacgattactcggtccctccttagcgactatgccacttggccttatgactactggggccaggggacccaggtcaccgtctcctca

SEQ ID NO: 154 - DNA, 381 bp, Artificial Sequence
  H11_C5
caggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcacaaacgtcgtatgtgagttttctccttagcgactatgccacttggccttatgactactggggccaggggacccaggtcaccgtctcctca

SEQ ID NO: 155 - DNA, 381 bp, Artificial Sequence
  H11_C2
caggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcaggtagtcgggcgacgcggtccctccttagcgactatgccacttggccttatgactactggggccaggggacccaggtcaccgtctcctca

SEQ ID NO: 156 - DNA, 381 bp, Artificial Sequence
  H11_B11
caggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcagagacgttgaatactcggtccctccttagcgactatgccacttggccttatgactactggggccaggggacccaggtcaccgtctcctca

SEQ ID NO: 157 - DNA, 381 bp, Artificial Sequence
  H11_A3
caggtgcagctggtggagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcacgttctgataatgtgcggtccctccttagcgactatgccacttggccttatgactactggggccaggggacccaggtcaccgtctcctca

SEQ ID NO: 158 - DNA, 381 bp, Artificial Sequence
  H11_D12
caggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcagattggggtgtgactcggtccctccttagcgactatgccacttggccttatgactactggggccaggggacccaggtcaccgtctcctca

SEQ ID NO: 159 - DNA, 381 bp, Artificial Sequence
  H11_D6
caggtgcagctggtggagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcaagttcttctgtgacgcggtccctccttagcgactatgccacttggccttatgactactggggccaggggacccaggtcaccgtctcctca

SEQ ID NO: 160 - DNA, 381 bp, Artificial Sequence
  H11_F8
caggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcagatgcttcggcgacgcggtccctccttagcgactatgccacttggccttatgactactggggccaggggacccaggtcaccgtctcctca

SEQ ID NO: 161 - DNA, 1068 bp, Artificial Sequence
  C5-Fc
caggtgcagctggtggagtctgggggaggctcggtgcaggctggggggtctctgacactctcctgtgtcgcctctggagtcactttgggacgtcatgccataggctggttccgccaggcccccgggaaggagcgtgagagagtctcgtgtattagaacatttgatggcatcacaagttatgtagagtccacgaagggccgattcaccatctccagtaacaatgccatgaacacggtgtatctgcaaatgaatagcctcaaacctgaagacacggccgtttatttctgtgcactgggagtgactgcagcctgttcagataatccctacttctggggccaggggacccaggtcaccgtctcctcagcgtcgaccgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 162 - DNA, 822 bp, Artificial Sequence
  C5-AAA-C5 (OmpA)
atgaaaaagacagctatcgcaattgcagtggccttggctggtttcgctaccgtagcgcaagctcaggtgcagctggtcgagtctgggggaggctcggtgcaggctggggggtctctgacactctcctgtgtcgcctctggagtcactttgggacgtcatgccataggctggttccgccaggcccccgggaaggagcgtgagagagtctcgtgtattagaacatttgatggcatcacaagttatgtagagtccacgaagggccgattcaccatctccagtaacaatgccatgaacacggtgtatctgcaaatgaatagcctcaaacctgaagacacggccgtttatttctgtgcactgggagtgactgcagcctgttcagataatccctacttctggggccaggggacccaggtcaccgtctcctcagcagcagcacaggtgcagctggtcgagtctgggggaggctcggtgcaggctggggggtctctgacactctcctgtgtcgcctctggagtcactttgggacgtcatgccataggctggttccgccaggcccccgggaaggagcgtgagagagtctcgtgtattagaacatttgatggcatcacaagttatgtagagtccacgaagggccgattcaccatctccagtaacaatgccatgaacacggtgtatctgcaaatgaatagcctcaaacctgaagacacggccgtttatttctgtgcactgggagtgactgcagcctgttcagataatccctacttctggggccaggggacccaggtcaccgtctcctcaaaacatcaccatcaccatcactaa

SEQ ID NO: 163 - DNA, 840 bp, Artificial Sequence
  C5-9GS-C5
atgaaaaagacagctatcgcaattgcagtggccttggctggtttcgctaccgtagcgcaagctcaggtgcagctggtcgagtctgggggaggctcggtgcaggctggggggtctctgacactctcctgtgtcgcctctggagtcactttgggacgtcatgccataggctggttccgccaggcccccgggaaggagcgtgagagagtctcgtgtattagaacatttgatggcatcacaagttatgtagagtccacgaagggccgattcaccatctccagtaacaatgccatgaacacggtgtatctgcaaatgaatagcctcaaacctgaagacacggccgtttatttctgtgcactgggagtgactgcagcctgttcagataatccctacttctggggccaggggacccaggtcaccgtctcctcaggcggcgggggatcaggtggtggcagtcaggtgcagctggtggagtctgggggaggctcggtgcaggctggggggtctctgacactctcctgtgtcgcctctggagtcactttgggacgtcatgccataggctggttccgccaggcccccgggaaggagcgtgagagagtctcgtgtattagaacatttgatggcatcacaagttatgtagagtccacgaagggccgattcaccatctccagtaacaatgccatgaacacggtgtatctgcaaatgaatagcctcaaacctgaagacacggccgtttatttctgtgcactgggagtgactgcagcctgttcagataatccctacttctggggccaggggacccaggtcaccgtctcctcaaaacatcaccatcaccatcactaa

SEQ ID NO: 164 - DNA, 1131 bp, Artificial Sequence
  C5-SUMO-C5
atgaaaaagacagctatcgcaattgcagtggccttggctggtttcgctaccgtagcgcaagctcaggtgcagctggtcgagtctgggggaggctcggtgcaggctggggggtctctgacactctcctgtgtcgcctctggagtcactttgggacgtcatgccataggctggttccgccaggcccccgggaaggagcgtgagagagtctcgtgtattagaacatttgatggcatcacaagttatgtagagtccacgaagggccgattcaccatctccagtaacaatgccatgaacacggtgtatctgcaaatgaatagcctcaaacctgaagacacggccgtttatttctgtgcactgggagtgactgcagcctgttcagataatccctacttctggggccaggggacccaggtcaccgtctcctcagggagcggttctggctccgatagcgaagtgaaccaggaagcgaaaccggaagttaaaccggaagtgaaaccggaaacccatattaatctgaaagttagcgacggcagcagcgaaatcttttttaaaattaaaaaaaccaccccgctgcgtcgcctgatggaagcctttgcgaaacgtcagggtaaagaaatggatagcctgcgctttctgtatgacggcatccgtattcaggccgatcagaccccggaagacctggatatggaagacaacgatattattgaagcgcatcgcgaacagatcggctcaggtagcgggtcgcaggtgcagctggtggagtctgggggaggctcggtgcaggctggggggtctctgacactctcctgtgtcgcctctggagtcactttgggacgtcatgccataggctggttccgccaggcccccgggaaggagcgtgagagagtctcgtgtattagaacatttgatggcatcacaagttatgtagagtccacgaagggccgattcaccatctccagtaacaatgccatgaacacggtgtatctgcaaatgaatagcctcaaacctgaagacacggccgtttatttctgtgcactgggagtgactgcagcctgttcagataatccctacttctggggccaggggacccaggtcaccgtctcctcaaaacatcaccatcaccatcactaa

SEQ ID NO: 165 - DNA, 1056 bp, Artificial Sequence
  C5-GSGSGS-SUMO-GSGSGS-C5
caggtgcagctggtggagtctgggggaggctcggtgcaggctggggggtctctgacactctcctgtgtcgcctctggagtcactttgggacgtcatgccataggctggttccgccaggcccccgggaaggagcgtgagagagtctcgtgtattagaacatttgatggcatcacaagttatgtagagtccacgaagggccgattcaccatctccagtaacaatgccatgaacacggtgtatctgcaaatgaatagcctcaaacctgaagacacggccgtttatttctgtgcactgggagtgactgcagcctgttcagataatccctacttctggggccaggggacccaggtcaccgtctcctcagggagcggttctggctccgatagcgaagtgaaccaggaagcgaaaccggaagttaaaccggaagtgaaaccggaaacccatattaatctgaaagttagcgacggcagcagcgaaatcttttttaaaattaaaaaaaccaccccgctgcgtcgcctgatggaagcctttgcgaaacgtcagggtaaagaaatggatagcctgcgctttctgtatgacggcatccgtattcaggccgatcagaccccggaagacctggatatggaagacaacgatattattgaagcgcatcgcgaacagatcggctcaggtagcgggtcgcaggtgcagctggtggagtctgggggaggattggtgcaggctgggggctctctaagactcgcttgtatagcctctggacgcaccttccatagctatgtcatggcctggttccgccaggctccagggaaggagcgtgagtttgtagcagctattagttggagtagtacaccgacatactatggagaatccgtgaagggccgattcaccatctccagagacaacgccaagaacacggtgtatctgcaaatgaaccgcctgaaacctgaggacacggccgtttatttctgtgcagcagaccggggtgaaagttactactacactcgacccaccgagtatgaattctggggccaggggacccaggtcaccgtctcctca

SEQ ID NO: 166 - DNA, 1056 bp, Artificial Sequence
  F2-SUMO-C5
caggtgcagctggtggagtctgggggaggattggtgcaggctgggggctctctaagactcgcttgtatagcctctggacgcaccttccatagctatgtcatggcctggttccgccaggctccagggaaggagcgtgagtttgtagcagctattagttggagtagtacaccgacatactatggagaatccgtgaagggccgattcaccatctccagagacaacgccaagaacacggtgtatctgcaaatgaaccgcctgaaacctgaggacacggccgtttatttctgtgcagcagaccggggtgaaagttactactacactcgacccaccgagtatgaattctggggccaggggacccaggtcaccgtctcctcagggagcggttctggctccgatagcgaagtgaaccaggaagcgaaaccggaagttaaaccggaagtgaaaccggaaacccatattaatctgaaagttagcgacggcagcagcgaaatcttttttaaaattaaaaaaaccaccccgctgcgtcgcctgatggaagcctttgcgaaacgtcagggtaaagaaatggatagcctgcgctttctgtatgacggcatccgtattcaggccgatcagaccccggaagacctggatatggaagacaacgatattattgaagcgcatcgcgaacagatcggctcaggtagcgggtcgcaggtgcagctggtggagtctgggggaggctcggtgcaggctggggggtctctgacactctcctgtgtcgcctctggagtcactttgggacgtcatgccataggctggttccgccaggcccccgggaaggagcgtgagagagtctcgtgtattagaacatttgatggcatcacaagttatgtagagtccacgaagggccgattcaccatctccagtaacaatgccatgaacacggtgtatctgcaaatgaatagcctcaaacctgaagacacggccgtttatttctgtgcactgggagtgactgcagcctgttcagataatccctacttctggggccaggggacccaggtcaccgtctcctca

SEQ ID NO: 167 - DNA, 1053 bp, Artificial Sequence
  C1-SUMO-C5
caggtgcagctggtggagtctgggggaggcttggtgcagcctgggggctctctgagactctcctgtgcagcctctggattcactaatgatttttatagcatcgcgtggttccgccaggccccaggaaaggagcgtgagggggtctcatggcttagtgtcagtgataataccccaacctacgtagactccgtgaaggaccggttcaccatctccagacacaacgccaacaacaccgtgtacctgcaaatgaacatgctgaaacctgaggacacggccatttactattgtgcagcaggacgcttcgcgggaagggatacttggccctcgtcctatgattactggggccaggggacccaggtcaccgtctcctcagggagcggttctggctccgatagcgaagtgaaccaggaagcgaaaccggaagttaaaccggaagtgaaaccggaaacccatattaatctgaaagttagcgacggcagcagcgaaatcttttttaaaattaaaaaaaccaccccgctgcgtcgcctgatggaagcctttgcgaaacgtcagggtaaagaaatggatagcctgcgctttctgtatgacggcatccgtattcaggccgatcagaccccggaagacctggatatggaagacaacgatattattgaagcgcatcgcgaacagatcggctcaggtagcgggtcgcaggtgcagctggtggagtctgggggaggctcggtgcaggctggggggtctctgacactctcctgtgtcgcctctggagtcactttgggacgtcatgccataggctggttccgccaggcccccgggaaggagcgtgagagagtctcgtgtattagaacatttgatggcatcacaagttatgtagagtccacgaagggccgattcaccatctccagtaacaatgccatgaacacggtgtatctgcaaatgaatagcctcaaacctgaagacacggccgtttatttctgtgcactgggagtgactgcagcctgttcagataatccctacttctggggccaggggacccaggtcaccgtctcctca

SEQ ID NO: 168 - DNA, 1071 bp, Artificial Sequence
  C1-SUMO-H3
caggtgcagctggtggagtctgggggaggcttggtgcagcctgggggctctctgagactctcctgtgcagcctctggattcactaatgatttttatagcatcgcgtggttccgccaggccccaggaaaggagcgtgagggggtctcatggcttagtgtcagtgataataccccaacctacgtagactccgtgaaggaccggttcaccatctccagacacaacgccaacaacaccgtgtacctgcaaatgaacatgctgaaacctgaggacacggccatttactattgtgcagcaggacgcttcgcgggaagggatacttggccctcgtcctatgattactggggccaggggacccaggtcaccgtctcctcagggagcggttctggctccgatagcgaagtgaaccaggaagcgaaaccggaagttaaaccggaagtgaaaccggaaacccatattaatctgaaagttagcgacggcagcagcgaaatcttttttaaaattaaaaaaaccaccccgctgcgtcgcctgatggaagcctttgcgaaacgtcagggtaaagaaatggatagcctgcgctttctgtatgacggcatccgtattcaggccgatcagaccccggaagacctggatatggaagacaacgatattattgaagcgcatcgcgaacagatcggctcaggtagcgggtcgcaggtgcagctggtggagtctgggggaggattggtgaagactgggggctctctgagactctcctgtgcagcctctggccgcaccttcagtacctacagcatgggctggttccgccaggctccagggaaggagcgtgagtttgtagcaggtatgcgctggacgggtagtagtacattctactcagactccgtgaagggccgattcaccgtctccagaaacaacgccaaggacacggtgtatctgcacatgaacagcctgaaacctgaggacacggccgtttattactgtgcaatcacgactatcgtaagagcttactatactgagtataccgaagctgactttggttcctggggccaggggacccaggtcaccgtctcctca

SEQ ID NO: 169 - DNA, 705 bp, Artificial Sequence
  hIgG1-Fc
gcgtcgaccgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 170 - protein, 356 aa, Artificial Sequence
  C5 fused to the Fc region of hIgG1
QVQLVESGGGSVQAGGSLTLSCVASGVTLGRHAIGWFRQAPGKERERVSCIRTFDGITSYVESTKGRFTISSNNAMNTVYLQMNSLKPEDTAVYFCALGVTAACSDNPYFWGQGTQVTVSSASTEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 273 aa, Artificial Sequence
  C5-AAA-C5
MKKTAIAIAVALAGFATVAQAQVQLVESGGGSVQAGGSLTLSCVASGVTLGRHAIGWFRQAPGKERERVSCIRTFDGITSYVESTKGRFTISSNNAMNTVYLQMNSLKPEDTAVYFCALGVTAACSDNPYFWGQGTQVTVSSAAAQVQLVESGGGSVQAGGSLTLSCVASGVTLGRHAIGWFRQAPGKERERVSCIRTFDGITSYVESTKGRFTISSNNAMNTVYLQMNSLKPEDTAVYFCALGVTAACSDNPYFWGQGTQVTVSSKHHHHHH

SEQ ID NO: 172 - protein, 279 aa, Artificial Sequence
  C5-9GS-C5
MKKTAIAIAVALAGFATVAQAQVQLVESGGGSVQAGGSLTLSCVASGVTLGRHAIGWFRQAPGKERERVSCIRTFDGITSYVESTKGRFTISSNNAMNTVYLQMNSLKPEDTAVYFCALGVTAACSDNPYFWGQGTQVTVSSGGGGSGGGSQVQLVESGGGSVQAGGSLTLSCVASGVTLGRHAIGWFRQAPGKERERVSCIRTFDGITSYVESTKGRFTISSNNAMNTVYLQMNSLKPEDTAVYFCALGVTAACSDNPYFWGQGTQVTVSSKHHHHHH

SEQ ID NO: 173 - protein, 376 aa, Artificial Sequence
  C5-GSGSGS-SUMO-GSGSGS-C5
MKKTAIAIAVALAGFATVAQAQVQLVESGGGSVQAGGSLTLSCVASGVTLGRHAIGWFRQAPGKERERVSCIRTFDGITSYVESTKGRFTISSNNAMNTVYLQMNSLKPEDTAVYFCALGVTAACSDNPYFWGQGTQVTVSSGSGSGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGSGSGSQVQLVESGGGSVQAGGSLTLSCVASGVTLGRHAIGWFRQAPGKERERVSCIRTFDGITSYVESTKGRFTISSNNAMNTVYLQMNSLKPEDTAVYFCALGVTAACSDNPYFWGQGTQVTVSSKHHHHHH

SEQ ID NO: 174 - protein, 352 aa, Artificial Sequence
  C5-GSGSGS-SUMO-GSGSGS-F2
QVQLVESGGGSVQAGGSLTLSCVASGVTLGRHAIGWFRQAPGKERERVSCIRTFDGITSYVESTKGRFTISSNNAMNTVYLQMNSLKPEDTAVYFCALGVTAACSDNPYFWGQGTQVTVSSGSGSGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGSGSGSQVQLVESGGGLVQAGGSLRLACIASGRTFHSYVMAWFRQAPGKEREFVAAISWSSTPTYYGESVKGRFTISRDNAKNTVYLQMNRLKPEDTAVYFCAADRGESYYYTRPTEYEFWGQGTQVTVSS

SEQ ID NO: 175 - protein, 352 aa, Artificial Sequence
  F2-GSGSGS-SUMO-GSGSGS-C5
QVQLVESGGGLVQAGGSLRLACIASGRTFHSYVMAWFRQAPGKEREFVAAISWSSTPTYYGESVKGRFTISRDNAKNTVYLQMNRLKPEDTAVYFCAADRGESYYYTRPTEYEFWGQGTQVTVSSGSGSGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGSGSGSQVQLVESGGGSVQAGGSLTLSCVASGVTLGRHAIGWFRQAPGKERERVSCIRTFDGITSYVESTKGRFTISSNNAMNTVYLQMNSLKPEDTAVYFCALGVTAACSDNPYFWGQGTQVTVSS

SEQ ID NO: 176 - protein, 351 aa, Artificial Sequence
  C1-GSGSGS-SUMO-GSGSGS-C5
QVQLVESGGGLVQPGGSLRLSCAASGFTNDFYSIAWFRQAPGKEREGVSWLSVSDNTPTYVDSVKDRFTISRHNANNTVYLQMNMLKPEDTAIYYCAAGRFAGRDTWPSSYDYWGQGTQVTVSSGSGSGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGSGSGSQVQLVESGGGSVQAGGSLTLSCVASGVTLGRHAIGWFRQAPGKERERVSCIRTFDGITSYVESTKGRFTISSNNAMNTVYLQMNSLKPEDTAVYFCALGVTAACSDNPYFWGQGTQVTVSS

SEQ ID NO: 177 - protein, 356 aa, Artificial Sequence
  C1-GSGSGS-SUMO-GSGSGS-H3
QVQLVESGGGLVQPGGSLRLSCAASGFTNDFYSIAWFRQAPGKEREGVSWLSVSDNTPTYVDSVKDRFTISRHNANNTVYLQMNMLKPEDTAIYYCAAGRFAGRDTWPSSYDYWGQGTQVTVSSGSGSGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGSGSGSQVQLVESGGGLVKTGGSLRLSCAASGRTFSTYSMGWFRQAPGKEREFVAGMRWTGSSTFYSDSVKGRFTVSRNNAKDTVYLHMNSLKPEDTAVYYCAITTIVRAYYTEYTEADFGSWGQGTQVTVS

SEQ ID NO: 178 - DNA, 1071 bp, Artificial Sequence
  C1-SUMO-H11-H4
caggtgcagctggtggagtctgggggaggcttggtgcagcctgggggctctctgagactctcctgtgcagcctctggattcactaatgatttttatagcatcgcgtggttccgccaggccccaggaaaggagcgtgagggggtctcatggcttagtgtcagtgataataccccaacctacgtagactccgtgaaggaccggttcaccatctccagacacaacgccaacaacaccgtgtacctgcaaatgaacatgctgaaacctgaggacacggccatttactattgtgcagcaggacgcttcgcgggaagggatacttggccctcgtcctatgattactggggccaggggacccaggtcaccgtctcctcagggagcggttctggctccgatagcgaagtgaaccaggaagcgaaaccggaagttaaaccggaagtgaaaccggaaacccatattaatctgaaagttagcgacggcagcagcgaaatcttttttaaaattaaaaaaaccaccccgctgcgtcgcctgatggaagcctttgcgaaacgtcagggtaaagaaatggatagcctgcgctttctgtatgacggcatccgtattcaggccgatcagaccccggaagacctggatatggaagacaacgatattattgaagcgcatcgcgaacagatcggctcaggtagcgggtcgcaggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcaggttctaagattacgcggtccctccttagcgactatgccacttggccttatgactactggggccaggggacccaggtcaccgtctcctca

SEQ ID NO: 179 - protein, 357 aa, Artificial Sequence
  C1-GSGSGS-SUMO-GSGSGS-H11-H4
QVQLVESGGGLVQPGGSLRLSCAASGFTNDFYSIAWFRQAPGKEREGVSWLSVSDNTPTYVDSVKDRFTISRHNANNTVYLQMNMLKPEDTAIYYCAAGRFAGRDTWPSSYDYWGQGTQVTVSSGSGSGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGSGSGSQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCAQTHYVSYLLSDYATWPYDYWGQGTQVTVSS

SEQ ID NO: 180 - protein, 235 aa, Artificial Sequence
  hIgG1-Fc
ASTEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - DNA, 735 bp, Artificial Sequence
  C5-AAA-C5
caggtgcagctggtcgagtctgggggaggctcggtgcaggctggggggtctctgacactctcctgtgtcgcctctggagtcactttgggacgtcatgccataggctggttccgccaggcccccgggaaggagcgtgagagagtctcgtgtattagaacatttgatggcatcacaagttatgtagagtccacgaagggccgattcaccatctccagtaacaatgccatgaacacggtgtatctgcaaatgaatagcctcaaacctgaagacacggccgtttatttctgtgcactgggagtgactgcagcctgttcagataatccctacttctggggccaggggacccaggtcaccgtctcctcagcagcagcacaggtgcagctggtcgagtctgggggaggctcggtgcaggctggggggtctctgacactctcctgtgtcgcctctggagtcactttgggacgtcatgccataggctggttccgccaggcccccgggaaggagcgtgagagagtctcgtgtattagaacatttgatggcatcacaagttatgtagagtccacgaagggccgattcaccatctccagtaacaatgccatgaacacggtgtatctgcaaatgaatagcctcaaacctgaagacacggccgtttatttctgtgcactgggagtgactgcagcctgttcagataatccctacttctggggccaggggacccaggtcaccgtctcctca

SEQ ID NO: 182 - protein, 245 aa, Artificial Sequence
  C5-AAA-C5
QVQLVESGGGSVQAGGSLTLSCVASGVTLGRHAIGWFRQAPGKERERVSCIRTFDGITSYVESTKGRFTISSNNAMNTVYLQMNSLKPEDTAVYFCALGVTAACSDNPYFWGQGTQVTVSSAAAQVQLVESGGGSVQAGGSLTLSCVASGVTLGRHAIGWFRQAPGKERERVSCIRTFDGITSYVESTKGRFTISSNNAMNTVYLQMNSLKPEDTAVYFCALGVTAACSDNPYFWGQGTQVTVSS

SEQ ID NO: 183 - DNA, 753 bp, Artificial Sequence
  C5-9GS-C5
caggtgcagctggtcgagtctgggggaggctcggtgcaggctggggggtctctgacactctcctgtgtcgcctctggagtcactttgggacgtcatgccataggctggttccgccaggcccccgggaaggagcgtgagagagtctcgtgtattagaacatttgatggcatcacaagttatgtagagtccacgaagggccgattcaccatctccagtaacaatgccatgaacacggtgtatctgcaaatgaatagcctcaaacctgaagacacggccgtttatttctgtgcactgggagtgactgcagcctgttcagataatccctacttctggggccaggggacccaggtcaccgtctcctcaggcggcgggggatcaggtggtggcagtcaggtgcagctggtggagtctgggggaggctcggtgcaggctggggggtctctgacactctcctgtgtcgcctctggagtcactttgggacgtcatgccataggctggttccgccaggcccccgggaaggagcgtgagagagtctcgtgtattagaacatttgatggcatcacaagttatgtagagtccacgaagggccgattcaccatctccagtaacaatgccatgaacacggtgtatctgcaaatgaatagcctcaaacctgaagacacggccgtttatttctgtgcactgggagtgactgcagcctgttcagataatccctacttctggggccaggggacccaggtcaccgtctcctca

SEQ ID NO: 184 - protein, 251 aa, Artificial Sequence
  C5-9GS-C5
QVQLVESGGGSVQAGGSLTLSCVASGVTLGRHAIGWFRQAPGKERERVSCIRTFDGITSYVESTKGRFTISSNNAMNTVYLQMNSLKPEDTAVYFCALGVTAACSDNPYFWGQGTQVTVSSGGGGSGGGSQVQLVESGGGSVQAGGSLTLSCVASGVTLGRHAIGWFRQAPGKERERVSCIRTFDGITSYVESTKGRFTISSNNAMNTVYLQMNSLKPEDTAVYFCALGVTAACSDNPYFWGQGTQVTVSS

SEQ ID NO: 185 - DNA, 1044 bp, Artificial Sequence
  C5-SUMO-C5
caggtgcagctggtcgagtctgggggaggctcggtgcaggctggggggtctctgacactctcctgtgtcgcctctggagtcactttgggacgtcatgccataggctggttccgccaggcccccgggaaggagcgtgagagagtctcgtgtattagaacatttgatggcatcacaagttatgtagagtccacgaagggccgattcaccatctccagtaacaatgccatgaacacggtgtatctgcaaatgaatagcctcaaacctgaagacacggccgtttatttctgtgcactgggagtgactgcagcctgttcagataatccctacttctggggccaggggacccaggtcaccgtctcctcagggagcggttctggctccgatagcgaagtgaaccaggaagcgaaaccggaagttaaaccggaagtgaaaccggaaacccatattaatctgaaagttagcgacggcagcagcgaaatcttttttaaaattaaaaaaaccaccccgctgcgtcgcctgatggaagcctttgcgaaacgtcagggtaaagaaatggatagcctgcgctttctgtatgacggcatccgtattcaggccgatcagaccccggaagacctggatatggaagacaacgatattattgaagcgcatcgcgaacagatcggctcaggtagcgggtcgcaggtgcagctggtggagtctgggggaggctcggtgcaggctggggggtctctgacactctcctgtgtcgcctctggagtcactttgggacgtcatgccataggctggttccgccaggcccccgggaaggagcgtgagagagtctcgtgtattagaacatttgatggcatcacaagttatgtagagtccacgaagggccgattcaccatctccagtaacaatgccatgaacacggtgtatctgcaaatgaatagcctcaaacctgaagacacggccgtttatttctgtgcactgggagtgactgcagcctgttcagataatccctacttctggggccaggggacccaggtcaccgtctcctca

SEQ ID NO: 186 - protein, 348 aa, Artificial Sequence
  C5-GSGSGS-SUMO-GSGSGS-C5
QVQLVESGGGSVQAGGSLTLSCVASGVTLGRHAIGWFRQAPGKERERVSCIRTFDGITSYVESTKGRFTISSNNAMNTVYLQMNSLKPEDTAVYFCALGVTAACSDNPYFWGQGTQVTVSSGSGSGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGSGSGSQVQLVESGGGSVQAGGSLTLSCVASGVTLGRHAIGWFRQAPGKERERVSCIRTFDGITSYVESTKGRFTISSNNAMNTVYLQMNSLKPEDTAVYFCALGVTAACSDNPYFWGQGTQVTVSS

SEQ ID NO: 187 - DNA, 1146 bp, Artificial Sequence
  C5-6GS-C5-6GS-C5
caggtgcagctggtcgagtctgggggaggctcggtgcaggctggggggtctctgacactctcctgtgtcgcctctggagtcactttgggacgtcatgccataggctggttccgccaggcccccgggaaggagcgtgagagagtctcgtgtattagaacatttgatggcatcacaagttatgtagagtccacgaagggccgattcaccatctccagtaacaatgccatgaacacggtgtatctgcaaatgaatagcctcaaacctgaagacacggccgtttatttctgtgcactgggagtgactgcagcctgttcagataatccctacttctggggccaggggacccaggtcaccgtctcctcagggagcggttctggctcccaggtgcagctggtggagtctgggggaggctcggtgcaggctggggggtctctgacactctcctgtgtcgcctctggagtcactttgggacgtcatgccataggctggttccgccaggcccccgggaaggagcgtgagagagtctcgtgtattagaacatttgatggcatcacaagttatgtagagtccacgaagggccgattcaccatctccagtaacaatgccatgaacacggtgtatctgcaaatgaatagcctcaaacctgaagacacggccgtttatttctgtgcactgggagtgactgcagcctgttcagataatccctacttctggggccaggggacccaggtcaccgtctcctcaggctcaggtagcgggtcgcaggtgcagctggtcgagtctgggggaggctcggtgcaggctggggggtctctgacactctcctgtgtcgcctctggagtcactttgggacgtcatgccataggctggttccgccaggcccccgggaaggagcgtgagagagtctcgtgtattagaacatttgatggcatcacaagttatgtagagtccacgaagggccgattcaccatctccagtaacaatgccatgaacacggtgtatctgcaaatgaatagcctcaaacctgaagacacggccgtttatttctgtgcactgggagtgactgcagcctgttcagataatccctacttctggggccaggggacccaggtcaccgtctcctcaaaacatcaccatcaccatcac

SEQ ID NO: 188 - protein, 382 aa, Artificial Sequence
  C5-6GS-C5-6GS-C5
QVQLVESGGGSVQAGGSLTLSCVASGVTLGRHAIGWFRQAPGKERERVSCIRTFDGITSYVESTKGRFTISSNNAMNTVYLQMNSLKPEDTAVYFCALGVTAACSDNPYFWGQGTQVTVSSGSGSGSQVQLVESGGGSVQAGGSLTLSCVASGVTLGRHAIGWFRQAPGKERERVSCIRTFDGITSYVESTKGRFTISSNNAMNTVYLQMNSLKPEDTAVYFCALGVTAACSDNPYFWGQGTQVTVSSGSGSGSQVQLVESGGGSVQAGGSLTLSCVASGVTLGRHAIGWFRQAPGKERERVSCIRTFDGITSYVESTKGRFTISSNNAMNTVYLQMNSLKPEDTAVYFCALGVTAACSDNPYFWGQGTQVTVSSKHHHHHH

SEQ ID NO: 189 - protein, 375 aa, Artificial Sequence
  C5-6GS-C5-6GS-C5
QVQLVESGGGSVQAGGSLTLSCVASGVTLGRHAIGWFRQAPGKERERVSCIRTFDGITSYVESTKGRFTISSNNAMNTVYLQMNSLKPEDTAVYFCALGVTAACSDNPYFWGQGTQVTVSSGSGSGSQVQLVESGGGSVQAGGSLTLSCVASGVTLGRHAIGWFRQAPGKERERVSCIRTFDGITSYVESTKGRFTISSNNAMNTVYLQMNSLKPEDTAVYFCALGVTAACSDNPYFWGQGTQVTVSSGSGSGSQVQLVESGGGSVQAGGSLTLSCVASGVTLGRHAIGWFRQAPGKERERVSCIRTFDGITSYVESTKGRFTISSNNAMNTVYLQMNSLKPEDTAVYFCALGVTAACSDNPYFWGQGTQVTVSS

SEQ ID NO: 190 - protein, 8 aa, Artificial Sequence
  NbSA_A10 CDR1
GRTFSTTR

SEQ ID NO: 191 - protein, 8 aa, Artificial Sequence
  NbSA_A10 CDR2
IFLNTGTT

SEQ ID NO: 192 - protein, 18 aa, Artificial Sequence
  NbSA_A10 CDR3
AAGRFSAAPLTQSTAFES

SEQ ID NO: 193 - protein, 8 aa, Artificial Sequence
  NbSA_D10 CDR1
GRTSSDYS

SEQ ID NO: 194 - protein, 8 aa, Artificial Sequence
  NbSA_D10 CDR2
LAWTVGAT

SEQ ID NO: 195 - protein, 17 aa, Artificial Sequence
  NbSA_D10 CDR3
AGRYGAGLGFTERIYDY

SEQ ID NO: 196 - protein, 8 aa, Artificial Sequence
  A8 CDR1
GGTFSTAA

SEQ ID NO: 197 - protein, 8 aa, Artificial Sequence
  A8 CDR2
IGWRGVRT

SEQ ID NO: 198 - protein, 18 aa, Artificial Sequence
  A8 CDR3
AASVGNYGLPWAHFEYDF

SEQ ID NO: 199 - protein, 7 aa, Artificial Sequence
  3_C5 CDR1
GRTLSMR

SEQ ID NO: 200 - protein, 8 aa, Artificial Sequence
  3_C5 CDR2
INWSSGSI

SEQ ID NO: 201 - protein, 15 aa, Artificial Sequence
  3_C5 CDR3
AVQVDIGGYLDGYDY

SEQ ID NO: 202 - protein, 8 aa, Artificial Sequence
  8_G11 CDR1
GRTITEYT

SEQ ID NO: 203 - protein, 8 aa, Artificial Sequence
  8_G11 CDR2
ISKSTDST

SEQ ID NO: 204 - protein, 17 aa, Artificial Sequence
  8_G11 CDR3
AAGSFYGRDSDAGGYDY

SEQ ID NO: 205 - protein, 8 aa, Artificial Sequence
  12_F11 CDR1
GGAFSTSA

SEQ ID NO: 206 - protein, 8 aa, Artificial Sequence
  12_F11 CDR2
IGWRGVRT

SEQ ID NO: 207 - protein, 18 aa, Artificial Sequence
  12_F11 CDR3
AASDGNYGLPWAHFEYDF

SEQ ID NO: 208 - DNA, 372 bp, Artificial Sequence
  NbSA_A10
caggtgcagctggtcgagtctgggggaggcttggtgcaggctggggactctctgagactctcctgtgcagcctctggacgtaccttcagtaccactcgcatggcctggttccgccagcctccagggaaggagcgcgagtttgtagcggcgatttttctgaatactggcacgacatactatgcagatcccgtgaaggaccgattcgccatctccagagacaatgccaaaaacacggtgtatctgcaaatgaacagcctgaaacctgaggacacggccgtttattactgcgcagcgggtcggttcagcgctgctccgcttacacagtcaactgcctttgagtcctggggccaggggacccaggtcaccgtctcc

SEQ ID NO: 209 - protein, 125 aa, Artificial Sequence
  NbSA_A10
QVQLVESGGGLVQAGDSLRLSCAASGRTFSTTRMAWFRQPPGKEREFVAAIFLNTGTTYYADPVKDRFAISRDNAKNTVYLQMNSLKPEDTAVYYCAAGRFSAAPLTQSTAFESWGQGTQVTVSA

SEQ ID NO: 210 - DNA, 372 bp, Artificial Sequence
  NbSA_D10
caggtgcagctggtcgagtctgggggaggattggtacaggttgggggctctctgagactctcctgtgcagtctctggacgcaccagcagtgactattccgtgggctggttccgccgggctcaagggaaggagcgtgagtttgtggctgctctggcgtggactgttggcgccacacactatggagactccgtgaagggccgattcaccgtctccagagacaacgccaagaacatggtgtatctgcaaatagacagcctgaaacctgaagacacgggcgtttattactgtgcaggtcgatatggagcgggattggggttcaccgaaagaatctatgactactggggccaggggacccaggtcaccgtttcctca

SEQ ID NO: 211 - protein, 124 aa, Artificial Sequence
  NbSA_D10
QVQLVESGGGLVQVGGSLRLSCAVSGRTSSDYSVGWFRRAQGKEREFVAALAWTVGATHYGDSVKGRFTVSRDNAKNMVYLQIDSLKPEDTGVYYCAGRYGAGLGFTERIYDYWGQGTQVTVSS

SEQ ID NO: 212 - DNA, 375 bp, Artificial Sequence
  A8
caggtgcagctggtcgagtctgggggaggattggtgcaggctggaggctctctgagactctcctgtgcagcctctggaggcaccttcagtaccgctgccatgggctggttccgccaggctccaggcgaggagcgtgagtgtgtagcatctataggctggagaggtgttagaacatggtatgcagactccgtgaagggccgatttaccatctccagagataatccccagaatacggtgtatttgcagatgaacaacctgaaatctggcgacacggccgtttattactgcgcagcctcagtcggcaactacgggttgccgtgggcccacttcgaatatgacttctggggccaggggatccaggtcaccgtgtcgtca

SEQ ID NO: 213 - protein, 125 aa, Artificial Sequence
  A8
QVQLVESGGGLVQAGGSLRLSCAASGGTFSTAAMGWFRQAPGEERECVASIGWRGVRTWYADSVKGRFTISRDNPQNTVYLQMNNLKSGDTAVYYCAASVGNYGLPWAHFEYDFWGQGIQVTVSS

SEQ ID NO: 214 - DNA, 381 bp, Artificial Sequence
  3_C5
cagccggccatggcccaggtgcagctggtcgagtctgggggaggattggtgcaggctgggggctccctgagactctcctgtgcagcctctggacgcaccttgagtatgcgtcgcatgagctggttccgccaggctccagggaaggagcgtgagtttgtagccactattaactggagtagtggtagtatatacgttacagactccgtgaaggaccgattcaccatctccagagacaacgccgagaacacggtgcatctgcaaatgaacatgctgaaacctgaggacacggccgtttattcgtgtgcagtccaagtcgatattggggggtacctcgacggctatgactactggggccaggggacccaggtcaccgtctcctca

SEQ ID NO: 215 - protein, 122 aa, Artificial Sequence
  3_C5
QVQLVESGGGLVQAGGSLRLSCAASGRTLSMRRMSWFRQAPGKEREFVATINWSSGSIYVTDSVKDRFTISRDNAENTVHLQMNMLKPEDTAVYSCAVQVDIGGYLDGYDYWGQGTQVTVSS

SEQ ID NO: 216 - DNA, 372 bp, Artificial Sequence
  8_G11
caggtgcagctggtcgagtctgggggaggattggtgcaggctgggggctctctgagactctcctgtgcagcctctggacgcaccatcactgaatataccatagggtggttccgccaggctccagggaaggagcgtgagtttgtaacacttattagcaagagtactgatagtacatgggatgcagactccgtgaagggccgattcaccatcgccagagattacgccaagaacacggtgtatctgcaaatgaacagcctgaaacctgaggacacggccgtttattactgtgcagctggctccttttacgggcgcgattcggatgctgggggctatgactactggggccaggggacccaggtcaccgtctcctca

SEQ ID NO: 217 - protein, 124 aa, Artificial Sequence
  8_G11
QVQLVESGGGLVQAGGSLRLSCAASGRTITEYTIGWFRQAPGKEREFVTLISKSTDSTWDADSVKGRFTIARDYAKNTVYLQMNSLKPEDTAVYYCAAGSFYGRDSDAGGYDYWGQGTQVTVSS

SEQ ID NO: 218 - DNA, 375 bp, Artificial Sequence
  12_F11
caggtgcagctggtcgagtctggaggaggattggtgcaggctggaggctctctgagactctcctgtgcagcctctggaggcgccttcagtacctctgccatgggctggttccgccaggctccagggaaggaacgtgagtttgtcgcagttataggctggagaggtgttagaacatactatgcagactccgtgaagggccgtttcaccatcgccagagataatccccagaacagggtgtatctggagatgagcagcctgaaatctgacgacacgggcgtttatttctgtgcagcctcagacggcaactacggattgccgtgggcccatttcgagtatgacttctggggccaggggatccaggtcaccgtctcctca

SEQ ID NO: 219 - protein, 124 aa, Artificial Sequence
  12_F11
QVQLVESGGGLVQAGGSLRLSCAASGGAFSTSAMGWFRQAPGKEREFVAVIGWRGVRTYYADSVKGRFTIARDNPQNRVYLEMSSLKSDDTGVYFCAASDGNYGLPWAHFEYDFWGQGIQVTSS

SEQ ID NO: 220 - protein, 124 aa, Artificial Sequence
  C1 alternative sequence (N76Q),
QVQLVESGGGLVQPGGSLRLSCAASGFTNDFYSIAWFRQAPGKEREGVSWLSVSDNTPTYVDSVKDRFTISRHNAQNTVYLQMNMLKPEDTAIYYCAAGRFAGRDTWPSSYDYWGQGTQVTVSS

SEQ ID NO: 221 - protein, 394 aa, Artificial Sequence
  Trivalent A8
QVQLVESGGGLVQAGGSLRLSCAASGGTFSTAAMGWFRQAPGEERECVASIGWRGVRTWYADSVKGRFTISRDNPQNTVYLQMNNLKSGDTAVYYCAASVGNYGLPWAHFEYDFWGQGIQVTVSSGSGSGSQVQLVESGGGLVQAGGSLRLSCAASGGTFSTAAMGWFRQAPGEERECVASIGWRGVRTWYADSVKGRFTISRDNPQNTVYLQMNNLKSGDTAVYYCAASVGNYGLPWAHFEYDFWGQGIQVTVSSGSGSGSQVQLVESGGGLVQAGGSLRLSCAASGGTFSTAAMGWFRQAPGEERECVASIGWRGVRTWYADSVKGRFTISRDNPQNTVYLQMNNLKSGDTAVYYCAASVGNYGLPWAHFEYDFWGQGTQVTVSSKHHHHHH

SEQ ID NO: 222 - protein, 387 aa, Artificial Sequence
  Trivalent A8
QVQLVESGGGLVQAGGSLRLSCAASGGTFSTAAMGWFRQAPGEERECVASIGWRGVRTWYADSVKGRFTISRDNPQNTVYLQMNNLKSGDTAVYYCAASVGNYGLPWAHFEYDFWGQGIQVTVSSGSGSGSQVQLVESGGGLVQAGGSLRLSCAASGGTFSTAAMGWFRQAPGEERECVASIGWRGVRTWYADSVKGRFTISRDNPQNTVYLQMNNLKSGDTAVYYCAASVGNYGLPWAHFEYDFWGQGIQVTVSSGSGSGSQVQLVESGGGLVQAGGSLRLSCAASGGTFSTAAMGWFRQAPGEERECVASIGWRGVRTWYADSVKGRFTISRDNPQNTVYLQMNNLKSGDTAVYYCAASVGNYGLPWAHFEYDFWGQGIQVTVSS

SEQ ID NO: 223 - DNA, 1152 bp, Artificial Sequence
  Trivalent C1
caggtgcagctggtggagtctgggggaggcttggtgcagcctgggggctctctgagactctcctgtgcagcctctggattcactaatgatttttatagcatcgcgtggttccgccaggccccaggaaaggagcgtgagggggtctcatggcttagtgtcagtgataataccccaacctacgtagactccgtgaaggaccggttcaccatctccagacacaacgccaacaacaccgtgtacctgcaaatgaacatgctgaaacctgaggacacggccatttactattgtgcagcaggacgcttcgcgggaagggatacttggccctcgtcctatgattactggggccaggggacccaggtcaccgtctcctcagggagcggttctggctcccaggtgcagctggtggagtctgggggaggcttggtgcagcctgggggctctctgagactctcctgtgcagcctctggattcactaatgatttttatagcatcgcgtggttccgccaggccccaggaaaggagcgtgagggggtctcatggcttagtgtcagtgataataccccaacctacgtagactccgtgaaggaccggttcaccatctccagacacaacgccaacaacaccgtgtacctgcaaatgaacatgctgaaacctgaggacacggccatttactattgtgcagcaggacgcttcgcgggaagggatacttggccctcgtcctatgattactggggccaggggacccaggtcaccgtctcctcagggagcggttctggctcccaggtgcagctggtggagtctgggggaggcttggtgcagcctgggggctctctgagactctcctgtgcagcctctggattcactaatgatttttatagcatcgcgtggttccgccaggccccaggaaaggagcgtgagggggtctcatggcttagtgtcagtgataataccccaacctacgtagactccgtgaaggaccggttcaccatctccagacacaacgccaacaacaccgtgtacctgcaaatgaacatgctgaaacctgaggacacggccatttactattgtgcagcaggacgcttcgcgggaagggatacttggccctcgtcctatgattactggggccaggggacccaggtcaccgtctcctca

SEQ ID NO: 224 - protein, 384 aa, Artificial Sequence
  Trivalent C1
QVQLVESGGGLVQPGGSLRLSCAASGFTNDFYSIAWFRQAPGKEREGVSWLSVSDNTPTYVDSVKDRFTISRHNAQNTVYLQMNMLKPEDTAIYYCAAGRFAGRDTWPSSYDYWGQGTQVTVSSGSGSGSQVQLVESGGGLVQPGGSLRLSCAASGFTNDFYSIAWFRQAPGKEREGVSWLSVSDNTPTYVDSVKDRFTISRHNAQNTVYLQMNMLKPEDTAIYYCAAGRFAGRDTWPSSYDYWGQGTQVTVSSGSGSGSQVQLVESGGGLVQPGGSLRLSCAASGFTNDFYSIAWFRQAPGKEREGVSWLSVSDNTPTYVDSVKDRFTISRHNAQNTVYLQMNMLKPEDTAIYYCAAGRFAGRDTWPSSYDYWGQGTQVTVSS

SEQ ID NO: 225 - protein, 384 aa, Artificial Sequence
  Trivalent C1
QVQLVESGGGLVQPGGSLRLSCAASGFTNDFYSIAWFRQAPGKEREGVSWLSVSDNTPTYVDSVKDRFTISRHNANNTVYLQMNMLKPEDTAIYYCAAGRFAGRDTWPSSYDYWGQGTQVTVSSGSGSGSQVQLVESGGGLVQPGGSLRLSCAASGFTNDFYSIAWFRQAPGKEREGVSWLSVSDNTPTYVDSVKDRFTISRHNANNTVYLQMNMLKPEDTAIYYCAAGRFAGRDTWPSSYDYWGQGTQVTVSSGSGSGSQVQLVESGGGLVQPGGSLRLSCAASGFTNDFYSIAWFRQAPGKEREGVSWLSVSDNTPTYVDSVKDRFTISRHNANNTVYLQMNMLKPEDTAIYYCAAGRFAGRDTWPSSYDYWGQGTQVTVSS

SEQ ID NO: 226 - DNA, 1197 bp, Artificial Sequence
  Trivalent F2
caggtgcagctggtggagtctgggggaggattggtgcaggctgggggctctctaagactcgcttgtatagcctctggacgcaccttccatagctatgtcatggcctggttccgccaggctccagggaaggagcgtgagtttgtagcagctattagttggagtagtacaccgacatactatggagaatccgtgaagggccgattcaccatctccagagacaacgccaagaacacggtgtatctgcaaatgaaccgcctgaaacctgaggacacggccgtttatttctgtgcagcagaccggggtgaaagttactactacactcgacccaccgagtatgaattctggggccaggggacccaggtcaccgtctcctcagggagcggttctggctccgggagcggttctggctcccaggtgcagctggtggagtctgggggaggattggtgcaggctgggggctctctaagactcgcttgtatagcctctggacgcaccttccatagctatgtcatggcctggttccgccaggctccagggaaggagcgtgagtttgtagcagctattagttggagtagtacaccgacatactatggagaatccgtgaagggccgattcaccatctccagagacaacgccaagaacacggtgtatctgcaaatgaaccgcctgaaacctgaggacacggccgtttatttctgtgcagcagaccggggtgaaagttactactacactcgacccaccgagtatgaattctggggccaggggacccaggtcaccgtctcctcagggagcggttctggctccgggagcggttctggctcccaggtgcagctggtggagtctgggggaggattggtgcaggctgggggctctctaagactcgcttgtatagcctctggacgcaccttccatagctatgtcatggcctggttccgccaggctccagggaaggagcgtgagtttgtagcagctattagttggagtagtacaccgacatactatggagaatccgtgaagggccgattcaccatctccagagacaacgccaagaacacggtgtatctgcaaatgaaccgcctgaaacctgaggacacggccgtttatttctgtgcagcagaccggggtgaaagttactactacactcgacccaccgagtatgaattctggggccaggggacccaggtcaccgtctcctca

SEQ ID NO: 227 - protein, 399 aa, Artificial Sequence
  Trivalent F2
QVQLVESGGGLVQAGGSLRLACIASGRTFHSYVMAWFRQAPGKEREFVAAISWSSTPTYYGESVKGRFTISRDNAKNTVYLQMNRLKPEDTAVYFCAADRGESYYYTRPTEYEFWGQGTQVTVSSGSGSGSGSGSGSQVQLVESGGGLVQAGGSLRLACIASGRTFHSYVMAWFRQAPGKEREFVAAISWSSTPTYYGESVKGRFTISRDNAKNTVYLQMNRLKPEDTAVYFCAADRGESYYYTRPTEYEFWGQGTQVTVSSGSGSGSGSGSGSQVQLVESGGGLVQAGGSLRLACIASGRTFHSYVMAWFRQAPGKEREFVAAISWSSTPTYYGESVKGRFTISRDNAKNTVYLQMNRLKPEDTAVYFCAADRGESYYYTRPTEYEFWGQGTQVTVSS

SEQ ID NO: 228 - DNA, 1179 bp, Artificial Sequence
  Trivalent H3
caggtgcagctggtggagtctgggggaggattggtgaagactgggggctctctgagactctcctgtgcagcctctggccgcaccttcagtacctacagcatgggctggttccgccaggctccagggaaggagcgtgagtttgtagcaggtatgcgctggacgggtagtagtacattctactcagactccgtgaagggccgattcaccgtctccagaaacaacgccaaggacacggtgtatctgcacatgaacagcctgaaacctgaggacacggccgtttattactgtgcaatcacgactatcgtaagagcttactatactgagtataccgaagctgactttggttcctggggccaggggacccaggtcaccgtctcctcgggagcggttctggctccacaggtgcagctggtggagtctgggggaggattggtgaagactgggggctctctgagactctcctgtgcagcctctggccgcaccttcagtacctacagcatgggctggttccgccaggctccagggaaggagcgtgagtttgtagcaggtatgcgctggacgggtagtagtacattctactcagactccgtgaagggccgattcaccgtctccagaaacaacgccaaggacacggtgtatctgcacatgaacagcctgaaacctgaggacacggccgtttattactgtgcaatcacgactatcgtaagagcttactatactgagtataccgaagctgactttggttcctggggccaggggacccaggtcaccgtctcctcagggagcggttctggctcccaggtgcagctggtggagtctgggggaggattggtgaagactgggggctctctgagactctcctgtgcagcctctggccgcaccttcagtacctacagcatgggctggttccgccaggctccagggaaggagcgtgagtttgtagcaggtatgcgctggacgggtagtagtacattctactcagactccgtgaagggccgattcaccgtctccagaaacaacgccaaggacacggtgtatctgcacatgaacagcctgaaacctgaggacacggccgtttattactgtgcaatcacgactatcgtaagagcttactatactgagtataccgaagctgactttggttcctggggccaggggacccaggtcaccgtctcctca

SEQ ID NO: 229 - protein, 393 aa, Artificial Sequence
  Trivalent H3
QVQLVESGGGLVKTGGSLRLSCAASGRTFSTYSMGWFRQAPGKEREFVAGMRWTGSSTFYSDSVKGRFTVSRNNAKDTVYLHMNSLKPEDTAVYYCAITTIVRAYYTEYTEADFGSWGQGTQVTVSSGAVLAPQVQLVESGGGLVKTGGSLRLSCAASGRTFSTYSMGWFRQAPGKEREFVAGMRWTGSSTFYSDSVKGRFTVSRNNAKDTVYLHMNSLKPEDTAVYYCAITTIVRAYYTEYTEADFGSWGQGTQVTVSSGSGSGSQVQLVESGGGLVKTGGSLRLSCAASGRTFSTYSMGWFRQAPGKEREFVAGMRWTGSSTFYSDSVKGRFTVSRNNAKDTVYLHMNSLKPEDTAVYYCAITTIVRAYYTEYTEADFGSWGQGTQVTVSS

SEQ ID NO: 230 - DNA, 1086 bp, Artificial Sequence
  Trivalent A8
ggaggcaccttcagtaccgctgccatgggctggttccgccaggctccaggcgaggagcgtgagtgtgtagcatctataggctggagaggtgttagaacatggtatgcagactccgtgaagggccgatttaccatctccagagataatccccagaatacggtgtatttgcagatgaacaacctgaaatctggcgacacggccgtttattactgcgcagcctcagtcggcaactacgggttgccgtgggcccacttcgaatatgacttctggggccaggggatccaggtcaccgtgtcgtcagggagcggttctggctcccaggtgcagctggtcgagtctgggggaggattggtgcaggctggaggctctctgagactctcctgtgcagcctctggaggcaccttcagtaccgctgccatgggctggttccgccaggctccaggcgaggagcgtgagtgtgtagcatctataggctggagaggtgttagaacatggtatgcagactccgtgaagggccgatttaccatctccagagataatccccagaatacggtgtatttgcagatgaacaacctgaaatctggcgacacggccgtttattactgcgcagcctcagtcggcaactacgggttgccgtgggcccacttcgaatatgacttctggggccaggggatccaggtcaccgtgtcgtcagggagcggttctggctcccaggtgcagctggtcgagtctgggggaggattggtgcaggctggaggctctctgagactctcctgtgcagcctctggaggcaccttcagtaccgctgccatgggctggttccgccaggctccaggcgaggagcgtgagtgtgtagcatctataggctggagaggtgttagaacatggtatgcagactccgtgaagggccgatttaccatctccagagataatccccagaatacggtgtatttgcagatgaacaacctgaaatctggcgacacggccgtttattactgcgcagcctcagtcggcaactacgggttgccgtgggcccacttcgaatatgacttctggggccaggggatccaggtcaccgtgtcgtca

SEQ ID NO: 231 - protein, 1273 aa, Severe acute respiratory syndrome coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 232 - protein, 160 aa, Severe acute respiratory syndrome coronavirus
RFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGY

SEQ ID NO: 233 - protein, 152 aa, Severe acute respiratory syndrome coronavirus
LYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLH

SEQ ID NO: 234 - protein, 352 aa, Artificial Sequence
  VHH_72-SUMO-C5
QVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSSGSGSGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGSGSGSQVQLVESGGGSVQAGGSLTLSCVASGVTLGRHAIGWFRQAPGKERERVSCIRTFDGITSYVESTKGRFTISSNNAMNTVYLQMNSLKPEDTAVYFCALGVTAACSDNPYFWGQGTQVTVSS

SEQ ID NO: 235 - protein, 8 aa, Artificial Sequence
  VHH_H6 CDR1
ESSLAPYR

SEQ ID NO: 236 - protein, 10 aa, Artificial Sequence
  VHH_H6 CDR2
ISRDAHPTST

SEQ ID NO: 237 - protein, 17 aa, Artificial Sequence
  VHH_H6 CDR3
ATDLGGYCSDSNYPRAW

SEQ ID NO: 238 - DNA, 378 bp, Artificial Sequence
  VHH_H6
caggtgcagctggtcgagtctgggggaggcttggtgcagcctggggggtctctgacactctcctgtgtagcgtcggaatcatctttggcgccttatcgcgtagcctggttccgtcaggccccagggaaggagcgcgagggggtctcatgtattagtcgtgatgcacatcctactagtacatactatacagcctccgtgaagggccgattcaccatgtccagagacaatgccaagaacacggtgtatctgcaaatgaacagcctgaaaccatcggacacggccgtttattactgtgcgacagatttgggaggatactgttcggattctaattatccccgcgcctggtggggccaggggacccaggtcaccgtctcctca

SEQ ID NO: 239 - protein, 126 aa, Artificial Sequence
  VHH_H6
QVQLVESGGGLVQPGGSLTLSCVASESSLAPYRVAWFRQAPGKEREGVSCISRDAHPTSTYYTASVKGRFTMSRDNAKNTVYLQMNSLKPSDTAVYYCATDLGGYCSDSNYPRAWWGQGTQVTVSS

SEQ ID NO: 240 - protein, 101 aa, Homo sapiens
MSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGHSTV

SEQ ID NO: 241 - protein, 76 aa, Homo sapiens
MSDQDSSEIHFKVKMTTHLKKLKESYCQRQGVPMNSLRFLFEGQRIADNHTPKELGMEEEDVIEVYQEQTGGHSTV

SEQ ID NO: 242 - protein, 95 aa, Homo sapiens
MADEKPKEGVKTENNDHINLKVAGQDGSVVQFKIKRHTPLSKLMKAYCERQGLSMRQIRFRFDGQPINETDTPAQLEMEDEDTIDVFQQQTGGVY

SEQ ID NO: 243 - protein, 71 aa, Homo sapiens
MADEKPKEGVKTENNDHINLKVAGQDGSVVQFKIKRHTPLSKLMKAYCERQLEMEDEDTIDVFQQQTGGVY

SEQ ID NO: 244 - protein, 103 aa, Homo sapiens
MSEEKPKEGVKTENDHINLKVAGQDGSVVQFKIKRHTPLSKLMKAYCERQGLSMRQIRFRFDGQPINETDTPAQLEMEDEDTIDVFQQQTGGVPESSLAGHSF

SEQ ID NO: 245 - protein, 39 aa, Homo sapiens
QVRHLAPPQSLPVCALVLCVPGIPRARASRGWTQMQLPE

SEQ ID NO: 246 - protein, 95 aa, Homo sapiens
MANEKPTEEVKTENNNHINLKVAGQDGSVVQFKIKRQTPLSKLMKAYCEPRGLSMKQIRFRFGGQPISGTDKPAQLEMEDEDTIDVFQQPTGGVY

SEQ ID NO: 247 - DNA, 1776 bp, Artificial Sequence
  F2-GSGSGS-SUMO-GSGSGS-VHH_H6
caggtgcagctggtggagtctgggggaggattggtgcaggctgggggctctctaagactcgcttgtatagcctctggacgcaccttccatagctatgtcatggcctggttccgccaggctccagggaaggagcgtgagtttgtagcagctattagttggagtagtacaccgacatactatggagaatccgtgaagggccgattcaccatctccagagacaacgccaagaacacggtgtatctgcaaatgaaccgcctgaaacctgaggacacggccgtttatttctgtgcagcagaccggggtgaaagttactactacactcgacccaccgagtatgaattctggggccaggggacccaggtcaccgtctcctcagggagcggttctggctccgatagcgaagtgaaccaggaagcgaaaccggaagttaaaccggaagtgaaaccggaaacccatattaatctgaaagttagcgacggcagcagcgaaatcttttttaaaattaaaaaaaccaccccgctgcgtcgcctgatggaagcctttgcgaaacgtcagggtaaagaaatggatagcctgcgctttctgtatgacggcatccgtattcaggccgatcagaccccggaagacctggatatggaagacaacgatattattgaagcgcatcgcgaacagatcggctcaggtagcgggtcgcaggtgcagctggtcgagtctgggggaggcttggtgcagcctggggggtctctgacactctcctgtgtagcgtcggaatcatctttggcgccttatcgcgtagcctggttccgtcaggccccagggaaggagcgcgagggggtctcatgtattagtcgtgatgcacatcctactagtacatactatacagcctccgtgaagggccgattcaccatgtccagagacaatgccaagaacacggtgtatctgcaaatgaacagcctgaaaccatcggacacggccgtttattactgtgcgacagatttgggaggatactgttcggattctaattatccccgcgcctggtggggccaggggacccaggtcaccgtctcctcagcgtcgaccgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 248 - protein, 592 aa, Artificial Sequence
  F2-GSGSGS-SUMO-GSGSGS-VHH_H6
QVQLVESGGGLVQAGGSLRLACIASGRTFHSYVMAWFRQAPGKEREFVAAISWSSTPTYYGESVKGRFTISRDNAKNTVYLQMNRLKPEDTAVYFCAADRGESYYYTRPTEYEFWGQGTQVTVSSGSGSGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGSGSGSQVQLVESGGGLVQPGGSLTLSCVASESSLAPYRVAWFRQAPGKEREGVSCISRDAHPTSTYYTASVKGRFTMSRDNAKNTVYLQMNSLKPSDTAVYYCATDLGGYCSDSNYPRAWWGQGTQVTVSSASTEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK